SEQUENCE LISTING (36 sequences)

SEQ ID NO: 1 - DNA, 29903 bp, Coronavirus 2
  source 1..29903: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 2 - DNA, 2143 bp, Coronavirus 2
  source 1..2143: mol_type = other DNA
tctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttt

SEQ ID NO: 3 - DNA, 1092 bp, Coronavirus 2
  source 1..1092: mol_type = other DNA
tctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagata

SEQ ID NO: 4 - DNA, 3822 bp, Coronavirus 2
  source 1..3822: mol_type = other DNA
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 5 - protein, 1273 aa, Coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 6 - DNA, 669 bp, Coronavirus 2
  source 1..669: mol_type = other DNA
atggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaa

SEQ ID NO: 7 - protein, 222 aa, Coronavirus 2
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 8 - protein, 805 aa, Homo sapiens [sequence omitted]

SEQ ID NO: 9 - DNA, 3339 bp, Homo sapiens
  source 1..3339: mol_type = other DNA
agtctagggaaagtcattcagtggatgtgatcttggctcacaggggacgatgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccatatggctgattgtttttggagttgtgatgggagtgatagtggttggcattgtcatcctgatcttcactgggatcagagatcggaagaagaaaaataaagcaagaagtggagaaaatccttatgcctccatcgatattagcaaaggagaaaataatccaggattccaaaacactgatgatgttcagacctccttttagaaaaatctatgtttttcctcttgaggtgattttgttgtatgtaaatgttaatttcatggtatagaaaatataagatgataaagatatcattaaatgtcaaaactatgactctgttcagaaaaaaaattgtccaaagacaacatggccaaggagagagcatcttcattgacattgctttcagtatttatttctgtctctggatttgacttctgttctgtttcttaataaggattttgtattagagtatattagggaaagtgtgtatttggtctcacaggctgttcagggataatctaaatgtaaatgtctgttgaatttctgaagttgaaaacaaggatatatcattggagcaagtgttggatcttgtatggaatatggatggatcacttgtaaggacagtgcctgggaactggtgtagctgcaaggattgagaatggcatgcattagctcactttcatttaatccattgtcaaggatgacatgctttcttcacagtaactcagttcaagtactatggtgatttgcctacagtgatgtttggaatcgatcatgctttcttcaaggtgacaggtctaaagagagaagaatccagggaacaggtagaggacattgctttttcacttccaaggtgcttgatcaacatctccctgacaacacaaaactagagccaggggcctccgtgaactcccagagcatgcctgatagaaactcatttctactgttctctaactgtggagtgaatggaaattccaactgtatgttcaccctctgaagtgggtacccagtctcttaaatcttttgtatttgctcacagtgtttgagcagtgctgagcacaaagcagacactcaataaatgctagatttacacactc

SEQ ID NO: 10 - protein, 492 aa, Homo sapiens
MALNSGSPPAIGPYYENHGYQPENPYPAQPTVVPTVYEVHPAQYYPSPVPQYAPRVLTQASNPVVCTQPKSPSGTVCTSKTKKALCITLTLGTFLVGAALAAGLLWKFMGSKCSNSGIECDSSGTCINPSNWCDGVSHCPGGEDENRCVRLYGPNFILQVYSSQRKSWHPVCQDDWNENYGRAACRDMGYKNNFYSSQGIVDDSGSTSFMKLNTSAGNVDIYKKLYHSDACSSKAVVSLRCIACGVNLNSSRQSRIVGGESALPGAWPWQVSLHVQNVHVCGGSIITPEWIVTAAHCVEKPLNNPWHWTAFAGILRQSFMFYGAGYQVEKVISHPNYDSKTKNNDIALMKLQKPLTFNDLVKPVCLPNPGMMLQPEQLCWISGWGATEEKGKTSEVLNAAKVLLIETQRCNSRYVYDNLITPAMICAGFLQGNVDSCQGDSGGPLVTSKNNIWWLIGDTSWGSGCAKAYRPGVYGNVMVFTDWIYRQMRADG

SEQ ID NO: 11 - DNA, 3450 bp, Homo sapiens
  source 1..3450: mol_type = other DNA
gagtaggcgcgagctaagcaggaggcggaggcggaggcggagggcgaggggcggggagcgccgcctggagcgcggcaggtcatattgaacattccagatacctatcattactcgatgctgttgataacagcaagatggctttgaactcagggtcaccaccagctattggaccttactatgaaaaccatggataccaaccggaaaacccctatcccgcacagcccactgtggtccccactgtctacgaggtgcatccggctcagtactacccgtcccccgtgccccagtacgccccgagggtcctgacgcaggcttccaaccccgtcgtctgcacgcagcccaaatccccatccgggacagtgtgcacctcaaagactaagaaagcactgtgcatcaccttgaccctggggaccttcctcgtgggagctgcgctggccgctggcctactctggaagttcatgggcagcaagtgctccaactctgggatagagtgcgactcctcaggtacctgcatcaacccctctaactggtgtgatggcgtgtcacactgccccggcggggaggacgagaatcggtgtgttcgcctctacggaccaaacttcatccttcaggtgtactcatctcagaggaagtcctggcaccctgtgtgccaagacgactggaacgagaactacgggcgggcggcctgcagggacatgggctataagaataatttttactctagccaaggaatagtggatgacagcggatccaccagctttatgaaactgaacacaagtgccggcaatgtcgatatctataaaaaactgtaccacagtgatgcctgttcttcaaaagcagtggtttctttacgctgtatagcctgcggggtcaacttgaactcaagccgccagagcaggattgtgggcggcgagagcgcgctcccgggggcctggccctggcaggtcagcctgcacgtccagaacgtccacgtgtgcggaggctccatcatcacccccgagtggatcgtgacagccgcccactgcgtggaaaaacctcttaacaatccatggcattggacggcatttgcggggattttgagacaatctttcatgttctatggagccggataccaagtagaaaaagtgatttctcatccaaattatgactccaagaccaagaacaatgacattgcgctgatgaagctgcagaagcctctgactttcaacgacctagtgaaaccagtgtgtctgcccaacccaggcatgatgctgcagccagaacagctctgctggatttccgggtggggggccaccgaggagaaagggaagacctcagaagtgctgaacgctgccaaggtgcttctcattgagacacagagatgcaacagcagatatgtctatgacaacctgatcacaccagccatgatctgtgccggcttcctgcaggggaacgtcgattcttgccagggtgacagtggagggcctctggtcacttcgaagaacaatatctggtggctgataggggatacaagctggggttctggctgtgccaaagcttacagaccaggagtgtacgggaatgtgatggtattcacggactggatttatcgacaaatgagggcagacggctaatccacatggtcttcgtccttgacgtcgttttacaagaaaacaatggggctggttttgcttccccgtgcatgatttactcttagagatgattcagaggtcacttcatttttattaaacagtgaacttgtctggctttggcactctctgccattctgtgcaggctgcagtggctcccctgcccagcctgctctccctaaccccttgtccgcaaggggtgatggccggctggttgtgggcactggcggtcaagtgtggaggagaggggtggaggctgccccattgagatcttcctgctgagtcctttccaggggccaattttggatgagcatggagctgtcacctctcagctgctggatgacttgagatgaaaaaggagagacatggaaagggagacagccaggtggcacctgcagcggctgccctctggggccacttggtagtgtccccagcctacctctccacaaggggattttgctgatgggttcttagagccttagcagccctggatggtggccagaaataaagggaccagcccttcatgggtggtgacgtggtagtcacttgtaaggggaacagaaacatttttgttcttatggggtgagaatatagacagtgcccttggtgcgagggaagcaattgaaaaggaacttgccctgagcactcctggtgcaggtctccacctgcacattgggtggggctcctgggagggagactcagccttcctcctcatcctccctgaccctgctcctagcaccctggagagtgcacatgccccttggtcctggcagggcgccaagtctggcaccatgttggcctcttcaggcctgctagtcactggaaattgaggtccatgggggaaatcaaggatgctcagtttaaggtacactgtttccatgttatgtttctacacattgctacctcagtgctcctggaaacttagcttttgatgtctccaagtagtccaccttcatttaactctttgaaactgtatcatctttgccaagtaagagtggtggcctatttcagctgctttgacaaaatgactggctcctgacttaacgttctataaatgaatgtgctgaagcaaagtgcccatggtggcggcgaagaagagaaagatgtgttttgttttggactctctgtggtcccttccaatgctgtgggtttccaaccaggggaagggtcccttttgcattgccaagtgccataaccatgagcactactctaccatggttctgcctcctggccaagcaggctggtttgcaagaatgaaatgaatgattctacagctaggacttaaccttgaaatggaaagtcatgcaatcccatttgcaggatctgtctgtgcacatgcctctgtagagagcagcattcccagggaccttggaaacagttggcactgtaaggtgcttgctccccaagacacatcctaaaaggtgttgtaatggtgaaaacgtcttccttctttattgccccttcttatttatgtgaacaactgtttgtctttttttgtatcttttttaaactgtaaagttcaattgtgaaaatgaatatcatgcaaataaattatgcaatttttttttcaaagtaactactgcatctttgaagttctgcctggtgagtaggaccagcctccatttccttataagggggtgatgttgaggctgctggtcagaggaccaaaggtgaggcaaggccagacttggtgctcctgtggttggtgccctcagttcctgcagcctgtcctgttggagaggtccctcaaatgactccttcttattattctattagtctgtttccatgctcctaataaagacatacccaagactgcaattta

SEQ ID NO: 12 - protein, 4405 aa, Coronavirus 2 [sequence omitted]

SEQ ID NO: 13 - protein, 932 aa, Coronavirus 2
SADAQSFLNRVCGVSAARLTPCGTGTSTDVVYRAFDIYNDKVAGFAKFLKTNCCRFQEKDEDDNLIDSYFVVKRHTFSNYQHEETIYNLLKDCPAVAKHDFFKFRIDGDMVPHISRQRLTKYTMADLVYALRHFDEGNCDTLKEILVTYNCCDDDYFNKKDWYDFVENPDILRVYANLGERVRQALLKTVQFCDAMRNAGIVGVLTLDNQDLNGNWYDFGDFIQTTPGSGVPVVDSYYSLLMPILTLTRALTAESHVDTDLTKPYIKWDLLKYDFTEERLKLFDRYFKYWDQTYHPNCVNCLDDRCILHCANFNVLFSTVFPPTSFGPLVRKIFVDGVPFVVSTGYHFRELGVVHNQDVNLHSSRLSFKELLVYAADPAMHAASGNLLLDKRTTCFSVAALTNNVAFQTVKPGNFNKDFYDFAVSKGFFKEGSSVELKHFFFAQDGNAAISDYDYYRYNLPTMCDIRQLLFVVEVVDKYFDCYDGGCINANQVIVNNLDKSAGFPFNKWGKARLYYDSMSYEDQDALFAYTKRNVIPTITQMNLKYAISAKNRARTVAGVSICSTMTNRQFHQKLLKSIAATRGATVVIGTSKFYGGWHNMLKTVYSDVENPHLMGWDYPKCDRAMPNMLRIMASLVLARKHTTCCSLSHRFYRLANECAQVLSEMVMCGGSLYVKPGGTSSGDATTAYANSVFNICQAVTANVNALLSTDGNKIADKYVRNLQHRLYECLYRNRDVDTDFVNEFYAYLRKHFSMMILSDDAVVCFNSTYASQGLVASIKNFKSVLYYQNNVFMSEAKCWTETDLTKGPHEFCSQHTMLVKQGDDYVYLPYPDPSRILGAGCFVDDIVKTDGTLMIERFVSLAIDAYPLTKHPNQEYADVFHLYLQYIRKLHDELTGHMLDMYSVMLTNDNTSRYWEPEFYEAMYTPHTVLQ

SEQ ID NO: 14 - protein, 601 aa, Coronavirus 2
AVGACVLCNSQTSLRCGACIRRPFLCCKCCYDHVISTSHKLVLSVNPYVCNAPGCDVTDVTQLYLGGMSYYCKSHKPPISFPLCANGQVFGLYKNTCVGSDNVTDFNAIATCDWTNAGDYILANTCTERLKLFAAETLKATEETFKLSYGIATVREVLSDRELHLSWEVGKPRPPLNRNYVFTGYRVTKNSKVQIGEYTFEKGDYGDAVVYRGTTTYKLNVGDYFVLTSHTVMPLSAPTLVPQEHYVRITGLYPTLNISDEFSSNVANYQKVGMQKYSTLQGPPGTGKSHFAIGLALYYPSARIVYTACSHAAVDALCEKALKYLPIDKCSRIIPARARVECFDKFKVNSTLEQYVFCTVNALPETTADIVVFDEISMATNYDLSVVNARLRAKHYVYIGDPAQLPAPRTLLTKGTLEPEYFNSVCRLMKTIGPDMFLGTCRRCPAEIVDTVSALVYDNKLKAHKDKSAQCFKMFYKGVITHDVSSAINRPQIGVVREFLTRNPAWRKAVFISPYNSQNAVASKILGLPTQTVDSSQGSEYDYVIFTQTTETAHSCNVNRFNVAITRAKVGILCIMSDRDLYDKLQFTSLEIPRRNVATLQ

SEQ ID NO: 15 - protein, 254 aa, Coronavirus 2
PNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLE

SEQ ID NO: 16 - protein, 610 aa, Coronavirus 2
CDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLH

SEQ ID NO: 17 - protein, 1945 aa, Coronavirus 2
APTKVTFGDDTVIEVQGYKSVNITFELDERIDKVLNEKCSAYTVELGTEVNEFACVVADAVIKTLQPVSELLTPLGIDLDEWSMATYYLFDESGEFKLASHMYCSFYPPDEDEEEGDCEEEEFEPSTQYEYGTEDDYQGKPLEFGATSAALQPEEEQEEDWLDDDSQQTVGQQDGSEDNQTTTIQTIVEVQPQLEMELTPVVQTIEVNSFSGYLKLTDNVYIKNADIVEEAKKVKPTVVVNAANVYLKHGGGVAGALNKATNNAMQVESDDYIATNGPLKVGGSCVLSGHNLAKHCLHVVGPNVNKGEDIQLLKSAYENFNQHEVLLAPLLSAGIFGADPIHSLRVCVDTVRTNVYLAVFDKNLYDKLVSSFLEMKSEKQVEQKIAEIPKEEVKPFITESKPSVEQRKQDDKKIKACVEEVTTTLEETKFLTENLLLYIDINGNLHPDSATLVSDIDITFLKKDAPYIVGDVVQEGVLTAVVIPTKKAGGTTEMLAKALRKVPTDNYITTYPGQGLNGYTVEEAKTVLKKCKSAFYILPSIISNEKQEILGTVSWNLREMLAHAEETRKLMPVCVETKAIVSTIQRKYKGIKIQEGVVDYGARFYFYTSKTTVASLINTLNDLNETLVTMPLGYVTHGLNLEEAARYMRSLKVPATVSVSSPDAVTAYNGYLTSSSKTPEEHFIETISLAGSYKDWSYSGQSTQLGIEFLKRGDKSVYYTSNPTTFHLDGEVITFDNLKTLLSLREVRTIKVFTTVDNINLHTQVVDMSMTYGQQFGPTYLDGADVTKIKPHNSHEGKTFYVLPNDDTLRVEAFEYYHTTDPSFLGRYMSALNHTKKWKYPQVNGLTSIKWADNNCYLATALLTLQQIELKFNPPALQDAYYRARAGEAANFCALILAYCNKTVGELGDVRETMSYLFQHANLDSCKRVLNVVCKTCGQQQTTLKGVEAVMYMGTLSYEQFKKGVQIPCTCGKQATKYLVQQESPFVMMSAPPAQYELKHGTFTCASEYTGNYQCGHYKHITSKETLYCIDGALLTKSSEYKGPITDVFYKENSYTTTIKPVTYKLDGVVCTEIDPKLDNYYKKDNSYFTEQPIDLVPNQPYPNASFDNFKFVCDNIKFADDLNQLTGYKKPASRELKVTFFPDLNGDVVAIDYKHYTPSFKKGAKLLHKPIVWHVNNATNKATYKPNTWCIRCLWSTKPVETSNSFDVLKSEDAQGMDNLACEDLKPVSEEVVENPTIQKDVLECNVKTTEVVGDIILKPANNSLKITEEVGHTDLMAAYVDNSSLTIKKPNELSRVLGLKTLATHGLAAVNSVPWDTIANYAKPFLNKVVSTTTNIVTRCLNRVCTNYMPYFFTLLLQLCTFTRSTNSRIKASMPTTIAKNTVKSVGKFCLEASFNYLKSPNFSKLINIIIWFLLLSVCLGSLIYSTAALGVLMSNLGMPSYCTGYREGYLNSTNVTIATYCTGSIPCSVCLSGLDSLDTYPSLETIQITISSFKWDLTAFGLVAEWFLAYILFTRFFYVLGLAAIMQLFFSYFAVHFISNSWLMWLIINLVQMAPISAMVRMYIFFASFYYVWKSYVHVVDGCNSSTCMMCYKRNRATRVECTTIVNGVRRSFYVYANGGKGFCKLHNWNCVNCDTFCAGSTFISDEVARDLSLQFKRPINPTDQSSYIVDSVTVKNGSIHLYFDKAGQKTYERHSLSHFVNLDNLRANNTKGSLPINVIVFDGKSKCEESSAKSASVYYSQLMCQPILLLDQALVSDVGDSAEVAVKMFDAYVNTFSSTFNVPMEKLKTLVATAEAELAKNVSLDNVLSTFISAARQGFVDSDVETKDVVECLKLSHQSDIEVTGDSCNNYMLTYNKVENMTPRDLGACIDCSARHINAQVAKSHNIALIWNVKDFMSLSEQLRKQIRSAAKKNNLPFKLTCATTRQVVNVVTTKIALKGG

SEQ ID NO: 18 - protein, 500 aa, Coronavirus 2
KIVNNWLKQLIKVTLVFLFVAAIFYLITPVHVMSKHTDFSSEIIGYKAIDGGVTRDIASTDTCFANKHADFDTWFSQRGGSYTNDKACPLIAAVITREVGFVVPGLPGTILRTTNGDFLHFLPRVFSAVGNICYTPSKLIEYTDFATSACVLAAECTIFKDASGKPVPYCYDTNVLEGSVAYESLRPDTRYVLMDGSIIQFPNTYLEGSVRVVTTFDSEYCRHGTCERSEAGVCVSTSGRWVLNNDYYRSLPGVFCGVDAVNLLTNMFTPLIQPIGALDISASIVAGGIVAIVVTCLAYYFMRFRRAFGEYSHVVAFNTLLFLMSFTVLCLTPVYSFLPGVYSVIYLYLTFYLTNDVSFLAHIQWMVMFTPLVPFWITIAYIICISTKHFYWFFSNYLKRRVVFNGVSFSTFEEAALCTFLLNKEMYLKLRSDVLLPLTQYNRYLALYNKYKYFSGAMDTTSYREAACCHLAKALNDFSNSGSDVLYQPPQTSITSAVLQ

SEQ ID NO: 19 - protein, 275 aa, Coronavirus 2
MDLFMRIFTIGTVTLKQGEIKDATPSDFVRATATIPIQASLPFGWLIVGVALLAVFQSASKIITLKKRWQLALSKGVHFVCNLLLLFVTVYSHLLLVAAGLEAPFLYLYALVYFLQSINFVRIIMRLWLCWKCRSKNPLLYDANYFLCWHTNCYDYCIPYNSVTSSIVITSGDGTTSPISEHDYQIGGYTEKWESGVKDCVVLHSYFTSDYYQLYSTQLSTDTGVEHVTFFIYNKIVDEPEEHVQIHTIDGSSGVVNPVMEPIYDEPTTTTSVPL

SEQ ID NO: 20 - protein, 75 aa, Coronavirus 2
MYSFVSEETGTLIVNSVLLFLAFVVFLLVTLAILTALRLCAYCCNIVNVSLVKPSFYVYSRVKNLNSSRVPDLLV

SEQ ID NO: 21 - protein, 222 aa, Coronavirus 2; identical to SEQ ID NO: 7

SEQ ID NO: 22 - protein, 61 aa, Coronavirus 2
MFHLVDFQVTIAEILLIIMRTFKVSIWNLDYIINLIIKNLSKSLTENKYSQLDEEQPMEID

SEQ ID NO: 23 - protein, 121 aa, Coronavirus 2
MKIILFLALITLATCELYHYQECVRGTTVLLKEPCSSGTYEGNSPFHPLADNKFALTCFSTQFAFACPDGVKHVYQLRARSVSPKLFIRQEEVQELYSPIFLIVAAIVFITLCFTLKRKTE

SEQ ID NO: 24 - protein, 43 aa, Coronavirus 2
MIELSLIDFYLCFLAFLLFLVLIMLIIFWFSLELQDHNETCHA

SEQ ID NO: 25 - protein, 121 aa, Coronavirus 2
MKFLVFLGIITTVAAFHQECSLQSCTQHQPYVVDDPCPIHFYSKWYIRVGARKSAPLIELCVDEAGSKSPIQYIDIGNYTVSCLPFTINCQEPKLGSLVVRCSFYEDFLEYHDVRVVLDFI

SEQ ID NO: 26 - protein, 419 aa, Coronavirus 2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 27 - protein, 38 aa, Coronavirus 2
MGYINVFAFPFTIYSLLLCRMNSRNYIAQVDVVNFNLT

SEQ ID NO: 28 - DNA, 36 bp, Coronavirus 2
  source 1..36: mol_type = other DNA
ttgtgcagaatgaattctcgtaactacatagcacaa

SEQ ID NO: 29 - DNA, 29 bp, Coronavirus 2
  source 1..29: mol_type = other DNA
taactacatagcacaagtagatgtagtta

SEQ ID NO: 30 - protein, 113 aa, Coronavirus 2
NNELSPVALRQMSCAAGTTQTACTDDNALAYYNTTKGGRFVLALLSDLQDLKWARFPKSDGTGTIYTELEPPCRFVTDTPKGPKVKYLYFIKGLNNLNRGMVLGSLAATVRLQ

SEQ ID NO: 31 - skipped

SEQ ID NO: 32 - protein, 346 aa, Coronavirus 2
SLENVAFNVVNKGHFDGQQGEVPVSIINNTVYTKVDGVDVELFENKTTLPVNVAFELWAKRNIKPVPEVKILNNLGVDIAANTVIWDYKRDAPAHISTIGVCSMTDIAKKPTETICAPLTVFFDGRVDGQVDLFRNARNGVLITEGSVKGLQPSVGPKQASLNGVTLIGEAVKTQFNYYKKVDGVVQQLPETYFTQSRNLQEFKPRSQMEIDFLELAMDEFIERYKLEGYAFEHIVYGDFSHSQLGGLHLLIGLAKRFKESPFELEDFIPMDSTVKNYFITDAQTGSSKCVCSVIDLLLDDFVEIIKSQDLSVVSKVVKVTIDYTEISFMLWCKDGHVETFYPKLQ

SEQ ID NO: 33 - protein, 299 aa, Coronavirus 2
MSSQAWQPGVAMPNLYKMQRMLLEKCDLQNYGDSATLPKGIMMNVAKYTQLCQYLNTLTLAVPYNMRVIHFGAGSDKGVAPGTAVLRQWLPTGTLLVDSDLNDFVSDADSTLIGDCATVHTANKWDLIISDMYDPKTKNVTKENDSKEGFFTYICGFIQQKLALGGSVAIKITEHSWNADLYKLMGHFAWWTAFVTNVNASSSEAFLIGCNYLGKPREQIDGYVMHANYIFWRNTNPIQLSSYSLFDMSKFPLKLRGTAVMSLKEGQINDMILSLLSKGRLIIRENNRVVISSDVLVNN

SEQ ID NO: 34 - RNA, 798 nt, Coronavirus 2
  source 1..798: mol_type = other RNA
atgtttgtgtttcttgtgctgctgcctcttgtgtcttctcagtgtgtggtgagatttccaaatattacaaatctgtgtccatttggagaagtgtttaatgcaacaagatttgcatctgtgtatgcatggaatagaaaaagaatttctaattgtgtggctgattattctgtgctgtataatagtgcttctttttccacatttaaatgttatggagtgtctccaacaaaattaaatgatttatgttttacaaatgtgtatgctgattcttttgtgatcagaggtgatgaagtgagacagattgcccccggacagacaggaaaaattgctgattacaattacaaactgcctgatgattttacaggatgtgtgattgcttggaattctaataatttagattctaaagtgggaggaaattacaattatctgtacagactgtttagaaaatcaaatctgaaaccttttgaaagagatatttcaacagaaatttatcaggctggatcaacaccttgtaatggagtggaaggatttaattgttattttccattacagagctatggatttcagccaaccaatggtgtgggatatcagccatatagagtggtggtgctgtcttttgaactgctgcatgcacctgcaacagtgtgtggacctaaaggctcccccggctccggctccggatctggttatattcctgaagctccaagagatgggcaagcttacgttcgtaaagatggcgaatgggtattactttctacctttttaggccggtccctggaggtgctgttccagggccccggc

SEQ ID NO: 35 - protein, 266 aa, Coronavirus
MFVFLVLLPLVSSQCVVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKGSPGSGSGSGYIPEAPRDGQAYVRKDGEWVLLSTFLGRSLEVLFQGPG

SEQ ID NO: 36 - protein, 41 aa, synthetic construct
GSGYIPEAPRDGQAYVRKDGEWVLLSTFLGRSLEVLFQGPG

(19) United States
(12) Patent Application Publication
(10) Pub. No.: US 2025/0297277 A1
(43) Pub. Date: Sep. 25, 2025

(54) SARS-CoV-2 Virus-Like Particles

(71) Applicants: The J. David Gladstone Intitutes, a testamentary trust established under the Will of J. David, San Francisco, CA (US); The Regents of the University of California, Oakland, CA (US)

(72) Inventors: Muhammad Abdullah Syed, San Francisco, CA (US); Jennifer A. Doudna, Oakland, CA (US)

(21) Appl. No.: 18/294,189

(22) PCT Filed: Aug. 4, 2022
(86) PCT No.: PCT/US2022/074504

§ 371 Date: Feb. 1, 2024

Related U.S. Application Data
(60) Provisional application No. 63/229,141, filed on Aug. 4, 2021.

(51) Int. Cl. C12N 15/86; A61K 39/00; A61K 39/215; C07K 14/005; G01N 33/569

(52) U.S. Cl. CPC C12N 15/86; A61K 39/215; C07K 14/005; G01N 33/56983; A61K 2039/5258; C12N 2770/20022; C12N 2770/20023; C12N 2770/20034; C12N 2770/20043; G01N 2333/165

(57) ABSTRACT

Provided herein are SARS-CoV-2 virus-like particles as well as methods and compositions for generating SARS-CoV-2 virus-like particles. The SARS-CoV-2 virus-like particles can load and deliver transcripts (including engineered transcripts that can include therapeutic agents) into cells expressing SARS-CoV-2 entry factors. The SARS-CoV-2 virus-like particles are also useful for detecting immune response in antibodies from subjects.

### CROSS REFERENCE TO RELATED APPLICATIONS

This application is a U.S. National Stage Filing under 35 U.S.C. 371 from International Patent Application Serial No. PCT/US2022/074504, filed Aug. 4, 2022, published on Feb. 9, 2023 as WO2023/015232, which application claims the benefit of priority to U.S. Provisional Patent Application Ser. No. 63/229,141, filed Aug. 4, 2021, the complete disclosures of which are incorporated herein by reference in their entireties.

### GOVERNMENT FUNDING

This invention was made with government support under R21 AI159666 awarded by the National Institutes of Health. The government has certain rights in the invention.

### INCORPORATION BY REFERENCE OF SEQUENCE LISTING PROVIDED AS AN XML FILE

A Sequence Listing is provided herewith as an xml file, “2258818.xml” created on Aug. 2, 2022, and having a size of 94,712 bytes. The content of the xml file is incorporated by reference herein in its entirety.

### BACKGROUND

The World Health Organization has declared Covid-19 a global pandemic. A highly infectious coronavirus, officially called SARS-CoV-2, causes the Covid-19 disease. Even with the most effective containment strategies, the spread of the Covid-19 respiratory disease has only been slowed. While the available vaccines are still useful, new variants and mutants of SARS-CoV-2 continually arise.

Such newly evolved SARS-CoV-2 variants are driving ongoing outbreaks of COVID-19 around the world. Efforts to determine why these viral variants have improved fitness are limited to mutations in the viral spike (S) protein and viral entry steps using non-SARS-CoV-2 viral particles engineered to display the spike protein. More efficient methods for identifying and evaluating new and existing strains of SARS-CoV-2 can facilitate development of new and better treatments for SARS-CoV-2 infection.

### SUMMARY

Described herein are SARS-CoV-2 virus-like particles that can load and deliver transcripts (including engineered transcripts) into cells expressing SARS-CoV-2 receptors. Methods of making and using the SARS-CoV-2 virus-like particles are also described herein

The manufacturing methods are rapid and scalable. Such methods can include providing packaging signals for different SARS-CoV-2 strains and screening of SARS-CoV-2 mutations to determine their impact on viral assembly and viral entry. Various RNAs can be delivered to cells using the SARS-CoV-2 virus-like particles. The delivered RNA can be any type of RNA-including exogenous RNAs. In some cases, the delivered RNA can encode a therapeutic protein or the delivered RNA can be an inhibitory RNA that reduces infection. The methods can also include screening for inhibitors of SARS-CoV-2 budding, SARS-CoV-2 entry, and SARS-CoV-2 uncoating. Naturally arising and engineered mutations within SARS-CoV-2 can be evaluated to identify variants of concern.

Described herein are nucleic acids that include a SARS-CoV-2 packaging signal sequence segment that can be linked to a heterologous nucleic acid. The SARS-CoV-2 packaging signal sequence can be a nucleic acid segment having positions 20080-21171 (SEQ ID NO:3) of the SARS-CoV-2 genome (termed herein the PS9 region) or nucleic acid having nucleotides 20080-22222 (SEQ ID NO:2) of the SARS-CoV-2 genome referred to as “T20.” The nucleic acids can include a promoter or internal ribosome entry site (IRES) operably linked to the SARS-CoV-2 packaging signal sequence segment and to the heterologous nucleic acid. The heterologous nucleic acid can encode a heterologous protein such as a detectable signal protein, therapeutic agent, antigenic protein, or an antibody (e.g., an antibody fragment). For example, the heterologous nucleic acid can encode an anti-Spike antibody or antibody fragment. In another example, the heterologous nucleic acid can encode a viral antigen. In some cases, the heterologous nucleic acid encodes an inhibitory nucleic acid that binds to a segment of a SARS-CoV-2 RNA.

The nucleic acids that include a SARS-CoV-2 packaging signal sequence segment linked to a heterologous nucleic acid can be incorporated into one or more cells (receptor cells or host cells). Such nucleic acids are heterologous to the cells. The cells can also express a SARS-CoV-2 spike (S) protein, SARS-CoV-2 membrane (M) protein, SARS-CoV-2 envelope (E) protein, and SARS-CoV-2 nucleocapsid (N) protein to thereby generate the SARS-CoV-2 virus-like particles containing the SARS-CoV-2 packaging signal sequence segment with the heterologous nucleic acid.

In some cases, the SARS-CoV-2 spike (S) protein, the SARS-CoV-2 membrane (M) protein, the SARS-CoV-2 envelope (E) protein, or the SARS-CoV-2 nucleocapsid (N) protein has one or more mutations. Such mutations can be relative to a reference ancestral SARS-CoV-2 spike (S) protein, SARS-CoV-2 membrane (M) protein, SARS-CoV-2 envelope (E) protein, or SARS-CoV-2 nucleocapsid (N) protein sequence, for example, a SARS-CoV-2 sequence provided herein as SEQ ID NO:1. The SARS-CoV-2 spike (S) coding region, the SARS-CoV-2 membrane (M) coding region, the SARS-CoV-2 envelope (E) coding region, or the SARS-CoV-2 nucleocapsid (N) coding region expressed by the cells can have a mutation compared to their respective coding regions in SEQ ID NO:1. In some cases, the SARS-CoV-2 spike (S) protein has a mutation compared to a SARS-CoV-2 spike (S) protein with a D614G mutation.

Also described herein are expression systems that can include one or more expression cassettes, where each expression cassette has a promoter or an internal ribosome entry site (IRES) operably linked to one or more of the following nucleic acids that encode:
an RNA comprising a SARS-CoV-2 packaging signal sequence segment linked to a heterologous nucleic acid;
a SARS-CoV-2 spike (S) protein;
a SARS-CoV-2 membrane (M) protein;
a SARS-CoV-2 envelope (E) protein; and
a SARS-CoV-2 nucleocapsid (N) protein.

One or more of the SARS-CoV-2 spike (S) protein, the SARS-CoV-2 membrane (M) protein, the SARS-CoV-2 envelope (E) protein, or the SARS-CoV-2 nucleocapsid (N) protein can have a mutation.

Also described herein are kits that can include one or more containers containing one or more components of the expression systems.

Methods are also described herein that include comprising transfecting a cell (e.g., a host cell) with at least one expression cassette or expression vector, wherein the at least one expression cassette or expression vector comprises a promoter or internal ribosome entry site (IRES) operably linked to at least one of the following heterologous nucleic acids:
a nucleic acid comprising a SARS-CoV-2 packaging signal sequence segment linked to a heterologous nucleic acid;
a nucleic acid encoding SARS-CoV-2 spike (S) protein;
a nucleic acid encoding SARS-CoV-2 membrane (M) protein;
a nucleic acid encoding SARS-CoV-2 envelope (E) protein,
a nucleic acid encoding SARS-CoV-2 nucleocapsid (N) protein;
or a combination thereof.

The cell expresses at least one of the following: an RNA comprising a SARS-CoV-2 packaging signal sequence segment linked to the heterologous nucleic acid; a SARS-CoV-2 spike (S) protein; a SARS-CoV-2 membrane (M) protein; a SARS-CoV-2 envelope (E) protein, a SARS-CoV-2 nucleocapsid (N) protein, or a combination thereof.

The method can generate SARS-CoV-2 virus-like-particles. When making virus-like-particles, the cell express: the SARS-CoV-2 packaging signal sequence segment linked to the heterologous nucleic acid; the SARS-CoV-2 spike (S) protein; a SARS-CoV-2 membrane (M) protein; the SARS-CoV-2 envelope (E) protein; and the SARS-CoV-2 nucleocapsid (N) protein. When the heterologous nucleic acid encodes a heterologous protein, the signal protein can provide a detectable signal. The signal level from the detectable signal can be a measure of the extent of virus-like-particle assembly, packaging, and/or cellular entry.

The SARS-CoV-2 virus-like-particles are also useful for evaluating immune responses against SARS-CoV-2 and for treating subjects who exhibit reduced immunity against SARS-CoV-2 compared to a control or cut-off level of immunity. Methods for evaluating immune responses against SARS-CoV-2 involve testing whether a subject has sufficient antibodies against SARS-CoV-2 to inhibit or prevent entry, assembly, or expression of SARS-CoV-2 virus-like-particles relative to a control or cut-off level. For example, such a method can involve contacting SARS-CoV-2 virus-like-particles with a serum sample from a subject, and a population of receptor cells; and measuring detectable signal levels produced by detectable signal protein. The methods can further include administering a SARS-CoV-2 vaccine to one or more subjects whose antibodies emit a lower detectable signal level than a control or cut-off signal level. In some cases, the SARS-CoV-2 vaccine can be a Moderna or Pfizer vaccine. In other cases, the SARS-CoV-2 vaccine is not a Moderna or Pfizer vaccine.

### DESCRIPTION OF THE FIGURES

FIG. 1A-1N illustrate the design and characterization of SARS-CoV-2 virus-like particles (abbreviated SC2-VLPs). FIG. 1A shows a schematic of the SARS-CoV-2 virus, the SC2-VLPs, the SARS-CoV-2 genome, and the expression vector design. FIG. 1B illustrates the process flow for generating and detecting luciferase encoding SARS-CoV-2 virus-like particles. FIG. 1C graphically illustrates induced luciferase expression measured as relative luminescent units (RLU) detected in receiver cells (293T overexpressing ACE2 and TMPRSS2) from “Standard” SARS-CoV-2 virus-like particles containing S, M, N, E and luciferase-T20 transcript, as well as various virus-like-particles (VLPs) lacking one of these components. FIG. 1D graphically illustrates that an N-terminal or C-terminal strep-tag on the membrane protein abrogates SC2-VLP induced luciferase expression in receptor cells (293T overexpressing ACE2 and TMPRSS2). FIG. 1E illustrates that optimal luciferase expression requires a narrow range of spike plasmid concentrations corresponding to about Ing of plasmid in a 24-well. FIG. 1F is a schematic illustrating purification methods for SARS-CoV-2 virus-like particles. FIG. 1G shows a Western blot illustrating spike and N proteins in pellets purified from standard SARS-CoV-2 virus-like particles and conditions that did not induce luciferase expression in receiver cells. FIG. 1H is a schematic illustrating sucrose gradient centrifugation methods for separating SARS-CoV-2 virus-like particles. FIG. 1I illustrates induced luciferase expression from sucrose gradient fractions of SARS-CoV-2 virus-like particles. FIG. 1J illustrates relative luminescence units measured from Vero E6 cells incubated with supernatants containing SARS-CoV-2 virus-like particles as well as supernatants of cells missing either S, M, N, E or the packaging signal (PS). FIG. 1K illustrates luminescence from receiver cells after incubation with supernatants containing SARS-CoV-2 virus-like particles, as well as supernatants from cells transfected with the following N-containing tags: either a mNG11-N tag (N with amino-terminal mNG11 tag) or a N-2xStrep tag (N with carboxy-terminal 2xStrep tag). FIG. 1L schematically illustrates the structure of a transfer plasmid encoding luciferase and the T20 (SARS-CoV-2 packaging) region within its 3′ untranslated region (UTR). FIG. 1M graphically illustrates luminescence induced in receiver cells from SARS-CoV-2 VLPs after treatment with ribonuclease (RNase) or 1-4 cycles of freeze-thaw (FT) or incubation at 55° C. and 70° C., respectively. All values were normalized to the original supernatant. Lentiviral particles encoding luciferase are shown as a comparison. FIG. 1N graphically illustrates luminescence induced from SARS-CoV-2 VLPs purified/concentrated using different methods compared to total protein measurement from the same samples using bicinchoninic acid (BCA) assay.

FIG. 2A-2F illustrate the location of the SARS-CoV-2 packaging signal. FIG. 2A illustrates an arrayed screen for determining the location of the SARS-CoV-2 packaging signal using SARS-CoV-2 virus-like particles. Two kilobase (2 kB) tiled segments of the SARS-CoV-2 genome were cloned into the 3′UTR of the luciferase plasmid, attempts were made to generate VLPs, potential VLPs were introduced into a second set of receiver/receptor cells, and light was detected from the second set of cells when VLPs were actually generated. FIG. 2B graphically illustrates induced luciferase expression in receiver cells by SARS-CoV-2 virus-like particles containing different tiles from the SARS-CoV-2 genome FIG. 2C shows a heatmap to facilitate visualization of the data from FIG. 2B. The heatmap shows the locations of tiled segments relative to the SARS-CoV-2 genome. The darkness of the heatmap segments indicates the level of luminescence of receiver cells for each tile, where the luminescence levels were normalized to expression for luciferase plasmid containing no insert. As illustrated the darkest segment spans the T20 genomic segment. FIG. 2D graphically illustrates luminescence from smaller segments of the SARS-CoV-2 genome used to further narrow down the location of the packaging signal. As illustrated, the PS9 region exhibited the highest levels of luminescence. FIG. 2E is a heatmap showing the locations of the smaller segments of the SARS-CoV-2 genome to facilitate visualization of the data from FIG. 2D. The nucleotide positions of the T20 and PS9 regions in the SARS-CoV-2 are shown below the graph. FIG. 2F graphically illustrates results of flow cytometry analysis of GFP expression for 293T ACE2/TMPRSS2 cells incubated with SARS-CoV-2 VLPs encoding GFP-PS9, GFP (no packaging signal), or no VLPs (blank).

FIG. 3A-3G illustrate the effect of amino acid changes in the spike protein on SARS-CoV-2 VLP (SC2-VLP) induced luminescence. FIG. 3A shows a heatmap of observed mutations within the spike protein as of July 2021. Each row corresponds to a variant of concern or variant of interest shown on left and each column indicates observed mutations shown at top. Colors indicate prevalence of each mutation and arrows at bottom indicate the mutations that were tested. FIG. 3B is a schematic illustrating cloning and testing of each variant for formation of SARS-CoV-2 VLPs. FIG. 3C graphically illustrates normalized relative luminescence for 15 spike mutants in an initial screen where the observed luminescence levels were compared to the luminescence of a reference ancestral SARS-CoV-2 spike protein containing the D614G mutation. FIG. 3D graphically illustrates normalized relative luminescence for SARS-CoV-2 spike mutants evaluated over a range of plasmid dilutions with all other plasmids maintained at the same concentration. FIG. 3E illustrates the effects of spike mutations on SC2-VLP induced luminescence. Induced luminescence is shown from receiver cells incubated with SC2-VLPs containing varying concentrations and mutations within the SARS-CoV-2 Spike protein. The Spike mutations are listed to the right. Spike-encoding plasmid concentrations ranging from 0.1 ng to 12.5 ng were added to each well of a 24-well plate. Total DNA used for transfection (N, M-IRES-E, T20) was 1 μg for each well. FIG. 3F-3G illustrate the minimal sequence required for specific packaging into SC2-VLPs. FIG. 3F graphically illustrates induced luminescence in receiver cells after incubation with different SC2-VLPs, where each VLP contained a transcript expressing luciferase and a different segment of the SARS-CoV-2 genome. The positions of the transcript segments from SARS-CoV-2 are shown graphically in FIGS. 2C, 2E, and 3G. FIG. 3G us a heatmap illustrating different segments from SARS-CoV-2 while the darkness of the segments indicates the observed luminescence normalized to the T20 transcript, where darker segments exhibit more luminescence.

FIG. 4A-4I illustrate the effects of amino acid changes in the N protein on SC2-VLP induced luminescence. FIG. 4A shows a map of the region of SARS-CoV-2 encoding the N protein, with the locations of observed N protein mutations identified. FIG. 4B shows a heatmap of observed mutations within the N protein as of July 2021. Each row corresponds to a variant of concern or variant of interest shown on left and each column indicates a particular mutation at top. The shaded darkness indicates prevalence of each mutation and arrows indicate mutations that were tested, with darker shading indicating increased prevalence. FIG. 4C is a schematic illustrating methods for screening N mutations using SC2-VLPs. FIG. 4D graphically illustrates the normalized luminescence observed in an initial screen of fifteen N mutants compared to the reference Wuhan Hu-1 N sequence (WT). FIG. 4E graphically illustrates the normalized luminescence observed for six N mutants re-tested for luciferase expression after preparation in a larger batch. FIG. 4F graphically illustrates the relative N protein expression in packaging cells normalized to WT using GAPDH as a loading control as assessed by western blot analysis. FIG. 4G is a schematic illustrating methods for isolating purified VLPs for analysis (e.g., by western and northern blots). FIG. 4H shows a Western blot (protein) and a Northern blot (RNA) of isolated VLPs generated from the six N mutants as well as controls and blanks. One mL of a batch of lentivirus was added to each sample before ultracentrifugation to allow p24 to be used as a loading control. Anti-N antibody (abcam, ab273434) binds to C-terminal domain of the N protein, which does not contain any of the mutations tested. FIG. 4I shows a western blot illustrating expression levels of nucleocapsid (N protein) mutants. Western blot of lysates from packaging cells transfected with N mutations stained using anti-N antibody (top) and anti-GAPDH antibody (bottom). Expression levels are similar between mutants and do not correlate with induced luminescence from SC2-VLPs made from these mutants.

FIG. 5A-5C graphically illustrate the luminescence measured as a function of VLPs generated with the component protein shown, in a background of B.1 genes. FIG. 5A graphically illustrates the luminescence measured from receiver cells contacted with SC2-VLPs having different SARS-CoV-2 variant spike proteins where the luminescence was normalized to receiver cells contacted with SC2-VLPs having SARS-CoV-2 B.1 proteins. FIG. 5B graphically illustrates the luminescence measured from receiver cells contacted with SC2-VLPs having different SARS-CoV-2 variant N proteins where the luminescence was normalized to receiver cells contacted with SC2-VLPs having SARS-CoV-2 B. 1 proteins. FIG. 5C graphically illustrates the luminescence measured from receiver cells contacted with SC2-VLPs having different SARS-CoV-2 variant M and/or E proteins where the luminescence was normalized to receiver cells contacted with SC2-VLPs having SARS-CoV-2 B.1 proteins.

FIG. 6A-6L illustrate that patient antisera exhibit varying levels of neutralization of infections by SARS-CoV-2 VLPs generated with different Spike proteins. FIG. 6A graphically illustrates 50% neutralization titers of sera isolated from individuals vaccinated using the Pfizer/BioNTech vaccine. Neutralization curves were determined using VLPs with S-proteins from B.1, Delta, or Omicron SARS-CoV-2 variants. FIG. 6B graphically illustrates 50% neutralization titers of sera isolated from individuals vaccinated using the Moderna vaccine. Neutralization curves were determined using VLPs with S-proteins from B.1, Delta, or Omicron variants. FIG. 6C graphically illustrates 50% neutralization titers of sera isolated from individuals vaccinated using the Johnson and Johnson vaccine. Neutralization curves were determined using VLPs with S-proteins from B.1, Delta, or Omicron variants. FIG. 6D graphically illustrates 50% neutralization titers of sera isolated from convalescent COVID-19 patients. Neutralization curves were determined using VLPs with S-proteins from B.1, Delta, or Omicron variants. FIG. 6E graphically illustrates 50% neutralization titers of sera isolated from individuals vaccinated using the Pfizer/BioNTech vaccine. Neutralization curves were determined using VLPs with S-proteins from B.1, Omicron, Omicron class 1 (OmC1), or Omicron class 3 (OmC3) variants. FIG. 6F graphically illustrates 50% neutralization titers of sera isolated from individuals vaccinated using the Moderna vaccine. Neutralization curves were determined using VLPs with S-proteins from B.1, Omicron, Omicron class 1 (OmC1), or Omicron class 3 (OmC3) variants. FIG. 6G graphically illustrates 50% neutralization titers of sera isolated from individuals vaccinated using the Johnson and Johnson vaccine. Neutralization curves were determined using VLPs with S-proteins from B.1, Omicron, Omicron class 1 (OmC1), or Omicron class 3 (OmC3) variants. FIG. 6H graphically illustrates 50% neutralization titers of sera isolated from convalescent COVID-19 patients Neutralization curves were determined using VLPs with S-proteins from B.1, Omicron, Omicron class 1 (OmC1), or Omicron class 3 (OmC3) variants. FIG. 6I graphically illustrates 50% neutralization titers of sera isolated at 16 or 21 days after individuals were boosted with a third dose of the Pfizer/BioNTech vaccine when tested against VLPs displaying the B. 1 spike protein. FIG. 6J graphically illustrates 50% neutralization titers of sera isolated at 16 or 21 days after individuals were boosted with a third dose of the Pfizer/BioNTech vaccine when tested against VLPs displaying the Delta spike protein. FIG. 6K graphically illustrates 50% neutralization titers of sera isolated at 16 or 21 days after individuals were boosted with a third dose of the Pfizer/BioNTech vaccine when tested against VLPs displaying the Omicron spike protein. FIG. 6L graphically illustrates 50% neutralization titers of sera isolated at 21 days after individuals were boosted with a third dose of the Pfizer/BioNTech vaccine when tested against VLPs displaying the B.1, Delta, or Omicron spike proteins. *p<0.05, **p<0.01, ***p<0.001, ****p<0.0001 evaluated using Friedman's exact test for repeated measures.

FIG. 7A-7E illustrate antibody neutralization of VLPs generated with different S genes. FIG. 7A shows neutralization curves and IC50 values of Casirivimab and Imdevimab monoclonal antibodies against the B.1 Spike protein variant. FIG. 7B shows neutralization curves and IC50 values of Casirivimab and Imdevimab against the Delta Spike protein variant. FIG. 7C shows neutralization curves and IC50 values of Casirivimab and Imdevimab against the Omicron Spike protein variant. FIG. 7D shows neutralization curves and IC50 values of Casirivimab and Imdevimab against the Omicron Spike protein variant with Class 1 mutations. FIG. 7E shows neutralization curves and IC50 values of Casirivimab and Imdevimab against the Omicron Spike protein variant with Class 3 mutations.

FIG. 8A-8E illustrate neutralizing antibody levels in the sera of fully vaccinated, uninfected individuals when evaluated against SARS-CoV-2 VLPs and live SARS-CoV-2 virions. FIG. 8A shows box-violin plots illustrating median neutralizing antibody titers of serum from vaccinated, unboosted individuals when evaluated using VLPs (left) and live virus (right) in assays against the SARS-CoV-2 WA-1 ancestral lineage (wild type [WT]) and Delta SARS-CoV-2 variant. FIG. 8B shows box-violin plots illustrating median neutralizing antibody titers of serum from vaccinated, unboosted individuals when evaluated using VLPs (left) and live virus (right) in assays against the SARS-CoV-2 WA-1 ancestral lineage (wild type [WT]) and Omicron SARS-CoV-2 variant. FIG. 8C shows box-violin plots illustrating median neutralizing antibody titers of serum from vaccinated and boosted individuals when evaluated using VLP (left) and live virus (right) in assays against the SARS-CoV-2 WA-1 ancestral lineage (wild type [WT]) and Delta SARS-CoV-2 variant FIG. 8D shows box-violin plots illustrating median neutralizing antibody titers of serum from vaccinated and boosted individuals when evaluated using VLP (left) and live virus (right) in assays against the SARS-CoV-2 WA-1 ancestral lineage (wild type [WT]) and Omicron SARS-CoV-2 variant. FIG. 8E shows longitudinal box-violin plots of VLP titers against Delta (top) and Omicron (bottom) SARS-CoV-2 strains stratified by time ranges following completion of a primary vaccine series. For box-violin plots, the median is represented by a thick black line inside the box, boxes represent the first to third quartiles, whiskers represent the minimum and maximum values, and the width of each curve corresponds with the approximate frequency of data points in each region.

### DETAILED DESCRIPTION

Methods, expression systems, and constructs are described herein for generating SARS-CoV-2 virus-like particles that load and deliver engineered transcripts into cells. The methods and constructs are useful for analysis of viral assembly, stability and entry of different SARS-CoV-2 strains (including various variant and mutant strains) and for identifying agents that can modify SARS-CoV-2 viral assembly, stability and entry.

Understanding the molecular determinants of SARS-CoV-2 viral fitness is central to effective vaccine and therapeutic development. The emergence of viral variants including Delta and Omicron underscores the need to assess both infectivity and antibody neutralization, but biosafety level 3 (BSL-3) handling requirements slow the pace of research on intact SARS-CoV-2. Although vesicular stomatitis virus (VSV) and lentivirus pseudotyped with the SARS-CoV-2 spike (S) protein enable evaluation of S-mediated cell binding and entry via the ACE2 and TMPRSS2 receptors, they cannot determine effects of mutations outside the S gene (Crawford et al. Viruses 12 (2020); Plante et al., Nature 592:116-121 (2021).

To address these challenges, SARS-CoV-2 virus-like particles (SC2-VLPs) were developed as described herein that include viral structural proteins and a packaging signal-containing messenger RNA that together form RNA-loaded capsids capable of spike-dependent cell transduction. This system faithfully reports the impact of mutations in viral structural proteins that are observed in live-virus infections, enabling rapid testing of SARS-CoV-2 structural gene variants for their impact on both infection efficiency and antibody or antiserum neutralization.

SARS-CoV-2 has four major viral structural proteins: the spike (S), the membrane (M), the envelope (E), and the nucleocapsid (N) proteins. These proteins contribute to the assembly, packaging and cellular entry for SARS-CoV-2.

The methods described herein include expressing a nucleic acid that includes both a SARS-CoV-2 packaging signal sequence linked to a heterologous nucleic acid in cells that also express each of the SARS-CoV-2 spike (S), membrane (M), envelope (E), and nucleocapsid (N) proteins. The SARS-CoV-2 packaging signal sequence linked to a heterologous nucleic acid can include a promoter to facilitate expression the packaging signal and the heterologous nucleic acid.

The heterologous nucleic acid can encode one or more coding regions and/or types of RNA. The encoded proteins and RNAs encoded can encode therapeutic agents and inhibitors useful for treating viral infection. The encoded RNAs and proteins can also encode proteins that facilitate evaluation of different viral strains. Examples of proteins that can be encoded by the heterologous nucleic acid include one or more antibodies, antigens, signal-producing proteins, and/or viral replication proteins.

For example, the heterologous nucleic acid can encode SARS-CoV-2 replication proteins (e.g. SARS-CoV-2 nsp1-16), Venezuelan equine encephalitis virus (VEEV) replication protein (nsP1-4) in one engineered transcript along with the packaging signal. The replication protein-packaging signal transcript is incorporated into the VLP and is delivered into a cell. When such viral replication proteins are present, the VLP can undergo a single round of replication and infection. Cells infected with VLPs encoding replication proteins cannot generate virus or more VLPs, so the infection/VLPs do not spread to other cells. The advantage is that even if only one VLP enters a cell, the replicase (replication) protein(s) make many copies of the engineered transcript generating high levels of whichever proteins are encoded by the heterologous nucleic acid. In the vaccine field, this strategy is called “self-amplifying RNA” or “self-replicating RNA.”

The heterologous nucleic acid can encode the viral replication proteins along with one or more other proteins, including therapeutic proteins, antigens, antibodies, signal proteins, and the like Therapeutic proteins can include agents such as lopinavir/ritonavir, remdesivir, favipiravir, interferon, ribavirin, tocilizumab, sarilumab, or combinations thereof. The antigens can include viral proteins such as spike protein antigens (e.g., peptides from the spike protein), or other viral structural proteins. The antibodies can be anti-viral antibodies, for example, anti-spike protein antibodies.

In some cases the heterologous nucleic acid includes a detectable signal protein coding region. As used herein, the “detectable signal protein” is any protein that provides a detectable signal. The signal can be a visible color, a visible light, or light emitted in the ultraviolet or infrared wavelengths of light. The signal can be fluorescent light. The signal is detectable, for example, by light microscopy and/or by any light detector.

Co-expression of the SARS-CoV-2 packaging signal sequence linked to the detectable signal protein sequence in cells that also express the 2 spike (S), membrane (M), envelope (E), and nucleocapsid (N) proteins generates SARS-CoV-2 virus-like-particles. The signal protein can provide a signal from within cells that produce the virus-like-particles. The signal level is a measure of the extent of virus-like-particle production and/or cellular entry.

One or more of the SARS-CoV-2 spike (S) protein, membrane (M) protein, envelope (E) protein, or nucleocapsid (N) protein used in the expression system can be a variant or mutant protein. For example, the SARS-CoV-2 spike (S) protein, membrane (M) protein, envelope (E) protein, or nucleocapsid (N) protein can be a mutant or variant compared to a segment of the SARS-CoV-2 sequence provided herein as SEQ ID NO:1. In some cases, the methods include culturing the cells in a test agent. The effects of the test agent upon virus-like-particle assembly, packaging, and/or cellular entry can be used to identify useful agents for modulating (e.g., inhibiting) SARS-CoV-2 assembly, packaging, and/or cellular entry.

For example, an expression system that includes one or more expression cassettes encoding a SARS-CoV-2 packaging signal sequence-detectable signal protein coding region, a SARS-CoV-2 spike (S) protein, a SARS-CoV-2 membrane (M) protein, a SARS-CoV-2 envelope (E) protein, and SARS-CoV-2 nucleocapsid (N) protein can be introduced into a host cell. In some cases, the expression cassettes or expression vectors encoding the SARS-CoV-2 packaging signal sequence-detectable signal protein coding region, the SARS-CoV-2 spike (S) protein, the SARS-CoV-2 membrane (M) protein, the SARS-CoV-2 envelope (E) protein, and the SARS-CoV-2 nucleocapsid (N) protein are introduced in equimolar amounts into a host cell. In other cases, one or more of the expression cassettes or expression vectors encoding the SARS-CoV-2 packaging signal sequence, the detectable signal protein coding region, the SARS-CoV-2 spike (S) protein, the SARS-CoV-2 membrane (M) protein, the SARS-CoV-2 envelope (E) protein, and the SARS-CoV-2 nucleocapsid (N) protein are introduced in non-equimolar amounts into a host cell. These cells may be referred to as transfected cells. The SARS-CoV-2 packaging signal sequence and the detectable signal protein coding region can be operably linked. The expression cassettes encoding such a SARS-CoV-2 packaging signal sequence-detectable signal protein coding region, the SARS-CoV-2 spike (S) protein, the SARS-CoV-2 membrane (M) protein, the SARS-CoV-2 envelope (E) protein, and the SARS-CoV-2 nucleocapsid (N) protein can be within a single expression vector. Alternatively, the expression cassettes encoding the SARS-CoV-2 packaging signal sequence-detectable signal protein coding region, the SARS-CoV-2 spike (S) protein, the SARS-CoV-2 membrane (M) protein, the SARS-CoV-2 envelope (E) protein, and the SARS-CoV-2 nucleocapsid (N) protein can be in two or more separate expression vectors.

Transfected cells (host cells) expressing the SARS-CoV-2 packaging signal sequence-detectable signal protein coding region, the SARS-CoV-2 spike (S) protein, the SARS-CoV-2 membrane (M) protein, the SARS-CoV-2 envelope (E) protein, and the SARS-CoV-2 nucleocapsid (N) protein can produce (e.g, shed) SARS-CoV-2 virus-like particles. Such SARS-CoV-2 virus-like particles can be collected and/or separated from the transfected cells.

The transfected cells and/or host cells can be of any cell type that can be transfected and express the SARS-CoV-2 packaging signal sequence-detectable signal protein coding region, the SARS-CoV-2 spike (S) protein, the SARS-CoV-2 membrane (M) protein, the SARS-CoV-2 envelope (E) protein, and the SARS-CoV-2 nucleocapsid (N) protein.

In some cases the transfected cells and/or the SARS-CoV-2 virus-like particles are contacted with receptor cells. Receptor cells have a receptor for SARS-CoV-2 but in some cases may not express SARS-CoV-2 viral proteins before contact with the transfected cells and/or the SARS-CoV-2 virus-like particles. After the receptor cells are contacted with the transfected cells and/or the SARS-CoV-2 virus-like particles, the receptor cells can express at least the heterologous protein. For example, the receptor cells can express the detectable signal protein, which emits a signal indicating that the receptor cells were ‘infected’ with the SARS-CoV-2 virus-like particles.

The receptor and/or transfected host cells can be of any cell type. However, the receptor cells should express a receptor for SARS-CoV-2. An example of a receptor for SARS-CoV-2 is a human ACE2 receptor. The receptor and/or host cells can express TMPRSS2. Examples of cells that are susceptible to SARS-CoV-2 are described by Wang et al., Emerg Infect Dis. 27(5):1380-1392 (May 2021). In some cases, the receptor and/or host cells can be 293T cells. In some cases, the receptor and/or host cells can be other cell types, including for example one more cell types from a patient or human suspected of being susceptible to SARS-CoV-2 infection.

The host cells or transfected host cells can be incubated in culture media for a time and under conditions sufficient for expression of the SARS-CoV-2 packaging signal sequence-detectable signal protein coding region, the SARS-CoV-2 spike (S) protein, the SARS-CoV-2 membrane (M) protein, the SARS-CoV-2 envelope (E) protein, and the SARS-CoV-2 nucleocapsid (N) protein.

The culture media can be a mammalian cell culture medium. Examples include DMEM and RPMI 1640 cell media. The media can contain fetal serum, such as fetal bovine serum. In some cases, the media can contain antibiotics such as penicillin and/or streptomycin. The media can be changed at regular intervals, such as at 12 hour intervals, daily intervals, 48 hour intervals, or other intervals.

Virus-like-particles (VLPs) can be collected from the cell medium within 12 to 72 hours after transfection.

To distinguish virus-like-particles (VLPs) from cells, cellular debris, and other debris, a signal from the detectable signal protein can be detected. In some cases, various reagents can be used to elicit or enhance the signal.

The intensity of the signal is, as illustrated herein, directly correlated with the number or quantity of virus-like-particles (VLPs). Hence, a standard curve of signal intensity versus the number or quantity of virus-like-particles (VLPs) can be used to determine an unknown number of virus-like-particles (VLPs).

Test agents can be introduced at various steps and at various times during the preparation of the VLPS. The ability of the test agents to modulate or inhibit VLP formation can be assessed by comparing the number or amounts of VLP produced in the presence or absence of one or more test agents.

The virus-like-particles (VLPs) can be collected by any convenient means. Culture media containing VLPs can be filtered, precipitated with polyethylene glycol (PEG), or subjected to sucrose gradient centrifugation as illustrated herein.

VLPs can incubated with receptor cells for a time and under conditions sufficient for attachment and take up of the VLPs by the cells. Test agents can also be mixed with the VLPs and the cells to evaluate whether the test agent(s) can reduce or inhibit VLP uptake by the cells.

A variety of test agents can be tested to identify compounds that reduce SARS-CoV-2 viral (VLP) packaging, cellular entry, and viral replication, or a combination thereof in the assay methods described herein compared to a control assays without the test compound(s). For example, one or more small molecules, antibodies, nucleic acids, carbohydrates, proteins, peptides, or a combination thereof can be tested in the assays.

Also described herein are screening methods that can be used to identify useful small molecules, polypeptides, anti-SARS-CoV-2 antibodies, SARS-CoV-2 inhibitory nucleic acids, and combinations thereof. Such useful small molecules, polypeptides, antibodies, and inhibitory nucleic acids can be screened for inhibiting VLP assembly, for inhibiting VLP packaging, for binding to the SARS-CoV-2 VLPS, for inhibiting the binding of VLPs to cells, for inhibiting VLP cellular entry, or a combination thereof. The small molecules, polypeptides, and antibodies can also be evaluated as therapeutics for treating the short-term and the long-term symptoms of SARS-CoV-2 infection. For example, the small molecules, polypeptides, antibodies, inhibitory nucleic acids can also be tested to ascertain if they can reduce adverse symptoms of SARS-CoV-2 infection such as inflammation and oxidative stress in the brain, gut, kidneys, vascular system, lungs, or a combination thereof.

The methods can involve contacting one or more test agents with (a) one or more VLPs; or (b) one or more cells that express the SARS-CoV-2 packaging signal sequence-heterologous nucleic acid as well as the SARS-CoV-2 spike (S), membrane (M), envelope (E), and nucleocapsid (N) proteins. Such a test agent/VLP/cell mixture can then be evaluated for VLP assembly, VLP packaging, VLP cellular entry, VLP reproduction, or a combination thereof. Such detection can involve detecting a signal, or the level of signal, from a detectable signal protein encoded by the SARS-CoV-2 packaging signal sequence-heterologous nucleic acid.

Test agents that do bind to inhibit VLP assembly, VLP packaging, VIP cellular entry, VLP reproduction, or a combination thereof can also be administered to an animal that is infected with SARS-CoV-2 virus. The effects of the test agents on the course of SARS-CoV-2 infection in the animal can then be determined. For example, the methods can also include determining whether the test agent can reduce inflammation and/or oxidative stress associated with the SARS-CoV-2 infection within the animal. For example, the methods can include determining whether the test agent can reduce inflammation and/or oxidative stress in the brain, gut, kidneys, vascular system, and/or the lungs of animals infected with SARS-CoV-2 virus.

### SARS-CoV-2 Packaging Signal Constructs

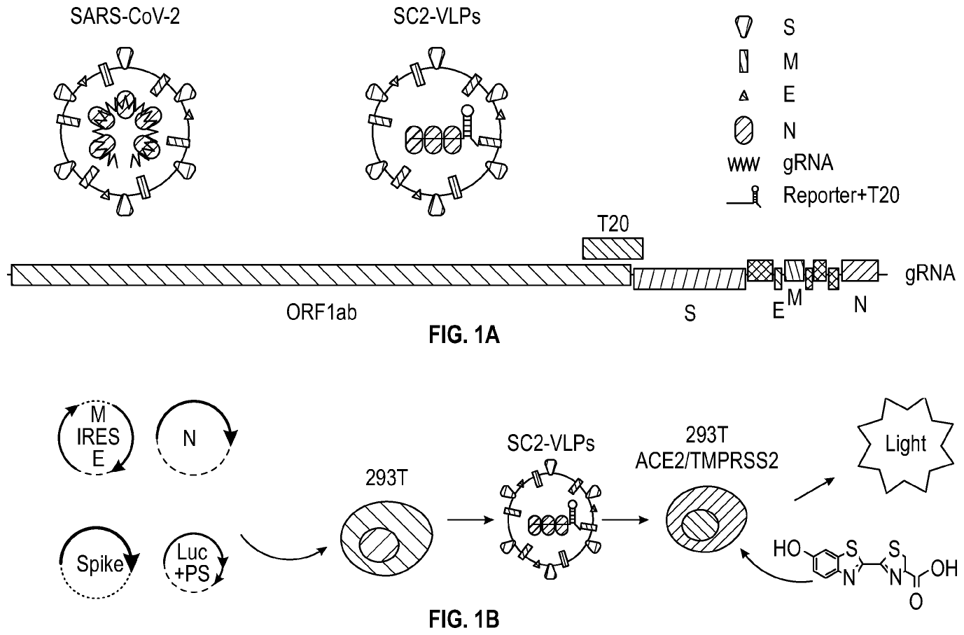

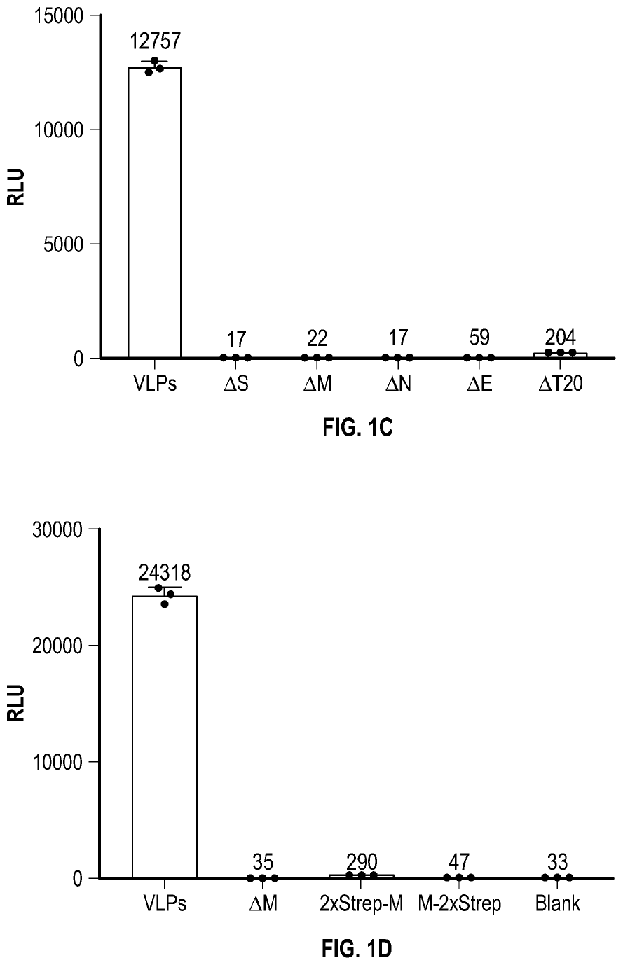

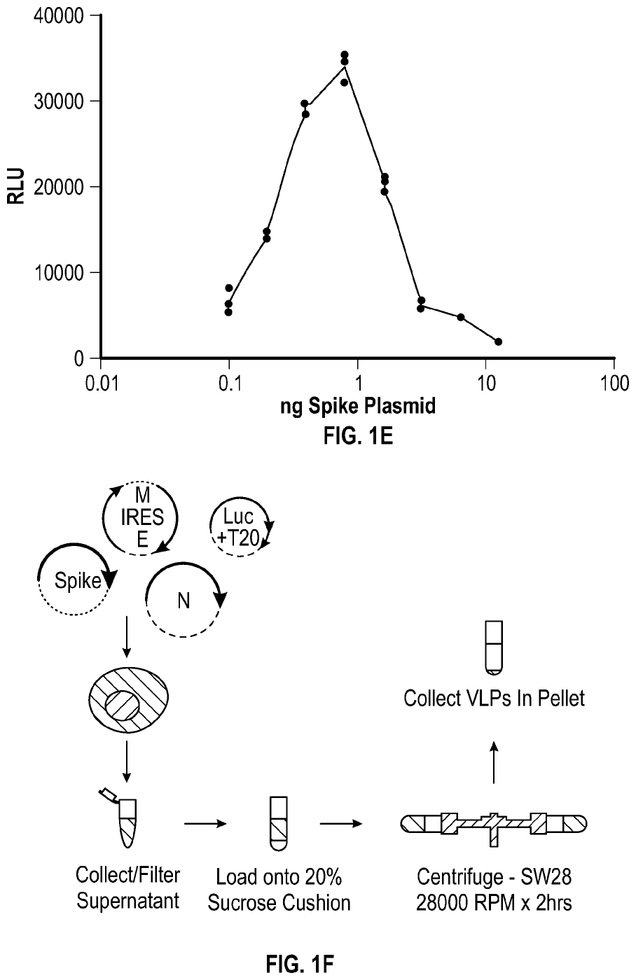

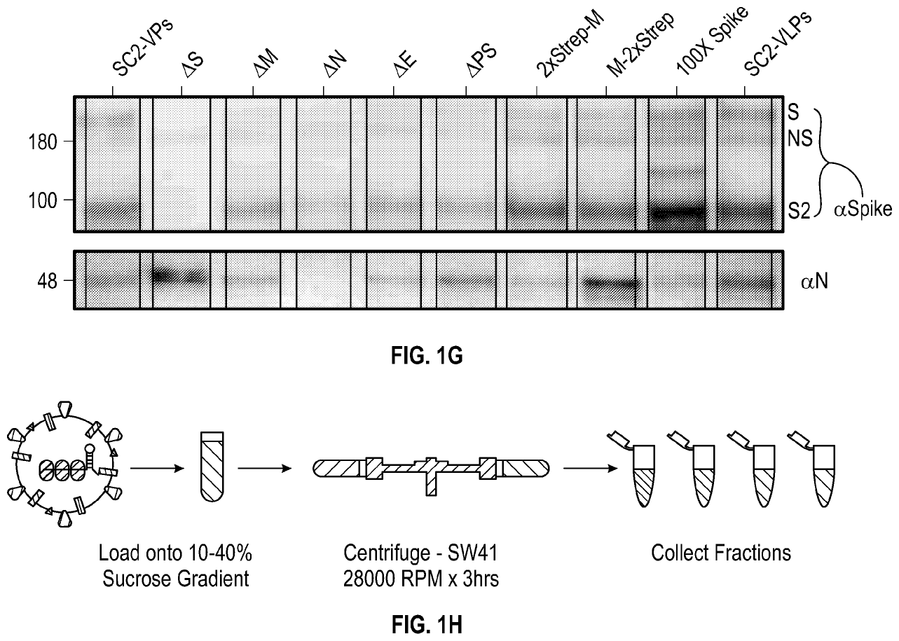

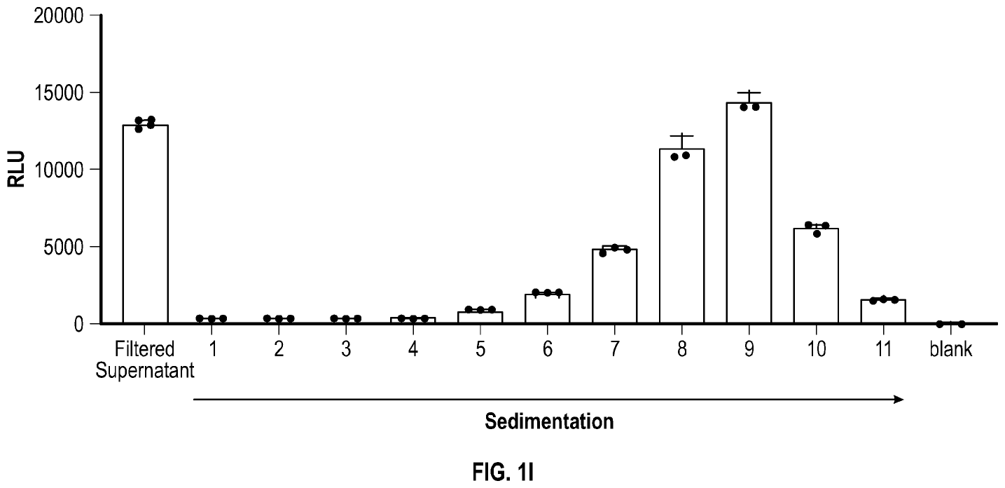

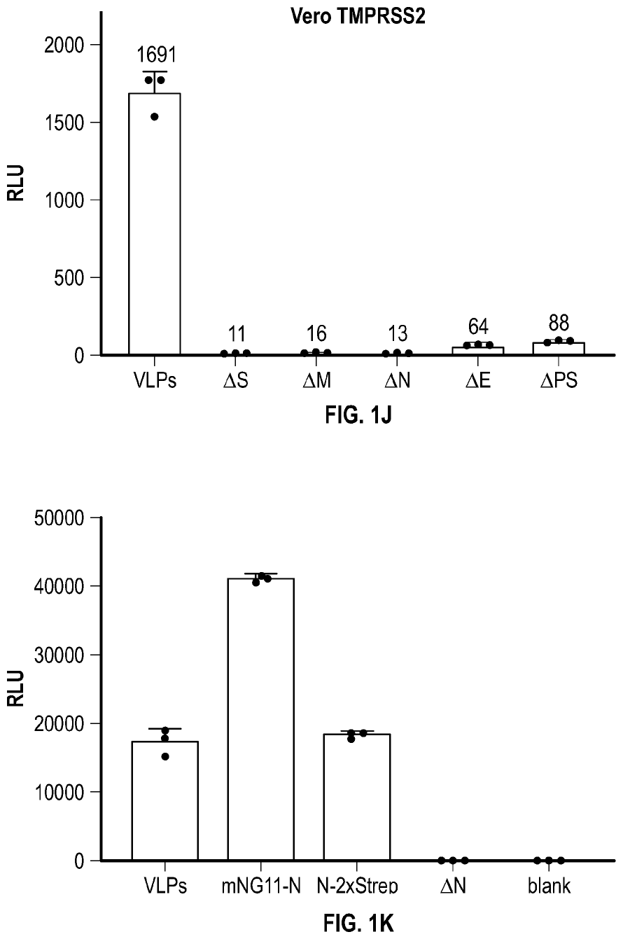

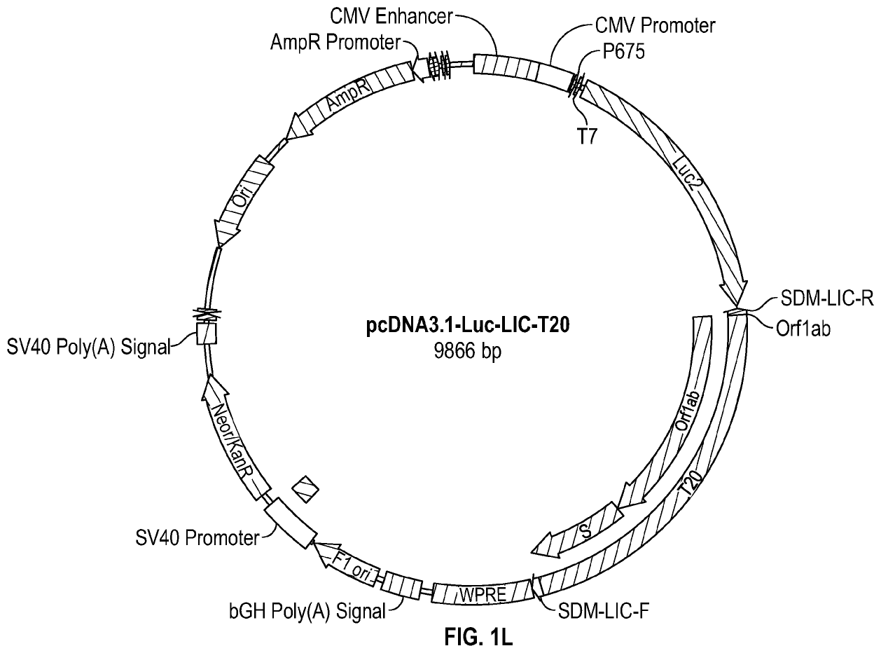

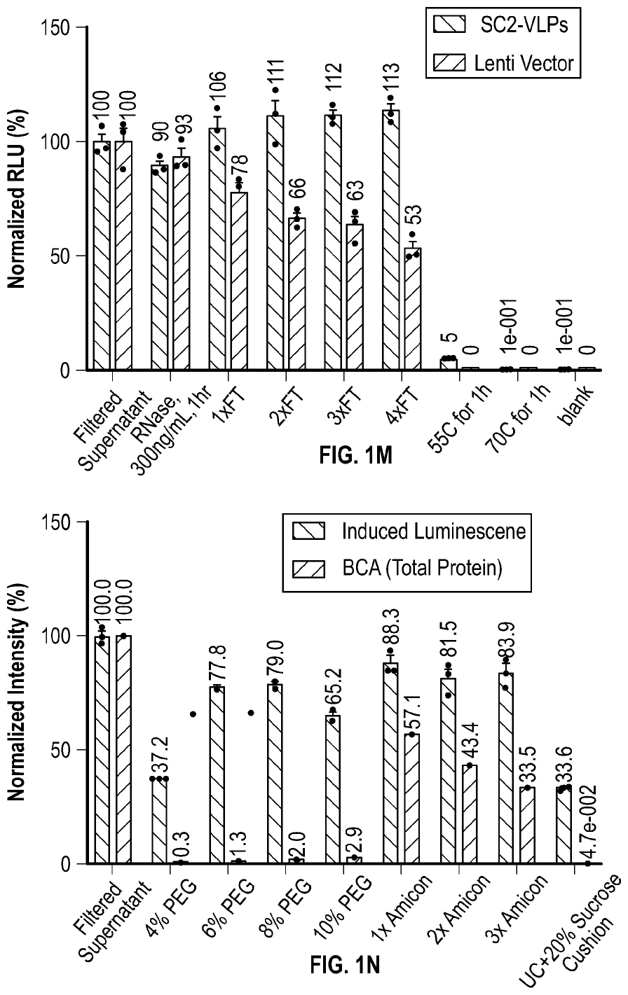

The inventors hypothesized that the SARS-CoV-2 packaging signal might reside within genomic fragment “T20” (nucleotides 20080-22222) encoding non-structural protein 15 (nsp15) and nsp16 (FIG. 1A). A sequence for the SARS-CoV-2 nucleic acid sequence available as accession number NC_045512.2 at the NCBI website (and provided herein as SEQ ID NO:1). The segment from the accession number NC_045512.2 sequence that includes the “T20” genomic fragment (nucleotides 20080-22222) that encodes non-structural protein 15 (nsp15) and nsp16 is provided below as SEQ ID NO:2.

| | |
|---|---|
| 20080 | T |
| 20081 | CTGTAGGTCC CAAACAAGCT AGTCTTAATG GAGTCACATT |
| 20121 | AATTGGAGAA GCCGTAAAAA CACAGTTCAA TTATTATAAG |
| 20161 | AAAGTTGATG GTGTTGTCCA ACAATTACCT GAAACTTACT |
| 20201 | TTACTCAGAG TAGAAATTTA CAAGAATTTA AACCCAGGAG |
| 20241 | TCAAATGGAA ATTGATTTCT TAGAATTAGC TATGGATGAA |
| 20281 | TTCATTGAAC GGTATAAATT AGAAGGCTAT GCCTTCGAAC |
| 20321 | ATATCGTTTA TGGAGATTTT AGTCATAGTC AGTTAGGTGG |
| 20361 | TTTACATCTA CTGATTGGAC TAGCTAAACG TTTTAAGGAA |
| 20401 | TCACCTTTTG AATTAGAAGA TTTTATTCCT ATGGACAGTA |
| 20441 | CAGTTAAAAA CTATTTCATA ACAGATGCGC AAACAGGTTC |
| 20481 | ATCTAAGTGT GTGTGTTCTG TTATTGATTT ATTACTTGAT |
| 20521 | GATTTTGTTG AAATAATAAA ATCCCAAGAT TTATCTGTAG |
| 20561 | TTTCTAAGGT TGTCAAAGTG ACTATTGACT ATACAGAAAT |
| 20601 | TTCATTTATG CTTTGGTGTA AAGATGGCCA TGTAGAAACA |
| 20641 | TTTTACCCAA AATTACAATC TAGTCAAGCG TGGCAACCGG |
| 20681 | GTGTTGCTAT GCCTAATCTT TACAAAATGC AAAGAATGCT |
| 20721 | ATTAGAAAAG TGTGACCTTC AAAATTATGG TGATAGTGCA |
| 20761 | ACATTACCTA AAGGCATAAT GATGAATGTC GCAAAATATA |
| 20801 | CTCAACTGTG TCAATATTTA AACACATTAA CATTAGCTGT |
| 20841 | ACCCTATAAT ATGAGAGTTA TACATTTTGG TGCTGGTTCT |
| 20881 | GATAAAGGAG TTGCACCAGG TACAGCTGTT TTAAGACAGT |
| 20921 | GGTTGCCTAC GGGTACGCTG CTTGTCGATT CAGATCTTAA |
| 20961 | TGACTTTGTC TCTGATGCAG ATTCAACTTT GATTGGTGAT |
| 21001 | TGTGCAACTG TACATACAGC TAATAAATGG GATCTCATTA |
| 21041 | TTAGTGATAT GTACGACCCT AAGACTAAAA ATGTTACAAA |
| 21081 | AGAAAATGAC TCTAAAGAGG GTTTTTTCAC TTACATTTGT |
| 21121 | GGGTTTATAC AACAAAAGCT AGCTCTTGGA GGTTCCGTGG |
| 21161 | CTATAAAGAT AACAGAACAT TCTTGGAATG CTGATCTTTA |
| 21201 | TAAGCTCATG GGACACTTCG CATGGTGGAC AGCCTTTGTT |
| 21241 | ACTAATGTGA ATGCGTCATC ATCTGAAGCA TTTTTAATTG |
| 21281 | GATGTAATTA TCTTGGCAAA CCACGCGAAC AAATAGATGG |
| 21321 | TTATGTCATG CATGCAAATT ACATATTTTG GAGGAATACA |
| 21361 | AATCCAATTC AGTTGTCTTC CTATTCTTTA TTTGACATGA |
| 21401 | GTAAATTTCC CCTTAAATTA AGGGGTACTG CTGTTATGTC |
| 21441 | TTTAAAAGAA GGTCAAATCA ATGATATGAT TTTATCTCTT |
| 21481 | CTTAGTAAAG GTAGACTTAT AATTAGAGAA AACAACAGAG |
| 21521 | TTGTTATTTC TAGTGATGTT CTTGTTAACA ACTAAACGAA |
| 21561 | CAATGTTTGT TTTTCTTGTT TTATTGCCAC TAGTCTCTAG |
| 21601 | TCAGTGTGTT AATCTTACAA CCAGAACTCA ATTACCCCCT |
| 21641 | GCATACACTA ATTCTTTCAC ACGTGGTGTT TATTACCCTG |
| 21681 | ACAAAGTTTT CAGATCCTCA GTTTTACATT CAACTCAGGA |
| 21721 | CTTGTTCTTA CCTTTCTTTT CCAATGTTAC TTGGTTCCAT |
| 21761 | GCTATACATG TCTCTGGGAC CAATGGTACT AAGAGGTTTG |
| 21801 | ATAACCCTGT CCTACCATTT AATGATGGTG TTTATTTTGC |
| 21841 | TTCCACTGAG AAGTCTAACA TAATAAGAGG CTGGATTTTT |
| 21881 | GGTACTACTT TAGATTCGAA GACCCAGTCC CTACTTATTG |
| 21921 | TTAATAACGC TACTAATGTT GTTATTAAAG TCTGTGAATT |
| 21961 | TCAATTTTGT AATGATCCAT TTTTGGGTGT TTATTACCAC |
| 22001 | AAAAACAACA AAAGTTGGAT GGAAAGTGAG TTCAGAGTTT |
| 22041 | ATTCTAGTGC GAATAATTGC ACTTTTGAAT ATGTCTCTCA |
| 22081 | GCCTTTTCTT ATGGACCTTG AAGGAAAACA GGGTAATTTC |
| 22121 | AAAAATCTTA GGGAATTTGT GTTTAAGAAT ATTGATGGTT |
| 22161 | ATTTTAAAAT ATATTCTAAG CACACGCCTA TTAATTTAGT |
| 22201 | GCGTGATCTC CCTCAGGGTT TT |

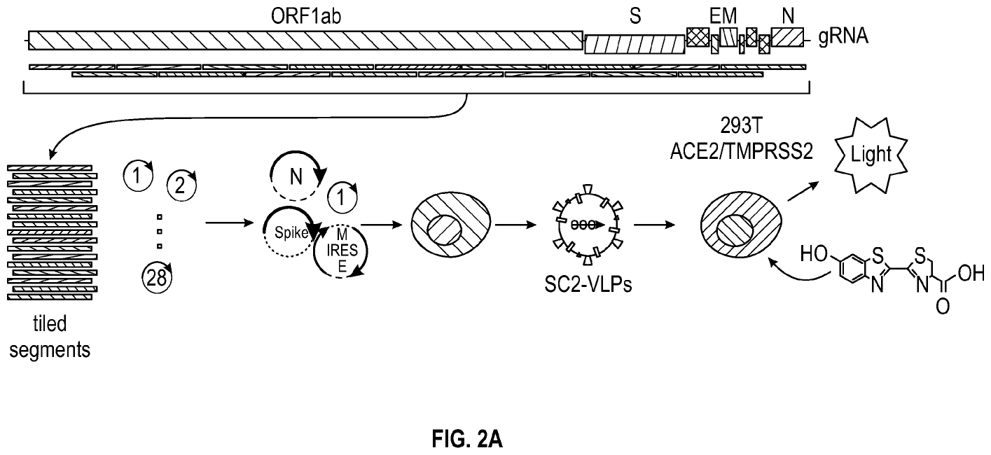

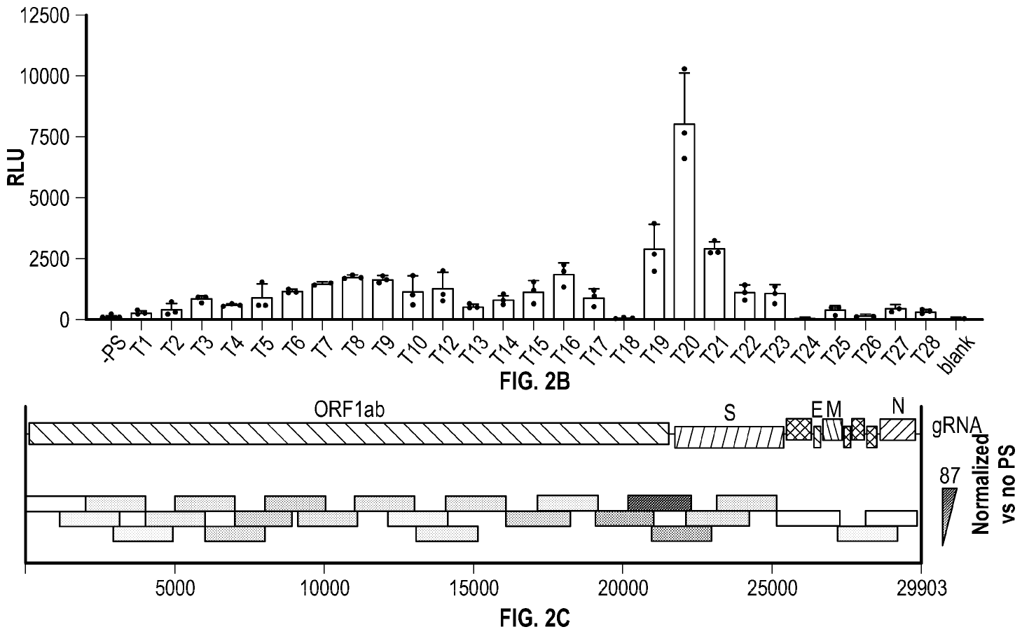

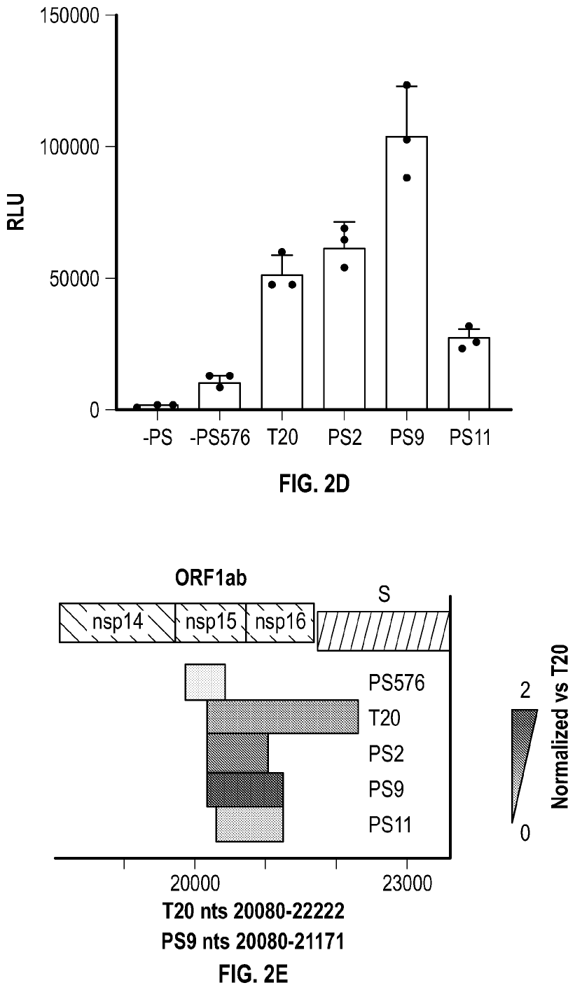

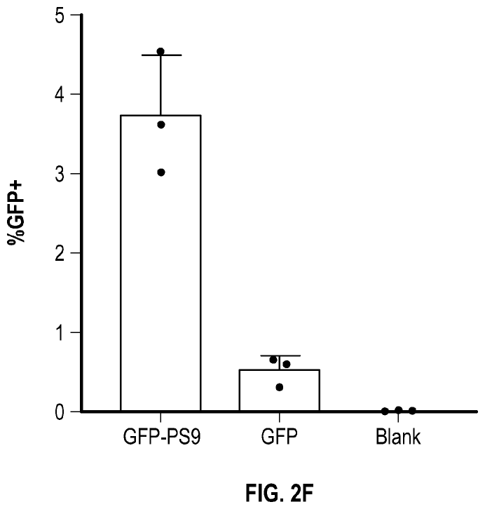

The T20 sequence shown above is an example of a packaging signal that can be used. However, the invention can also be practiced with packaging signals that have one or more deletions, nucleotide substitutions, or nucleotide insertions. For example, the inventors found that the highest packaging resulted from SARS-CoV-2 VLPs encoding nucleotide sequence that included positions 20080-21171 of the SARS-CoV-2 genome (termed PS9) as the packaging signal (FIG. 2D). The sequence of the PS9 packaging signal is shown below as SEQ ID NO:3.

| | |
|---|---|
| 20080 | T |
| 20081 | CTGTAGGTCC CAAACAAGCT AGTCTTAATG GAGTCACATT |
| 20121 | AATTGGAGAA GCCGTAAAAA CACAGTTCAA TTATTATAAG |
| 20161 | AAAGTTGATG GTGTTGTCCA ACAATTACCT GAAACTTACT |
| 20201 | TTACTCAGAG TAGAAATTTA CAAGAATTTA AACCCAGGAG |
| 20241 | TCAAATGGAA ATTGATTTCT TAGAATTAGC TATGGATGAA |
| 20281 | TTCATTGAAC GGTATAAATT AGAAGGCTAT GCCTTCGAAC |
| 20321 | ATATCGTTTA TGGAGATTTT AGTCATAGTC AGTTAGGTGG |
| 20361 | TTTACATCTA CTGATTGGAC TAGCTAAACG TTTTAAGGAA |
| 20401 | TCACCTTTTG AATTAGAAGA TTTTATTCCT ATGGACAGTA |
| 20441 | CAGTTAAAAA CTATTTCATA ACAGATGCGC AAACAGGTTC |
| 20481 | ATCTAAGTGT GTGTGTTCTG TTATTGATTT ATTACTTGAT |
| 20521 | GATTTTGTTG AAATAATAAA ATCCCAAGAT TTATCTGTAG |
| 20561 | TTTCTAAGGT TGTCAAAGTG ACTATTGACT ATACAGAAAT |
| 20601 | TTCATTTATG CTTTGGTGTA AAGATGGCCA TGTAGAAACA |
| 20641 | TTTTACCCAA AATTACAATC TAGTCAAGCG TGGCAACCGG |
| 20681 | GTGTTGCTAT GCCTAATCTT TACAAAATGC AAAGAATGCT |
| 20721 | ATTAGAAAAG TGTGACCTTC AAAATTATGG TGATAGTGCA |
| 20761 | ACATTACCTA AAGGCATAAT GATGAATGTC GCAAAATATA |
| 20801 | CTCAACTGTG TCAATATTTA AACACATTAA CATTAGCTGT |
| 20841 | ACCCTATAAT ATGAGAGTTA TACATTTTGG TGCTGGTTCT |
| 20881 | GATAAAGGAG TTGCACCAGG TACAGCTGTT TTAAGACAGT |
| 20921 | GGTTGCCTAC GGGTACGCTG CTTGTCGATT CAGATCTTAA |
| 20961 | TGACTTTGTC TCTGATGCAG ATTCAACTTT GATTGGTGAT |
| 21001 | TGTGCAACTG TACATACAGC TAATAAATGG GATCTCATTA |
| 21041 | TTAGTGATAT GTACGACCCT AAGACTAAAA ATGTTACAAA |
| 21081 | AGAAAATGAC TCTAAAGAGG GTTTTTTCAC TTACATTTGT |
| 21121 | GGGTTTATAC AACAAAAGCT AGCTCTTGGA GGTTCCGTGG |
| 21161 | CTATAAAGAT A |

These SARS-CoV-2 packaging signals encodes a portion of the ORF1ab polyprotein. For example, both of these SARS-CoV-2 packaging signals encode at least a portion of the nsp15 protein (FIG. 2E). The T20 packaging signal also encodes the majority of the nsp16 protein (FIG. 2E).

The packaging signal nucleic acid is linked to an expression cassette that encodes a signal protein (also called a marker protein). The segment encoding the signal protein is operably linked to a promoter.

The signal protein can be a luminescent protein, a fluorescent protein, or any protein that provides a detectable signal upon expression in the cell containing the packaging signal-signal protein construct. Examples of signal proteins include luciferase, aequorin, green fluorescent protein (GFP), EGFP, Emerald, Superfolder GFP, Azami Green, mWasabi, TagGFP, TurboGFP, AcGFP, ZsGreen, T-Sapphire, EBFP, EBFP2, Azurite, mTagBFP, ECFP, mECFP, Cerulean, m Turquoise, CyPet, AmCyan1, Midori-Ishi Cyan, TagCFP, mTFP1 (Teal), EYFP, Topaz, Venus, mCitrine, YPet, TagYFP, PhiYFP, ZsYellow1, mBanana, Kusabira Orange, Kusabira Orange2, mOrange, mOrange2, dTomato, dTomato-Tandem, TagRFP, TagRFP-T, DsRed, DsRed2, DsRed-Express (T1), DsRed-Monomer, mTangerine, mRuby, mApple, mStrawberry, AsRed2, mRFP1, JRed, mCherry, HcRed1, mRaspberry, dKeima-Tandem, HcRed-Tandem, mPlum, AQ143, or combinations thereof. In some cases, luciferase is used. Examples of luciferases that can be used include Firefly luciferase (from Photinus pyralis), Renilla Luciferase (from Renilla reniformis), or Nanoluc (from Oplophorus gracilis). The HiBiT system, based on the split luciferase complementation of two NanoLuc fragments, can also be used. The HiBIT system involves a 1.3-kDa peptide (11 amino acids) that is capable of producing bright luminescence through interaction with an 18-kDa polypeptide named Large BiT (LgBiT).

### SARS-CoV-2 Structural Protein Constructs

In addition to the packaging signal constructs, generation of the SARS-CoV-2 virus-like particles requires cells to expression of four SARS-CoV-2 structural proteins: the SARS-CoV-2 spike (S) protein, membrane (M) protein, envelope (E) protein, and nucleocapsid (N) protein.

An example of a SARS-CoV-2 viral sequence is provided herein as SEQ ID NO:1. The SARS-CoV-2 spike (S) protein can be encoded by an open reading frame at about positions 21563-25384 (gene S) of the SEQ ID NO:1 sequence. This nucleic acid, which encodes a SARS-CoV-2 spike (S) protein, is shown below as SEQ ID NO:4.

| | |
|---|---|
| 21563 | ATGTTTGT TTTTCTTGTT TTATTGCCAC TAGTCTCTAG |
| 21601 | TCAGTGTGTT AATCTTACAA CCAGAACTCA ATTACCCCCT |
| 21641 | GCATACACTA ATTCTTTCAC ACGTGGTGTT TATTACCCTG |
| 21681 | ACAAAGTTTT CAGATCCTCA GTTTTACATT CAACTCAGGA |
| 21721 | CTTGTTCTTA CCTTTCTTTT CCAATGTTAC TTGGTTCCAT |
| 21761 | GCTATACATG TCTCTGGGAC CAATGGTACT AAGAGGTTTG |
| 21801 | ATAACCCTGT CCTACCATTT AATGATGGTG TTTATTTTGC |
| 21841 | TTCCACTGAG AAGTCTAACA TAATAAGAGG CTGGATTTTT |
| 21881 | GGTACTACTT TAGATTCGAA GACCCAGTCC CTACTTATTG |
| 21921 | TTAATAACGC TACTAATGTT GTTATTAAAG TCTGTGAATT |
| 21961 | TCAATTTTGT AATGATCCAT TTTTGGGTGT TTATTACCAC |
| 22001 | AAAAACAACA AAAGTTGGAT GGAAAGTGAG TTCAGAGTTT |
| 22041 | ATTCTAGTGC GAATAATTGC ACTTTTGAAT ATGTCTCTCA |
| 22081 | GCCTTTTCTT ATGGACCTTG AAGGAAAACA GGGTAATTTC |
| 22121 | AAAAATCTTA GGGAATTTGT GTTTAAGAAT ATTGATGGTT |
| 22161 | ATTTTAAAAT ATATTCTAAG CACACGCCTA TTAATTTAGT |
| 22201 | GCGTGATCTC CCTCAGGGTT TTTCGGCTTT AGAACCATTG |
| 22241 | GTAGATTTGC CAATAGGTAT TAACATCACT AGGTTTCAAA |
| 22281 | CTTTACTTGC TTTACATAGA AGTTATTTGA CTCCTGGTGA |
| 22321 | TTCTTCTTCA GGTTGGACAG CTGGTGCTGC AGCTTATTAT |
| 22361 | GTGGGTTATC TTCAACCTAG GACTTTTCTA TTAAAATATA |
| 22401 | ATGAAAATGG AACCATTACA GATGCTGTAG ACTGTGCACT |
| 22441 | TGACCCTCTC TCAGAAACAA AGTGTACGTT GAAATCCTTC |
| 22481 | ACTGTAGAAA AAGGAATCTA TCAAACTTCT AACTTTAGAG |
| 22521 | TCCAACCAAC AGAATCTATT GTTAGATTTC CTAATATTAC |
| 22561 | AAACTTGTGC CCTTTTGGTG AAGTTTTTAA CGCCACCAGA |
| 22601 | TTTGCATCTG TTTATGCTTG GAACAGGAAG AGAATCAGCA |
| 22641 | ACTGTGTTGC TGATTATTCT GTCCTATATA ATTCCGCATC |
| 22681 | ATTTTCCACT TTTAAGTGTT ATGGAGTGTC TCCTACTAAA |
| 22721 | TTAAATGATC TCTGCTTTAC TAATGTCTAT GCAGATTCAT |
| 22761 | TTGTAATTAG AGGTGATGAA GTCAGACAAA TCGCTCCAGG |
| 22801 | GCAAACTGGA AAGATTGCTG ATTATAATTA TAAATTACCA |
| 22841 | GATGATTTTA CAGGCTGCGT TATAGCTTGG AATTCTAACA |
| 22881 | ATCTTGATTC TAAGGTTGGT GGTAATTATA ATTACCTGTA |
| 22921 | TAGATTGTTT AGGAAGTCTA ATCTCAAACC TTTTGAGAGA |
| 22961 | GATATTTCAA CTGAAATCTA TCAGGCCGGT AGCACACCTT |
| 23001 | GTAATGGTGT TGAAGGTTTT AATTGTTACT TTCCTTTACA |
| 23041 | ATCATATGGT TTCCAACCCA CTAATGGTGT TGGTTACCAA |
| 23081 | CCATACAGAG TAGTAGTACT TTCTTTTGAA CTTCTACATG |
| 23121 | CACCAGCAAC TGTTTGTGGA CCTAAAAAGT CTACTAATTT |
| 23161 | GGTTAAAAAC AAATGTGTCA ATTTCAACTT CAATGGTTTA |
| 23201 | ACAGGCACAG GTGTTCTTAC TGAGTCTAAC AAAAAGTTTC |
| 23241 | TGCCTTTCCA ACAATTTGGC AGAGACATTG CTGACACTAC |
| 23281 | TGATGCTGTC CGTGATCCAC AGACACTTGA GATTCTTGAC |
| 23321 | ATTACACCAT GTTCTTTTGG TGGTGTCAGT GTTATAACAC |
| 23361 | CAGGAACAAA TACTTCTAAC CAGGTTGCTG TTCTTTATCA |
| 23401 | GGATGTTAAC TGCACAGAAG TCCCTGTTGC TATTCATGCA |
| 23441 | GATCAACTTA CTCCTACTTG GCGTGTTTAT TCTACAGGTT |
| 23481 | CTAATGTTTT TCAAACACGT GCAGGCTGTT TAATAGGGGC |
| 23521 | TGAACATGTC AACAACTCAT ATGAGTGTGA CATACCCATT |
| 23561 | GGTGCAGGTA TATGCGCTAG TTATCAGACT CAGACTAATT |
| 23601 | CTCCTCGGCG GGCACGTAGT GTAGCTAGTC AATCCATCAT |
| 23641 | TGCCTACACT ATGTCACTTG GTGCAGAAAA TTCAGTTGCT |
| 23681 | TACTCTAATA ACTCTATTGC CATACCCACA AATTTTACTA |
| 23721 | TTAGTGTTAC CACAGAAATT CTACCAGTGT CTATGACCAA |
| 23761 | GACATCAGTA GATTGTACAA TGTACATTTG TGGTGATTCA |
| 23801 | ACTGAATGCA GCAATCTTTT GTTGCAATAT GGCAGTTTTT |
| 23841 | GTACACAATT AAACCGTGCT TTAACTGGAA TAGCTGTTGA |
| 23881 | ACAAGACAAA AACACCCAAG AAGTTTTTGC ACAAGTCAAA |
| 23921 | CAAATTTACA AAACACCACC AATTAAAGAT TTTGGTGGTT |
| 23961 | TTAATTTTTC ACAAATATTA CCAGATCCAT CAAAACCAAG |
| 24001 | CAAGAGGTCA TTTATTGAAG ATCTACTTTT CAACAAAGTG |
| 24041 | ACACTTGCAG ATGCTGGCTT CATCAAACAA TATGGTGATT |
| 24081 | GCCTTGGTGA TATTGCTGCT AGAGACCTCA TTTGTGCACA |
| 24121 | AAAGTTTAAC GGCCTTACTG TTTTGCCACC TTTGCTCACA |
| 24161 | GATGAAATGA TTGCTCAATA CACTTCTGCA CTGTTAGCGG |
| 24201 | GTACAATCAC TTCTGGTTGG ACCTTTGGTG CAGGTGCTGC |
| 24241 | ATTACAAATA CCATTTGCTA TGCAAATGGC TTATAGGTTT |
| 24281 | AATGGTATTG GAGTTACACA GAATGTTCTC TATGAGAACC |
| 24321 | AAAAATTGAT TGCCAACCAA TTTAATAGTG CTATTGGCAA |
| 24361 | AATTCAAGAC TCACTTTCTT CCACAGCAAG TGCACTTGGA |
| 24401 | AAACTTCAAG ATGTGGTCAA CCAAAATGCA CAAGCTTTAA |
| 24441 | ACACGCTTGT TAAACAACTT AGCTCCAATT TTGGTGCAAT |
| 24481 | TTCAAGTGTT TTAAATGATA TCCTTTCACG TCTTGACAAA |
| 24521 | GTTGAGGCTG AAGTGCAAAT TGATAGGTTG ATCACAGGCA |
| 24561 | GACTTCAAAG TTTGCAGACA TATGTGACTC AACAATTAAT |
| 24601 | TAGAGCTGCA GAAATCAGAG CTTCTGCTAA TCTTGCTGCT |
| 24641 | ACTAAAATGT CAGAGTGTGT ACTTGGACAA TCAAAAAGAG |
| 24681 | TTGATTTTTG TGGAAAGGGC TATCATCTTA TGTCCTTCCC |
| 24721 | TCAGTCAGCA CCTCATGGTG TAGTCTTCTT GCATGTGACT |
| 24761 | TATGTCCCTG CACAAGAAAA GAACTTCACA ACTGCTCCTG |
| 24801 | CCATTTGTCA TGATGGAAAA GCACACTTTC CTCGTGAAGG |
| 24841 | TGTCTTTGTT TCAAATGGCA CACACTGGTT TGTAACACAA |
| 24881 | AGGAATTTTT ATGAACCACA AATCATTACT ACAGACAACA |
| 24921 | CATTTGTGTC TGGTAACTGT GATGTTGTAA TAGGAATTGT |
| 24961 | CAACAACACA GTTTATGATC CTTTGCAACC TGAATTAGAC |
| 25001 | TCATTCAAGG AGGAGTTAGA TAAATATTTT AAGAATCATA |
| 25041 | CATCACCAGA TGTTGATTTA GGTGACATCT CTGGCATTAA |
| 25081 | TGCTTCAGTT GTAAACATTC AAAAAGAAAT TGACCGCCTC |
| 25121 | AATGAGGTTG CCAAGAATTT AAATGAATCT CTCATCGATC |
| 25161 | TCCAAGAACT TGGAAAGTAT GAGCAGTATA TAAAATGGCC |
| 25201 | ATGGTACATT TGGCTAGGTT TTATAGCTGG CTTGATTGCC |
| 25241 | ATAGTAATGG TGACAATTAT GCTTTGCTGT ATGACCAGTT |
| 25281 | GCTGTAGTTG TCTCAAGGGC TGTTGTTCTT GTGGATCCTG |
| 25321 | CTGCAAATTT GATGAAGACG ACTCTGAGCC AGTGCTCAAA |
| 25361 | GGAGTCAAAT TACATTACAC ATAA |

The spike (S) protein encoded by this nucleic acid sequence has the following amino acid sequence (SEQ ID NO:5, shown below).

| | |
|---|---|
| 1 | MFVFLVLLPL VSSQCVNLTT RTQLPPAYTN SFTRGVYYPD |
| 41 | KVFRSSVLHS TQDLFLPFFS NVTWFHAIHV SGINGTKRED |
| 81 | NPVLPENDGV YFASTEKSNI IRGWIFGTTL DSKTQSLLIV |
| 121 | NNATNVVIKV CEFQFCNDPF LGVYYHKNNK SWMESEFRVY |
| 161 | SSANNCTFEY VSQPFLMDLE GKQGNEKNLR EFVEKNIDGY |
| 201 | FKIYSKHTPI NLVRDLPQGF SALEPLVDLP IGINITRFQT |
| 241 | LLALHRSYLT PGDSSSGWTA GAAAYYVGYL QPRTELLKYN |
| 281 | ENGTITDAVD CALDPLSETK CTLKSFTVEK GIYQTSNERV |
| 321 | QPTESIVRFP NITNLCPFGE VENATRFASV YAWNRKRISN |
| 361 | CVADYSVLYN SASFSTFKCY GVSPTKLNDL CFTNVYADSF |
| 401 | VIRGDEVRQI APGQTGKIAD YNYKLPDDET GCVIAWNSNN |
| 441 | LDSKVGGNYN YLYRLFRKSN LKPFERDIST EIYQAGSTPC |
| 481 | NGVEGENCYF PLQSYGFQPT NGVGYQPYRV VVLSFELLHA |
| 521 | PATVCGPKKS TNLVKNKCVN FNENGLIGTG VLTESNKKEL |
| 561 | PFQQFGRDIA DTTDAVRDPQ TLEILDITPC SFGGVSVITP |
| 601 | GINTSNQVAV LYQDVNCTEV PVAIHADQLT PTWRVYSTGS |
| 641 | NVFQTRAGCL IGAEHVNNSY ECDIPIGAGI CASYQTQTNS |
| 681 | PRRARSVASQ SIIAYTMSLG AENSVAYSNN SIAIPTNFTI |
| 721 | SVTTEILPVS MTKTSVDCTM YICGDSTECS NLLLQYGSFC |
| 761 | TQLNRALTGI AVEQDKNTQE VEAQVKQIYK TPPIKDEGGE |
| 801 | NFSQILPDPS KPSKRSFIED LLENKVTLAD AGFIKQYGDC |
| 841 | LGDIAARDLI CAQKENGLTV LPPLLTDEMI AQYTSALLAG |
| 881 | TITSGWTFGA GAALQIPFAM QMAYRENGIG VTQNVLYENQ |
| 921 | KLIANQFNSA IGKIQDSLSS TASALGKLQD VVNQNAQALN |
| 961 | TLVKQLSSNF GAISSVLNDI LSRLDKVEAE VQIDRLITGR |
| 1001 | LQSLQTYVTQ QLIRAAEIRA SANLAATKMS ECVLGQSKRV |
| 1041 | DFCGKGYHLM SFPQSAPHGV VFLHVTYVPA QEKNETTAPA |
| 1081 | ICHDGKAHFP REGVFVSNGT HWFVTQRNFY EPQIITTDNT |
| 1121 | FVSGNCDVVI GIVNNTVYDP LQPELDSFKE ELDKYFKNHT |
| 1161 | SPDVDLGDIS GINASVVNIQ KEIDRLNEVA KNLNESLIDL |
| 1201 | QELGKYEQYI KWPWYIWLGF IAGLIAIVMV TIMLCCMTSC |
| 1241 | CSCLKGCCSC GSCCKFDEDD SEPVLKGVKL HYT |

The example of a SARS-CoV-2 viral sequence provided herein as SEQ ID NO:1 includes an open reading frame at about positions 26523-27191 that encodes an M protein (ORF5); this M protein encoding nucleic acid is shown below as SEQ ID NO:6.

| | |
|---|---|
| 26523 | ATGGCAGA TTCCAACGGT ACTATTACCG TTGAAGAGCT |
| 26561 | TAAAAAGCTC CTTGAACAAT GGAACCTAGT AATAGGTTTC |
| 26601 | CTATTCCTTA CATGGATTTG TCTTCTACAA TTTGCCTATG |
| 26641 | CCAACAGGAA TAGGTTTTTG TATATAATTA AGTTAATTTT |
| 26681 | CCTCTGGCTG TTATGGCCAG TAACTTTAGC TTGTTTTGTG |
| 26721 | CTTGCTGCTG TTTACAGAAT AAATTGGATC ACCGGTGGAA |
| 26761 | TTGCTATCGC AATGGCTTGT CTTGTAGGCT TGATGTGGCT |
| 26801 | CAGCTACTTC ATTGCTTCTT TCAGACTGTT TGCGCGTACG |
| 26841 | CGTTCCATGT GGTCATTCAA TCCAGAAACT AACATTCTTC |
| 26881 | TCAACGTGCC ACTCCATGGC ACTATTCTGA CCAGACCGCT |
| 26921 | TCTAGAAAGT GAACTCGTAA TCGGAGCTGT GATCCTTCGT |
| 26961 | GGACATCTTC GTATTGCTGG ACACCATCTA GGACGCTGTG |
| 27001 | ACATCAAGGA CCTGCCTAAA GAAATCACTG TTGCTACATC |
| 27041 | ACGAACGCTT TCTTATTACA AATTGGGAGC TTCGCAGCGT |
| 27081 | GTAGCAGGTG ACTCAGGTTT TGCTGCATAC AGTCGCTACA |
| 27121 | GGATTGGCAA CTATAAATTA AACACAGACC ATTCCAGTAG |
| 27161 | CAGTGACAAT ATTGCTTTGC TTGTACAGTA A |

The open reading frame at about positions 27202-27191 of SEQ ID NO:1 encodes an M protein (ORF5) shown below as SEQ ID NO:7.

| | |
|---|---|
| 1 | MADSNGTITV EELKKLLEQW NLVIGFLFLT WICLLQFAYA |
| 41 | NRNRFLYIIK LIFLWLLWPV TLACFVLAAV YRINWITGGI |
| 81 | AIAMACLVGL MWLSYFIASF RLFARTRSMW SENPETNILL |
| 121 | NVPLHGTILT RPLLESELVI GAVILRGHLR IAGHHLGRCD |
| 161 | IKDLPKEITV ATSRTLSYYK LGASQRVAGD SGFAAYSRYR |
| 201 | IGNYKLNTDH SSSSDNIALL VQ |

Cells expressing the SARS-CoV-2 packaging signal sequence linked to a detectable signal protein coding region, as well as the SARS-CoV-2 spike (S) protein, membrane (M) protein, envelope (E) protein, and nucleocapsid (N) protein should also express angiotensin converting enzyme 2 (ACE2) receptor, and Transmembrane Serine Protease 2 (encoded by the TMPRSS2 gene). The ACE2 receptor acts as a receptor for the SARS-CoV-2 spike (S) protein, while TMPRSS2 protein cleaves the spike protein, facilitating viral entry and viral activation. Both the ACE2 receptor and the TMPRSS2 protein also facilitate entry and production of the SARS-CoV-2 virus-like particles described herein.

Cells can be selected for use that endogenously express ACE2 receptors and TMPRSS2 proteins. Alternatively, cells can be engineered to express the ACE2 receptor and TMPRSS2 proteins.

Humans can express different isoforms and variants of ACE2 receptors. For example, there are at least six human ACE2 receptor isoform sequences provided in the NCBI database (accession nos. NP_001358344.1, NP_068576.1, NP_001373188.1, NP_001373189.1, NP_001375381.1, and NP_001376331.1). The cells described herein can express any of these ACE2 receptor isoforms.

One example of a human ACE2 receptor sequence has NCBI accession no. NP_001358344.1, shown below as SEQ ID NO:8.

| | |
|---|---|
| 1 | MSSSSWILLS LVAVTAAQST IEEQAKTFLD KENHEAEDLE |
| 41 | YQSSLASWNY NINITEENVQ NMNNAGDKWS AFLKEQSTLA |
| 81 | QMYPLQEIQN LTVKLQLQAL QQNGSSVLSE DKSKRINTIL |
| 121 | NTMSTIYSTG KVCNPDNPQE CLLLEPGLNE IMANSLDYNE |
| 161 | RLWAWESWRS EVGKQLRPLY EEYVVLKNEM ARANHYEDYG |
| 201 | DYWRGDYEVN GVDGYDYSRG QLIEDVEHTF EEIKPLYEHL |
| 241 | HAYVRAKLMN AYPSYISPIG CLPAHLLGDM WGREWINLYS |
| 281 | LTVPFGQKPN IDVTDAMVDQ AWDAQRIFKE AEKFFVSVGL |
| 321 | PNMTQGFWEN SMLTDPGNVQ KAVCHPTAWD LGKGDERILM |
| 361 | CTKVTMDDEL TAHHEMGHIQ YDMAYAAQPF LLRNGANEGF |
| 401 | HEAVGEIMSL SAATPKHLKS IGLLSPDFQE DNETEINFLL |
| 441 | KQALTIVGTL PFTYMLEKWR WMVFKGEIPK DQWMKKWWEM |
| 481 | KREIVGVVEP VPHDETYCDP ASLFHVSNDY SFIRYYTRTL |
| 521 | YQFQFQEALC QAAKHEGPLH KCDISNSTEA GQKLFNMLRL |
| 561 | GKSEPWTLAL ENVVGAKNMN VRPLLNYFEP LFTWLKDQNK |
| 601 | NSFVGWSTDW SPYADQSIKV RISLKSALGD KAYEWNDNEM |
| 641 | YLFRSSVAYA MRQYFLKVKN QMILFGEEDV RVANLKPRIS |
| 681 | FNFFVTAPKN VSDIIPRTEV EKAIRMSRSR INDAFRLNDN |
| 721 | SLEFLGIQPT LGPPNQPPVS IWLIVFGVVM GVIVVGIVIL |
| 761 | IFTGIRDRKK KNKARSGENP YASIDISKGE NNPGFQNTDD |
| 801 | VQTSE |

A nucleic acid (cDNA) that encodes the foregoing ACE2 receptor protein is available as NCBI accession no. NM_001371415.1, shown below as SEQ ID NO:9.

| | |
|---|---|
| 1 | AGTCTAGGGA AAGTCATTCA GTGGATGTGA TCTTGGCTCA |
| 41 | CAGGGGACGA TGTCAAGCTC TTCCTGGCTC CTTCTCAGCC |
| 81 | TTGTTGCTGT AACTGCTGCT CAGTCCACCA TTGAGGAACA |
| 121 | GGCCAAGACA TTTTTGGACA AGTTTAACCA CGAAGCCGAA |
| 161 | GACCTGTTCT ATCAAAGTTC ACTTGCTTCT TGGAATTATA |
| 201 | ACACCAATAT TACTGAAGAG AATGTCCAAA ACATGAATAA |
| 241 | TGCTGGGGAC AAATGGTCTG CCTTTTTAAA GGAACAGTCC |
| 281 | ACACTTGCCC AAATGTATCC ACTACAAGAA ATTCAGAATC |
| 321 | TCACAGTCAA GCTTCAGCTG CAGGCTCTTC AGCAAAATGG |
| 361 | GTCTTCAGTG CTCTCAGAAG ACAAGAGCAA ACGGTTGAAC |
| 401 | ACAATTCTAA ATACAATGAG CACCATCTAC AGTACTGGAA |
| 441 | AAGTTTGTAA CCCAGATAAT CCACAAGAAT GCTTATTACT |
| 481 | TGAACCAGGT TTGAATGAAA TAATGGCAAA CAGTTTAGAC |
| 521 | TACAATGAGA GGCTCTGGGC TTGGGAAAGC TGGAGATCTG |
| 561 | AGGTCGGCAA GCAGCTGAGG CCATTATATG AAGAGTATGT |
| 601 | GGTCTTGAAA AATGAGATGG CAAGAGCAAA TCATTATGAG |
| 641 | GACTATGGGG ATTATTGGAG AGGAGACTAT GAAGTAAATG |
| 681 | GGGTAGATGG CTATGACTAC AGCCGCGGCC AGTTGATTGA |
| 721 | AGATGTGGAA CATACCTTTG AAGAGATTAA ACCATTATAT |
| 761 | GAACATCTTC ATGCCTATGT GAGGGCAAAG TTGATGAATG |
| 801 | CCTATCCTTC CTATATCAGT CCAATTGGAT GCCTCCCTGC |
| 841 | TCATTTGCTT GGTGATATGT GGGGTAGATT TTGGACAAAT |
| 881 | CTGTACTCTT TGACAGTTCC CTTTGGACAG AAACCAAACA |
| 921 | TAGATGTTAC TGATGCAATG GTGGACCAGG CCTGGGATGC |
| 961 | ACAGAGAATA TTCAAGGAGG CCGAGAAGTT CTTTGTATCT |
| 1001 | GTTGGTCTTC CTAATATGAC TCAAGGATTC TGGGAAAATT |
| 1041 | CCATGCTAAC GGACCCAGGA AATGTTCAGA AAGCAGTCTG |
| 1081 | CCATCCCACA GCTTGGGACC TGGGGAAGGG CGACTTCAGG |
| 1121 | ATCCTTATGT GCACAAAGGT GACAATGGAC GACTTCCTGA |
| 1161 | CAGCTCATCA TGAGATGGGG CATATCCAGT ATGATATGGC |
| 1201 | ATATGCTGCA CAACCTTTTC TGCTAAGAAA TGGAGCTAAT |
| 1241 | GAAGGATTCC ATGAAGCTGT TGGGGAAATC ATGTCACTTT |
| 1281 | CTGCAGCCAC ACCTAAGCAT TTAAAATCCA TTGGTCTTCT |
| 1321 | GTCACCCGAT TTTCAAGAAG ACAATGAAAC AGAAATAAAC |
| 1361 | TTCCTGCTCA AACAAGCACT CACGATTGTT GGGACTCTGC |
| 1401 | CATTTACTTA CATGTTAGAG AAGTGGAGGT GGATGGTCTT |
| 1441 | TAAAGGGGAA ATTCCCAAAG ACCAGTGGAT GAAAAAGTGG |
| 1481 | TGGGAGATGA AGCGAGAGAT AGTTGGGGTG GTGGAACCTG |
| 1521 | TGCCCCATGA TGAAACATAC TGTGACCCCG CATCTCTGTT |
| 1561 | CCATGTTTCT AATGATTACT CATTCATTCG ATATTACACA |
| 1601 | AGGACCCTTT ACCAATTCCA GTTTCAAGAA GCACTTTGTC |
| 1641 | AAGCAGCTAA ACATGAAGGC CCTCTGCACA AATGTGACAT |
| 1681 | CTCAAACTCT ACAGAAGCTG GACAGAAACT GTTCAATATG |
| 1721 | CTGAGGCTTG GAAAATCAGA ACCCTGGACC CTAGCATTGG |
| 1761 | AAAATGTTGT AGGAGCAAAG AACATGAATG TAAGGCCACT |
| 1801 | GCTCAACTAC TTTGAGCCCT TATTTACCTG GCTGAAAGAC |
| 1841 | CAGAACAAGA ATTCTTTTGT GGGATGGAGT ACCGACTGGA |
| 1881 | GTCCATATGC AGACCAAAGC ATCAAAGTGA GGATAAGCCT |
| 1921 | AAAATCAGCT CTTGGAGATA AAGCATATGA ATGGAACGAC |
| 1961 | AATGAAATGT ACCTGTTCCG ATCATCTGTT GCATATGCTA |
| 2001 | TGAGGCAGTA CTTTTTAAAA GTAAAAAATC AGATGATTCT |
| 2041 | TTTTGGGGAG GAGGATGTGC GAGTGGCTAA TTTGAAACCA |
| 2081 | AGAATCTCCT TTAATTTCTT TGTCACTGCA CCTAAAAATG |
| 2121 | TGTCTGATAT CATTCCTAGA ACTGAAGTTG AAAAGGCCAT |
| 2161 | CAGGATGTCC CGGAGCCGTA TCAATGATGC TTTCCGTCTG |
| 2201 | AATGACAACA GCCTAGAGTT TCTGGGGATA CAGCCAACAC |
| 2241 | TTGGACCTCC TAACCAGCCC CCTGTTTCCA TATGGCTGAT |
| 2281 | TGTTTTTGGA GTTGTGATGG GAGTGATAGT GGTTGGCATT |
| 2321 | GTCATCCTGA TCTTCACTGG GATCAGAGAT CGGAAGAAGA |
| 2361 | AAAATAAAGC AAGAAGTGGA GAAAATCCTT ATGCCTCCAT |
| 2401 | CGATATTAGC AAAGGAGAAA ATAATCCAGG ATTCCAAAAC |
| 2441 | ACTGATGATG TTCAGACCTC CTTTTAGAAA AATCTATGTT |
| 2481 | TTTCCTCTTG AGGTGATTTT GTTGTATGTA AATGTTAATT |
| 2521 | TCATGGTATA GAAAATATAA GATGATAAAG ATATCATTAA |
| 2561 | ATGTCAAAAC TATGACTCTG TTCAGAAAAA AAATTGTCCA |
| 2601 | AAGACAACAT GGCCAAGGAG AGAGCATCTT CATTGACATT |
| 2641 | GCTTTCAGTA TTTATTTCTG TCTCTGGATT TGACTTCTGT |
| 2681 | TCTGTTTCTT AATAAGGATT TTGTATTAGA GTATATTAGG |
| 2721 | GAAAGTGTGT ATTTGGTCTC ACAGGCTGTT CAGGGATAAT |
| 2761 | CTAAATGTAA ATGTCTGTTG AATTTCTGAA GTTGAAAACA |
| 2801 | AGGATATATC ATTGGAGCAA GTGTTGGATC TTGTATGGAA |
| 2841 | TATGGATGGA TCACTTGTAA GGACAGTGCC TGGGAACTGG |
| 2881 | TGTAGCTGCA AGGATTGAGA ATGGCATGCA TTAGCTCACT |
| 2921 | TTCATTTAAT CCATTGTCAA GGATGACATG CTTTCTTCAC |
| 2961 | AGTAACTCAG TTCAAGTACT ATGGTGATTT GCCTACAGTG |
| 3001 | ATGTTTGGAA TCGATCATGC TTTCTTCAAG GTGACAGGTC |
| 3041 | TAAAGAGAGA AGAATCCAGG GAACAGGTAG AGGACATTGC |
| 3081 | TTTTTCACTT CCAAGGTGCT TGATCAACAT CTCCCTGACA |
| 3121 | ACACAAAACT AGAGCCAGGG GCCTCCGTGA ACTCCCAGAG |
| 3161 | CATGCCTGAT AGAAACTCAT TTCTACTGTT CTCTAACTGT |
| 3201 | GGAGTGAATG GAAATTCCAA CTGTATGTTC ACCCTCTGAA |
| 3241 | GTGGGTACCC AGTCTCTTAA ATCTTTTGTA TTTGCTCACA |
| 3281 | GTGTTTGAGC AGTGCTGAGC ACAAAGCAGA CACTCAATAA |
| 3321 | ATGCTAGATT TACACACTC |

Similarly, humans can express different isoforms and variants of TMPRSS2. For example, there are at least three human TMPRSS2 protein sequence isoforms provided in the NCBI database (accession nos. NP_005647.3, NP_001128571.1, and NP_001369649.1). The cells described herein can express any of these TMPRSS2 isoforms.

One example of a human TMPRSS2 sequence has NCBI accession no. NP_005647.3, shown below as SEQ ID NO:10.

| | |
|---|---|
| 1 | MALNSGSPPA IGPYYENHGY QPENPYPAQP TVVPTVYEVH |
| 41 | PAQYYPSPVP QYAPRVLTQA SNPVVCTQPK SPSGTVCTSK |
| 81 | TKKALCITLT LGTFLVGAAL AAGLLWKEMG SKCSNSGIEC |
| 121 | DSSGTCINPS NWCDGVSHCP GGEDENRCVR LYGPNFILQV |
| 161 | YSSQRKSWHP VCQDDWNENY GRAACRDMGY KNNFYSSQGI |
| 201 | VDDSGSTSFM KLNTSAGNVD IYKKLYHSDA CSSKAVVSLR |
| 241 | CIACGVNINS SRQSRIVGGE SALPGAWPWQ VSLHVQNVHV |
| 281 | CGGSIITPEW IVTAAHCVEK PLNNPWHWTA FAGILRQSEM |
| 321 | FYGAGYQVEK VISHPNYDSK TKNNDIALMK LQKPLTENDL |
| 361 | VKPVCLPNPG MMLQPEQLCW ISGWGATEEK GKTSEVLNAA |
| 401 | KVLLIETQRC NSRYVYDNLI TPAMICAGEL QGNVDSCQGD |
| 441 | SGGPLVTSKN NIWWLIGDTS WGSGCAKAYR PGVYGNVMVE |
| 481 | TDWIYRQMRA DG |

A nucleic acid (cDNA) that encodes the foregoing TMPRSS2 protein is available as NCBI accession no. NM_005656.4, shown below as SEQ ID NO:11.

| | |
|---|---|
| 1 | GAGTAGGCGC GAGCTAAGCA GGAGGCGGAG GCGGAGGCGG |
| 41 | AGGGCGAGGG GCGGGGAGCG CCGCCTGGAG CGCGGCAGGT |
| 81 | CATATTGAAC ATTCCAGATA CCTATCATTA CTCGATGCTG |
| 121 | TTGATAACAG CAAGATGGCT TTGAACTCAG GGTCACCACC |
| 161 | AGCTATTGGA CCTTACTATG AAAACCATGG ATACCAACCG |
| 201 | GAAAACCCCT ATCCCGCACA GCCCACTGTG GTCCCCACTG |
| 241 | TCTACGAGGT GCATCCGGCT CAGTACTACC CGTCCCCCGT |
| 281 | GCCCCAGTAC GCCCCGAGGG TCCTGACGCA GGCTTCCAAC |
| 321 | CCCGTCGTCT GCACGCAGCC CAAATCCCCA TCCGGGACAG |
| 361 | TGTGCACCTC AAAGACTAAG AAAGCACTGT GCATCACCTT |
| 401 | GACCCTGGGG ACCTTCCTCG TGGGAGCTGC GCTGGCCGCT |
| 441 | GGCCTACTCT GGAAGTTCAT GGGCAGCAAG TGCTCCAACT |
| 481 | CTGGGATAGA GTGCGACTCC TCAGGTACCT GCATCAACCC |
| 521 | CTCTAACTGG TGTGATGGCG TGTCACACTG CCCCGGGGGG |
| 561 | GAGGACGAGA ATCGGTGTGT TCGCCTCTAC GGACCAAACT |
| 601 | TCATCCTTCA GGTGTACTCA TCTCAGAGGA AGTCCTGGCA |
| 641 | CCCTGTGTGC CAAGACGACT GGAACGAGAA CTACGGGCGG |
| 681 | GCGGCCTGCA GGGACATGGG CTATAAGAAT AATTTTTACT |
| 721 | CTAGCCAAGG AATAGTGGAT GACAGCGGAT CCACCAGCTT |
| 761 | TATGAAACTG AACACAAGTG CCGGCAATGT CGATATCTAT |
| 801 | AAAAAACTGT ACCACAGTGA TGCCTGTTCT TCAAAAGCAG |
| 841 | TGGTTTCTTT ACGCTGTATA GCCTGCGGGG TCAACTTGAA |
| 881 | CTCAAGCCGC CAGAGCAGGA TTGTGGGCGG CGAGAGCGCG |
| 921 | CTCCCGGGGG CCTGGCCCTG GCAGGTCAGC CTGCACGTCC |
| 961 | AGAACGTCCA CGTGTGCGGA GGCTCCATCA TCACCCCCGA |
| 1001 | GTGGATCGTG ACAGCCGCCC ACTGCGTGGA AAAACCTCTT |
| 1041 | AACAATCCAT GGCATTGGAC GGCATTTGCG GGGATTTTGA |
| 1081 | GACAATCTTT CATGTTCTAT GGAGCCGGAT ACCAAGTAGA |
| 1121 | AAAAGTGATT TCTCATCCAA ATTATGACTC CAAGACCAAG |
| 1161 | AACAATGACA TTGCGCTGAT GAAGCTGCAG AAGCCTCTGA |
| 1201 | CTTTCAACGA CCTAGTGAAA CCAGTGTGTC TGCCCAACCC |
| 1241 | AGGCATGATG CTGCAGCCAG AACAGCTCTG CTGGATTTCC |
| 1281 | GGGTGGGGGG CCACCGAGGA GAAAGGGAAG ACCTCAGAAG |
| 1321 | TGCTGAACGC TGCCAAGGTG CTTCTCATTG AGACACAGAG |
| 1361 | ATGCAACAGC AGATATGTCT ATGACAACCT GATCACACCA |
| 1401 | GCCATGATCT GTGCCGGCTT CCTGCAGGGG AACGTCGATT |
| 1441 | CTTGCCAGGG TGACAGTGGA GGGCCTCTGG TCACTTCGAA |
| 1481 | GAACAATATC TGGTGGCTGA TAGGGGATAC AAGCTGGGGT |
| 1521 | TCTGGCTGTG CCAAAGCTTA CAGACCAGGA GTGTACGGGA |
| 1561 | ATGTGATGGT ATTCACGGAC TGGATTTATC GACAAATGAG |
| 1601 | GGCAGACGGC TAATCCACAT GGTCTTCGTC CTTGACGTCG |
| 1641 | TTTTACAAGA AAACAATGGG GCTGGTTTTG CTTCCCCGTG |
| 1681 | CATGATTTAC TCTTAGAGAT GATTCAGAGG TCACTTCATT |
| 1721 | TTTATTAAAC AGTGAACTTG TCTGGCTTTG GCACTCTCTG |
| 1761 | CCATTCTGTG CAGGCTGCAG TGGCTCCCCT GCCCAGCCTG |
| 1801 | CTCTCCCTAA CCCCTTGTCC GCAAGGGGTG ATGGCCGGCT |
| 1841 | GGTTGTGGGC ACTGGCGGTC AAGTGTGGAG GAGAGGGGTG |
| 1881 | GAGGCTGCCC CATTGAGATC TTCCTGCTGA GTCCTTTCCA |
| 1921 | GGGGCCAATT TTGGATGAGC ATGGAGCTGT CACCTCTCAG |
| 1961 | CTGCTGGATG ACTTGAGATG AAAAAGGAGA GACATGGAAA |
| 2001 | GGGAGACAGC CAGGTGGCAC CTGCAGCGGC TGCCCTCTGG |
| 2041 | GGCCACTTGG TAGTGTCCCC AGCCTACCTC TCCACAAGGG |
| 2081 | GATTTTGCTG ATGGGTTCTT AGAGCCTTAG CAGCCCTGGA |
| 2121 | TGGTGGCCAG AAATAAAGGG ACCAGCCCTT CATGGGTGGT |
| 2161 | GACGTGGTAG TCACTTGTAA GGGGAACAGA AACATTTTTG |
| 2201 | TTCTTATGGG GTGAGAATAT AGACAGTGCC CTTGGTGCGA |
| 2241 | GGGAAGCAAT TGAAAAGGAA CTTGCCCTGA GCACTCCTGG |
| 2281 | TGCAGGTCTC CACCTGCACA TTGGGTGGGG CTCCTGGGAG |
| 2321 | GGAGACTCAG CCTTCCTCCT CATCCTCCCT GACCCTGCTC |
| 2361 | CTAGCACCCT GGAGAGTGCA CATGCCCCTT GGTCCTGGCA |
| 2401 | GGGCGCCAAG TCTGGCACCA TGTTGGCCTC TTCAGGCCTG |
| 2441 | CTAGTCACTG GAAATTGAGG TCCATGGGGG AAATCAAGGA |
| 2481 | TGCTCAGTTT AAGGTACACT GTTTCCATGT TATGTTTCTA |
| 2521 | CACATTGCTA CCTCAGTGCT CCTGGAAACT TAGCTTTTGA |
| 2561 | TGTCTCCAAG TAGTCCACCT TCATTTAACT CTTTGAAACT |
| 2601 | GTATCATCTT TGCCAAGTAA GAGTGGTGGC CTATTTCAGC |
| 2641 | TGCTTTGACA AAATGACTGG CTCCTGACTT AACGTTCTAT |
| 2681 | AAATGAATGT GCTGAAGCAA AGTGCCCATG GTGGCGGCGA |
| 2721 | AGAAGAGAAA GATGTGTTTT GTTTTGGACT CTCTGTGGTC |
| 2761 | CCTTCCAATG CTGTGGGTTT CCAACCAGGG GAAGGGTCCC |
| 2801 | TTTTGCATTG CCAAGTGCCA TAACCATGAG CACTACTCTA |
| 2841 | CCATGGTTCT GCCTCCTGGC CAAGCAGGCT GGTTTGCAAG |
| 2881 | AATGAAATGA ATGATTCTAC AGCTAGGACT TAACCTTGAA |
| 2921 | ATGGAAAGTC ATGCAATCCC ATTTGCAGGA TCTGTCTGTG |
| 2961 | CACATGCCTC TGTAGAGAGC AGCATTCCCA GGGACCTTGG |
| 3001 | AAACAGTTGG CACTGTAAGG TGCTTGCTCC CCAAGACACA |
| 3041 | TCCTAAAAGG TGTTGTAATG GTGAAAACGT CTTCCTTCTT |
| 3081 | TATTGCCCCT TCTTATTTAT GTGAACAACT GTTTGTCTTT |
| 3121 | TTTTGTATCT TTTTTAAACT GTAAAGTTCA ATTGTGAAAA |
| 3161 | TGAATATCAT GCAAATAAAT TATGCAATTT TTTTTTCAAA |
| 3201 | GTAACTACTG CATCTTTGAA GTTCTGCCTG GTGAGTAGGA |
| 3241 | CCAGCCTCCA TTTCCTTATA AGGGGGTGAT GTTGAGGCTG |
| 3281 | CTGGTCAGAG GACCAAAGGT GAGGCAAGGC CAGACTTGGT |
| 3321 | GCTCCTGTGG TTGGTGCCCT CAGTTCCTGC AGCCTGTCCT |
| 3361 | GTTGGAGAGG TCCCTCAAAT GACTCCTTCT TATTATTCTA |
| 3401 | TTAGTCTGTT TCCATGCTCC TAATAAAGAC ATACCCAAGA |
| 3441 | CTGCAATTTA |

### Expression Systems

Nucleic acid segments that include one or more of the SARS-CoV-2 packaging signal sequence-detectable signal protein coding regions, SARS-CoV-2 spike (S) coding regions, SARS-CoV-2 membrane (M) coding regions, SARS-CoV-2 envelope (E) coding regions, or SARS-CoV-2 nucleocapsid (N) coding regions can be inserted into or employed with any suitable expression system. In some cases, one or more cells express each of an encoded SARS-CoV-2 packaging signal sequence-detectable signal protein coding region, SARS-CoV-2 spike (S) coding region, SARS-CoV-2 membrane (M) coding region, SARS-CoV-2 envelope (E) coding region, and SARS-CoV-2 nucleocapsid (N) coding region.

Useful quantities of one or more of the SARS-CoV-2 packaging signal sequence-detectable signal protein coding regions, SARS-CoV-2 spike (S) coding regions, SARS-CoV-2 membrane (M) coding regions, SARS-CoV-2 envelope (E) coding regions, or SARS-CoV-2 nucleocapsid (N) coding regions can also be generated from such expression systems.

Recombinant expression of nucleic acids are usefully accomplished by incorporating the nucleic acids into a vector, such as a plasmid. The vector can include a promoter operably linked to nucleic acid segment encoding one or more of the SARS-CoV-2 packaging signal sequence-detectable signal protein coding regions, SARS-CoV-2 spike (S) coding regions, SARS-CoV-2 membrane (M) coding regions, SARS-CoV-2 envelope (E) coding regions, or SARS-CoV-2 nucleocapsid (N) coding regions. In some cases, expression of the SARS-CoV-2 packaging signal sequence-detectable signal protein coding regions, SARS-CoV-2 spike (S) coding regions, SARS-CoV-2 membrane (M) coding regions, SARS-CoV-2 envelope (E) coding regions, or SARS-CoV-2 nucleocapsid (N) coding regions are each driven by a separate promoter. In some cases, expression of one or more of the SARS-CoV-2 packaging signal sequence-detectable signal protein coding regions, SARS-CoV-2 spike (S) coding regions, SARS-CoV-2 membrane (M) coding regions, SARS-CoV-2 envelope (E) coding regions, or SARS-CoV-2 nucleocapsid (N) coding regions are each driven by the same promoter. However, it can be useful in some cases to modulate the expression of one or a few of the SARS-CoV-2 spike (S) coding regions, SARS-CoV-2 membrane (M) coding regions, SARS-CoV-2 envelope (E) coding regions, or SARS-CoV-2 nucleocapsid (N) coding regions relative to the others.

The expression cassette, expression vector, and sequences incorporated into the cassette or vector can be heterologous. As used herein, the term “heterologous” when used in reference to an expression cassette, expression vector, regulatory sequence, promoter, or nucleic acid refers to an expression cassette, expression vector, regulatory sequence, or nucleic acid that has been manipulated in some way. For example, a heterologous promoter can be a promoter that is not naturally linked to a nucleic acid of interest, or that has been introduced into cells by cell transformation procedures. A heterologous nucleic acid or promoter also includes a nucleic acid or promoter that is native to a virus or an organism but that has been altered in some way (e.g., placed within an expression vector or expression cassette, placed in a different chromosomal location, mutated, added in multiple copies, linked to a non-native promoter or enhancer sequence, etc.). Heterologous nucleic acids may comprise sequences that comprise cDNA forms. Heterologous coding regions can be distinguished from endogenous coding regions, for example, when the heterologous coding regions are joined to nucleotide sequences comprising regulatory elements such as promoters that are not found naturally associated with the coding region, or when the heterologous coding regions are associated with portions of a chromosome not found in nature (e.g., genes expressed in loci where the protein encoded by the coding region is not normally expressed). Similarly, heterologous promoters can be promoters that at linked to a coding region to which they are not linked in nature.

As used herein, an expression vector, or vector, refers to any carrier containing exogenous DNA. Thus, vectors are agents that transport the exogenous nucleic acid into a cell without degradation and include a promoter yielding expression of the nucleic acid in the cells into which it is delivered. Vectors include but are not limited to plasmids, viral nucleic acids, viruses, phage nucleic acids, phages, cosmids, and artificial chromosomes.

A variety of prokaryotic and eukaryotic expression vectors suitable for carrying, encoding and/or expressing the SARS-CoV-2 packaging signal sequence-detectable signal protein coding regions, SARS-CoV-2 spike (S) coding regions, SARS-CoV-2 membrane (M) coding regions, SARS-CoV-2 envelope (E) coding regions, or SARS-CoV-2 nucleocapsid (N) coding regions can be used. Such expression vectors include, for example, pET, pET3d, pCR2.1, pBAD, pUC, and yeast vectors. The vectors can be used, for example, in a variety of in vivo and in vitro situations.

Viral vectors that can be employed include those relating to lentivirus, adenovirus, adeno-associated virus, herpes virus, vaccinia virus, polio virus, AIDS virus, neuronal trophic virus, Sindbis and other viruses. Also useful are any viral families which share the properties of these viruses which make them suitable for use as vectors. Retroviral vectors that can be employed include those described in by Verma, I. M., Retroviral vectors for gene transfer. In Microbiology-1985, American Society for Microbiology, pp. 229-232, Washington, (1985). For example, such retroviral vectors can include Murine Maloney Leukemia virus, MMLV, and other retroviruses that express desirable properties. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral nucleic acid.

The vectors employed can include other elements required for transcription and translation. A variety of regulatory elements can be included in the expression cassettes and/or expression vectors, including promoters, enhancers, translational initiation sequences, internal ribosome entry sites, transcription termination sequences and other elements.

A “promoter” contains core elements required for basic interaction of RNA polymerase and transcription factors and can contain upstream elements and response elements. Promoters generally include one or more sequence segments of DNA that function when in a relatively fixed location in regard to the transcription start site. For example, the promoter can be upstream of the nucleic acid segment encoding one or more the SARS-CoV-2 packaging signal sequence-detectable signal protein coding regions, SARS-CoV-2 spike (S) coding regions, SARS-CoV-2 membrane (M) coding regions, SARS-CoV-2 envelope (E) coding regions, or SARS-CoV-2 nucleocapsid (N) coding regions, or a combination thereof. An internal ribosome entry site, abbreviated IRES, is an RNA sequence element that allows for translation initiation in cap-independent manner directly from an RNA, thereby allowing synthesis of a protein.

“Enhancer” generally refers to a sequence of DNA that functions at no fixed distance from the transcription start site and can be either 5′ or 3′ to the transcription unit. Furthermore, enhancers can be within an intron as well as within the coding sequence itself. They are usually between 10 and 300 by in length, and they function in cis. Enhancers function to increase transcription from nearby promoters.

Enhancers, like promoters, also often contain response elements that mediate the regulation of transcription. Enhancers often determine the regulation of expression. Expression vectors used in eukaryotic host cells (yeast, fungi, insect, plant, animal, human or nucleated cells) can also contain sequences for the termination of transcription, which can affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs.

The expression of the SARS-CoV-2 packaging signal sequence-detectable signal protein coding regions, SARS-CoV-2 spike (S) coding regions, SARS-CoV-2 membrane (M) coding regions, SARS-CoV-2 envelope (E) coding regions, or SARS-CoV-2 nucleocapsid (N) coding regions from one or more expression cassettes or expression vectors can be controlled by any promoter capable of expression in prokaryotic cells or eukaryotic cells. Examples of prokaryotic promoters that can be used include, but are not limited to, SP6, T7, T5, tac, bla, trp, gal, lac, or maltose promoters. Vectors for bacterial expression include pGEX-5X-3, and for eukaryotic expression include pCIneo-CMV.

Examples of eukaryotic promoters that can be used include, but are not limited to, constitutive promoters, e.g., viral promoters such as CMV, SV40 and RSV promoters, as well as regulatable promoters, e.g., an inducible or repressible promoter such as the tet promoter, the hsp70 promoter and a synthetic promoter regulated by CRE. In some cases the 5′ or 3′ untranslated region of a virus (5′UTR or 3′UTR, respectively) includes a promoter, and such UTR regions can be used as promoters to drive expression. For example, a segment of a SARS-CoV-2 5′UTR or 3′UTR can be used as a promoter to drive one or more of the SARS-CoV-2 packaging signal sequence-detectable signal protein coding regions, SARS-CoV-2 spike (S) coding regions, SARS-CoV-2 membrane (M) coding regions, SARS-CoV-2 envelope (E) coding regions, or SARS-CoV-2 nucleocapsid (N) coding regions.

The expression cassettes or vectors can include nucleic acid sequence encoding a detectable signal protein or other marker product. Such a signal protein or marker product can be used to determine if one or more vectors or expression cassettes encoding the SARS-CoV-2 packaging signal sequence-detectable signal protein coding regions, SARS-CoV-2 spike (S) coding regions, SARS-CoV-2 membrane (M) coding regions, SARS-CoV-2 envelope (E) coding regions, or SARS-CoV-2 nucleocapsid (N) coding regions has been delivered to the cell, and once delivered, is being expressed.

Signal protein or marker genes can include the E. coli lacZ gene which encodes luciferase, aequorin, green fluorescent protein (GFP), EGFP, Emerald, Superfolder GFP, Azami Green, mWasabi, TagGFP, TurboGFP, AcGFP, ZsGreen, T-Sapphire, EBFP, EBFP2, Azurite, mTagBFP, ECFP, mECFP, Cerulean, mTurquoise, CyPet, AmCyan1, Midori-Ishi Cyan, TagCFP, mTFP1 (Teal), EYFP, Topaz, Venus, mCitrine, YPet, TagYFP, Phi YFP, ZsYellow1, mBanana, Kusabira Orange, Kusabira Orange2, mOrange, mOrange2, dTomato, dTomato-Tandem, TagRFP, TagRFP-T, DsRed, DsRed2, DsRed-Express (T1), DsRed-Monomer, mTangerine, mRuby, mApple, mStrawberry, AsRed2, mRFP1, JRed, mCherry, HcRed1, mRaspberry, dKeima-Tandem, HcRed-Tandem, mPlum, AQ143, β-galactosidase, or combinations thereof.

In some embodiments the marker can be a selectable marker. When such selectable markers are successfully transferred into a host cell, the transformed host cell can survive if placed under selective pressure. There are two widely used distinct categories of selective regimes. The first category is based on a cell's metabolism and the use of a mutant cell line which lacks the ability to grow independent of a supplemented media. The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin (Southern P. and Berg, P., J. Molec. Appl. Genet. 1:327 (1982)), mycophenolic acid, (Mulligan, R. C. and Berg, P. Science 209:1422 (1980)) or hygromycin, (Sugden, B. et al., Mol. Cell. Biol. 5:410-413 (1985)).

Gene transfer can be obtained using direct transfer of genetic material, in but not limited to, plasmids, viral vectors, viral nucleic acids, phage nucleic acids, phages, cosmids, and artificial chromosomes, or via transfer of genetic material in cells or carriers such as cationic liposomes. Such methods are available in the art and readily adaptable for use in the method described herein. Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)). Appropriate means for transfection, including viral vectors, chemical transfectants, or physico-mechanical methods such as use of polyethylenimine (PEI; a stable cationic polymer), electroporation and direct diffusion of DNA. Such methods are described by, for example, by Wolff, J. A., et al, Science, 247, 1465-1468, (1990), and Wolff, J. A. Nature, 352, 815-818, (1991).

For example, the nucleic acid molecules, expression cassette and/or vectors encoding the SARS-CoV-2 packaging signal sequence-detectable signal protein coding regions, SARS-CoV-2 spike (S) coding regions, SARS-CoV-2 membrane (M) coding regions, SARS-CoV-2 envelope (E) coding regions, or SARS-CoV-2 nucleocapsid (N) coding regions can be introduced to one or more cells by any method including, but not limited to, calcium-mediated transformation, electroporation, microinjection, lipofection, particle bombardment and the like. The cells can also be expanded in culture and the expression of the SARS-CoV-2 packaging signal sequence-detectable signal protein coding regions, SARS-CoV-2 spike (S) coding regions, SARS-CoV-2 membrane (M) coding regions, SARS-CoV-2 envelope (E) coding regions, and SARS-CoV-2 nucleocapsid (N) coding regions can be detected by a signal from the signal protein or the marker product.

Western blot, Northern blot, polymerase chain reaction and other available procedures can be used to detect and/or quantify expression of one or more of the individual RNA or protein products of a SARS-CoV-2 packaging signal sequence-detectable signal protein coding region, SARS-CoV-2 spike (S) coding region, SARS-CoV-2 membrane (M) coding region, SARS-CoV-2 envelope (E) coding region, or SARS-CoV-2 nucleocapsid (N) coding region.

One or more transgenic vectors or cells with one or more heterologous expression cassettes or expression vectors can express the encoded SARS-CoV-2 packaging signal sequence-detectable signal protein coding regions, SARS-CoV-2 spike (S) proteins, SARS-CoV-2 membrane (M) proteins, SARS-CoV-2 envelope (E) proteins, and SARS-CoV-2 nucleocapsid (N) proteins. In some cases, one or more cells express each of an encoded SARS-CoV-2 packaging signal sequence-detectable signal protein coding region, SARS-CoV-2 spike (S) coding region, SARS-CoV-2 membrane (M) coding region, SARS-CoV-2 envelope (E) coding region, and SARS-CoV-2 nucleocapsid (N) coding region.

A transgenic cell can produce virus-like particles that include the SARS-CoV-2 packaging signal sequence-detectable signal protein coding region (e.g., as an RNA), SARS-CoV-2 spike (S) protein, SARS-CoV-2 membrane (M) protein, SARS-CoV-2 envelope (E) protein, and SARS-CoV-2 nucleocapsid (N) protein.

### SARS-CoV-2 Virus

The SARS-CoV-2 virus has a single-stranded RNA genome with about 29891 nucleotides, that encode about 9860 amino acids. A SARS-CoV-2 selected RNA genome can be copied and made into a DNA by reverse transcription and formation of a cDNA. A linear SARS-CoV-2 DNA can be circularized by ligation of SARS-CoV-2 DNA ends.

A DNA sequence for the SARS-CoV-2 genome, with coding regions, is available as accession number NC_045512.2 from the NCBI website and shown below as SEQ ID NO:1.

| | |
|---|---|
| 1 | ATTAAAGGTT TATACCTTCC CAGGTAACAA ACCAACCAAC |
| 41 | TTTCGATCTC TTGTAGATCT GTTCTCTAAA CGAACTTTAA |
| 81 | AATCTGTGTG GCTGTCACTC GGCTGCATGC TTAGTGCACT |
| 121 | CACGCAGTAT AATTAATAAC TAATTACTGT CGTTGACAGG |
| 161 | ACACGAGTAA CTCGTCTATC TTCTGCAGGC TGCTTACGGT |
| 201 | TTCGTCCGTG TTGCAGCCGA TCATCAGCAC ATCTAGGTTT |
| 241 | CGTCCGGGTG TGACCGAAAG GTAAGATGGA GAGCCTTGTC |
| 281 | CCTGGTTTCA ACGAGAAAAC ACACGTCCAA CTCAGTTTGC |
| 321 | CTGTTTTACA GGTTCGCGAC GTGCTCGTAC GTGGCTTTGG |
| 361 | AGACTCCGTG GAGGAGGTCT TATCAGAGGC ACGTCAACAT |
| 401 | CTTAAAGATG GCACTTGTGG CTTAGTAGAA GTTGAAAAAG |
| 441 | GCGTTTTGCC TCAACTTGAA CAGCCCTATG TGTTCATCAA |
| 481 | ACGTTCGGAT GCTCGAACTG CACCTCATGG TCATGTTATG |
| 521 | GTTGAGCTGG TAGCAGAACT CGAAGGCATT CAGTACGGTC |
| 561 | GTAGTGGTGA GACACTTGGT GTCCTTGTCC CTCATGTGGG |
| 601 | CGAAATACCA GTGGCTTACC GCAAGGTTCT TCTTCGTAAG |
| 641 | AACGGTAATA AAGGAGCTGG TGGCCATAGT TACGGCGCCG |
| 681 | ATCTAAAGTC ATTTGACTTA GGCGACGAGC TTGGCACTGA |
| 721 | TCCTTATGAA GATTTTCAAG AAAACTGGAA CACTAAACAT |
| 761 | AGCAGTGGTG TTACCCGTGA ACTCATGCGT GAGCTTAACG |
| 801 | GAGGGGCATA CACTCGCTAT GTCGATAACA ACTTCTGTGG |
| 841 | CCCTGATGGC TACCCTCTTG AGTGCATTAA AGACCTTCTA |
| 881 | GCACGTGCTG GTAAAGCTTC ATGCACTTTG TCCGAACAAC |
| 921 | TGGACTTTAT TGACACTAAG AGGGGTGTAT ACTGCTGCCG |
| 961 | TGAACATGAG CATGAAATTG CTTGGTACAC GGAACGTTCT |
| 1001 | GAAAAGAGCT ATGAATTGCA GACACCTTTT GAAATTAAAT |
| 1041 | TGGCAAAGAA ATTTGACACC TTCAATGGGG AATGTCCAAA |
| 1081 | TTTTGTATTT CCCTTAAATT CCATAATCAA GACTATTCAA |
| 1121 | CCAAGGGTTG AAAAGAAAAA GCTTGATGGC TTTATGGGTA |
| 1161 | GAATTCGATC TGTCTATCCA GTTGCGTCAC CAAATGAATG |
| 1201 | CAACCAAATG TGCCTTTCAA CTCTCATGAA GTGTGATCAT |
| 1241 | TGTGGTGAAA CTTCATGGCA GACGGGCGAT TTTGTTAAAG |
| 1281 | CCACTTGCGA ATTTTGTGGC ACTGAGAATT TGACTAAAGA |
| 1321 | AGGTGCCACT ACTTGTGGTT ACTTACCCCA AAATGCTGTT |
| 1361 | GTTAAAATTT ATTGTCCAGC ATGTCACAAT TCAGAAGTAG |
| 1401 | GACCTGAGCA TAGTCTTGCC GAATACCATA ATGAATCTGG |
| 1441 | CTTGAAAACC ATTCTTCGTA AGGGTGGTCG CACTATTGCC |
| 1481 | TTTGGAGGCT GTGTGTTCTC TTATGTTGGT TGCCATAACA |
| 1521 | AGTGTGCCTA TTGGGTTCCA CGTGCTAGCG CTAACATAGG |
| 1561 | TTGTAACCAT ACAGGTGTTG TTGGAGAAGG TTCCGAAGGT |
| 1601 | CTTAATGACA ACCTTCTTGA AATACTCCAA AAAGAGAAAG |
| 1641 | TCAACATCAA TATTGTTGGT GACTTTAAAC TTAATGAAGA |
| 1681 | GATCGCCATT ATTTTGGCAT CTTTTTCTGC TTCCACAAGT |
| 1721 | GCTTTTGTGG AAACTGTGAA AGGTTTGGAT TATAAAGCAT |
| 1761 | TCAAACAAAT TGTTGAATCC TGTGGTAATT TTAAAGTTAC |
| 1801 | AAAAGGAAAA GCTAAAAAAG GTGCCTGGAA TATTGGTGAA |
| 1841 | CAGAAATCAA TACTGAGTCC TCTTTATGCA TTTGCATCAG |
| 1881 | AGGCTGCTCG TGTTGTACGA TCAATTTTCT CCCGCACTCT |
| 1921 | TGAAACTGCT CAAAATTCTG TGCGTGTTTT ACAGAAGGCC |
| 1961 | GCTATAACAA TACTAGATGG AATTTCACAG TATTCACTGA |
| 2001 | GACTCATTGA TGCTATGATG TTCACATCTG ATTTGGCTAC |
| 2041 | TAACAATCTA GTTGTAATGG CCTACATTAC AGGTGGTGTT |
| 2081 | GTTCAGTTGA CTTCGCAGTG GCTAACTAAC ATCTTTGGCA |
| 2121 | CTGTTTATGA AAAACTCAAA CCCGTCCTTG ATTGGCTTGA |
| 2161 | AGAGAAGTTT AAGGAAGGTG TAGAGTTTCT TAGAGACGGT |
| 2201 | TGGGAAATTG TTAAATTTAT CTCAACCTGT GCTTGTGAAA |
| 2241 | TTGTCGGTGG ACAAATTGTC ACCTGTGCAA AGGAAATTAA |
| 2281 | GGAGAGTGTT CAGACATTCT TTAAGCTTGT AAATAAATTT |
| 2321 | TTGGCTTTGT GTGCTGACTC TATCATTATT GGTGGAGCTA |
| 2361 | AACTTAAAGC CTTGAATTTA GGTGAAACAT TTGTCACGCA |
| 2401 | CTCAAAGGGA TTGTACAGAA AGTGTGTTAA ATCCAGAGAA |
| 2441 | GAAACTGGCC TACTCATGCC TCTAAAAGCC CCAAAAGAAA |
| 2481 | TTATCTTCTT AGAGGGAGAA ACACTTCCCA CAGAAGTGTT |
| 2521 | AACAGAGGAA GTTGTCTTGA AAACTGGTGA TTTACAACCA |
| 2561 | TTAGAACAAC CTACTAGTGA AGCTGTTGAA GCTCCATTGG |
| 2601 | TTGGTACACC AGTTTGTATT AACGGGCTTA TGTTGCTCGA |
| 2641 | AATCAAAGAC ACAGAAAAGT ACTGTGCCCT TGCACCTAAT |
| 2681 | ATGATGGTAA CAAACAATAC CTTCACACTC AAAGGCGGTG |
| 2721 | CACCAACAAA GGTTACTTTT GGTGATGACA CTGTGATAGA |
| 2761 | AGTGCAAGGT TACAAGAGTG TGAATATCAC TTTTGAACTT |
| 2801 | GATGAAAGGA TTGATAAAGT ACTTAATGAG AAGTGCTCTG |
| 2841 | CCTATACAGT TGAACTCGGT ACAGAAGTAA ATGAGTTCGC |
| 2881 | CTGTGTTGTG GCAGATGCTG TCATAAAAAC TTTGCAACCA |
| 2921 | GTATCTGAAT TACTTACACC ACTGGGCATT GATTTAGATG |
| 2961 | AGTGGAGTAT GGCTACATAC TACTTATTTG ATGAGTCTGG |
| 3001 | TGAGTTTAAA TTGGCTTCAC ATATGTATTG TTCTTTCTAC |
| 3041 | CCTCCAGATG AGGATGAAGA AGAAGGTGAT TGTGAAGAAG |
| 3081 | AAGAGTTTGA GCCATCAACT CAATATGAGT ATGGTACTGA |
| 3121 | AGATGATTAC CAAGGTAAAC CTTTGGAATT TGGTGCCACT |
| 3161 | TCTGCTGCTC TTCAACCTGA AGAAGAGCAA GAAGAAGATT |
| 3201 | GGTTAGATGA TGATAGTCAA CAAACTGTTG GTCAACAAGA |
| 3241 | CGGCAGTGAG GACAATCAGA CAACTACTAT TCAAACAATT |
| 3281 | GTTGAGGTTC AACCTCAATT AGAGATGGAA CTTACACCAG |
| 3321 | TTGTTCAGAC TATTGAAGTG AATAGTTTTA GTGGTTATTT |
| 3361 | AAAACTTACT GACAATGTAT ACATTAAAAA TGCAGACATT |
| 3401 | GTGGAAGAAG CTAAAAAGGT AAAACCAACA GTGGTTGTTA |
| 3441 | ATGCAGCCAA TGTTTACCTT AAACATGGAG GAGGTGTTGC |
| 3481 | AGGAGCCTTA AATAAGGCTA CTAACAATGC CATGCAAGTT |
| 3521 | GAATCTGATG ATTACATAGC TACTAATGGA CCACTTAAAG |
| 3561 | TGGGTGGTAG TTGTGTTTTA AGCGGACACA ATCTTGCTAA |
| 3601 | ACACTGTCTT CATGTTGTCG GCCCAAATGT TAACAAAGGT |
| 3641 | GAAGACATTC AACTTCTTAA GAGTGCTTAT GAAAATTTTA |
| 3681 | ATCAGCACGA AGTTCTACTT GCACCATTAT TATCAGCTGG |
| 3721 | TATTTTTGGT GCTGACCCTA TACATTCTTT AAGAGTTTGT |
| 3761 | GTAGATACTG TTCGCACAAA TGTCTACTTA GCTGTCTTTG |
| 3801 | ATAAAAATCT CTATGACAAA CTTGTTTCAA GCTTTTTGGA |
| 3841 | AATGAAGAGT GAAAAGCAAG TTGAACAAAA GATCGCTGAG |
| 3881 | ATTCCTAAAG AGGAAGTTAA GCCATTTATA ACTGAAAGTA |
| 3921 | AACCTTCAGT TGAACAGAGA AAACAAGATG ATAAGAAAAT |
| 3961 | CAAAGCTTGT GTTGAAGAAG TTACAACAAC TCTGGAAGAA |
| 4001 | ACTAAGTTCC TCACAGAAAA CTTGTTACTT TATATTGACA |
| 4041 | TTAATGGCAA TCTTCATCCA GATTCTGCCA CTCTTGTTAG |
| 4081 | TGACATTGAC ATCACTTTCT TAAAGAAAGA TGCTCCATAT |
| 4121 | ATAGTGGGTG ATGTTGTTCA AGAGGGTGTT TTAACTGCTG |
| 4161 | TGGTTATACC TACTAAAAAG GCTGGTGGCA CTACTGAAAT |
| 4201 | GCTAGCGAAA GCTTTGAGAA AAGTGCCAAC AGACAATTAT |
| 4241 | ATAACCACTT ACCCGGGTCA GGGTTTAAAT GGTTACACTG |
| 4281 | TAGAGGAGGC AAAGACAGTG CTTAAAAAGT GTAAAAGTGC |
| 4321 | CTTTTACATT CTACCATCTA TTATCTCTAA TGAGAAGCAA |
| 4361 | GAAATTCTTG GAACTGTTTC TTGGAATTTG CGAGAAATGC |
| 4401 | TTGCACATGC AGAAGAAACA CGCAAATTAA TGCCTGTCTG |
| 4441 | TGTGGAAACT AAAGCCATAG TTTCAACTAT ACAGCGTAAA |
| 4481 | TATAAGGGTA TTAAAATACA AGAGGGTGTG GTTGATTATG |
| 4521 | GTGCTAGATT TTACTTTTAC ACCAGTAAAA CAACTGTAGC |
| 4561 | GTCACTTATC AACACACTTA ACGATCTAAA TGAAACTCTT |
| 4601 | GTTACAATGC CACTTGGCTA TGTAACACAT GGCTTAAATT |
| 4641 | TGGAAGAAGC TGCTCGGTAT ATGAGATCTC TCAAAGTGCC |
| 4681 | AGCTACAGTT TCTGTTTCTT CACCTGATGC TGTTACAGCG |
| 4721 | TATAATGGTT ATCTTACTTC TTCTTCTAAA ACACCTGAAG |
| 4761 | AACATTTTAT TGAAACCATC TCACTTGCTG GTTCCTATAA |
| 4801 | AGATTGGTCC TATTCTGGAC AATCTACACA ACTAGGTATA |
| 4841 | GAATTTCTTA AGAGAGGTGA TAAAAGTGTA TATTACACTA |
| 4881 | GTAATCCTAC CACATTCCAC CTAGATGGTG AAGTTATCAC |
| 4921 | CTTTGACAAT CTTAAGACAC TTCTTTCTTT GAGAGAAGTG |
| 4961 | AGGACTATTA AGGTGTTTAC AACAGTAGAC AACATTAACC |
| 5001 | TCCACACGCA AGTTGTGGAC ATGTCAATGA CATATGGACA |
| 5041 | ACAGTTTGGT CCAACTTATT TGGATGGAGC TGATGTTACT |
| 5081 | AAAATAAAAC CTCATAATTC ACATGAAGGT AAAACATTTT |
| 5121 | ATGTTTTACC TAATGATGAC ACTCTACGTG TTGAGGCTTT |
| 5161 | TGAGTACTAC CACACAACTG ATCCTAGTTT TCTGGGTAGG |
| 5201 | TACATGTCAG CATTAAATCA CACTAAAAAG TGGAAATACC |
| 5241 | CACAAGTTAA TGGTTTAACT TCTATTAAAT GGGCAGATAA |
| 5281 | CAACTGTTAT CTTGCCACTG CATTGTTAAC ACTCCAACAA |
| 5321 | ATAGAGTTGA AGTTTAATCC ACCTGCTCTA CAAGATGCTT |
| 5361 | ATTACAGAGC AAGGGCTGGT GAAGCTGCTA ACTTTTGTGC |
| 5401 | ACTTATCTTA GCCTACTGTA ATAAGACAGT AGGTGAGTTA |
| 5441 | GGTGATGTTA GAGAAACAAT GAGTTACTTG TTTCAACATG |
| 5481 | CCAATTTAGA TTCTTGCAAA AGAGTCTTGA ACGTGGTGTG |
| 5521 | TAAAACTTGT GGACAACAGC AGACAACCCT TAAGGGTGTA |
| 5561 | GAAGCTGTTA TGTACATGGG CACACTTTCT TATGAACAAT |
| 5601 | TTAAGAAAGG TGTTCAGATA CCTTGTACGT GTGGTAAACA |
| 5641 | AGCTACAAAA TATCTAGTAC AACAGGAGTC ACCTTTTGTT |
| 5681 | ATGATGTCAG CACCACCTGC TCAGTATGAA CTTAAGCATG |
| 5721 | GTACATTTAC TTGTGCTAGT GAGTACACTG GTAATTACCA |
| 5761 | GTGTGGTCAC TATAAACATA TAACTTCTAA AGAAACTTTG |
| 5801 | TATTGCATAG ACGGTGCTTT ACTTACAAAG TCCTCAGAAT |
| 5841 | ACAAAGGTCC TATTACGGAT GTTTTCTACA AAGAAAACAG |
| 5881 | TTACACAACA ACCATAAAAC CAGTTACTTA TAAATTGGAT |
| 5921 | GGTGTTGTTT GTACAGAAAT TGACCCTAAG TTGGACAATT |
| 5961 | ATTATAAGAA AGACAATTCT TATTTCACAG AGCAACCAAT |
| 6001 | TGATCTTGTA CCAAACCAAC CATATCCAAA CGCAAGCTTC |
| 6041 | GATAATTTTA AGTTTGTATG TGATAATATC AAATTTGCTG |
| 6081 | ATGATTTAAA CCAGTTAACT GGTTATAAGA AACCTGCTTC |
| 6121 | AAGAGAGCTT AAAGTTACAT TTTTCCCTGA CTTAAATGGT |
| 6161 | GATGTGGTGG CTATTGATTA TAAACACTAC ACACCCTCTT |
| 6201 | TTAAGAAAGG AGCTAAATTG TTACATAAAC CTATTGTTTG |
| 6241 | GCATGTTAAC AATGCAACTA ATAAAGCCAC GTATAAACCA |
| 6281 | AATACCTGGT GTATACGTTG TCTTTGGAGC ACAAAACCAG |
| 6321 | TTGAAACATC AAATTCGTTT GATGTACTGA AGTCAGAGGA |
| 6361 | CGCGCAGGGA ATGGATAATC TTGCCTGCGA AGATCTAAAA |
| 6401 | CCAGTCTCTG AAGAAGTAGT GGAAAATCCT ACCATACAGA |
| 6441 | AAGACGTTCT TGAGTGTAAT GTGAAAACTA CCGAAGTTGT |
| 6481 | AGGAGACATT ATACTTAAAC CAGCAAATAA TAGTTTAAAA |
| 6521 | ATTACAGAAG AGGTTGGCCA CACAGATCTA ATGGCTGCTT |
| 6561 | ATGTAGACAA TTCTAGTCTT ACTATTAAGA AACCTAATGA |
| 6601 | ATTATCTAGA GTATTAGGTT TGAAAACCCT TGCTACTCAT |
| 6641 | GGTTTAGCTG CTGTTAATAG TGTCCCTTGG GATACTATAG |
| 6681 | CTAATTATGC TAAGCCTTTT CTTAACAAAG TTGTTAGTAC |
| 6721 | AACTACTAAC ATAGTTACAC GGTGTTTAAA CCGTGTTTGT |
| 6761 | ACTAATTATA TGCCTTATTT CTTTACTTTA TTGCTACAAT |
| 6801 | TGTGTACTTT TACTAGAAGT ACAAATTCTA GAATTAAAGC |
| 6841 | ATCTATGCCG ACTACTATAG CAAAGAATAC TGTTAAGAGT |
| 6881 | GTCGGTAAAT TTTGTCTAGA GGCTTCATTT AATTATTTGA |
| 6921 | AGTCACCTAA TTTTTCTAAA CTGATAAATA TTATAATTTG |
| 6961 | GTTTTTACTA TTAAGTGTTT GCCTAGGTTC TTTAATCTAC |
| 7001 | TCAACCGCTG CTTTAGGTGT TTTAATGTCT AATTTAGGCA |
| 7041 | TGCCTTCTTA CTGTACTGGT TACAGAGAAG GCTATTTGAA |
| 7081 | CTCTACTAAT GTCACTATTG CAACCTACTG TACTGGTTCT |
| 7121 | ATACCTTGTA GTGTTTGTCT TAGTGGTTTA GATTCTTTAG |
| 7161 | ACACCTATCC TTCTTTAGAA ACTATACAAA TTACCATTTC |
| 7201 | ATCTTTTAAA TGGGATTTAA CTGCTTTTGG CTTAGTTGCA |
| 7241 | GAGTGGTTTT TGGCATATAT TCTTTTCACT AGGTTTTTCT |
| 7281 | ATGTACTTGG ATTGGCTGCA ATCATGCAAT TGTTTTTCAG |
| 7321 | CTATTTTGCA GTACATTTTA TTAGTAATTC TTGGCTTATG |
| 7361 | TGGTTAATAA TTAATCTTGT ACAAATGGCC CCGATTTCAG |
| 7401 | CTATGGTTAG AATGTACATC TTCTTTGCAT CATTTTATTA |
| 7441 | TGTATGGAAA AGTTATGTGC ATGTTGTAGA CGGTTGTAAT |
| 7481 | TCATCAACTT GTATGATGTG TTACAAACGT AATAGAGCAA |
| 7521 | CAAGAGTCGA ATGTACAACT ATTGTTAATG GTGTTAGAAG |
| 7561 | GTCCTTTTAT GTCTATGCTA ATGGAGGTAA AGGCTTTTGC |
| 7601 | AAACTACACA ATTGGAATTG TGTTAATTGT GATACATTCT |
| 7641 | GTGCTGGTAG TACATTTATT AGTGATGAAG TTGCGAGAGA |
| 7681 | CTTGTCACTA CAGTTTAAAA GACCAATAAA TCCTACTGAC |
| 7721 | CAGTCTTCTT ACATCGTTGA TAGTGTTACA GTGAAGAATG |
| 7761 | GTTCCATCCA TCTTTACTTT GATAAAGCTG GTCAAAAGAC |
| 7801 | TTATGAAAGA CATTCTCTCT CTCATTTTGT TAACTTAGAC |
| 7841 | AACCTGAGAG CTAATAACAC TAAAGGTTCA TTGCCTATTA |
| 7881 | ATGTTATAGT TTTTGATGGT AAATCAAAAT GTGAAGAATC |
| 7921 | ATCTGCAAAA TCAGCGTCTG TTTACTACAG TCAGCTTATG |
| 7961 | TGTCAACCTA TACTGTTACT AGATCAGGCA TTAGTGTCTG |
| 8001 | ATGTTGGTGA TAGTGCGGAA GTTGCAGTTA AAATGTTTGA |
| 8041 | TGCTTACGTT AATACGTTTT CATCAACTTT TAACGTACCA |
| 8081 | ATGGAAAAAC TCAAAACACT AGTTGCAACT GCAGAAGCTG |
| 8121 | AACTTGCAAA GAATGTGTCC TTAGACAATG TCTTATCTAC |
| 8161 | TTTTATTTCA GCAGCTCGGC AAGGGTTTGT TGATTCAGAT |
| 8201 | GTAGAAACTA AAGATGTTGT TGAATGTCTT AAATTGTCAC |
| 8241 | ATCAATCTGA CATAGAAGTT ACTGGCGATA GTTGTAATAA |
| 8281 | CTATATGCTC ACCTATAACA AAGTTGAAAA CATGACACCC |
| 8321 | CGTGACCTTG GTGCTTGTAT TGACTGTAGT GCGCGTCATA |
| 8361 | TTAATGCGCA GGTAGCAAAA AGTCACAACA TTGCTTTGAT |
| 8401 | ATGGAACGTT AAAGATTTCA TGTCATTGTC TGAACAACTA |
| 8441 | CGAAAACAAA TACGTAGTGC TGCTAAAAAG AATAACTTAC |
| 8481 | CTTTTAAGTT GACATGTGCA ACTACTAGAC AAGTTGTTAA |
| 8521 | TGTTGTAACA ACAAAGATAG CACTTAAGGG TGGTAAAATT |
| 8561 | GTTAATAATT GGTTGAAGCA GTTAATTAAA GTTACACTTG |
| 8601 | TGTTCCTTTT TGTTGCTGCT ATTTTCTATT TAATAACACC |
| 8641 | TGTTCATGTC ATGTCTAAAC ATACTGACTT TTCAAGTGAA |
| 8681 | ATCATAGGAT ACAAGGCTAT TGATGGTGGT GTCACTCGTG |
| 8721 | ACATAGCATC TACAGATACT TGTTTTGCTA ACAAACATGC |
| 8761 | TGATTTTGAC ACATGGTTTA GCCAGCGTGG TGGTAGTTAT |
| 8801 | ACTAATGACA AAGCTTGCCC ATTGATTGCT GCAGTCATAA |
| 8841 | CAAGAGAAGT GGGTTTTGTC GTGCCTGGTT TGCCTGGCAC |
| 8881 | GATATTACGC ACAACTAATG GTGACTTTTT GCATTTCTTA |
| 8921 | CCTAGAGTTT TTAGTGCAGT TGGTAACATC TGTTACACAC |
| 8961 | CATCAAAACT TATAGAGTAC ACTGACTTTG CAACATCAGC |
| 9001 | TTGTGTTTTG GCTGCTGAAT GTACAATTTT TAAAGATGCT |
| 9041 | TCTGGTAAGC CAGTACCATA TTGTTATGAT ACCAATGTAC |
| 9081 | TAGAAGGTTC TGTTGCTTAT GAAAGTTTAC GCCCTGACAC |
| 9121 | ACGTTATGTG CTCATGGATG GCTCTATTAT TCAATTTCCT |
| 9161 | AACACCTACC TTGAAGGTTC TGTTAGAGTG GTAACAACTT |
| 9201 | TTGATTCTGA GTACTGTAGG CACGGCACTT GTGAAAGATC |
| 9241 | AGAAGCTGGT GTTTGTGTAT CTACTAGTGG TAGATGGGTA |
| 9281 | CTTAACAATG ATTATTACAG ATCTTTACCA GGAGTTTTCT |
| 9321 | GTGGTGTAGA TGCTGTAAAT TTACTTACTA ATATGTTTAC |
| 9361 | ACCACTAATT CAACCTATTG GTGCTTTGGA CATATCAGCA |
| 9401 | TCTATAGTAG CTGGTGGTAT TGTAGCTATC GTAGTAACAT |
| 9441 | GCCTTGCCTA CTATTTTATG AGGTTTAGAA GAGCTTTTGG |
| 9481 | TGAATACAGT CATGTAGTTG CCTTTAATAC TTTACTATTC |
| 9521 | CTTATGTCAT TCACTGTACT CTGTTTAACA CCAGTTTACT |
| 9561 | CATTCTTACC TGGTGTTTAT TCTGTTATTT ACTTGTACTT |
| 9601 | GACATTTTAT CTTACTAATG ATGTTTCTTT TTTAGCACAT |
| 9641 | ATTCAGTGGA TGGTTATGTT CACACCTTTA GTACCTTTCT |
| 9681 | GGATAACAAT TGCTTATATC ATTTGTATTT CCACAAAGCA |
| 9721 | TTTCTATTGG TTCTTTAGTA ATTACCTAAA GAGACGTGTA |
| 9761 | GTCTTTAATG GTGTTTCCTT TAGTACTTTT GAAGAAGCTG |
| 9801 | CGCTGTGCAC CTTTTTGTTA AATAAAGAAA TGTATCTAAA |
| 9841 | GTTGCGTAGT GATGTGCTAT TACCTCTTAC GCAATATAAT |
| 9881 | AGATACTTAG CTCTTTATAA TAAGTACAAG TATTTTAGTG |
| 9921 | GAGCAATGGA TACAACTAGC TACAGAGAAG CTGCTTGTTG |
| 9961 | TCATCTCGCA AAGGCTCTCA ATGACTTCAG TAACTCAGGT |
| 10001 | TCTGATGTTC TTTACCAACC ACCACAAACC TCTATCACCT |
| 10041 | CAGCTGTTTT GCAGAGTGGT TTTAGAAAAA TGGCATTCCC |
| 10081 | ATCTGGTAAA GTTGAGGGTT GTATGGTACA AGTAACTTGT |
| 10121 | GGTACAACTA CACTTAACGG TCTTTGGCTT GATGACGTAG |
| 10161 | TTTACTGTCC AAGACATGTG ATCTGCACCT CTGAAGACAT |
| 10201 | GCTTAACCCT AATTATGAAG ATTTACTCAT TCGTAAGTCT |
| 10241 | AATCATAATT TCTTGGTACA GGCTGGTAAT GTTCAACTCA |
| 10281 | GGGTTATTGG ACATTCTATG CAAAATTGTG TACTTAAGCT |
| 10321 | TAAGGTTGAT ACAGCCAATC CTAAGACACC TAAGTATAAG |
| 10361 | TTTGTTCGCA TTCAACCAGG ACAGACTTTT TCAGTGTTAG |
| 10401 | CTTGTTACAA TGGTTCACCA TCTGGTGTTT ACCAATGTGC |
| 10441 | TATGAGGCCC AATTTCACTA TTAAGGGTTC ATTCCTTAAT |
| 10481 | GGTTCATGTG GTAGTGTTGG TTTTAACATA GATTATGACT |
| 10521 | GTGTCTCTTT TTGTTACATG CACCATATGG AATTACCAAC |
| 10561 | TGGAGTTCAT GCTGGCACAG ACTTAGAAGG TAACTTTTAT |
| 10601 | GGACCTTTTG TTGACAGGCA AACAGCACAA GCAGCTGGTA |
| 10641 | CGGACACAAC TATTACAGTT AATGTTTTAG CTTGGTTGTA |
| 10681 | CGCTGCTGTT ATAAATGGAG ACAGGTGGTT TCTCAATCGA |
| 10721 | TTTACCACAA CTCTTAATGA CTTTAACCTT GTGGCTATGA |
| 10761 | AGTACAATTA TGAACCTCTA ACACAAGACC ATGTTGACAT |
| 10801 | ACTAGGACCT CTTTCTGCTC AAACTGGAAT TGCCGTTTTA |
| 10841 | GATATGTGTG CTTCATTAAA AGAATTACTG CAAAATGGTA |
| 10881 | TGAATGGACG TACCATATTG GGTAGTGCTT TATTAGAAGA |
| 10921 | TGAATTTACA CCTTTTGATG TTGTTAGACA ATGCTCAGGT |
| 10961 | GTTACTTTCC AAAGTGCAGT GAAAAGAACA ATCAAGGGTA |
| 11001 | CACACCACTG GTTGTTACTC ACAATTTTGA CTTCACTTTT |
| 11041 | AGTTTTAGTC CAGAGTACTC AATGGTCTTT GTTCTTTTTT |
| 11081 | TTGTATGAAA ATGCCTTTTT ACCTTTTGCT ATGGGTATTA |
| 11121 | TTGCTATGTC TGCTTTTGCA ATGATGTTTG TCAAACATAA |
| 11161 | GCATGCATTT CTCTGTTTGT TTTTGTTACC TTCTCTTGCC |
| 11201 | ACTGTAGCTT ATTTTAATAT GGTCTATATG CCTGCTAGTT |
| 11241 | GGGTGATGCG TATTATGACA TGGTTGGATA TGGTTGATAC |
| 11281 | TAGTTTGTCT GGTTTTAAGC TAAAAGACTG TGTTATGTAT |
| 11321 | GCATCAGCTG TAGTGTTACT AATCCTTATG ACAGCAAGAA |
| 11361 | CTGTGTATGA TGATGGTGCT AGGAGAGTGT GGACACTTAT |
| 11401 | GAATGTCTTG ACACTCGTTT ATAAAGTTTA TTATGGTAAT |
| 11441 | GCTTTAGATC AAGCCATTTC CATGTGGGCT CTTATAATCT |
| 11481 | CTGTTACTTC TAACTACTCA GGTGTAGTTA CAACTGTCAT |
| 11521 | GTTTTTGGCC AGAGGTATTG TTTTTATGTG TGTTGAGTAT |
| 11561 | TGCCCTATTT TCTTCATAAC TGGTAATACA CTTCAGTGTA |
| 11601 | TAATGCTAGT TTATTGTTTC TTAGGCTATT TTTGTACTTG |
| 11641 | TTACTTTGGC CTCTTTTGTT TACTCAACCG CTACTTTAGA |
| 11681 | CTGACTCTTG GTGTTTATGA TTACTTAGTT TCTACACAGG |
| 11721 | AGTTTAGATA TATGAATTCA CAGGGACTAC TCCCACCCAA |
| 11761 | GAATAGCATA GATGCCTTCA AACTCAACAT TAAATTGTTG |
| 11801 | GGTGTTGGTG GCAAACCTTG TATCAAAGTA GCCACTGTAC |
| 11841 | AGTCTAAAAT GTCAGATGTA AAGTGCACAT CAGTAGTCTT |
| 11881 | ACTCTCAGTT TTGCAACAAC TCAGAGTAGA ATCATCATCT |
| 11921 | AAATTGTGGG CTCAATGTGT CCAGTTACAC AATGACATTC |
| 11961 | TCTTAGCTAA AGATACTACT GAAGCCTTTG AAAAAATGGT |
| 12001 | TTCACTACTT TCTGTTTTGC TTTCCATGCA GGGTGCTGTA |
| 12041 | GACATAAACA AGCTTTGTGA AGAAATGCTG GACAACAGGG |
| 12081 | CAACCTTACA AGCTATAGCC TCAGAGTTTA GTTCCCTTCC |
| 12121 | ATCATATGCA GCTTTTGCTA CTGCTCAAGA AGCTTATGAG |
| 12161 | CAGGCTGTTG CTAATGGTGA TTCTGAAGTT GTTCTTAAAA |
| 12201 | AGTTGAAGAA GTCTTTGAAT GTGGCTAAAT CTGAATTTGA |
| 12241 | CCGTGATGCA GCCATGCAAC GTAAGTTGGA AAAGATGGCT |
| 12281 | GATCAAGCTA TGACCCAAAT GTATAAACAG GCTAGATCTG |
| 12321 | AGGACAAGAG GGCAAAAGTT ACTAGTGCTA TGCAGACAAT |
| 12361 | GCTTTTCACT ATGCTTAGAA AGTTGGATAA TGATGCACTC |
| 12401 | AACAACATTA TCAACAATGC AAGAGATGGT TGTGTTCCCT |
| 12441 | TGAACATAAT ACCTCTTACA ACAGCAGCCA AACTAATGGT |
| 12481 | TGTCATACCA GACTATAACA CATATAAAAA TACGTGTGAT |
| 12521 | GGTACAACAT TTACTTATGC ATCAGCATTG TGGGAAATCC |
| 12561 | AACAGGTTGT AGATGCAGAT AGTAAAATTG TTCAACTTAG |
| 12601 | TGAAATTAGT ATGGACAATT CACCTAATTT AGCATGGCCT |
| 12641 | CTTATTGTAA CAGCTTTAAG GGCCAATTCT GCTGTCAAAT |
| 12681 | TACAGAATAA TGAGCTTAGT CCTGTTGCAC TACGACAGAT |
| 12721 | GTCTTGTGCT GCCGGTACTA CACAAACTGC TTGCACTGAT |
| 12761 | GACAATGCGT TAGCTTACTA CAACACAACA AAGGGAGGTA |
| 12801 | GGTTTGTACT TGCACTGTTA TCCGATTTAC AGGATTTGAA |
| 12841 | ATGGGCTAGA TTCCCTAAGA GTGATGGAAC TGGTACTATC |
| 12881 | TATACAGAAC TGGAACCACC TTGTAGGTTT GTTACAGACA |
| 12921 | CACCTAAAGG TCCTAAAGTG AAGTATTTAT ACTTTATTAA |
| 12961 | AGGATTAAAC AACCTAAATA GAGGTATGGT ACTTGGTAGT |
| 13001 | TTAGCTGCCA CAGTACGTCT ACAAGCTGGT AATGCAACAG |
| 13041 | AAGTGCCTGC CAATTCAACT GTATTATCTT TCTGTGCTTT |
| 13081 | TGCTGTAGAT GCTGCTAAAG CTTACAAAGA TTATCTAGCT |
| 13121 | AGTGGGGGAC AACCAATCAC TAATTGTGTT AAGATGTTGT |
| 13161 | GTACACACAC TGGTACTGGT CAGGCAATAA CAGTTACACC |
| 13201 | GGAAGCCAAT ATGGATCAAG AATCCTTTGG TGGTGCATCG |
| 13241 | TGTTGTCTGT ACTGCCGTTG CCACATAGAT CATCCAAATC |
| 13281 | CTAAAGGATT TTGTGACTTA AAAGGTAAGT ATGTACAAAT |
| 13321 | ACCTACAACT TGTGCTAATG ACCCTGTGGG TTTTACACTT |
| 13361 | AAAAACACAG TCTGTACCGT CTGCGGTATG TGGAAAGGTT |
| 13401 | ATGGCTGTAG TTGTGATCAA CTCCGCGAAC CCATGCTTCA |
| 13441 | GTCAGCTGAT GCACAATCGT TTTTAAACGG GTTTGCGGTG |
| 13481 | TAAGTGCAGC CCGTCTTACA CCGTGCGGCA CAGGCACTAG |
| 13521 | TACTGATGTC GTATACAGGG CTTTTGACAT CTACAATGAT |
| 13561 | AAAGTAGCTG GTTTTGCTAA ATTCCTAAAA ACTAATTGTT |
| 13601 | GTCGCTTCCA AGAAAAGGAC GAAGATGACA ATTTAATTGA |
| 13641 | TTCTTACTTT GTAGTTAAGA GACACACTTT CTCTAACTAC |
| 13681 | CAACATGAAG AAACAATTTA TAATTTACTT AAGGATTGTC |
| 13721 | CAGCTGTTGC TAAACATGAC TTCTTTAAGT TTAGAATAGA |
| 13761 | CGGTGACATG GTACCACATA TATCACGTCA ACGTCTTACT |
| 13801 | AAATACACAA TGGCAGACCT CGTCTATGCT TTAAGGCATT |
| 13841 | TTGATGAAGG TAATTGTGAC ACATTAAAAG AAATACTTGT |
| 13881 | CACATACAAT TGTTGTGATG ATGATTATTT CAATAAAAAG |
| 13921 | GACTGGTATG ATTTTGTAGA AAACCCAGAT ATATTACGCG |
| 13961 | TATACGCCAA CTTAGGTGAA CGTGTACGCC AAGCTTTGTT |
| 14001 | AAAAACAGTA CAATTCTGTG ATGCCATGCG AAATGCTGGT |
| 14041 | ATTGTTGGTG TACTGACATT AGATAATCAA GATCTCAATG |
| 14081 | GTAACTGGTA TGATTTCGGT GATTTCATAC AAACCACGCC |
| 14121 | AGGTAGTGGA GTTCCTGTTG TAGATTCTTA TTATTCATTG |
| 14161 | TTAATGCCTA TATTAACCTT GACCAGGGCT TTAACTGCAG |
| 14201 | AGTCACATGT TGACACTGAC TTAACAAAGC CTTACATTAA |
| 14241 | GTGGGATTTG TTAAAATATG ACTTCACGGA AGAGAGGTTA |
| 14281 | AAACTCTTTG ACCGTTATTT TAAATATTGG GATCAGACAT |
| 14321 | ACCACCCAAA TTGTGTTAAC TGTTTGGATG ACAGATGCAT |
| 14361 | TCTGCATTGT GCAAACTTTA ATGTTTTATT CTCTACAGTG |
| 14401 | TTCCCACCTA CAAGTTTTGG ACCACTAGTG AGAAAAATAT |
| 14441 | TTGTTGATGG TGTTCCATTT GTAGTTTCAA CTGGATACCA |
| 14481 | CTTCAGAGAG CTAGGTGTTG TACATAATCA GGATGTAAAC |
| 14521 | TTACATAGCT CTAGACTTAG TTTTAAGGAA TTACTTGTGT |
| 14561 | ATGCTGCTGA CCCTGCTATG CACGCTGCTT CTGGTAATCT |
| 14601 | ATTACTAGAT AAACGCACTA CGTGCTTTTC AGTAGCTGCA |
| 14641 | CTTACTAACA ATGTTGCTTT TCAAACTGTC AAACCCGGTA |
| 14681 | ATTTTAACAA AGACTTCTAT GACTTTGCTG TGTCTAAGGG |
| 14721 | TTTCTTTAAG GAAGGAAGTT CTGTTGAATT AAAACACTTC |
| 14761 | TTCTTTGCTC AGGATGGTAA TGCTGCTATC AGCGATTATG |
| 14801 | ACTACTATCG TTATAATCTA CCAACAATGT GTGATATCAG |
| 14841 | ACAACTACTA TTTGTAGTTG AAGTTGTTGA TAAGTACTTT |
| 14881 | GATTGTTACG ATGGTGGCTG TATTAATGCT AACCAAGTCA |
| 14921 | TCGTCAACAA CCTAGACAAA TCAGCTGGTT TTCCATTTAA |
| 14961 | TAAATGGGGT AAGGCTAGAC TTTATTATGA TTCAATGAGT |
| 15001 | TATGAGGATC AAGATGCACT TTTCGCATAT ACAAAACGTA |
| 15041 | ATGTCATCCC TACTATAACT CAAATGAATC TTAAGTATGC |
| 15081 | CATTAGTGCA AAGAATAGAG CTCGCACCGT AGCTGGTGTC |
| 15121 | TCTATCTGTA GTACTATGAC CAATAGACAG TTTCATCAAA |
| 15161 | AATTATTGAA ATCAATAGCC GCCACTAGAG GAGCTACTGT |
| 15201 | AGTAATTGGA ACAAGCAAAT TCTATGGTGG TTGGCACAAC |
| 15241 | ATGTTAAAAA CTGTTTATAG TGATGTAGAA AACCCTCACC |
| 15281 | TTATGGGTTG GGATTATCCT AAATGTGATA GAGCCATGCC |
| 15321 | TAACATGCTT AGAATTATGG CCTCACTTGT TCTTGCTCGC |
| 15361 | AAACATACAA CGTGTTGTAG CTTGTCACAC CGTTTCTATA |
| 15401 | GATTAGCTAA TGAGTGTGCT CAAGTATTGA GTGAAATGGT |
| 15441 | CATGTGTGGC GGTTCACTAT ATGTTAAACC AGGTGGAACC |
| 15481 | TCATCAGGAG ATGCCACAAC TGCTTATGCT AATAGTGTTT |
| 15521 | TTAACATTTG TCAAGCTGTC ACGGCCAATG TTAATGCACT |
| 15561 | TTTATCTACT GATGGTAACA AAATTGCCGA TAAGTATGTC |
| 15601 | CGCAATTTAC AACACAGACT TTATGAGTGT CTCTATAGAA |
| 15641 | ATAGAGATGT TGACACAGAC TTTGTGAATG AGTTTTACGC |
| 15681 | ATATTTGCGT AAACATTTCT CAATGATGAT ACTCTCTGAC |
| 15721 | GATGCTGTTG TGTGTTTCAA TAGCACTTAT GCATCTCAAG |
| 15761 | GTCTAGTGGC TAGCATAAAG AACTTTAAGT CAGTTCTTTA |
| 15801 | TTATCAAAAC AATGTTTTTA TGTCTGAAGC AAAATGTTGG |
| 15841 | ACTGAGACTG ACCTTACTAA AGGACCTCAT GAATTTTGCT |
| 15881 | CTCAACATAC AATGCTAGTT AAACAGGGTG ATGATTATGT |
| 15921 | GTACCTTCCT TACCCAGATC CATCAAGAAT CCTAGGGGCC |
| 15961 | GGCTGTTTTG TAGATGATAT CGTAAAAACA GATGGTACAC |
| 16001 | TTATGATTGA ACGGTTCGTG TCTTTAGCTA TAGATGCTTA |
| 16041 | CCCACTTACT AAACATCCTA ATCAGGAGTA TGCTGATGTC |
| 16081 | TTTCATTTGT ACTTACAATA CATAAGAAAG CTACATGATG |
| 16121 | AGTTAACAGG ACACATGTTA GACATGTATT CTGTTATGCT |
| 16161 | TACTAATGAT AACACTTCAA GGTATTGGGA ACCTGAGTTT |
| 16201 | TATGAGGCTA TGTACACACC GCATACAGTC TTACAGGCTG |
| 16241 | TTGGGGCTTG TGTTCTTTGC AATTCACAGA CTTCATTAAG |
| 16281 | ATGTGGTGCT TGCATACGTA GACCATTCTT ATGTTGTAAA |
| 16321 | TGCTGTTACG ACCATGTCAT ATCAACATCA CATAAATTAG |
| 16361 | TCTTGTCTGT TAATCCGTAT GTTTGCAATG CTCCAGGTTG |
| 16401 | TGATGTCACA GATGTGACTC AACTTTACTT AGGAGGTATG |
| 16441 | AGCTATTATT GTAAATCACA TAAACCACCC ATTAGTTTTC |
| 16481 | CATTGTGTGC TAATGGACAA GTTTTTGGTT TATATAAAAA |
| 16521 | TACATGTGTT GGTAGCGATA ATGTTACTGA CTTTAATGCA |
| 16561 | ATTGCAACAT GTGACTGGAC AAATGCTGGT GATTACATTT |
| 16601 | TAGCTAACAC CTGTACTGAA AGACTCAAGC TTTTTGCAGC |
| 16641 | AGAAACGCTC AAAGCTACTG AGGAGACATT TAAACTGTCT |
| 16681 | TATGGTATTG CTACTGTACG TGAAGTGCTG TCTGACAGAG |
| 16721 | AATTACATCT TTCATGGGAA GTTGGTAAAC CTAGACCACC |
| 16761 | ACTTAACCGA AATTATGTCT TTACTGGTTA TCGTGTAACT |
| 16801 | AAAAACAGTA AAGTACAAAT AGGAGAGTAC ACCTTTGAAA |
| 16841 | AAGGTGACTA TGGTGATGCT GTTGTTTACC GAGGTACAAC |
| 16881 | AACTTACAAA TTAAATGTTG GTGATTATTT TGTGCTGACA |
| 16921 | TCACATACAG TAATGCCATT AAGTGCACCT ACACTAGTGC |
| 16961 | CACAAGAGCA CTATGTTAGA ATTACTGGCT TATACCCAAC |
| 17001 | ACTCAATATC TCAGATGAGT TTTCTAGCAA TGTTGCAAAT |
| 17041 | TATCAAAAGG TTGGTATGCA AAAGTATTCT ACACTCCAGG |
| 17081 | GACCACCTGG TACTGGTAAG AGTCATTTTG CTATTGGCCT |
| 17121 | AGCTCTCTAC TACCCTTCTG CTCGCATAGT GTATACAGCT |
| 17161 | TGCTCTCATG CCGCTGTTGA TGCACTATGT GAGAAGGCAT |
| 17201 | TAAAATATTT GCCTATAGAT AAATGTAGTA GAATTATACC |
| 17241 | TGCACGTGCT CGTGTAGAGT GTTTTGATAA ATTCAAAGTG |
| 17281 | AATTCAACAT TAGAACAGTA TGTCTTTTGT ACTGTAAATG |
| 17321 | CATTGCCTGA GACGACAGCA GATATAGTTG TCTTTGATGA |
| 17361 | AATTTCAATG GCCACAAATT ATGATTTGAG TGTTGTCAAT |
| 17401 | GCCAGATTAC GTGCTAAGCA CTATGTGTAC ATTGGCGACC |
| 17441 | CTGCTCAATT ACCTGCACCA CGCACATTGC TAACTAAGGG |
| 17481 | CACACTAGAA CCAGAATATT TCAATTCAGT GTGTAGACTT |
| 17521 | ATGAAAACTA TAGGTCCAGA CATGTTCCTC GGAACTTGTC |
| 17561 | GGCGTTGTCC TGCTGAAATT GTTGACACTG TGAGTGCTTT |
| 17601 | GGTTTATGAT AATAAGCTTA AAGCACATAA AGACAAATCA |
| 17641 | GCTCAATGCT TTAAAATGTT TTATAAGGGT GTTATCACGC |
| 17681 | ATGATGTTTC ATCTGCAATT AACAGGCCAC AAATAGGCGT |
| 17721 | GGTAAGAGAA TTCCTTACAC GTAACCCTGC TTGGAGAAAA |
| 17761 | GCTGTCTTTA TTTCACCTTA TAATTCACAG AATGCTGTAG |
| 17801 | CCTCAAAGAT TTTGGGACTA CCAACTCAAA CTGTTGATTC |
| 17841 | ATCACAGGGC TCAGAATATG ACTATGTCAT ATTCACTCAA |
| 17881 | ACCACTGAAA CAGCTCACTC TTGTAATGTA AACAGATTTA |
| 17921 | ATGTTGCTAT TACCAGAGCA AAAGTAGGCA TACTTTGCAT |
| 17961 | AATGTCTGAT AGAGACCTTT ATGACAAGTT GCAATTTACA |
| 18001 | AGTCTTGAAA TTCCACGTAG GAATGTGGCA ACTTTACAAG |
| 18041 | CTGAAAATGT AACAGGACTC TTTAAAGATT GTAGTAAGGT |
| 18081 | AATCACTGGG TTACATCCTA CACAGGCACC TACACACCTC |
| 18121 | AGTGTTGACA CTAAATTCAA AACTGAAGGT TTATGTGTTG |
| 18161 | ACATACCTGG CATACCTAAG GACATGACCT ATAGAAGACT |
| 18201 | CATCTCTATG ATGGGTTTTA AAATGAATTA TCAAGTTAAT |
| 18241 | GGTTACCCTA ACATGTTTAT CACCCGCGAA GAAGCTATAA |
| 18281 | GACATGTACG TGCATGGATT GGCTTCGATG TCGAGGGGTG |
| 18321 | TCATGCTACT AGAGAAGCTG TTGGTACCAA TTTACCTTTA |
| 18361 | CAGCTAGGTT TTTCTACAGG TGTTAACCTA GTTGCTGTAC |
| 18401 | CTACAGGTTA TGTTGATACA CCTAATAATA CAGATTTTTC |
| 18441 | CAGAGTTAGT GCTAAACCAC CGCCTGGAGA TCAATTTAAA |
| 18481 | CACCTCATAC CACTTATGTA CAAAGGACTT CCTTGGAATG |
| 18521 | TAGTGCGTAT AAAGATTGTA CAAATGTTAA GTGACACACT |
| 18561 | TAAAAATCTC TCTGACAGAG TCGTATTTGT CTTATGGGCA |
| 18601 | CATGGCTTTG AGTTGACATC TATGAAGTAT TTTGTGAAAA |
| 18641 | TAGGACCTGA GCGCACCTGT TGTCTATGTG ATAGACGTGC |
| 18681 | CACATGCTTT TCCACTGCTT CAGACACTTA TGCCTGTTGG |
| 18721 | CATCATTCTA TTGGATTTGA TTACGTCTAT AATCCGTTTA |
| 18761 | TGATTGATGT TCAACAATGG GGTTTTACAG GTAACCTACA |
| 18801 | AAGCAACCAT GATCTGTATT GTCAAGTCCA TGGTAATGCA |
| 18841 | CATGTAGCTA GTTGTGATGC AATCATGACT AGGTGTCTAG |
| 18881 | CTGTCCACGA GTGCTTTGTT AAGCGTGTTG ACTGGACTAT |
| 18921 | TGAATATCCT ATAATTGGTG ATGAACTGAA GATTAATGCG |
| 18961 | GCTTGTAGAA AGGTTCAACA CATGGTTGTT AAAGCTGCAT |
| 19001 | TATTAGCAGA CAAATTCCCA GTTCTTCACG ACATTGGTAA |
| 19041 | CCCTAAAGCT ATTAAGTGTG TACCTCAAGC TGATGTAGAA |
| 19081 | TGGAAGTTCT ATGATGCACA GCCTTGTAGT GACAAAGCTT |
| 19121 | ATAAAATAGA AGAATTATTC TATTCTTATG CCACACATTC |
| 19161 | TGACAAATTC ACAGATGGTG TATGCCTATT TTGGAATTGC |
| 19201 | AATGTCGATA GATATCCTGC TAATTCCATT GTTTGTAGAT |
| 19241 | TTGACACTAG AGTGCTATCT AACCTTAACT TGCCTGGTTG |
| 19281 | TGATGGTGGC AGTTTGTATG TAAATAAACA TGCATTCCAC |
| 19321 | ACACCAGCTT TTGATAAAAG TGCTTTTGTT AATTTAAAAC |
| 19361 | AATTACCATT TTTCTATTAC TCTGACAGTC CATGTGAGTC |
| 19401 | TCATGGAAAA CAAGTAGTGT CAGATATAGA TTATGTACCA |
| 19441 | CTAAAGTCTG CTACGTGTAT AACACGTTGC AATTTAGGTG |
| 19481 | GTGCTGTCTG TAGACATCAT GCTAATGAGT ACAGATTGTA |
| 19521 | TCTCGATGCT TATAACATGA TGATCTCAGC TGGCTTTAGC |
| 19561 | TTGTGGGTTT ACAAACAATT TGATACTTAT AACCTCTGGA |
| 19601 | ACACTTTTAC AAGACTTCAG AGTTTAGAAA ATGTGGCTTT |
| 19641 | TAATGTTGTA AATAAGGGAC ACTTTGATGG ACAACAGGGT |
| 19681 | GAAGTACCAG TTTCTATCAT TAATAACACT GTTTACACAA |
| 19721 | AAGTTGATGG TGTTGATGTA GAATTGTTTG AAAATAAAAC |
| 19761 | AACATTACCT GTTAATGTAG CATTTGAGCT TTGGGCTAAG |
| 19801 | CGCAACATTA AACCAGTACC AGAGGTGAAA ATACTCAATA |
| 19841 | ATTTGGGTGT GGACATTGCT GCTAATACTG TGATCTGGGA |
| 19881 | CTACAAAAGA GATGCTCCAG CACATATATC TACTATTGGT |
| 19921 | GTTTGTTCTA TGACTGACAT AGCCAAGAAA CCAACTGAAA |
| 19961 | CGATTTGTGC ACCACTCACT GTCTTTTTTG ATGGTAGAGT |
| 20001 | TGATGGTCAA GTAGACTTAT TTAGAAATGC CCGTAATGGT |
| 20041 | GTTCTTATTA CAGAAGGTAG TGTTAAAGGT TTACAACCAT |
| 20081 | CTGTAGGTCC CAAACAAGCT AGTCTTAATG GAGTCACATT |
| 20121 | AATTGGAGAA GCCGTAAAAA CACAGTTCAA TTATTATAAG |
| 20161 | AAAGTTGATG GTGTTGTCCA ACAATTACCT GAAACTTACT |
| 20201 | TTACTCAGAG TAGAAATTTA CAAGAATTTA AACCCAGGAG |
| 20241 | TCAAATGGAA ATTGATTTCT TAGAATTAGC TATGGATGAA |
| 20281 | TTCATTGAAC GGTATAAATT AGAAGGCTAT GCCTTCGAAC |
| 20321 | ATATCGTTTA TGGAGATTTT AGTCATAGTC AGTTAGGTGG |
| 20361 | TTTACATCTA CTGATTGGAC TAGCTAAACG TTTTAAGGAA |
| 20401 | TCACCTTTTG AATTAGAAGA TTTTATTCCT ATGGACAGTA |
| 20441 | CAGTTAAAAA CTATTTCATA ACAGATGCGC AAACAGGTTC |
| 20481 | ATCTAAGTGT GTGTGTTCTG TTATTGATTT ATTACTTGAT |
| 20521 | GATTTTGTTG AAATAATAAA ATCCCAAGAT TTATCTGTAG |
| 20561 | TTTCTAAGGT TGTCAAAGTG ACTATTGACT ATACAGAAAT |
| 20601 | TTCATTTATG CTTTGGTGTA AAGATGGCCA TGTAGAAACA |
| 20641 | TTTTACCCAA AATTACAATC TAGTCAAGCG TGGCAACCGG |
| 20681 | GTGTTGCTAT GCCTAATCTT TACAAAATGC AAAGAATGCT |
| 20721 | ATTAGAAAAG TGTGACCTTC AAAATTATGG TGATAGTGCA |
| 20761 | ACATTACCTA AAGGCATAAT GATGAATGTC GCAAAATATA |
| 20801 | CTCAACTGTG TCAATATTTA AACACATTAA CATTAGCTGT |
| 20841 | ACCCTATAAT ATGAGAGTTA TACATTTTGG TGCTGGTTCT |
| 20881 | GATAAAGGAG TTGCACCAGG TACAGCTGTT TTAAGACAGT |
| 20921 | GGTTGCCTAC GGGTACGCTG CTTGTCGATT CAGATCTTAA |
| 20961 | TGACTTTGTC TCTGATGCAG ATTCAACTTT GATTGGTGAT |
| 21001 | TGTGCAACTG TACATACAGC TAATAAATGG GATCTCATTA |
| 21041 | TTAGTGATAT GTACGACCCT AAGACTAAAA ATGTTACAAA |
| 21081 | AGAAAATGAC TCTAAAGAGG GTTTTTTCAC TTACATTTGT |
| 21121 | GGGTTTATAC AACAAAAGCT AGCTCTTGGA GGTTCCGTGG |
| 21161 | CTATAAAGAT AACAGAACAT TCTTGGAATG CTGATCTTTA |
| 21201 | TAAGCTCATG GGACACTTCG CATGGTGGAC AGCCTTTGTT |
| 21241 | ACTAATGTGA ATGCGTCATC ATCTGAAGCA TTTTTAATTG |
| 21281 | GATGTAATTA TCTTGGCAAA CCACGCGAAC AAATAGATGG |
| 21321 | TTATGTCATG CATGCAAATT ACATATTTTG GAGGAATACA |
| 21361 | AATCCAATTC AGTTGTCTTC CTATTCTTTA TTTGACATGA |
| 21401 | GTAAATTTCC CCTTAAATTA AGGGGTACTG CTGTTATGTC |
| 21441 | TTTAAAAGAA GGTCAAATCA ATGATATGAT TTTATCTCTT |
| 21481 | CTTAGTAAAG GTAGACTTAT AATTAGAGAA AACAACAGAG |
| 21521 | TTGTTATTTC TAGTGATGTT CTTGTTAACA ACTAAACGAA |
| 21561 | CAATGTTTGT TTTTCTTGTT TTATTGCCAC TAGTCTCTAG |
| 21601 | TCAGTGTGTT AATCTTACAA CCAGAACTCA ATTACCCCCT |
| 21641 | GCATACACTA ATTCTTTCAC ACGTGGTGTT TATTACCCTG |
| 21681 | ACAAAGTTTT CAGATCCTCA GTTTTACATT CAACTCAGGA |
| 21721 | CTTGTTCTTA CCTTTCTTTT CCAATGTTAC TTGGTTCCAT |
| 21761 | GCTATACATG TCTCTGGGAC CAATGGTACT AAGAGGTTTG |
| 21801 | ATAACCCTGT CCTACCATTT AATGATGGTG TTTATTTTGC |
| 21841 | TTCCACTGAG AAGTCTAACA TAATAAGAGG CTGGATTTTT |
| 21881 | GGTACTACTT TAGATTCGAA GACCCAGTCC CTACTTATTG |
| 21921 | TTAATAACGC TACTAATGTT GTTATTAAAG TCTGTGAATT |
| 21961 | TCAATTTTGT AATGATCCAT TTTTGGGTGT TTATTACCAC |
| 22001 | AAAAACAACA AAAGTTGGAT GGAAAGTGAG TTCAGAGTTT |
| 22041 | ATTCTAGTGC GAATAATTGC ACTTTTGAAT ATGTCTCTCA |
| 22081 | GCCTTTTCTT ATGGACCTTG AAGGAAAACA GGGTAATTTC |
| 22121 | AAAAATCTTA GGGAATTTGT GTTTAAGAAT ATTGATGGTT |
| 22161 | ATTTTAAAAT ATATTCTAAG CACACGCCTA TTAATTTAGT |
| 22201 | GCGTGATCTC CCTCAGGGTT TTTCGGCTTT AGAACCATTG |
| 22241 | GTAGATTTGC CAATAGGTAT TAACATCACT AGGTTTCAAA |
| 22281 | CTTTACTTGC TTTACATAGA AGTTATTTGA CTCCTGGTGA |
| 22321 | TTCTTCTTCA GGTTGGACAG CTGGTGCTGC AGCTTATTAT |
| 22361 | GTGGGTTATC TTCAACCTAG GACTTTTCTA TTAAAATATA |
| 22401 | ATGAAAATGG AACCATTACA GATGCTGTAG ACTGTGCACT |
| 22441 | TGACCCTCTC TCAGAAACAA AGTGTACGTT GAAATCCTTC |
| 22481 | ACTGTAGAAA AAGGAATCTA TCAAACTTCT AACTTTAGAG |
| 22521 | TCCAACCAAC AGAATCTATT GTTAGATTTC CTAATATTAC |
| 22561 | AAACTTGTGC CCTTTTGGTG AAGTTTTTAA CGCCACCAGA |
| 22601 | TTTGCATCTG TTTATGCTTG GAACAGGAAG AGAATCAGCA |
| 22641 | ACTGTGTTGC TGATTATTCT GTCCTATATA ATTCCGCATC |
| 22681 | ATTTTCCACT TTTAAGTGTT ATGGAGTGTC TCCTACTAAA |
| 22721 | TTAAATGATC TCTGCTTTAC TAATGTCTAT GCAGATTCAT |
| 22761 | TTGTAATTAG AGGTGATGAA GTCAGACAAA TCGCTCCAGG |
| 22801 | GCAAACTGGA AAGATTGCTG ATTATAATTA TAAATTACCA |
| 22841 | GATGATTTTA CAGGCTGCGT TATAGCTTGG AATTCTAACA |
| 22881 | ATCTTGATTC TAAGGTTGGT GGTAATTATA ATTACCTGTA |
| 22921 | TAGATTGTTT AGGAAGTCTA ATCTCAAACC TTTTGAGAGA |
| 22961 | GATATTTCAA CTGAAATCTA TCAGGCCGGT AGCACACCTT |
| 23001 | GTAATGGTGT TGAAGGTTTT AATTGTTACT TTCCTTTACA |
| 23041 | ATCATATGGT TTCCAACCCA CTAATGGTGT TGGTTACCAA |
| 23081 | CCATACAGAG TAGTAGTACT TTCTTTTGAA CTTCTACATG |
| 23121 | CACCAGCAAC TGTTTGTGGA CCTAAAAAGT CTACTAATTT |
| 23161 | GGTTAAAAAC AAATGTGTCA ATTTCAACTT CAATGGTTTA |
| 23201 | ACAGGCACAG GTGTTCTTAC TGAGTCTAAC AAAAAGTTTC |
| 23241 | TGCCTTTCCA ACAATTTGGC AGAGACATTG CTGACACTAC |
| 23281 | TGATGCTGTC CGTGATCCAC AGACACTTGA GATTCTTGAC |
| 23321 | ATTACACCAT GTTCTTTTGG TGGTGTCAGT GTTATAACAC |
| 23361 | CAGGAACAAA TACTTCTAAC CAGGTTGCTG TTCTTTATCA |
| 23401 | GGATGTTAAC TGCACAGAAG TCCCTGTTGC TATTCATGCA |
| 23441 | GATCAACTTA CTCCTACTTG GCGTGTTTAT TCTACAGGTT |
| 23481 | CTAATGTTTT TCAAACACGT GCAGGCTGTT TAATAGGGGC |
| 23521 | TGAACATGTC AACAACTCAT ATGAGTGTGA CATACCCATT |
| 23561 | GGTGCAGGTA TATGCGCTAG TTATCAGACT CAGACTAATT |
| 23601 | CTCCTCGGCG GGCACGTAGT GTAGCTAGTC AATCCATCAT |
| 23641 | TGCCTACACT ATGTCACTTG GTGCAGAAAA TTCAGTTGCT |
| 23681 | TACTCTAATA ACTCTATTGC CATACCCACA AATTTTACTA |
| 23721 | TTAGTGTTAC CACAGAAATT CTACCAGTGT CTATGACCAA |
| 23761 | GACATCAGTA GATTGTACAA TGTACATTTG TGGTGATTCA |
| 23801 | ACTGAATGCA GCAATCTTTT GTTGCAATAT GGCAGTTTTT |
| 23841 | GTACACAATT AAACCGTGCT TTAACTGGAA TAGCTGTTGA |
| 23881 | ACAAGACAAA AACACCCAAG AAGTTTTTGC ACAAGTCAAA |
| 23921 | CAAATTTACA AAACACCACC AATTAAAGAT TTTGGTGGTT |
| 23961 | TTAATTTTTC ACAAATATTA CCAGATCCAT CAAAACCAAG |
| 24001 | CAAGAGGTCA TTTATTGAAG ATCTACTTTT CAACAAAGTG |
| 24041 | ACACTTGCAG ATGCTGGCTT CATCAAACAA TATGGTGATT |
| 24081 | GCCTTGGTGA TATTGCTGCT AGAGACCTCA TTTGTGCACA |
| 24121 | AAAGTTTAAC GGCCTTACTG TTTTGCCACC TTTGCTCACA |
| 24161 | GATGAAATGA TTGCTCAATA CACTTCTGCA CTGTTAGCGG |
| 24201 | GTACAATCAC TTCTGGTTGG ACCTTTGGTG CAGGTGCTGC |
| 24241 | ATTACAAATA CCATTTGCTA TGCAAATGGC TTATAGGTTT |
| 24281 | AATGGTATTG GAGTTACACA GAATGTTCTC TATGAGAACC |
| 24321 | AAAAATTGAT TGCCAACCAA TTTAATAGTG CTATTGGCAA |
| 24361 | AATTCAAGAC TCACTTTCTT CCACAGCAAG TGCACTTGGA |
| 24401 | AAACTTCAAG ATGTGGTCAA CCAAAATGCA CAAGCTTTAA |
| 24441 | ACACGCTTGT TAAACAACTT AGCTCCAATT TTGGTGCAAT |
| 24481 | TTCAAGTGTT TTAAATGATA TCCTTTCACG TCTTGACAAA |
| 24521 | GTTGAGGCTG AAGTGCAAAT TGATAGGTTG ATCACAGGCA |
| 24561 | GACTTCAAAG TTTGCAGACA TATGTGACTC AACAATTAAT |
| 24601 | TAGAGCTGCA GAAATCAGAG CTTCTGCTAA TCTTGCTGCT |
| 24641 | ACTAAAATGT CAGAGTGTGT ACTTGGACAA TCAAAAAGAG |
| 24681 | TTGATTTTTG TGGAAAGGGC TATCATCTTA TGTCCTTCCC |
| 24721 | TCAGTCAGCA CCTCATGGTG TAGTCTTCTT GCATGTGACT |
| 24761 | TATGTCCCTG CACAAGAAAA GAACTTCACA ACTGCTCCTG |
| 24801 | CCATTTGTCA TGATGGAAAA GCACACTTTC CTCGTGAAGG |
| 24841 | TGTCTTTGTT TCAAATGGCA CACACTGGTT TGTAACACAA |
| 24881 | AGGAATTTTT ATGAACCACA AATCATTACT ACAGACAACA |
| 24921 | CATTTGTGTC TGGTAACTGT GATGTTGTAA TAGGAATTGT |
| 24961 | CAACAACACA GTTTATGATC CTTTGCAACC TGAATTAGAC |
| 25001 | TCATTCAAGG AGGAGTTAGA TAAATATTTT AAGAATCATA |
| 25041 | CATCACCAGA TGTTGATTTA GGTGACATCT CTGGCATTAA |
| 25081 | TGCTTCAGTT GTAAACATTC AAAAAGAAAT TGACCGCCTC |
| 25121 | AATGAGGTTG CCAAGAATTT AAATGAATCT CTCATCGATC |
| 25161 | TCCAAGAACT TGGAAAGTAT GAGCAGTATA TAAAATGGCC |
| 25201 | ATGGTACATT TGGCTAGGTT TTATAGCTGG CTTGATTGCC |
| 25241 | ATAGTAATGG TGACAATTAT GCTTTGCTGT ATGACCAGTT |
| 25281 | GCTGTAGTTG TCTCAAGGGC TGTTGTTCTT GTGGATCCTG |
| 25321 | CTGCAAATTT GATGAAGACG ACTCTGAGCC AGTGCTCAAA |
| 25361 | GGAGTCAAAT TACATTACAC ATAAACGAAC TTATGGATTT |
| 25401 | GTTTATGAGA ATCTTCACAA TTGGAACTGT AACTTTGAAG |
| 25441 | CAAGGTGAAA TCAAGGATGC TACTCCTTCA GATTTTGTTC |
| 25481 | GCGCTACTGC AACGATACCG ATACAAGCCT CACTCCCTTT |
| 25521 | CGGATGGCTT ATTGTTGGCG TTGCACTTCT TGCTGTTTTT |
| 25561 | CAGAGCGCTT CCAAAATCAT AACCCTCAAA AAGAGATGGC |
| 25601 | AACTAGCACT CTCCAAGGGT GTTCACTTTG TTTGCAACTT |
| 25641 | GCTGTTGTTG TTTGTAACAG TTTACTCACA CCTTTTGCTC |
| 25681 | GTTGCTGCTG GCCTTGAAGC CCCTTTTCTC TATCTTTATG |
| 25721 | CTTTAGTCTA CTTCTTGCAG AGTATAAACT TTGTAAGAAT |
| 25761 | AATAATGAGG CTTTGGCTTT GCTGGAAATG CCGTTCCAAA |
| 25801 | AACCCATTAC TTTATGATGC CAACTATTTT CTTTGCTGGC |
| 25841 | ATACTAATTG TTACGACTAT TGTATACCTT ACAATAGTGT |
| 25881 | AACTTCTTCA ATTGTCATTA CTTCAGGTGA TGGCACAACA |
| 25921 | AGTCCTATTT CTGAACATGA CTACCAGATT GGTGGTTATA |
| 25961 | CTGAAAAATG GGAATCTGGA GTAAAAGACT GTGTTGTATT |
| 26001 | ACACAGTTAC TTCACTTCAG ACTATTACCA GCTGTACTCA |
| 26041 | ACTCAATTGA GTACAGACAC TGGTGTTGAA CATGTTACCT |
| 26081 | TCTTCATCTA CAATAAAATT GTTGATGAGC CTGAAGAACA |
| 26121 | TGTCCAAATT CACACAATCG ACGGTTCATC CGGAGTTGTT |
| 26161 | AATCCAGTAA TGGAACCAAT TTATGATGAA CCGACGACGA |
| 26201 | CTACTAGCGT GCCTTTGTAA GCACAAGCTG ATGAGTACGA |
| 26241 | ACTTATGTAC TCATTCGTTT CGGAAGAGAC AGGTACGTTA |
| 26281 | ATAGTTAATA GCGTACTTCT TTTTCTTGCT TTCGTGGTAT |
| 26321 | TCTTGCTAGT TACACTAGCC ATCCTTACTG CGCTTCGATT |
| 26361 | GTGTGCGTAC TGCTGCAATA TTGTTAACGT GAGTCTTGTA |
| 26401 | AAACCTTCTT TTTACGTTTA CTCTCGTGTT AAAAATCTGA |
| 26441 | ATTCTTCTAG AGTTCCTGAT CTTCTGGTCT AAACGAACTA |
| 26481 | AATATTATAT TAGTTTTTCT GTTTGGAACT TTAATTTTAG |
| 26521 | CCATGGCAGA TTCCAACGGT ACTATTACCG TTGAAGAGCT |
| 26561 | TAAAAAGCTC CTTGAACAAT GGAACCTAGT AATAGGTTTC |
| 26601 | CTATTCCTTA CATGGATTTG TCTTCTACAA TTTGCCTATG |
| 26641 | CCAACAGGAA TAGGTTTTTG TATATAATTA AGTTAATTTT |
| 26681 | CCTCTGGCTG TTATGGCCAG TAACTTTAGC TTGTTTTGTG |
| 26721 | CTTGCTGCTG TTTACAGAAT AAATTGGATC ACCGGTGGAA |
| 26761 | TTGCTATCGC AATGGCTTGT CTTGTAGGCT TGATGTGGCT |
| 26801 | CAGCTACTTC ATTGCTTCTT TCAGACTGTT TGCGCGTACG |
| 26841 | CGTTCCATGT GGTCATTCAA TCCAGAAACT AACATTCTTC |
| 26881 | TCAACGTGCC ACTCCATGGC ACTATTCTGA CCAGACCGCT |
| 26921 | TCTAGAAAGT GAACTCGTAA TCGGAGCTGT GATCCTTCGT |
| 26961 | GGACATCTTC GTATTGCTGG ACACCATCTA GGACGCTGTG |
| 27001 | ACATCAAGGA CCTGCCTAAA GAAATCACTG TTGCTACATC |
| 27041 | ACGAACGCTT TCTTATTACA AATTGGGAGC TTCGCAGCGT |
| 27081 | GTAGCAGGTG ACTCAGGTTT TGCTGCATAC AGTCGCTACA |
| 27121 | GGATTGGCAA CTATAAATTA AACACAGACC ATTCCAGTAG |
| 27161 | CAGTGACAAT ATTGCTTTGC TTGTACAGTA AGTGACAACA |
| 27201 | GATGTTTCAT CTCGTTGACT TTCAGGTTAC TATAGCAGAG |
| 27241 | ATATTACTAA TTATTATGAG GACTTTTAAA GTTTCCATTT |
| 27281 | GGAATCTTGA TTACATCATA AACCTCATAA TTAAAAATTT |
| 27321 | ATCTAAGTCA CTAACTGAGA ATAAATATTC TCAATTAGAT |
| 27361 | GAAGAGCAAC CAATGGAGAT TGATTAAACG AACATGAAAA |
| 27401 | TTATTCTTTT CTTGGCACTG ATAACACTCG CTACTTGTGA |
| 27441 | GCTTTATCAC TACCAAGAGT GTGTTAGAGG TACAACAGTA |
| 27481 | CTTTTAAAAG AACCTTGCTC TTCTGGAACA TACGAGGGCA |
| 27521 | ATTCACCATT TCATCCTCTA GCTGATAACA AATTTGCACT |
| 27561 | GACTTGCTTT AGCACTCAAT TTGCTTTTGC TTGTCCTGAC |
| 27601 | GGCGTAAAAC ACGTCTATCA GTTACGTGCC AGATCAGTTT |
| 27641 | CACCTAAACT GTTCATCAGA CAAGAGGAAG TTCAAGAACT |
| 27681 | TTACTCTCCA ATTTTTCTTA TTGTTGCGGC AATAGTGTTT |
| 27721 | ATAACACTTT GCTTCACACT CAAAAGAAAG ACAGAATGAT |
| 27761 | TGAACTTTCA TTAATTGACT TCTATTTGTG CTTTTTAGCC |
| 27801 | TTTCTGCTAT TCCTTGTTTT AATTATGCTT ATTATCTTTT |
| 27841 | GGTTCTCACT TGAACTGCAA GATCATAATG AAACTTGTCA |
| 27881 | CGCCTAAACG AACATGAAAT TTCTTGTTTT CTTAGGAATC |
| 27921 | ATCACAACTG TAGCTGCATT TCACCAAGAA TGTAGTTTAC |
| 27961 | AGTCATGTAC TCAACATCAA CCATATGTAG TTGATGACCC |
| 28001 | GTGTCCTATT CACTTCTATT CTAAATGGTA TATTAGAGTA |
| 28041 | GGAGCTAGAA AATCAGCACC TTTAATTGAA TTGTGCGTGG |
| 28081 | ATGAGGCTGG TTCTAAATCA CCCATTCAGT ACATCGATAT |
| 28121 | CGGTAATTAT ACAGTTTCCT GTTTACCTTT TACAATTAAT |
| 28161 | TGCCAGGAAC CTAAATTGGG TAGTCTTGTA GTGCGTTGTT |
| 28201 | CGTTCTATGA AGACTTTTTA GAGTATCATG ACGTTCGTGT |
| 28241 | TGTTTTAGAT TTCATCTAAA CGAACAAACT AAAATGTCTG |
| 28281 | ATAATGGACC CCAAAATCAG CGAAATGCAC CCCGCATTAC |
| 28321 | GTTTGGTGGA CCCTCAGATT CAACTGGCAG TAACCAGAAT |
| 28361 | GGAGAACGCA GTGGGGCGCG ATCAAAACAA CGTCGGCCCC |
| 28401 | AAGGTTTACC CAATAATACT GCGTCTTGGT TCACCGCTCT |
| 28441 | CACTCAACAT GGCAAGGAAG ACCTTAAATT CCCTCGAGGA |
| 28481 | CAAGGCGTTC CAATTAACAC CAATAGCAGT CCAGATGACC |
| 28521 | AAATTGGCTA CTACCGAAGA GCTACCAGAC GAATTCGTGG |
| 28561 | TGGTGACGGT AAAATGAAAG ATCTCAGTCC AAGATGGTAT |
| 28601 | TTCTACTACC TAGGAACTGG GCCAGAAGCT GGACTTCCCT |
| 28641 | ATGGTGCTAA CAAAGACGGC ATCATATGGG TTGCAACTGA |
| 28681 | GGGAGCCTTG AATACACCAA AAGATCACAT TGGCACCCGC |
| 28721 | AATCCTGCTA ACAATGCTGC AATCGTGCTA CAACTTCCTC |
| 28761 | AAGGAACAAC ATTGCCAAAA GGCTTCTACG CAGAAGGGAG |
| 28801 | CAGAGGCGGC AGTCAAGCCT CTTCTCGTTC CTCATCACGT |
| 28841 | AGTCGCAACA GTTCAAGAAA TTCAACTCCA GGCAGCAGTA |
| 28881 | GGGGAACTTC TCCTGCTAGA ATGGCTGGCA ATGGCGGTGA |
| 28921 | TGCTGCTCTT GCTTTGCTGC TGCTTGACAG ATTGAACCAG |
| 28961 | CTTGAGAGCA AAATGTCTGG TAAAGGCCAA CAACAACAAG |
| 29001 | GCCAAACTGT CACTAAGAAA TCTGCTGCTG AGGCTTCTAA |
| 29041 | GAAGCCTCGG CAAAAACGTA CTGCCACTAA AGCATACAAT |
| 29081 | GTAACACAAG CTTTCGGCAG ACGTGGTCCA GAACAAACCC |
| 29121 | AAGGAAATTT TGGGGACCAG GAACTAATCA GACAAGGAAC |
| 29161 | TGATTACAAA CATTGGCCGC AAATTGCACA ATTTGCCCCC |
| 29201 | AGCGCTTCAG CGTTCTTCGG AATGTCGCGC ATTGGCATGG |
| 29241 | AAGTCACACC TTCGGGAACG TGGTTGACCT ACACAGGTGC |
| 29281 | CATCAAATTG GATGACAAAG ATCCAAATTT CAAAGATCAA |
| 29321 | GTCATTTTGC TGAATAAGCA TATTGACGCA TACAAAACAT |
| 29361 | TCCCACCAAC AGAGCCTAAA AAGGACAAAA AGAAGAAGGC |
| 29401 | TGATGAAACT CAAGCCTTAC CGCAGAGACA GAAGAAACAG |
| 29441 | CAAACTGTGA CTCTTCTTCC TGCTGCAGAT TTGGATGATT |
| 29481 | TCTCCAAACA ATTGCAACAA TCCATGAGCA GTGCTGACTC |
| 29521 | AACTCAGGCC TAAACTCATG CAGACCACAC AAGGCAGATG |
| 29561 | GGCTATATAA ACGTTTTCGC TTTTCCGTTT ACGATATATA |
| 29601 | GTCTACTCTT GTGCAGAATG AATTCTCGTA ACTACATAGC |
| 29641 | ACAAGTAGAT GTAGTTAACT TTAATCTCAC ATAGCAATCT |
| 29681 | TTAATCAGTG TGTAACATTA GGGAGGACTT GAAAGAGCCA |
| 29721 | CCACATTTTC ACCGAGGCCA CGCGGAGTAC GATCGAGTGT |
| 29761 | ACAGTGAACA ATGCTAGGGA GAGCTGCCTA TATGGAAGAG |
| 29801 | CCCTAATGTG TAAAATTAAT TTTAGTAGTG CTATCCCCAT |
| 29841 | GTGATTTTAA TAGCTTCTTA GGAGAATGAC AAAAAAAAAA |
| 29881 | AAAAAAAAAA AAAAAAAAAA AAA |

The SARS-CoV-2 can have a 5′ untranslated region (5′ UTR; also known as a leader sequence or leader RNA) at positions 1-265 of the SEQ ID NO:1 sequence. Such a 5′ UTR can include the region of an mRNA that is directly upstream from the initiation codon.

Similarly, the SARS-CoV-2 can have a 3′ untranslated region (3′ UTR) at positions 29675-29903. In positive strand RNA viruses, the 3′-UTR can play a role in viral RNA replication because the origin of the minus-strand RNA replication intermediate is at the 3′-end of the genome.

The SARS-CoV-2 genome encodes four major structural proteins: the spike (S) protein, nucleocapsid (N) protein, membrane (M) protein, and the envelope (E) protein. Some of these proteins are part of a large polyprotein, which is at positions 266-21555 of the SEQ ID NO:1 sequence, where this open reading frame is referred to as ORF1ab polyprotein and has SEQ ID NO:12, shown below.

| | |
|---|---|
| 1 | MESLVPGfNE KTHVQLSLPV LQVRDVLVRG FGDSVEEVLS |
| 41 | EARQHLKDGT CGLVEVEKGV LPQLEQPYVf IKRSDARTAP |
| 81 | HGHVMVELVA ELEGIQYGRS GETLGVLVPH VGEIPVAYRK |
| 121 | VLLRKNGNKG AGGHSYGADL KSFDLGDELG TDPYEDFQEN |
| 161 | WNTKHSSGVT RELMRELNGG AYTRYVDNNF CGPDGYPLEC |
| 201 | IKDLLARAGK ASCTLSEQLD FIDTKRGVYC CREHEHEIAW |
| 241 | YTERSEKSYE LQTPFEIKLA KKFDTfNGEC PNFVEPLNSI |
| 281 | IKTIQPRVEK KKLDGFMGRI RSVYPVASPN ECNQMCLSTL |
| 321 | MKCDHCGETS WQTGDFVKAT CEFCGTENLT KEGATTCGYL |
| 361 | PQNAVVKIYC PACHNSEVGP EHSLAEYHNE SGLKTILRKG |
| 401 | GRTIAFGGCV FSYVGCHNKC AYWVPRASAN IGCNHTGVVG |
| 441 | EGSEGLNDNL LEILQKEKVN INIVGDFKLN EEIAIILASF |
| 481 | SASTSAFVET VKGLDYKAFK QIVESCGNFK VTKGKAKKGA |
| 521 | WNIGEQKSIL SPLYAFASEA ARVVRSIFSR TLETAQNSVR |
| 561 | VLQKAAITIL DGISQYSLRL IDAMMFTSDL ATNNLVVMAY |
| 601 | ITGGVVQLTS QWLTNIFGTV YEKLKPVLDW LEEKFKEGVE |
| 641 | FLRDGWEIVK FISTCACEIV GGQIVTCAKE IKESVQTFFK |
| 681 | LVNKFLALCA DSIIIGGAKL KALNLGETFV THSKGLYRKC |
| 721 | VKSREETGLL MPLKAPKEII FLEGETLPTE VLTEEVVLKT |
| 761 | GDLQPLEQPT SEAVEAPLVG TPVCINGLML LEIKDTEKYC |
| 801 | ALAPNMMVTN NTFTLKGGAP TKVTFGDDTV IEVQGYKSVN |
| 841 | ITFELDERID KVLNEKCSAY TVELGTEVNE FACVVADAVI |
| 881 | KTLQPVSELL TPLGIDLDEW SMATYYLFDE SGEFKLASHM |
| 921 | YCSFYPPDED EEEGDCEEEE FEPSTQYEYG TEDDYQGKPL |
| 961 | EFGATSAALQ PEEEQEEDWL DDDSQQTVGQ QDGSEDNQTT |
| 1001 | TIQTIVEVQP QLEMELTPVV QTIEVNSFSG YLKLIDNVYI |
| 1041 | KNADIVEEAK KVKPTVVVNA ANVYLKHGGG VAGALNKATN |
| 1081 | NAMQVESDDY IAINGPLKVG GSCVLSGHNL AKHCLHVVGP |
| 1121 | NVNKGEDIQL LKSAYENFNQ HEVLLAPLLS AGIFGADPIH |
| 1161 | SLRVCVDTVR TNVYLAVFDK NLYDKLVSSF LEMKSEKQVE |
| 1201 | QKIAEIPKEE VKPFITESKP SVEQRKQDDK KIKACVEEVT |
| 1241 | TTLEETKFLT ENLLLYIDIN GNLHPDSATL VSDIDITFLK |
| 1281 | KDAPYIVGDV VQEGVLTAVV IPTKKAGGTT EMLAKALRKV |
| 1321 | PTDNYITTYP GQGLNGYTVE EAKTVLKKCK SAFYILPSII |
| 1361 | SNEKQEILGT VSWNLREMLA HAEETRKLMP VCVETKAIVS |
| 1401 | TIQRKYKGIK IQEGVVDYGA RFYFYTSKTT VASLINTIND |
| 1441 | LNETLVTMPL GYVTHGLNLE EAARYMRSLK VPATVSVSSP |
| 1481 | DAVTAYNGYL TSSSKTPEEH FIETISLAGS YKDWSYSGQS |
| 1521 | TQLGIEFLKR GDKSVYYTSN PTTFHLDGEV ITFDNLKTLL |
| 1561 | SLREVRTIKV FTTVDNINLH TQVVDMSMTY GQQFGPTYLD |
| 1601 | GADVTKIKPH NSHEGKTFYV LPNDDTLRVE AFEYYHTTDP |
| 1641 | SFLGRYMSAL NHTKKWKYPQ VNGLTSIKWA DNNCYLATAL |
| 1681 | LTLQQIELKF NPPALQDAYY RARAGEAANF CALILAYCNK |
| 1721 | TVGELGDVRE TMSYLFQHAN LDSCKRVLNV VCKTCGQQQT |
| 1761 | TLKGVEAVMY MGTLSYEQFK KGVQIPCTCG KQATKYLVQQ |
| 1801 | ESPFVMMSAP PAQYELKHGT FTCASEYTGN YQCGHYKHIT |
| 1841 | SKETLYCIDG ALLTKSSEYK GPITDVFYKE NSYTTTIKPV |
| 1881 | TYKLDGVVCT EIDPKLDNYY KKDNSYFTEQ PIDLVPNQPY |
| 1921 | PNASFDNFKF VCDNIKFADD LNQLTGYKKP ASRELKVTFF |
| 1961 | PDLNGDVVAI DYKHYTPSFK KGAKLLHKPI VWHVNNATNK |
| 2001 | ATYKPNTWCI RCLWSTKPVE TSNSFDVLKS EDAQGMDNLA |
| 2041 | CEDLKPVSEE VVENPTIQKD VLECNVKTTE VVGDIILKPA |
| 2081 | NNSLKITEEV GHTDLMAAYV DNSSLTIKKP NELSRVLGLK |
| 2121 | TLATHGLAAV NSVPWDTIAN YAKPFLNKVV STTTNIVTRC |
| 2161 | LNRVCTNYMP YFFTLLLQLC TFTRSTNSRI KASMPTTIAK |
| 2201 | NTVKSVGKFC LEASFNYLKS PNFSKLINII IWFLLLSVCL |
| 2241 | GSLIYSTAAL GVLMSNLGMP SYCTGYREGY LNSTNVTIAT |
| 2281 | YCTGSIPCSV CLSGLDSLDT YPSLETIQIT ISSFKWDLTA |
| 2321 | FGLVAEWFLA YILFTRFFYV LGLAAIMQLF FSYFAVHFIS |
| 2361 | NSWLMWLIIN LVQMAPISAM VRMYIFFASF YYVWKSYVHV |
| 2401 | VDGCNSSTCM MCYKRNRATR VECTTIVNGV RRSFYVYANG |
| 2441 | GKGFCKLHNW NCVNCDTFCA GSTFISDEVA RDLSLQFKRP |
| 2481 | INPTDQSSYI VDSVTVKNGS IHLYFDKAGQ KTYERHSLSH |
| 2521 | FVNLDNLRAN NTKGSLPINV IVFDGKSKCE ESSAKSASVY |
| 2561 | YSQLMCQPIL LLDQALVSDV GDSAEVAVKM FDAYVNTFSS |
| 2601 | TFNVPMEKLK TLVATAEAEL AKNVSLDNVL STFISAARQG |
| 2641 | FVDSDVETKD VVECLKLSHQ SDIEVTGDSC NNYMLTYNKV |
| 2481 | ENMTPRDLGA CIDCSARHIN AQVAKSHNIA LIWNVKDFMS |
| 2521 | LSEQLRKQIR SAAKKNNLPF KLTCATTRQV VNVVTTKIAL |
| 2561 | KGGKIVNNWL KQLIKVILVF LFVAAIFYLI TPVHVMSKHT |
| 2601 | DFSSEIIGYK AIDGGVTRDI ASTDTCFANK HADFDTWFSQ |
| 2641 | RGGSYTNDKA CPLIAAVITR EVGFVVPGLP GTILRTTNGD |
| 2681 | FLHFLPRVFS AVGNICYTPS KLIEYTDFAT SACVLAAECT |
| 2721 | IFKDASGKPV PYCYDTNVLE GSVAYESLRP DTRYVLMDGS |
| 2761 | IIQFPNTYLE GSVRVVTTFD SEYCRHGTCE RSEAGVCVST |
| 2801 | SGRWVLNNDY YRSLPGVFCG VDAVNLLTNM FTPLIQPIGA |
| 2841 | LDISASIVAG GIVAIVVTCL AYYFMRFRRA FGEYSHVVAF |
| 2881 | NTLLFLMSFT VLCLTPVYSF LPGVYSVIYL YLTFYLTNDV |
| 2921 | SFLAHIQWMV MFTPLVPFWI TIAYIICIST KHFYWFFSNY |
| 2961 | LKRRVVFNGV SFSTFEEAAL CTFLLNKEMY LKLRSDVLLP |
| 3001 | LTQYNRYLAL YNKYKYFSGA MDTTSYREAA CCHLAKALND |
| 3041 | FSNSGSDVLY QPPQTSITSA VLQSGFRKMA FPSGKVEGCM |
| 3081 | VQVTCGTTTL NGLWLDDVVY CPRHVICTSE DMLNPNYEDL |
| 3121 | LIRKSNHNFL VQAGNVQLRV IGHSMQNCVL KLKVDTANPK |
| 3161 | TPKYKFVRIQ PGQTFSVLAC YNGSPSGVYQ CAMRPNFTIK |
| 3201 | GSFLNGSCGS VGFNIDYDCV SFCYMHHMEL PTGVHAGTDL |
| 3241 | EGNFYGPFVD RQTAQAAGTD TTITVNVLAW LYAAVINGDR |
| 3281 | WFLNRFTTTL NDFNLVAMKY NYEPLTQDHV DILGPLSAQT |
| 3321 | GIAVLDMCAS LKELLQNGMN GRTILGSALL EDEFTPFDVV |
| 3361 | RQCSGVTFQS AVKRTIKGTH HWLLLTILTS LLVIVQSTQW |
| 3401 | SLFFFLYENA FLPFAMGIIA MSAFAMMFVK HKHAFLCLFL |
| 3441 | LPSLATVAYF NMVYMPASWV MRIMTWLDMV DTSLSGFKLK |
| 3481 | DCVMYASAVV LLILMTARTV YDDGARRVWT LMNVLTLVYK |
| 3521 | VYYGNALDQA ISMWALIISV TSNYSGVVTT VMFLARGIVF |
| 3561 | MCVEYCPIFF ITGNTLQCIM LVYCFLGYFC TCYFGLFCLL |
| 3601 | NRYFRLTLGV YDYLVSTQEF RYMNSQGLLP PKNSIDAFKL |
| 3641 | NIKLLGVGGK PCIKVATVQS KMSDVKCTSV VLLSVLQQLR |
| 3681 | VESSSKLWAQ CVQLHNDILL AKDTTEAFEK MVSLLSVLLS |
| 3721 | MQGAVDINKL CEEMLDNRAT LQAIASEFSS LPSYAAFATA |
| 3761 | QEAYEQAVAN GDSEVVLKKL KKSLNVAKSE FDRDAAMQRK |
| 3801 | LEKMADQAMT QMYKQARSED KRAKVISAMQ TMLFTMLRKL |
| 3841 | DNDALNNIIN NARDGCVPLN IIPLTTAAKL MVVIPDYNTY |
| 3881 | KNTCDGTTFT YASALWEIQQ VVDADSKIVQ LSEISMDNSP |
| 3921 | NLAWPLIVTA LRANSAVKLQ NNELSPVALR QMSCAAGTTQ |
| 3961 | TACTDDNALA YYNTTKGGRF VLALLSDLQD LKWARFPKSD |
| 4001 | GTGTIYTELE PPCRFVTDTP KGPKVKYLYF IKGLNNLNRG |
| 4041 | MVLGSLAATV RLQAGNATEV PANSTVLSFC AFAVDAAKAY |
| 4081 | KDYLASGGQP ITNCVKMLCT HTGTGQAITV TPEANMDQES |
| 4121 | FGGASCCLYC RCHIDHPNPK GFCDLKGKYV QIPTTCANDP |
| 4161 | VGFTLKNTVC TVCGMWKGYG CSCDQLREPM LQSADAQSFL |
| 4201 | NGFAV |

An RNA-dependent RNA polymerase is encoded at positions 13442-13468 and 13468-16236 of the SARS-CoV-2 SEQ ID NO:1 nucleic acid. This RNA-dependent RNA polymerase has been assigned NCBI accession number YP_009725307 and has the following sequence (SEQ ID NO:13)

| | |
|---|---|
| 1 | SADAQSFLNR VCGVSAARLT PCGTGTSTDV VYRAFDIYND |
| 41 | KVAGFAKFLK INCCRFQEKD EDDNLIDSYF VVKRHTFSNY |
| 81 | QHEETIYNLL KDCPAVAKHD FFKFRIDGDM VPHISRQRLT |
| 121 | KYTMADLVYA LRHFDEGNCD TLKEILVTYN CCDDDYFNKK |
| 161 | DWYDFVENPD ILRVYANLGE RVRQALLKTV QFCDAMRNAG |
| 201 | IVGVLTLDNQ DLNGNWYDFG DFIQTTPGSG VPVVDSYYSL |
| 241 | LMPILTLTRA LTAESHVDTD LTKPYIKWDL LKYDFTEERL |
| 281 | KLFDRYFKYW DQTYHPNCVN CLDDRCILHC ANFNVLFSTV |
| 321 | FPPTSFGPLV RKIFVDGVPF VVSTGYHFRE LGVVHNQDVN |
| 361 | LHSSRLSFKE LLVYAADPAM HAASGNLLLD KRTTCFSVAA |
| 401 | LTNNVAFQTV KPGNFNKDFY DFAVSKGFFK EGSSVELKHF |
| 441 | FFAQDGNAAI SDYDYYRYNL PTMCDIRQLL FVVEVVDKYF |
| 481 | DCYDGGCINA NQVIVNNLDK SAGFPFNKWG KARLYYDSMS |
| 521 | YEDQDALFAY TKRNVIPTIT QMNLKYAISA KNRARTVAGV |
| 561 | SICSTMTNRQ FHQKLLKSIA ATRGATVVIG TSKFYGGWHN |
| 601 | MLKTVYSDVE NPHLMGWDYP KCDRAMPNML RIMASLVLAR |
| 641 | KHTTCCSLSH RFYRLANECA QVLSEMVMCG GSLYVKPGGT |
| 681 | SSGDATTAYA NSVFNICQAV TANVNALLST DGNKIADKYV |
| 721 | RNLQHRLYEC LYRNRDVDTD FVNEFYAYLR KHFSMMILSD |
| 761 | DAVVCFNSTY ASQGLVASIK NFKSVLYYQN NVFMSEAKCW |
| 801 | TETDLTKGPH EFCSQHTMLV KQGDDYVYLP YPDPSRILGA |
| 841 | GCFVDDIVKT DGTLMIERFV SLAIDAYPLT KHPNQEYADV |
| 881 | FHLYLQYIRK LHDELTGHML DMYSVMLTND NTSRYWEPEF |
| 921 | YEAMYTPHTV LQ |

A helicase is encoded at positions 16237-18039 of the SARS-CoV-2 SEQ ID NO:1 nucleic acid. This helicase has been assigned NCBI accession number YP_009725308.1 and has the following sequence (SEQ ID NO:14).

| | |
|---|---|
| 1 | AVGACVLCNS QTSLRCGACI RRPFLCCKCC YDHVISTSHK |
| 41 | LVLSVNPYVC NAPGCDVTDV TQLYLGGMSY YCKSHKPPIS |
| 81 | FPLCANGQVF GLYKNTCVGS DNVTDFNAIA TCDWTNAGDY |
| 121 | ILANTCTERL KLFAAETLKA TEETFKLSYG IATVREVLSD |
| 161 | RELHLSWEVG KPRPPLNRNY VFTGYRVTKN SKVQIGEYTF |
| 201 | EKGDYGDAVV YRGTTTYKLN VGDYFVLTSH TVMPLSAPTL |
| 241 | VPQEHYVRIT GLYPTLNISD EFSSNVANYQ KVGMQKYSTL |
| 281 | QGPPGTGKSH FAIGLALYYP SARIVYTACS HAAVDALCEK |
| 321 | ALKYLPIDKC SRIIPARARV ECFDKFKVNS TLEQYVFCTV |
| 361 | NALPETTADI VVFDEISMAT NYDLSVVNAR LRAKHYVYIG |
| 401 | DPAQLPAPRT LLTKGTLEPE YFNSVCRLMK TIGPDMFLGT |
| 441 | CRRCPAEIVD TVSALVYDNK LKAHKDKSAQ CFKMFYKGVI |
| 481 | THDVSSAINR PQIGVVREFL TRNPAWRKAV FISPYNSQNA |
| 521 | VASKILGLPT QTVDSSQGSE YDYVIFTQTT ETAHSCNVNR |
| 561 | FNVAITRAKV GILCIMSDRD LYDKLQFTSL EIPRRNVATL |
| 601 | Q |

The SARS-CoV-2 can have an open reading frame at positions 21563-25384 (gene S) of the SEQ ID NO:1 sequence that can be referred to as GU280_gp02, where this open reading frame encodes a surface glycoprotein or a Spike glycoprotein (SEQ ID NO:5, shown below).

| | |
|---|---|
| 1 | MFVFLVLLPL VSSQCVNLTT RTQLPPAYTN SFTRGVYYPD |
| 41 | KVFRSSVLHS TQDLFLPFFS NVTWFHAIHV SGTNGTKRFD |
| 81 | NPVLPFNDGV YFASTEKSNI IRGWIFGTTL DSKTQSLLIV |
| 121 | NNATNVVIKV CEFQFCNDPF LGVYYHKNNK SWMESEFRVY |
| 161 | SSANNCTFEY VSQPFLMDLE GKQGNFKNLR EFVFKNIDGY |
| 201 | FKIYSKHTPI NLVRDLPQGF SALEPLVDLP IGINITRFQT |
| 241 | LLALHRSYLT PGDSSSGWTA GAAAYYVGYL QPRTFLLKYN |
| 281 | ENGTITDAVD CALDPLSETK CTLKSFTVEK GIYQTSNFRV |
| 321 | QPTESIVRFP NITNLCPFGE VFNATRFASV YAWNRKRISN |
| 361 | CVADYSVLYN SASFSTFKCY GVSPTKLNDL CFTNVYADSF |
| 401 | VIRGDEVRQI APGQTGKIAD YNYKLPDDFT GCVIAWNSNN |
| 441 | LDSKVGGNYN YLYRLFRKSN LKPFERDIST EIYQAGSTPC |
| 481 | NGVEGFNCYF PLQSYGFQPT NGVGYQPYRV VVLSFELLHA |
| 521 | PATVCGPKKS TNLVKNKCVN FNFNGLTGTG VLTESNKKFL |
| 561 | PFQQFGRDIA DTTDAVRDPQ TLEILDITPC SFGGVSVITP |
| 601 | GTNTSNQVAV LYQDVNCTEV PVAIHADQLT PTWRVYSTGS |
| 641 | NVFQTRAGCL IGAEHVNNSY ECDIPIGAGI CASYQTQTNS |
| 681 | PRRARSVASQ SIIAYTMSLG AENSVAYSNN SIAIPTNFTI |
| 721 | SVTTEILPVS MTKTSVDCTM YICGDSTECS NLLLQYGSFC |
| 761 | TQLNRALTGI AVEQDKNTQE VFAQVKQIYK TPPIKDFGGF |
| 801 | NFSQILPDPS KPSKRSFIED LLFNKVTLAD AGFIKQYGDC |
| 841 | LGDIAARDLI CAQKFNGLTV LPPLLTDEMI AQYTSALLAG |
| 881 | TITSGWTFGA GAALQIPFAM QMAYRFNGIG VTQNVLYENQ |
| 921 | KLIANQFNSA IGKIQDSLSS TASALGKLQD VVNQNAQALN |
| 961 | TLVKQLSSNF GAISSVLNDI LSRLDKVEAE VQIDRLITGR |
| 1001 | LQSLQTYVTQ QLIRAAEIRA SANLAATKMS ECVLGQSKRV |
| 1041 | DFCGKGYHLM SFPQSAPHGV VFLHVTYVPA QEKNFTTAPA |
| 1081 | ICHDGKAHFP REGVFVSNGT HWFVTQRNFY EPQIITTDNT |
| 1121 | FVSGNCDVVI GIVNNTVYDP LQPELDSFKE ELDKYFKNHT |
| 1161 | SPDVDLGDIS GINASVVNIQ KEIDRLNEVA KNLNESLIDL |
| 1201 | QELGKYEQYI KWPWYIWLGF IAGLIAIVMV TIMLCCMTSC |
| 1241 | CSCLKGCCSC GSCCKFDEDD SEPVLKGVKL HYT |

In some cases, the constructs and SARS-CoV-2 virus-like particles described herein can have a mutation or deletion of the SARS-CoV-2 Spike protein with SEQ ID NO:5. Such deletions/mutations can modulate or inactivate the function of the Spike protein. For example, in some cases deletions/mutations of the Spike protein can modulate interactions of the SARS-CoV-2 virus-like particles with receptor/receiver cells.

The S or spike protein is involved in facilitating entry of the SARS-CoV-2 into cells. It is composed of a short intracellular tail, a transmembrane anchor, and a large ectodomain that consists of a receptor binding S1 subunit and a membrane-fusing S2 subunit. The spike receptor binding domain can reside at amino acid positions 330-583 of the SEQ ID NO:5 spike protein (shown below as SEQ ID NO:15).

| | |
|---|---|
| 330 | P NITNLCPFGE VFNATRFASV YAWNRKRISN |
| 361 | CVADYSVLYN SASFSTFKCY GVSPTKLNDL CFTNVYADSF |
| 401 | VIRGDEVRQI APGQTGKIAD YNYKLPDDFT GCVIAWNSNN |
| 441 | LDSKVGGNYN YLYRLFRKSN LKPFERDIST EIYQAGSTPC |
| 481 | NGVEGFNCYF PLQSYGFQPT NGVGYQPYRV VVLSFELLHA |
| 521 | PATVCGPKKS TNLVKNKCVN FNFNGLTGTG VLTESNKKFL |
| 561 | PFQQFGRDIA DTTDAVRDPQ TLE |

Analysis of this receptor binding motif (RBM) in the spike protein showed that most of the amino acid residues essential for receptor binding were conserved between SARS-CoV and SARS-CoV-2, suggesting that the 2 CoV strains use the same host receptor for cell entry. The entry receptor utilized by SARS-CoV is the angiotensin-converting enzyme 2 (ACE-2).

The SARS-CoV-2 spike protein membrane-fusing S2 domain can be at positions 662-1270 of the SEQ ID NO:5 spike protein (shown below as SEQ ID NO:16).

| | |
|---|---|
| 662 | CDIPIGAGI CASYQTQTNS |
| 681 | PRRARSVASQ SIIAYTMSLG AENSVAYSNN SIAIPTNFTI |
| 721 | SVTTEILPVS MTKTSVDCTM YICGDSTECS NLLLQYGSFC |
| 761 | TQLNRALTGI AVEQDKNTQE VFAQVKQIYK TPPIKDFGGF |
| 801 | NFSQILPDPS KPSKRSFIED LLFNKVTLAD AGFIKQYGDC |
| 841 | LGDIAARDLI CAQKENGLTV LPPLLTDEMI AQYTSALLAG |
| 881 | TITSGWTFGA GAALQIPFAM QMAYRFNGIG VTQNVLYENQ |
| 921 | KLIANQFNSA IGKIQDSLSS TASALGKLQD VVNQNAQALN |
| 961 | TLVKQLSSNF GAISSVLNDI LSRLDKVEAE VQIDRLITGR |
| 1001 | LQSLQTYVTQ QLIRAAEIRA SANLAATKMS ECVLGQSKRV |
| 1041 | DFCGKGYHLM SFPQSAPHGV VFLHVTYVPA QEKNFTTAPA |
| 1081 | ICHDGKAHFP REGVFVSNGT HWFVTQRNFY EPQIITTDNT |
| 1121 | FVSGNCDVVI GIVNNTVYDP LQPELDSFKE ELDKYFKNHT |
| 1161 | SPDVDLGDIS GINASVVNIQ KEIDRLNEVA KNLNESLIDL |
| 1201 | QELGKYEQYI KWPWYIWLGF IAGLIAIVMV TIMLCCMTSC |
| 1241 | CSCLKGCCSC GSCCKFDEDD SEPVLKGVKL H |

The SARS-CoV-2 can have an open reading frame at positions 2720-8554 of the SEQ ID NO:1 sequence that can be referred to as nsp3, which includes transmembrane domain 1 (TM1). This nsp3 open reading frame with transmembrane domain 1 has NCBI accession no. YP_009725299.1 and is shown below as SEQ ID NO:17.

| | |
|---|---|
| 1 | APTKVTFGDD TVIEVQGYKS VNITFELDER IDKVLNEKCS |
| 41 | AYTVELGTEV NEFACVVADA VIKTLQPVSE LLTPLGIDLD |
| 81 | EWSMATYYLF DESGEFKLAS HMYCSFYPPD EDEEEGDCEE |
| 121 | EEFEPSTQYE YGTEDDYQGK PLEFGATSAA LQPEEEQEED |
| 161 | WLDDDSQQTV GQQDGSEDNQ TTTIQTIVEV QPQLEMELTP |
| 201 | VVQTIEVNSF SGYLKLTDNV YIKNADIVEE AKKVKPTVVV |
| 241 | NAANVYLKHG GGVAGALNKA TNNAMQVESD DYIATNGPLK |
| 281 | VGGSCVLSGH NLAKHCLHVV GPNVNKGEDI QLLKSAYENF |
| 321 | NQHEVLLAPL LSAGIFGADP IHSLRVCVDT VRTNVYLAVF |
| 361 | DKNLYDKLVS SFLEMKSEKQ VEQKIAEIPK EEVKPFITES |
| 401 | KPSVEQRKQD DKKIKACVEE VTTTLEETKF LTENLLLYID |
| 441 | INGNLHPDSA TLVSDIDITF LKKDAPYIVG DVVQEGVLTA |
| 481 | VVIPTKKAGG TTEMLAKALR KVPTDNYITT YPGQGLNGYT |
| 521 | VEEAKTVLKK CKSAFYILPS IISNEKQEIL GTVSWNLREM |
| 561 | LAHAEETRKL MPVCVETKAI VSTIQRKYKG IKIQEGVVDY |
| 601 | GARFYFYTSK TTVASLINTL NDLNETLVTM PLGYVTHGLN |
| 641 | LEEAARYMRS LKVPATVSVS SPDAVTAYNG YLTSSSKTPE |
| 681 | EHFIETISLA GSYKDWSYSG QSTQLGIEFL KRGDKSVYYT |
| 721 | SNPTTFHLDG EVITFDNIKT LLSLREVRTI KVFTTVDNIN |
| 761 | LHTQVVDMSM TYGQQFGPTY LDGADVTKIK PHNSHEGKTF |
| 801 | YVLPNDDTLR VEAFEYYHTT DPSFLGRYMS ALNHTKKWKY |
| 841 | PQVNGLTSIK WADNNCYLAT ALLTLQQIEL KFNPPALQDA |
| 881 | YYRARAGEAA NFCALILAYC NKTVGELGDV RETMSYLFQH |
| 921 | ANLDSCKRVL NVVCKTCGQQ QTTLKGVEAV MYMGTLSYEQ |
| 961 | FKKGVQIPCT CGKQATKYLV QQESPFVMMS APPAQYELKH |
| 1001 | GTFTCASEYT GNYQCGHYKH ITSKETLYCI DGALLIKSSE |
| 1041 | YKGPITDVFY KENSYTTTIK PVTYKLDGVV CTEIDPKLDN |
| 1081 | YYKKDNSYFT EQPIDLVPNQ PYPNASFDNF KFVCDNIKFA |
| 1121 | DDLNQLTGYK KPASRELKVT FFPDINGDVV AIDYKHYTPS |
| 1161 | FKKGAKLLHK PIVWHVNNAT NKATYKPNTW CIRCLWSTKP |
| 1201 | VETSNSFDVL KSEDAQGMDN LACEDLKPVS EEVVENPTIQ |
| 1241 | KDVLECNVKT TEVVGDIILK PANNSLKITE EVGHTDLMAA |
| 1281 | YVDNSSLTIK KPNELSRVLG LKTLATHGLA AVNSVPWDTI |
| 1321 | ANYAKPFLNK VVSTTTNIVT RCLNRVCTNY MPYFFTLLLQ |
| 1361 | LCTFTRSTNS RIKASMPTTI AKNTVKSVGK FCLEASFNYL |
| 1401 | KSPNFSKLIN IIIWFLLLSV CLGSLIYSTA ALGVLMSNLG |
| 1441 | MPSYCTGYRE GYLNSTNVTI ATYCTGSIPC SVCLSGLDSL |
| 1481 | DTYPSLETIQ ITISSFKWDL TAFGLVAEWF LAYILFTRFF |
| 1521 | YVLGLAAIMQ LFFSYFAVHF ISNSWLMWLI INLVQMAPIS |
| 1561 | AMVRMYIFFA SFYYVWKSYV HVVDGCNSST CMMCYKRNRA |
| 1601 | TRVECTTIVN GVRRSFYVYA NGGKGFCKLH NWNCVNCDTF |
| 1641 | CAGSTFISDE VARDLSLQFK RPINPTDQSS YIVDSVTVKN |
| 1681 | GSIHLYFDKA GQKTYERHSL SHFVNLDNLR ANNTKGSLPI |
| 1721 | NVIVFDGKSK CEESSAKSAS VYYSQLMCQP ILLLDQALVS |
| 1761 | DVGDSAEVAV KMFDAYVNTF SSTFNVPMEK LKTLVATAEA |
| 1801 | ELAKNVSLDN VLSTFISAAR QGFVDSDVET KDVVECLKLS |
| 1841 | HQSDIEVTGD SCNNYMLTYN KVENMTPRDL GACIDCSARH |
| 1881 | INAQVAKSHN IALIWNVKDF MSLSEQLRKQ IRSAAKKNNL |
| 1921 | PFKLTCATTR QVVNVVTTKI ALKGG |

The nsp3 protein has additional conserved domains including an N-terminal acidic (Ac), a predicted phosphoesterase, a papain-like proteinase, Y-domain, transmembrane domain 1 (TM1), and an adenosine diphosphate-ribose 1″-phosphatase (ADRP).

The SARS-CoV-2 can have an open reading frame at positions 8555-10054 of the SEQ ID NO:1 sequence that can be referred to as nsp4B_TM, which includes transmembrane domain 2 (TM2). This nsp4B_TM open reading frame with transmembrane domain 2 has NCBI accession no. YP_009725300 and is shown below as SEQ ID NO:18.

| | |
|---|---|
| 1 | KIVNNWLKQL IKVTLVFLFV AAIFYLITPV HVMSKHTDFS |
| 41 | SEIIGYKAID GGVTRDIAST DTCFANKHAD FDTWFSQRGG |
| 81 | SYTNDKACPL IAAVITREVG FVVPGLPGTI LRTTNGDFLH |
| 121 | FLPRVFSAVG NICYTPSKLI EYTDFATSAC VLAAECTIFK |
| 161 | DASGKPVPYC YDTNVLEGSV AYESLRPDTR YVLMDGSIIQ |
| 201 | FPNTYLEGSV RVVTTFDSEY CRHGTCERSE AGVCVSTSGR |
| 241 | WVLNNDYYRS LPGVFCGVDA VNLLTNMFTP LIQPIGALDI |
| 281 | SASIVAGGIV AIVVTCLAYY FMRFRRAFGE YSHVVAFNTL |
| 321 | LFLMSFTVLC LTPVYSFLPG VYSVIYLYLT FYLTNDVSFL |
| 361 | AHIQWMVMFT PLVPFWITIA YIICISTKHF YWFFSNYLKR |
| 401 | RVVFNGVSFS TFEEAALCTF LINKEMYLKL RSDVLLPLTQ |
| 441 | YNRYLALYNK YKYFSGAMDT TSYREAACCH LAKALNDFSN |
| 481 | SGSDVLYQPP QTSITSAVLQ |

The SARS-CoV-2 can have an open reading frame at positions 25393-26220 (ORF3a) of the SEQ ID NO:1 sequence that can be referred to as GU280_gp03 (SEQ ID NO:19, shown below).

| | |
|---|---|
| 1 | MDLFMRIFTI GTVTLKQGEI KDATPSDFVR ATATIPIQAS |
| 41 | LPFGWLIVGV ALLAVFQSAS KIITLKKRWQ LALSKGVHFV |
| 81 | CNLLLLFVTV YSHLLLVAAG LEAPFLYLYA LVYFLQSINF |
| 121 | VRIIMRLWLC WKCRSKNPLL YDANYFLCWH TNCYDYCIPY |
| 161 | NSVISSIVIT SGDGTTSPIS EHDYQIGGYT EKWESGVKDC |
| 201 | VVLHSYFTSD YYQLYSTQLS TDTGVEHVTF FIYNKIVDEP |
| 241 | EEHVQIHTID GSSGVVNPVM EPIYDEPTTT TSVPL |

In some cases, the constructs and SARS-CoV-2 virus-like particles described herein may not include portions that encode SEQ ID NO:19.

The SARS-CoV-2 can have an open reading frame at positions 26245-26472 (gene E) of the SEQ ID NO:1 sequence that can be referred to as GU280_gp04 (SEQ ID NO:20, shown below).

| | |
|---|---|
| 1 | MYSFVSEETG TLIVNSVLLF LAFVVFLLVT LAILTALRLC |
| 41 | AYCCNIVNVS LVKPSFYVYS RVKNLNSSRV PDLLV |

The SEQ ID NO:20 protein is a structural protein, for example, an envelope protein. In some cases, the constructs and SARS-CoV-2 virus-like particles described herein can encode or include a protein homologous to SEQ ID NO:20. In some cases, the constructs and SARS-CoV-2 virus-like particles described herein do not encode or include a protein homologous to SEQ ID NO:20.

The SARS-CoV-2 can have an open reading frame at positions 26523-27191 which encodes a M protein (Membrane protein; ORF5) of the SEQ ID NO:1 sequence that is typically referred to as the M protein but can also be referred to as GU280_gp05 (SEQ ID NO:21, shown below).

| | |
|---|---|
| 1 | MADSNGTITV EELKKLLEQW NLVIGFLFLT WICLLQFAYA |
| 41 | NRNRFLYIIK LIFLWLLWPV TLACFVLAAV YRINWITGGI |
| 121 | AIAMACLVGL MWLSYFIASF RLFARTRSMW SFNPETNILL |
| 161 | NVPLHGTILT RPLLESELVI GAVILRGHLR IAGHHLGRCD |
| 201 | IKDLPKEITV ATSRTLSYYK IGASQRVAGD SGFAAYSRYR |
| 241 | IGNYKLNTDH SSSSDNIA |
| 121 | LLVQ |

The SEQ ID NO:21 protein is a structural protein, for example, a membrane glycoprotein. In some cases, the constructs and SARS-CoV-2 virus-like particles described herein can encode or include a protein homologous to SEQ ID NO:21. In some cases, the constructs and SARS-CoV-2 virus-like particles described herein do not encode or include a protein homologous to SEQ ID NO:21.

The SARS-CoV-2 can have an open reading frame at positions 27202-27387 (ORF6) of the SEQ ID NO:1 sequence that can be referred to as GU280_gp06 (SEQ ID NO:22, shown below).

| | |
|---|---|
| 1 | MFHLVDFQVT IAEILLIIMR TFKVSIWNLD YIINLIIKNL |
| 41 | SKSLTENKYS QLDEEQPMEI D |

In some cases, the constructs and SARS-CoV-2 virus-like particles described herein may not encode or include a protein with homology to SEQ ID NO:22.

The SARS-CoV-2 can have an open reading frame at positions 27394-27759 (ORF7a) of the SEQ ID NO:1 sequence that can be referred to as GU280_gp07 (SEQ ID NO:23, shown below).

| | |
|---|---|
| 1 | MKIILFLALI TLATCELYHY QECVRGTTVL LKEPCSSGTY |
| 41 | EGNSPFHPLA DNKFALTCFS TQFAFACPDG VKHVYQLRAR |
| 121 | SVSPKLFIRQ EEVQELYSPI FLIVAAIVFI TLCFTLKRKT |
| 161 | E |

In some cases, the constructs and SARS-CoV-2 virus-like particles described herein may not encode or include a protein with homology to SEQ ID NO:23.

The SARS-CoV-2 can have an open reading frame at positions 27756-27887 (ORF7b) of the SEQ ID NO:1 sequence that can be referred to as GU280_gp08 (SEQ ID NO:24, shown below).

| | |
|---|---|
| 1 | MIELSLIDFY LCFLAFLLFL VLIMLIIFWF SLELQDHNET |
| 41 | CHA |

In some cases, the constructs and SARS-CoV-2 virus-like particles described herein may not encode or include a protein with homology to SEQ ID NO:24.

The SARS-CoV-2 can have an open reading frame at positions 27894-28259 (ORF8) of the SEQ ID NO:1 sequence that can be referred to as GU280_gp09 (SEQ ID NO:25, shown below).

| | |
|---|---|
| 1 | MKFLVFLGII TIVAAFHQEC SLQSCTQHQP YVVDDPCPIH |
| 41 | FYSKWYIRVG ARKSAPLIEL CVDEAGSKSP IQYIDIGNYT |
| 121 | VSCLPFTINC QEPKLGSLVV RCSFYEDFLE YHDVRVVLDE |
| 161 | I |

In some cases, the constructs and SARS-CoV-2 virus-like particles described herein may not encode or include a protein with homology to SEQ ID NO:25.

The nucleocapsid phosphoprotein (N protein) undergoes both self-association, interaction with other proteins, and interaction with RNA. The N protein is encoded within the SARS-CoV-2 genome at about positions 28274-29533 (gene N; ORF9) of the SEQ ID NO:1 sequence and is provided below as SEQ ID NO:26 (shown below).

| | |
|---|---|
| 1 | MSDNGPQNQR NAPRITEGGP SDSTGSNQNG ERSGARSKQR |
| 41 | RPQGLPNNTA SWFTALTQHG KEDLKEPRGQ GVPINTNSSP |
| 121 | DDQIGYYRRA TRRIRGGDGK MKDLSPRWYF YYLGTGPEAG |
| 161 | LPYGANKDGI IWVATEGALN TPKDHIGTRN PANNAAIVLQ |
| 201 | LPQGTTLPKG FYAEGSRGGS QASSRSSSRS RNSSRNSTPG |
| 241 | SSRGTSPARM AGNGGDAALA LLLLDRINQL ESKMSGKGQQ |
| 281 | QQGQTVTKKS AAEASKKPRQ KRTATKAYNV TQAFGRRGPE |
| 521 | QTQGNFGDQE LIRQGTDYKH WPQIAQFAPS ASAFFGMSRI |
| 561 | GMEVTPSGTW LTYTGAIKLD DKDPNEKDQV ILLNKHIDAY |
| 601 | KTEPPTEPKK DKKKKADETQ ALPQRQKKQQ TVILLPAADL |
| 641 | DDFSKQLQQS MSSADSTQA |

The SEQ ID NO:26 protein is a structural protein, for example, a nucleocapsid phosphoprotein. In some cases, the constructs and SARS-CoV-2 virus-like particles described herein can encode or include a protein homologous to SEQ ID NO:26. In some cases, the constructs and SARS-CoV-2 virus-like particles described herein do not encode or include a protein homologous to SEQ ID NO:26.

The SARS-CoV-2 can have an open reading frame at positions 29558-29674 (ORF10) of the SEQ ID NO:1 sequence that can be referred to as GU280_gp11 (SEQ ID NO:27, shown below).

| | |
|---|---|
| 1 | MGYINVFAFP FTIYSLLLCR MNSRNYIAQV DVVNENLT |

In some cases, the constructs and SARS-CoV-2 virus-like particles described herein may not encode or include a protein with homology to SEQ ID NO:27.

The SARS-CoV-2 can have a stem-loops at positions 29609-29644 and 29629-29657, which is within the encoded GU280_gp11. For example, the SARS-CoV-2 stem-loop at positions 29609-29644 is shown below as SEQ ID NO:28.

| | |
|---|---|
| 29601 | TT GTGCAGAATG AATTCTCGTA ACTACATAGC |
| 29641 | ACAA |

For example, the SARS-CoV-2 stem-loop at positions 29629-29657 is shown below as SEQ ID NO:29.

| | |
|---|---|
| 29629 | TA ACTACATAGC ACAAGTAGAT GTAGTTA |

In some cases, the constructs and SARS-CoV-2 virus-like particles described herein may not encode or include a protein with homology to SEQ ID NO:28 or 29. In some cases, the constructs and SARS-CoV-2 virus-like particles described herein do encode or include a protein with homology to SEQ ID NO:28 or 29.

The SARS-CoV-2 can have an open reading frame at positions 12686-13024 (nsp9) of the SEQ ID NO:1 sequence that encodes a ssRNA-binding protein with NCBI accession number YP_009725305.1, which has the following sequence (SEQ ID NO:30).

| | |
|---|---|
| 1 | NNELSPVALR QMSCAAGTTQ TACTDDNALA YYNTTKGGRE |
| 41 | VLALLSDLQD LKWARFPKSD GTGTIYTELE PPCRFVIDTP |
| 81 | KGPKVKYLYF IKGLNNLNRG MVLGSLAATV RLQ |

In some cases, the constructs and SARS-CoV-2 virus-like particles described herein may not encode or include a protein with homology to SEQ ID NO:30. In some cases, the constructs and SARS-CoV-2 virus-like particles described herein do encode or include a protein with homology to SEQ ID NO:30.

The constructs and/or SARS-CoV-2 virus-like particles described herein can have portions of the SARS-CoV-2 genome, where the deletions of the genome include at least 100, at least 500, at least 1000, at least 1500, at least 2000, at least 2500, at least 3000, at least 4000, at least 5000, at least 6000, at least 7000, at least 8000, at least 9000, at least 10,000, at least 11,000, at least 12,000, at least 13,000, at least 14,000, at least 15,000, at least 16,000, at least 17,000, at least 18,000, at least 19,000, at least 20,000, at least 21,000, at least 22,000, at least 23,000, at least 24,000, at least 25,000, at least 26,000, at least 27,000, at least 27500, or at least 28000 nucleotides of the SARS-CoV-2 genome.

The foregoing sequences are DNA sequences. The SARS-CoV-2 nucleic acids used in the compositions and methods described herein can be DNA or RNA versions of such sequences. The 3′ SARS-CoV-2 nucleic acids can include extended poly A sequences. For example, the extended poly-A sequences can have at least 100 adenine nucleotides to 250 adenine nucleotides. Such extended poly-A sequences can, for example, extend the half-life of the mRNA.

In addition, the SARS-CoV-2 genome can naturally have structural variations that are reflections of sequence variations. Hence, the SARS-CoV-2 used in the compositions and methods described herein can, for example, have one or more nucleotide or amino acid differences from the sequences shown as SEQ ID NO:1-30. In some cases, the SARS-CoV-2 used in the compositions and methods described herein can, for example, have two, three, four, five, six, seven, eight, nine, ten, fifteen, twenty, twenty-five, thirty, or more nucleotide or amino acid differences from the sequences shown as SEQ ID NO:1-30. Hence, prior to deletion any of the SARS-CoV-2 nucleic acids used in the methods and compositions described herein can be a DNA or RNA with at least 70%, or at least 75%, or at least 80%, or at least 85%, or at least 90%, or at least 95%, or at least 96%, or at least 97%, or at least 98%, or at least 99%, or at least 99.5% sequence identity to any of SEQ ID NO:1-30.

### Antibodies

The heterologous nucleic acid segment can include a coding region for at least one anti-SARS-CoV-2 antibody or anti-SARS-CoV-2 antibody fragment. VLPs that include such anti-SARS-CoV-2 coding regions can be used to reduce inflammation associated with SARS-CoV-2 infection, to inhibit SARS-CoV-2 viral assembly and SARS-CoV-2 cellular transmission. Hence, such VLPs can be used as therapeutic agents for treatment of SARS-CoV-2.

Antibodies can be raised against various epitopes of SARS-CoV-2 proteins, including the SARS-CoV-2 Spike protein, SARS-CoV-2 M protein, the SARS-CoV-2 E protein, the SARS-CoV-2 N protein, or a portion or epitope thereof. Some antibodies against SARS-CoV-2 may also be available commercially. However, the antibodies contemplated for treatment pursuant to the methods and compositions described herein are preferably human or humanized antibodies and are highly specific for their SARS-CoV-2 targets.

In some cases, the antibodies can be directed against the SARS-CoV-2 Spike protein. One example of a SARS-CoV-2 spike protein amino acid sequence is SEQ ID NO:5.

The Spike protein is responsible for facilitating entry of the SARS-CoV-2 into cells. It is composed of a short intracellular tail, a transmembrane anchor, and a large ectodomain that consists of a receptor binding S1 subunit and a membrane-fusing S2 subunit. The spike receptor binding domain can reside at amino acid positions 330-583 of the SEQ ID NO:5 spike protein (shown below as SEQ ID NO:15).

| | |
|---|---|
| 330 | P NITNLCPFGE VENATRFASV YAWNRKRISN |
| 361 | CVADYSVLYN SASFSTFKCY GVSPTKINDL CFTNVYADSE |
| 401 | VIRGDEVRQI APGQTGKIAD YNYKLPDDFT GCVIAWNSNN |
| 441 | LDSKVGGNYN YLYRLERKSN LKPFERDIST EIYQAGSTPC |
| 481 | NGVEGENCYF PLQSYGFQPT NGVGYQPYRV VVLSFELLHA |
| 521 | PATVCGPKKS TNLVKNKCVN FNENGLIGTG VLTESNKKEL |
| 561 | PFQQFGRDIA DTTDAVRDPQ TLE |

The entry receptor utilized by SARS-CoV-2 is the angiotensin-converting enzyme 2 (ACE-2). The SARS-CoV-2 spike protein membrane-fusing S2 domain may be at positions 662-1270 of the SEQ ID NO:5 spike protein (shown below as SEQ ID NO:16).

| | |
|---|---|
| 662 | CDIPIGAGI CASYQTQTNS |
| 681 | PRRARSVASQ SIIAYTMSLG AENSVAYSNN SIAIPTNFTI |
| 721 | SVTTEILPVS MTKTSVDCTM YICGDSTECS NLLLQYGSFC |
| 761 | TQLNRALIGI AVEQDKNTQE VFAQVKQIYK TPPIKDEGGE |
| 801 | NFSQILPDPS KPSKRSFIED LLENKVTLAD AGFIKQYGDC |
| 841 | LGDIAARDLI CAQKENGLTV LPPLLTDEMI AQYTSALLAG |
| 881 | TITSGWTFGA GAALQIPFAM QMAYRENGIG VTQNVLYENQ |
| 921 | KLIANQFNSA IGKIQDSLSS TASALGKLQD VVNQNAQALN |
| 961 | TLVKQLSSNF GAISSVLNDI LSRLDKVEAE VQIDRLITGR |
| 1001 | LQSLQTYVTQ QLIRAAEIRA SANLAATKMS ECVLGQSKRV |
| 1041 | DECGKGYHLM SFPQSAPHGV VFLHVTYVPA QEKNFTTAPA |
| 1081 | ICHDGKAHFP REGVFVSNGT HWFVTQRNFY EPQIITTDNT |
| 1121 | FVSGNCDVVI GIVNNTVYDP LQPELDSFKE ELDKYFKNHT |
| 1161 | SPDVDLGDIS GINASVVNIQ KEIDRLNEVA KNLNESLIDL |
| 1201 | QELGKYEQYI KWPWYIWLGF IAGLIAIVMV TIMLCCMTSC |
| 1241 | CSCLKGCCSC GSCCKEDEDD SEPVLKGVKL H |

The anti-SARS-CoV-2 Spike antibodies can bind to any of the foregoing portions or domains.

The antibodies may be monoclonal or polyclonal antibodies. Such antibodies may also be humanized or fully human monoclonal antibodies. The antibodies can exhibit one or more desirable functional properties, such as high affinity binding to SARS-CoV-2 or a specific SARS-CoV-2 protein, high affinity binding to SARS-CoV-2 spike protein, or the ability to inhibit binding of the SARS-CoV-2 spike protein to cells and/or to inhibit SARS-CoV-2 binding to cellular receptors.

Methods and compositions described herein can include antibodies that bind SARS-CoV-2 or a specific SARS-CoV-2 protein. For example, the antibodies can in some cases bind to SARS-CoV-2 spike protein. The antibodies can also bind to a combination of antibodies that bind to SARS-CoV-2 or a specific SARS-CoV-2 protein, or a combination where each antibody type can separately bind SARS-CoV-2 or a specific SARS-CoV-2 protein.

The term “antibody” as referred to herein includes whole antibodies and any antigen binding fragment (i.e., “antigen-binding portion”) or single chains thereof. An “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. The heavy chain constant region is comprised of three domains, C_{H1}, C_{H2} and Cin. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The term “antigen-binding portion” of an antibody (or simply “antibody portion”), as used herein, refers to one or more fragments of an antibody that retain the ability to specifically bind to an antigen (e.g. a peptide or domain of a specific SARS-CoV-2 protein). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region, (iii) a Fd fragment consisting of the V_{H} and C_{H1} domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

An “isolated antibody,” as used herein, is intended to refer to an antibody that is substantially free of other antibodies having different antigenic specificities (e.g., an isolated antibody that specifically binds SARS-CoV-2 or a specific SARS-CoV-2 protein is substantially free of antibodies that specifically bind antigens other than SARS-CoV-2 or a specific SARS-CoV-2 protein. An isolated antibody that specifically binds SARS-CoV-2 or a specific SARS-CoV-2 protein may, however, have cross-reactivity to other antigens, such as isoforms or mutant SARS-CoV-2 proteins. Moreover, an isolated antibody may be substantially free of other cellular material and/or chemicals.

The terms “monoclonal antibody” or “monoclonal antibody composition” as used herein refer to a preparation of antibody molecules of single molecular composition. A monoclonal antibody composition displays a single binding specificity and affinity for a particular epitope.

As used herein, a “polyclonal antibody” refers to refers to a mixture of antibodies that recognize one or more epitopes of a virus (e.g., any SARS-CoV-2 strain or variant). The antibodies can have different binding specificities and affinities for the one or more epitopes. Alternatively, a “polyclonal antibody” can refer to polyclonal antibodies derived from the serum of a subject (antiserum). In some cases, the subject has been inoculated with a mixture of antigens or RNAs, such as a SARS-CoV-2 vaccine. In other cases, the subject has not received a vaccine or a mixture of antigens, or a mixture of RNAs (e.g., is unvaccinated) In other cases, the subject has been infected with SARS-CoV-2. In other cases, the subject has not been infected with SARS-CoV-2 and/or has not received a vaccine or a mixture of antigens, or a mixture of RNAs (e.g., is unvaccinated), and these subjects can have negative control levels of polyclonal antibodies (or serve as a negative control antiserum).

The term “human antibody,” as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human germline immunoglobulin sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The term “recombinant human antibody,” as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{L} and V_{H} regions of the recombinant antibodies are sequences that, while derived from and related to human germline V_{L} and V_{H} sequences, may not naturally exist within the human antibody germline repertoire in vivo.

As used herein, “isotype” refers to the antibody class (e.g., IgM or IgG1) that is encoded by the heavy chain constant region genes.

The phrases “an antibody recognizing an antigen” and “an antibody specific for an antigen” are used interchangeably herein with the term “an antibody which binds specifically to an antigen.”

The term “human antibody derivatives” refers to any modified form of the human antibody, e.g., a conjugate of the antibody and another agent or antibody.

The term “humanized antibody” is intended to refer to antibodies in which CDR sequences derived from the germline of another mammalian species, such as a mouse, have been grafted onto human framework sequences. Additional framework region modifications may be made within the human framework sequences.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

As used herein, an antibody that “specifically binds to SARS-CoV-2 or a specific SARS-CoV-2 protein is intended to refer to an antibody that binds to SARS-CoV-2 or a specific SARS-CoV-2 protein with a K_{D} of 1×10⁻⁷M or less, more preferably 5×10⁻⁸ M or less, more preferably 1×10⁻⁸ M or less, more preferably 5×10⁻⁹ M or less, even more preferably between 1×10⁻⁸ M and 1×10⁻¹⁰ M or less.

The term “K_{assoc}” or “Kₐ,” as used herein, is intended to refer to the association rate of a particular antibody-antigen interaction, whereas the term “K_{dis}” or “K_{d},” as used herein, is intended to refer to the dissociation rate of a particular antibody-antigen interaction. The term “K_{D},” as used herein, is intended to refer to the dissociation constant, which is obtained from the ratio of K_{d} to Kₐ (i.e., K_{d}/Kₐ) and is expressed as a molar concentration (M). K_{D} values for antibodies can be determined using methods well established in the art. A preferred method for determining the K_{D} of an antibody is by using surface plasmon resonance, preferably using a biosensor system such as a Biacore™ system.

The antibodies of the invention are characterized by particular functional features or properties of the antibodies. For example, the antibodies bind specifically to SARS-CoV-2 or a specific SARS-CoV-2 protein. Preferably, an antibody of the invention binds to SARS-CoV-2 or a specific SARS-CoV-2 protein with high affinity, for example with a K_{D} of 1×10⁻⁷ M or less. The antibodies can exhibit one or more of the following characteristics:
(a) binds to SARS-CoV-2 or a SARS-CoV-2 protein with a K_{D} of 1×10⁻⁷ M or less;
(b) inhibits the binding of SARS-CoV-2 spike protein ACE2 receptor;
(c) inhibits SARS-CoV-2-related inflammation; or
(d) a combination thereof.

For example, the antibodies described herein can prevent greater than 30% binding, or greater than 40% binding, or greater than 50% binding, or greater than 60% binding, or greater than 70% binding, or greater than 80% binding, or greater than 90% binding of SARS-CoV-2 to cells or to the ACE2 receptor.

Assays to evaluate the binding ability of the antibodies to SARS-CoV-2 or a specific SARS-CoV-2 protein can be used, including for example, ELISAs, Western blots and RIAs. The binding kinetics (e.g., binding affinity) of the antibodies also can be assessed by standard assays known in the art, such as by Biacore™. analysis.

Given that each of the subject antibodies can bind to SARS-CoV-2 or a specific SARS-CoV-2 protein, the V_{L} and V_{H} sequences can be “mixed and matched” to create other binding molecules that bind to SARS-CoV-2 or a specific SARS-CoV-2 protein. The binding properties of such “mixed and matched” antibodies can be tested using the binding assays described above and assessed in assays described in the examples. When V_{L} and V_{H} chains are mixed and matched, a V_{H} sequence from a particular V_{H}/V_{L} pairing can be replaced with a structurally similar V_{H} sequence. Likewise, preferably a V_{L} sequence from a particular V_{H}/V_{L} pairing is replaced with a structurally similar V_{L} sequence.

Accordingly, in one aspect, the invention provides an isolated monoclonal antibody, or antigen binding portion thereof comprising:
(a) a heavy chain variable region comprising an amino acid sequence; and
(b) a light chain variable region comprising an amino acid sequence;
wherein the antibody specifically binds SARS-CoV-2 or a specific SARS-CoV-2 protein.

In some cases, the CDR3 domain, independently from the CDR1 and/or CDR2 domain(s), alone can determine the binding specificity of an antibody for a cognate antigen and that multiple antibodies can predictably be generated having the same binding specificity based on a common CDR3 sequence. See, for example, Klimka et al., British J. of Cancer 83 (2): 252-260 (2000) (describing the production of a humanized anti-CD30 antibody using only the heavy chain variable domain CDR3 of murine anti-CD30 antibody Ki-4); Beiboer et al., J. Mol. Biol. 296:833-849 (2000) (describing recombinant epithelial glycoprotein-2 (EGP-2) antibodies using only the heavy chain CDR3 sequence of the parental murine MOC-31 anti-EGP-2 antibody); Rader et al., Proc. Natl. Acad. Sci. U.S.A. 95:8910-8915 (1998) (describing a panel of humanized anti-integrin alphaᵥbeta₃ antibodies using a heavy and light chain variable CDR3 domain). Hence, in some cases a mixed and matched antibody or a humanized antibody contains a CDR3 antigen binding domain that is specific for SARS-CoV-2 or a specific SARS-CoV-2 protein.

### Inhibitory Nucleic Acids

Expression of SARS-CoV-2 RNA can be inhibited, for example by use of an inhibitory nucleic acid that specifically binds to SARS-CoV-2 RNA.

An inhibitory nucleic acid can have at least one segment that will hybridize to a segment of SARS-CoV-2 RNA under intracellular or stringent conditions. An inhibitory nucleic acid may hybridize to a SARS-CoV-2 RNA genomic, or a segment thereof. An inhibitory nucleic acid may be the heterologous nucleic acid that is part of the SARS-CoV-2 packaging signal sequence segment linked to a heterologous nucleic acid.

An inhibitory nucleic acid is a polymer of ribose nucleotides or deoxyribose nucleotides having more than 13 nucleotides in length. An inhibitory nucleic acid may include naturally occurring nucleotides; synthetic, modified, or pseudo-nucleotides such as phosphorothiolates; as well as nucleotides having a detectable label such as P³², biotin or digoxigenin. An inhibitory nucleic acid can reduce the expression and/or activity of a SARS-CoV-2 nucleic acid Such an inhibitory nucleic acid may be completely complementary to a segment of a SARS-CoV-2 nucleic acid (e.g., an RNA) that has infected a subject. Alternatively, some variability is permitted in the inhibitory nucleic acid sequences relative to SARS-CoV-2 sequences that infect a subject. An inhibitory nucleic acid can hybridize to a SARS-CoV-2 nucleic acid under intracellular conditions or under stringent hybridization conditions and is sufficiently complementary to inhibit expression of the endogenous SARS-CoV-2 nucleic acid. Intracellular conditions refer to conditions such as temperature, pH and salt concentrations typically found inside a cell, e.g. an animal or mammalian cell. One example of such an animal or mammalian cell is a myeloid progenitor cell. Another example of such an animal or mammalian cell is a more differentiated cell derived from a myeloid progenitor cell. Generally, stringent hybridization conditions are selected to be about 5° C. lower than the thermal melting point (Tₘ) for the specific sequence at a defined ionic strength and pH. However, stringent conditions encompass temperatures in the range of about 1° C. to about 20° C. lower than the thermal melting point of the selected sequence, depending upon the desired degree of stringency as otherwise qualified herein. Inhibitory oligonucleotides that comprise, for example, 2, 3, 4, or 5 or more stretches of contiguous nucleotides that are precisely complementary to a SARS-CoV-2 sequence, each separated by a stretch of contiguous nucleotides that are not complementary to adjacent sequences, can inhibit the function of one or more nucleic acids for any of the SARS-CoV-2 sequences described herein or any SARS-CoV-2 mutant or variant. In general, each stretch of contiguous nucleotides is at least 4, 5, 6, 7, or 8 or more nucleotides in length. Non-complementary intervening sequences may be 1, 2, 3, or 4 nucleotides in length. One skilled in the art can easily use the calculated melting point of an inhibitory nucleic acid hybridized to a sense nucleic acid to estimate the degree of mismatching that will be tolerated for inhibiting expression of a particular target nucleic acid. Inhibitory nucleic acids of the invention include, for example, a short hairpin RNA, a small interfering RNA, a ribozyme or an antisense nucleic acid molecule.

The inhibitory nucleic acid molecule may be single or double stranded (e.g. a small interfering RNA (siRNA)) and may function in an enzyme-dependent manner or by steric blocking. Inhibitory nucleic acid molecules that function in an enzyme-dependent manner include forms dependent on RNase H activity to degrade target mRNA. These include single-stranded DNA, RNA, and phosphorothioate molecules, as well as the double-stranded RNAi/siRNA system that involves target mRNA recognition through sense-antisense strand pairing followed by degradation of the target mRNA by the RNA-induced silencing complex. Steric blocking inhibitory nucleic acids, which are RNase-H independent, interfere with gene expression or other mRNA-dependent cellular processes by binding to a target mRNA and getting in the way of other processes. Steric blocking inhibitory nucleic acids include 2′-O alkyl (usually in chimeras with RNase-H dependent antisense), peptide nucleic acid (PNA), locked nucleic acid (LNA) and morpholino antisense.

Small interfering RNAs, for example, may be used to specifically reduce translation of SARS-CoV-2 protein such that translation of the encoded SARS-CoV-2 polypeptide is reduced. SiRNAs mediate post-transcriptional gene silencing in a sequence-specific manner. See, for example, website at invitrogen.com/site/us/en/home/Products-and-Services/Applications/rnai.html. Once incorporated into an RNA-induced silencing complex, siRNA mediate cleavage of the homologous endogenous mRNA transcript by guiding the complex to the homologous mRNA transcript, which is then cleaved by the complex. The siRNA may be homologous and/or complementary to any region of the SARS-CoV-2 transcript and/or any of the transcripts of the SARS-CoV-2. The region of homology may be 30 nucleotides or less in length, preferable less than 25 nucleotides, and more preferably about 21 to 23 nucleotides in length. SIRNA is typically double stranded and may have two-nucleotide 3′ overhangs, for example, 3′ overhanging UU dinucleotides. Methods for designing siRNAs are known to those skilled in the art. See, for example, Elbashir et al. Nature 411:494-498 (2001); Harborth et al. Antisense Nucleic Acid Drug Dev. 13:83-106 (2003).

The pSuppressorNeo vector for expressing hairpin siRNA, commercially available from IMGENEX (San Diego, California), can be used to generate siRNA for inhibiting replication or expression of SARS-CoV-2. The construction of the siRNA expression plasmid involves the selection of the target region of the mRNA, which can be a trial-and-error process. However, Elbashir et al. have provided guidelines that appear to work ˜80% of the time. Elbashir, S. M., et al., Analysis of gene function in somatic mammalian cells using small interfering RNAs. Methods, 2002. 26(2): p. 199-213. As siRNA can begin with AA, have 3′ UU overhangs for both the sense and antisense siRNA strands, and have an approximate 50% G/C content. An example of a sequence for a synthetic siRNA is 5′-AA (N19)UU, where N is any nucleotide in the mRNA sequence and should be approximately 50% G-C content. The selected sequence(s) can be compared to others in the human genome database to minimize homology to other known coding sequences (e.g., by Blast search, for example, through the NCBI website).

SiRNAs may be chemically synthesized, created by in vitro transcription, or expressed from an siRNA expression vector or a PCR expression cassette. See, e.g., website at invitrogen.com/site/us/en/home/Products-and-Services/Applications/rnai.html. When an siRNA is expressed from an expression vector or a PCR expression cassette, the insert encoding the siRNA may be expressed as an RNA transcript that folds into an siRNA hairpin. Thus, the RNA transcript may include a sense siRNA sequence that is linked to its reverse complementary antisense siRNA sequence by a spacer sequence that forms the loop of the hairpin as well as a string of U's at the 3′ end. The loop of the hairpin may be of any appropriate lengths, for example, 3 to 30 nucleotides in length, preferably, 3 to 23 nucleotides in length, and may be of various nucleotide sequences including, AUG, CCC, UUCG, CCACC, CTCGAG, AAGCUU, CCACACC and UUCAAGAGA (SEQ ID NO:31). SIRNAS also may be produced in vivo by cleavage of double-stranded RNA introduced directly or via a transgene or virus. Amplification by an RNA-dependent RNA polymerase may occur in some organisms.

An inhibitory nucleic acid such as a short hairpin RNA siRNA or an antisense oligonucleotide may be prepared using methods such as by expression from an expression vector or expression cassette that includes the sequence of the inhibitory nucleic acid. Alternatively, it may be prepared by chemical synthesis using naturally occurring nucleotides, modified nucleotides or any combinations thereof. In some embodiments, the inhibitory nucleic acids are made from modified nucleotides or non-phosphodiester bonds, for example, that are designed to increase biological stability of the inhibitory nucleic acid or to increase intracellular stability of the duplex formed between the inhibitory nucleic acid and the target SARS-CoV-2 nucleic acids.

An inhibitory nucleic acid may be prepared using available methods, for example, by expression from an expression vector encoding a complementarity sequence of the SARS-CoV-2 nucleic acids described herein. Alternatively, it may be prepared by chemical synthesis using naturally occurring nucleotides, modified nucleotides or any mixture of combination thereof. In some embodiments, the inhibitory nucleic acids described herein are made from modified nucleotides or non-phosphodiester bonds, for example, that are designed to increase biological stability of the inhibitory nucleic acids or to increase intracellular stability of the duplex formed between the inhibitory nucleic acids and other (e.g., endogenous) nucleic acids.

For example, the SARS-CoV-2 inhibitory nucleic acids can be peptide nucleic acids that have peptide bonds rather than phosphodiester bonds.

Naturally occurring nucleotides that can be employed in the SARS-CoV-2 inhibitory nucleic acids include the ribose or deoxyribose nucleotides adenosine, guanine, cytosine, thymine and uracil. Examples of modified nucleotides that can be employed in SARS-CoV-2 inhibitory nucleic acids include 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methythio-N6-isopentenyladeninje, uracil-5oxyacetic acid, wybutoxosine, pseudouracil, queosine, 2-thiocytosine, 5-methyl-2-thiouracil, 2-thiouracil, 4-thiouracil, 5-methyluracil, uracil-5-oxacetic acid methylester, uracil-5-oxacetic acid, 5-methyl-2-thiouracil, 3-(3-amino-3-N-2-carboxypropyl)uracil, (acp3)w, and 2,6-diaminopurine.

Thus, inhibitory nucleic acids of the SARS-CoV-2 described herein may include modified nucleotides, as well as natural nucleotides such as combinations of ribose and deoxyribose nucleotides. The inhibitory nucleic acids and may be of same length as wild type SARS-CoV-2 described herein. However, the SARS-CoV-2 inhibitory nucleic acids described herein can also be longer and include other useful sequences (e.g., a segment encoding a detectable signal protein). In some embodiments, the SARS-CoV-2 inhibitory nucleic acids described herein are somewhat shorter. For example, SARS-CoV-2 inhibitory nucleic acids of described herein can include a segment that has a nucleic acid sequence that can be missing up to 5 nucleotides, or missing up to 10 nucleotides, or missing up to 20 nucleotides, or missing up to 30 nucleotides, or missing up to 50 nucleotides, or missing up to 100 nucleotides from the 5′ or 3′ end of any of the SARS-CoV-2 described herein.

### Vaccination Methods

As shown herein, the SARS-CoV-2 virus-like particles can be used in methods to evaluate immune responses against SARS-CoV-2. In general, the methods involve evaluating whether subjects have antibodies against SARS-CoV-2 and/or quantifying the neutralization of SARS-CoV-2 virus-like particles by a subject's antibodies. Also, as illustrated herein, the immune responses of subjects can vary and such immune responses generally decline over time. Methods are therefore described herein for evaluating whether at least one subject can benefit from vaccination against SARS-CoV-2. Methods are also described herein for evaluating which type of vaccine formulation can be more effective against SARS-CoV-2 for at least one subject.

For example, a method is described herein that involves contacting at least one subject's antibodies (e.g., serum) with SARS-CoV-2 virus-like particles and a population of receptor cells to form an assay mixture, and quantifying a signal from the assay mixture (e.g., from the receptor cells). Control assays can be used that have no antibodies against SARS-CoV-2 and/or known amounts of antibodies against SARS-CoV-2. If a subject has low levels of antibodies that subject can be treated to improve his or her immune response against SARS-CoV-2, for example by administration of a previously administered vaccine (e.g., as a booster), or by administration of a new vaccine.

In some cases, the quantified signal level from an assay mixture can be compared to a mean control signal level such as a mean control level of a population of subjects newly vaccinated or newly boosted against SARS-CoV-2, for example a population of subjects newly vaccinated or newly boosted against SARS-CoV-2 by the Pfizer, Moderna, or Johnson & Johnson vaccines. A need for treatment of a subject can be determined by comparing that subject's quantified signal level to one or more mean control signal levels.

Subjects with low immune responses against SARS-CoV-2 (low quantified signal levels) can be vaccinated or boosted with a known vaccine such as any of the Pfizer, Moderna, or Johnson & Johnson vaccines. As illustrated herein, the Pfizer and Moderna vaccines tend to stimulate immune responses against SARS-CoV-2 better than the Johnson & Johnson vaccine. In some cases, such subjects are therefore vaccinated or boosted a Pfizer or Moderna vaccine.

The Pfizer BNT162b1 vaccine is a lipid-nanoparticle-formulated, nucleoside-modified mRNA vaccine that encodes the trimerized receptor-binding domain (RBD) of the spike glycoprotein of SARS-CoV-2. A sequence for the mRNA encoding the spike glycoprotein of SARS-CoV-2 is shown below (SEQ ID NO:34).

| | |
|---|---|
| 1 | AUGUUUGUGU UUCUUGUGCU GCUGCCUCUU GUGUCUUCUC |
| 41 | AGUGUGUGGU GAGAUUUCCA AAUAUUACAA AUCUGUGUCC |
| 81 | AUUUGGAGAA GUGUUUAAUG CAACAAGAUU UGCAUCUGUG |
| 121 | UAUGCAUGGA AUAGAAAAAG AAUUUCUAAU UGUGUGGCUG |
| 161 | AUUAUUCUGU GCUGUAUAAU AGUGCUUCUU UUUCCACAUU |
| 201 | UAAAUGUUAU GGAGUGUCUC CAACAAAAUU AAAUGAUUUA |
| 241 | UGUUUUACAA AUGUGUAUGC UGAUUCUUUU GUGAUCAGAG |
| 281 | GUGAUGAAGU GAGACAGAUU GCCCCCGGAC AGACAGGAAA |
| 321 | AAUUGCUGAU UACAAUUACA AACUGCCUGA UGAUUUUACA |
| 361 | GGAUGUGUGA UUGCUUGGAA UUCUAAUAAU UUAGAUUCUA |
| 401 | AAGUGGGAGG AAAUUACAAU UAUCUGUACA GACUGUUUAG |
| 441 | AAAAUCAAAU CUGAAACCUU UUGAAAGAGA UAUUUCAACA |
| 484 | GAAAUUUAUC AGGCUGGAUC AACACCUUGU AAUGGAGUGG |
| 521 | AAGGAUUUAA UUGUUAUUUU CCAUUACAGA GCUAUGGAUU |
| 561 | UCAGCCAACC AAUGGUGUGG GAUAUCAGCC AUAUAGAGUG |
| 601 | GUGGUGCUGU CUUUUGAACU GCUGCAUGCA CCUGCAACAG |
| 641 | UGUGUGGACC UAAAGGCUCC CCCGGCUCCG GCUCCGGAUC |
| 681 | UGGUUAUAUU CCUGAAGCUC CAAGAGAUGG GCAAGCUUAC |
| 721 | GUUCGUAAAG AUGGCGAAUG GGUAUUACUU UCUACCUUUU |
| 761 | UAGGCCGGUC CCUGGAGGUG CUGUUCCAGG GCCCCGGC |

This RNA encodes the following amino acid sequence (SEQ ID NO:35).

| | |
|---|---|
| 1 | MFVFLVLLPL VSSQCVVRFP NITNLCPFGE VENATRFASV |
| 41 | YAWNRKRISN CVADYSVLYN SASESTEKCY GVSPTKINDL |
| 81 | CFINVYADSF VIRGDEVRQI APGQTGKIAD YNYKLPDDFT |
| 121 | GCVIAWNSNN LDSKVGGNYN YLYRLERKSN LKPFERDIST |
| 161 | EIYQAGSTPC NGVEGENCYF PLQSYGFQPT NGVGYQPYRV |
| 201 | VVLSFELLHA PATVCGPKGS PGSGSGSGYI PEAPRDGQAY |
| 241 | VRKDGEWVLL STFLGRSLEV LFQGPG |

The Pfizer BNT162b1 lipid nanoparticles include a cationic lipid, a neutral lipid, a steroid, a polymer conjugated lipid; and the SARS-CoV-2 spike RNA. For example, the lipids can include ((4-hydroxybutyl) azanediyl)bis(hexane-6,1-diyl)bis; (2-hexyldecanoate), 2 [(polyethylene glycol)-2000]-N,N-ditetradecylacetamide; 1,2-distearoyl-snglycero-3-phosphocholine; cholesterol; and combinations thereof. In one embodiment, the cationic lipid is ALC-0315, the neutral lipid is distearoylphosphatidylcholine (DSPC), the steroid is cholesterol, and the polymer conjugated lipid is ALC-0159. The structure of ALC-0315 (available from Echelon Biosciences (echelon-inc.com/product/alc-0315)) is shown below.

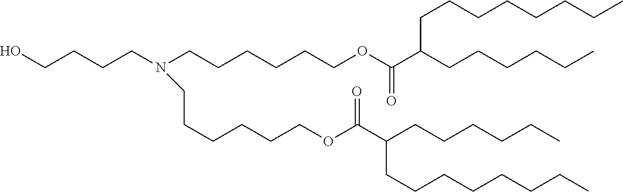 [SMILES: CCCCCCCCC(CCCCCC)C(=O)OCCCCCCN(CCCCO)CCCCCCOC(=O)C(CCCCCC)CCCCCCCC]

The mRNA of the BNT162b1 vaccine can also include a nucleoside 1-methyl-pseudouridine modified RNA. The mRNA of the BNT162b1 vaccine can also include a T4 fibritin-derived “foldon” trimerization domain to increase its immunogenicity. One example of such a foldon domain is shown below as SEQ ID NO:36.

GSGYIPEAPR DGQAYVRKDG EWVLLSTELG RSLEVLFQGP G

The Moderna vaccine can also include nanoparticles that include an mRNA that encodes a SARS-CoV-2 spike protein with lipids. The Moderna vaccine mRNA encodes a full-length SARS-CoV-2 spike protein modified with 2 proline substitutions within the heptad repeat 1 domain (S-2P). The lipids can include SM-102 (Heptadecan-9-yl 8-{(2-hydroxyethyl)[6-oxo-6-(undecyloxy)hexyl]amino}octanoate); 1,2-dimyristoyl-rac-glycero3-methoxypolyethylene glycol-2000 [PEG2000-DMG]; cholesterol; 1,2-distearoyl-snglycero-3-phosphocholine [DSPC]; and combinations thereof. SARS-CoV-2 virus-like-particles, the particles comprising at least one RNA comprising a SARS-CoV-2 packaging signal sequence segment linked to a heterologous nucleic acid, SARS-CoV-2 spike (S) proteins, SARS-CoV-2 membrane (M) proteins, SARS-CoV-2 envelope (E) proteins, and SARS-CoV-2 nucleocapsid (N) proteins.

In some cases, subjects with low immune responses against SARS-CoV-2 (low quantified signal levels) can be vaccinated or boosted with a new type of vaccine or immunological composition against SARS-CoV-2. Such a vaccine or immunological composition can include at least one RNA that encodes at least one SARS-CoV-2 spike, N, M, and/or E protein, where the spike protein does not have a SEQ ID NO:5, 34, or 35 sequence, the N protein does not have SEQ ID NO:26, the M protein does not have SEQ ID NO:7 or 21, and the E does not have SEQ ID NO:20. Such an immunological composition may provide enhanced immunity to SARS-CoV-2 variants. For example, the SARS-CoV-2 spike protein that does not have SEQ ID NO:5, 34, or 35 may have any of the amino acid substitutions or mutations listed in Table 2. For example, the SARS-CoV-2 N protein that does not have SEQ ID NO:26 may have any of the amino acid substitutions or mutations listed in Table 3. For example, the SARS-CoV-2 M protein that does not have SEQ ID NO:7 or 21 may have any of the amino acid substitutions or mutations listed in Table 4. For example, the SARS-CoV-2 E protein that does not have SEQ ID NO:20 may have any of the amino acid substitutions or mutations listed in Table 5.

Such a new type of vaccine or immunological composition can include any of the lipids described above for the Pfizer or Moderna vaccines. Such a new type of vaccine or immunological composition can also include one or more foldon domains. In addition, a new type of vaccine can be an RNA vaccine that can have one or more modified nucleotides and/or one or more modified phosphodiester bonds. For example, the modified phosphodiester bonds can be peptide bonds rather than phosphodiester bonds.

Examples of modified nucleotides that can be employed include 5-fluorouracil, 5-bromouracil, S-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methythio-N6-isopentenyladeninje, uracil-5oxyacetic acid, wybutoxosine, pseudouracil, queosine, 2-thiocytosine, 5-methyl-2-thiouracil, 2-thiouracil, 4-thiouracil, 5-methyluracil, uracil-5-oxacetic acid methylester, uracil-5-oxacetic acid, 5-methyl-2-thiouracil, 3-(3-amino-3-N-2-carboxypropyl)uracil, (acp3)w, and 2,6-diaminopurine.

### Compositions

The invention also relates to compositions containing one or more active agents such as any of the SARS-CoV-2 VLPs described herein, or any of the test agents that inhibit VLP assembly, VLP packaging, VLP replication, or VLP cellular entry. Such active agents can be a VLP, polypeptide, an antibody (or antibody mixture), a nucleic acid encoding a polypeptide (e.g., within an expression cassette or expression vector), an inhibitory nucleic acid, a small molecule, a compound identified by a method described herein, or a combination thereof.

In some cases, the active agent can be an agent that stimulates an immunological reaction against SARS-CoV-2. Such an immunological composition can include at least one SARS-CoV-2 spike, N, M, and/or E protein or at least one RNA that encodes at least one SARS-CoV-2 spike, N, M, and/or E protein, where the spike protein does not have a SEQ ID NO:5, 34, or 35 sequence, the N protein does not have SEQ ID NO:26, the M protein does not have SEQ ID NO:7 or 21, and the E does not have SEQ ID NO:20. Such an immunological composition may provide enhanced immunity to SARS-CoV-2 variants. For example, the SARS-CoV-2 spike protein that does not have SEQ ID NO:5, 34, or 35 may have any of the amino acid substitutions or mutations listed in Table 2. For example, the SARS-CoV-2 N protein that does not have SEQ ID NO:26 may have any of the amino acid substitutions or mutations listed in Table 3. For example, the SARS-CoV-2 M protein that does not have SEQ ID NO:7 or 21 may have any of the amino acid substitutions or mutations listed in Table 4. For example, the SARS-CoV-2 E protein that does not have SEQ ID NO:20 may have any of the amino acid substitutions or mutations listed in Table 5.

The compositions can be pharmaceutical compositions. In some embodiments, the compositions can include a pharmaceutically acceptable carrier. By “pharmaceutically acceptable” it is meant that a carrier, diluent, excipient, and/or salt is compatible with the other ingredients of the formulation, and not deleterious to the recipient thereof.

In some embodiments, the active agents of the invention are administered in a “therapeutically effective amount.” Such a therapeutically effective amount is an amount sufficient to obtain the desired physiological effect, such a reduction of at least one symptom of SARS-CoV-2 infection. For example, active agents can reduce the symptoms of SARS-CoV-2 infection by 5%, or 10%, or 15%, or 20%, or 25%, or 30%, or 35%, or 40%, or 45%, or 50%, or 55%, or 60%, or 65%, or % 70, or 80%, or 90%, 095%, or 97%, or 99%, or any numerical percentage between 5% and 100%. For example, symptoms of SARS-CoV-2 infection can also include inflammation, fever, chills, shortness of breath, difficulty breathing, fatigue, muscle aches, headache, loss of tase and/or smell, sore throat, congestion, runny nose, nausea, vomiting, diarrhea, and combinations thereof.

To achieve the desired effect(s), the active agents may be administered as single or divided dosages. For example, active agents can be administered in dosages of at least about 0.01 mg/kg to about 500 to 750 mg/kg, of at least about 0.01 mg/kg to about 300 to 500 mg/kg, at least about 0.1 mg/kg to about 100 to 300 mg/kg or at least about 1 mg/kg to about 50 to 100 mg/kg of body weight, although other dosages may provide beneficial results.

The amount or number of VLPs administered can vary but amounts in the range of about 106 to about 109 VLPs can be used. The cells are generally delivered in a physiological solution such as saline or buffered saline. The cells can also be delivered in a vehicle such as within a population of liposomes, exosomes or microvesicles.

The amount administered will vary depending on various factors including, but not limited to, the type of VLPs, small molecules, compounds, polypeptides, antibodies, or inhibitory nucleic acid chosen for administration, the disease, the weight, the physical condition, the health, and the age of the subject. Such factors can be readily determined by the clinician employing animal models or other test systems that are available in the art.

Administration of the active agents in accordance with the present invention may be in a single dose, in multiple doses, in a continuous or intermittent manner, depending, for example, upon the recipient's physiological condition, whether the purpose of the administration is therapeutic or prophylactic, and other factors known to skilled practitioners. The administration of the active agents and compositions of the invention may be essentially continuous over a preselected period of time or may be in a series of spaced doses. Both local and systemic administration is contemplated.

The composition can be formulated in any convenient form. To prepare the composition, VLPs, small molecules, compounds, polypeptides, antibodies, inhibitory nucleic acids, and other agents are synthesized or otherwise obtained, purified as necessary or desired. These VLPs, small molecules, compounds, polypeptides, antibodies, inhibitory nucleic acids, and other agents can be suspended in a pharmaceutically acceptable carrier and/or lyophilized or otherwise stabilized. The VLPs, small molecules, compounds, polypeptides, antibodies, inhibitory nucleic acids, and other agents, and combinations thereof can be adjusted to an appropriate concentration, and optionally combined with other agents. The absolute weight of a given VLPs, small molecules, compounds, polypeptides, antibodies, inhibitory nucleic acids, and other agents included in a unit dose can vary widely.

For example, about 0.01 to about 2 g, or about 0.1 to about 500 mg, of at least one VLP, small molecule, compound, polypeptide, antibody type, inhibitory nucleic acid, or other agent can be administered. Alternatively, the unit dosage can vary from about 0.01 g to about 50 g, from about 0.01 g to about 35 g, from about 0.1 g to about 25 g, from about 0.5 g to about 12 g, from about 0.5 g to about 8 g, from about 0.5 g to about 4 g, or from about 0.5 g to about 2 g.

Daily doses of the active agents of the invention can vary as well. Such daily doses can range, for example, from about 0.1 g/day to about 50 g/day, from about 0.1 g/day to about 25 g/day, from about 0.1 g/day to about 12 g/day, from about 0.5 g/day to about 8 g/day, from about 0.5 g/day to about 4 g/day, and from about 0.5 g/day to about 2 g/day.

It will be appreciated that the amount of active agent for use in treatment will vary not only with the particular carrier selected but also with the route of administration, the extent or severity of the subject's condition being treated and the age and condition of the patient. Ultimately the attendant health care provider can determine proper dosage. In addition, a pharmaceutical composition can be formulated as a single unit dosage form.

Thus, one or more suitable unit dosage forms comprising the active agent(s) can be administered by a variety of routes including parenteral (including subcutaneous, intravenous, intramuscular and intraperitoneal), oral, rectal, dermal, transdermal, intrathoracic, intrapulmonary and intranasal (respiratory) routes. The active agent(s) may also be formulated for sustained release (for example, using microencapsulation, see WO 94/07529, and U.S. Pat. No. 4,962,091). The formulations may, where appropriate, be conveniently presented in discrete unit dosage forms and may be prepared by any of the methods well known to the pharmaceutical arts. Such methods may include the step of mixing the active agent with liquid carriers, solid matrices, semi-solid carriers, finely divided solid carriers or combinations thereof, and then, if necessary, introducing or shaping the product into the desired delivery system. For example, the active agent(s) can be linked to a convenient carrier such as a nanoparticle, albumin, polyalkylene glycol, or be supplied in prodrug form. The active agent(s), and combinations thereof can be combined with a carrier and/or encapsulated in a vesicle such as a liposome.

The compositions of the invention may be prepared in many forms that include aqueous solutions, suspensions, tablets, hard or soft gelatin capsules, and liposomes and other slow-release formulations, such as shaped polymeric gels. Administration of active agents can also involve parenteral or local administration of the in an aqueous solution or sustained release vehicle.

In some cases the VLPs, small molecules, compounds, polypeptides, antibodies, inhibitory nucleic acids, and other agents, and combinations thereof and/or other agents can be formulated as a nasal spray or as an inhalable spray to be inhaled into the lungs.

While the active agent(s) and/or other agents can sometimes be administered in an oral dosage form, that oral dosage form can be formulated so as to protect the VLPs, small molecules, compounds, polypeptides, antibodies, inhibitory nucleic acids, and other agents, and combinations thereof, and combinations thereof provide therapeutic utility. For example, in some cases the VLPs, small molecules, compounds, polypeptides, antibodies, inhibitory nucleic acids, and other agents, and combinations thereof and/or other agents can be formulated for release into the intestine after passing through the stomach. Such formulations are described, for example, in U.S. Pat. No. 6,306,434 and in the references contained therein.

Liquid pharmaceutical compositions may be in the form of, for example, aqueous or oily suspensions, solutions, emulsions, syrups or elixirs, dry powders for constitution with water or other suitable vehicle before use. Such liquid pharmaceutical compositions may contain conventional additives such as suspending agents, emulsifying agents, non-aqueous vehicles (which may include edible oils), or preservatives. The pharmaceutical compositions may take such forms as suspensions, solutions, or emulsions in oily or aqueous vehicles, and may contain formulatory agents such as suspending, stabilizing and/or dispersing agents. Suitable carriers include saline solution, encapsulating agents (e.g., liposomes), and other materials. The active agent(s) and/or other agents can be formulated in dry form (e.g., in freeze-dried form), in the presence or absence of a carrier. If a carrier is desired, the carrier can be included in the pharmaceutical formulation, or can be separately packaged in a separate container, for addition to the agent that is packaged in dry form, in suspension or in soluble concentrated form in a convenient liquid.

An active agent(s) and/or other agents can be formulated for parenteral administration (e.g., by injection, for example, bolus injection or continuous infusion) and may be presented in unit dosage form in ampoules, prefilled syringes, small volume infusion containers or multi-dose containers with an added preservative.

The compositions can also contain other ingredients such as active agents, anti-viral agents, antibacterial agents, antimicrobial agents and/or preservatives.

The present description is further illustrated by the following examples, which should not be construed as limiting in any way. The contents of all cited references (including literature references, issued patents, published patent applications as cited throughout this application) are hereby expressly incorporated by reference.

### Example 1: Materials and Methods

Cloning for plasmids encoding structural proteins: pcDNA3.1 backbone plasmids were generated encoding N, and M-IRES-E. Sequences for E, M and N were PCR amplified from codon optimized plasmids were gifts from Nevan Krogan (Addgene plasmid #141385, 141386, 141391,). The pcDNA3.1-SARS2-Spike construct was a gift from Fang Li (Addgene plasmid #145032). Site directed mutagenesis (NEB) was used to remove the C9-tag and introduce the D614G mutation. Delta and Omicron structural protein were cloned ligating eBlocks (IDT) gene fragments following NEBuilder HiFi DNA (NEB E2621L) Assembly Reaction Protocol.

Cloning of SARS-Cov-2 genome tiled segments: RNA was extracted from SARS-CoV-2 (Washington isolate) viral supernatant inactivated in Trizol by phase separation. RNA was reverse transcribed using protoscript II (NEB) and tiled segments (T1-T28) were PCR amplified from cDNA using primers compatible with ligation independent cloning (LIC). Tiles were cloned into a plasmid containing luciferase with a LIC destination site in the 3′UTR

SARS-CoV-2 virus-like-particle (SC2-VLP) production: For a 6-well, plasmids SARS-Cov2-N (0.67), SARS-CoV2-M-IRES-E (0.33), SARS-CoV-2-Spike (0.0016) and Luc-T20 (1.0) at the indicated mass ratios for a total of 4 μg of DNA, which was diluted in 200 μL Opti-MEM. Twelve μg polyethylenimine (PEI) was diluted in 200 μL Opti-MEM and this mixtures was quickly added to the diluted plasmid mixture to complex the DNA. For a 24-well, plasmids CoV2-N (0.67), CoV2-M-IRES-E (0.33), CoV-2-Spike (0.006) and Luc-PS9 (1.0) at the indicated mass ratios for a total of 1 μg of DNA, which was diluted in 50 μL Opti-MEM. 3 μg PEI was diluted in 50 μL Opti-MEM and quickly added to the diluted plasmid mixture to complex the DNA. Transfection mixtures were incubated for 20 minutes at room temperature and then added dropwise to 293T cells in 0.5-2 mL of DMEM containing fetal bovine serum and penicillin/streptomycin. Media was changed after 24 hours of transfection and at 48 hours post-transfection, VLP-containing supernatant was collected and filtered using a 0.45 μm syringe filter. For other culture sizes, the mass of DNA used was 1 μg for 24-well, 4 μg for 6-well, 20 μg for 10-cm plate and 60 μg for 15-cm plate. Optimum volumes were 100 μL, 400 μL, 1 mL and 3 mL respectively and PEI was always used at 3:1 mass ratio.

Luciferase readout: In each well of a clear 96-well plate 50 μL of SC2-VLP containing supernatant was added to 50 L of cell suspension containing 30,000-50,000 receiver/receptor cells (293T ACE2/TMPRSS2) Cells were allowed to attach and take up VLPs overnight. Next day, supernatant was removed and cells were rinsed with 1×PBS and lysed in 20 μL passive lysis buffer (Promega) for 15 minutes at room temperature with gentle rocking. Lysates were transferred to an opaque white 96-well plate and 30-50 μL of reconstituted luciferase assay buffer was added and mixed with each lysate. Luminescence was measured immediately after mixing using a TECAN plate reader (in some cases with no attenuation and a luminescence integration time of 1 second.

VLP purification using sucrose cushion: SC2-VLP produced in 10-cm plates (10 mL of culture) were added to 13.2 mL ultracentrifuge tubes. 1 mL of 20% sucrose was underlaid using a 4″ blunt needle. VLPs were centrifuged for 2 hours at 28 000 RPM using a SW41 Ti swinging bucket rotor. Supernatant was removed and ultracentrifuge tubes were inverted for 5 minutes on a paper towel with gentle tapping to remove remaining supernatant. VLPs were resuspended in 50 μL phosphate buffered saline for further experiments.

SC2-VLP PEG precipitation: 0.136 volumes of polyethylene glycol stock (50% PEG, 2.2% NaCl) was added to filtered supernatant containing SC2-VLPs to achieve a final concentration of 6% PEG. Solution was mixed thoroughly and precipitation was allowed to proceed for 2 hrs at 4° C. and then centrifuged at 2 000 g for 20 minutes. Supernatant was discarded and VLPs were resuspended in PBS.

SC2-VLP concentration using Amicon filters: 0.5 mL filtered supernatant was added to 0.5 mL 100 kDa molecular weight cutoff Amicon filters and centrifuged for 30 minutes at 2 000 g Concentrate was diluted in 1×PBS containing 0.02% tween 20 for all wash steps.

Western blot cell lysate and VLPs. For western blots of lysates, media was removed and cells were rinsed with PBS. Cells were then lysed for 20 minutes in RIPA lysis buffer containing Halt protease and phosphatase inhibitor cocktail. For western blots of ultracentrifuge concentrated VLPs, 10 mL of VLP supernatant from a 10-cm plate was pelleted (28000 RPM, 2 hrs, SW41 Ti, 1 mL 20% sucrose cushion), the supernatant was discarded and VLPs were resuspended in 50 μL of PBS. 15 μL of concentrated VLPs were used to western blot. Laemmli loading buffer (1× final) and dithiothreitol (DTT, 40 mM final) was added to lysates or VLP solution and heated for 95° C. for 5 minutes to lyse VLPs and denature proteins. Samples were loaded on to 4-20% gradient gels or 12-40% gradient gels (Biorad) and transferred to a PVDF membrane (Biorad). Membrane was blocked in 10% NFDM and stained with primary antibody: anti-N (abcam ab273434, 1:500 dilution), anti-S (abcam ab272504, 1:1000), anti-GAPDH (Santa Cruz sc-365062, 1:1000), anti-p24 (Sigma, 1:2000) for 2 hours at room temperature. Blots were rinsed with TBS-T three times for 10 minutes each and stained with secondary (mouse: abcam ab205719, or rabbit: Invitrogen, 65-6120, 1:5000). Imaged using pierce chemiluminescence kit and Biorad Chemidoc imager.

Sucrose gradient fractionation: 10% to 40% sucrose gradient was prepared using a gradient mixer in 13.2 mL ultracentrifuge tubes. Concentrated and resuspended SC2-VLPs were overlaid on top of the gradient and centrifuged in a SW41 Ti rotor for 3 hours at 28 000 RPM. Gradient was fractionated from the bottom using a 4″ blunt needle and a peristaltic pump. For cell infection, each fraction was diluted 20× and added to 293T cells expressing ACE2/TMPRSS2. Luciferase signal was measured the next day.

GFP-VLPs and flow cytometry. GFP was cloned into the luciferase destination vector (Luc-no PS) and Luc-PS9 to generate GFP-LIC and GFP-PS9. VLPs were generated in 10-cm plates and concentrated through a 20% sucrose cushion. 50 μL of concentrated VLPs were added to each well of a 24-well plate along with 120,000 receiver cells (293T ACE2/TMPRSS2). Cells were incubated with VLPs overnight and GFP expression was measured the next day using flow cytometry.

Northern Blot: VLPs collected from a 10-cm plate were concentrated by ultracentrifugation through a 20% sucrose cushion (28000 RPM, 2 hrs, SW41 Ti). The supernatant was discarded and VLPs were resuspended in 50 μL of PBS. 20 μL of concentrated VLPs were used for Northern blotting. VLPs were lysed by adding 500 μL of Trizol (Sigma) and RNA was extracted by phase separation, precipitated with isopropanol with GlycoBlue and washed with 75% ethanol. RNA was resuspended in 30 μL of water, added to 30 μL 2×RNA Loading Dye (NEB) and denatured at 65° C. for 15 minutes then loaded onto a 1% agarose gel containing 1×MOPS and 4% formaldehyde. Samples were run at room temperature for 12 hrs at 20V and transferred by capillary action to Nylon membrane. The membrane was hybridized with a ³²P-labeled luciferase DNA probe (Promega) and visualized using a phosphoscreen on a Typhoon imager (GE).

Cell lines: Cells were maintained in a humidified incubator at 37° C. in 5% CO2 in the indicated media and passaged every 3-4 days. 293T cells were obtained from ATCC and maintained in DMEM with 10% FBS and 1% penicillin/streptomycin. 293T cells stably co-expressing ACE2 and TMPRSS2 were generated through sequential transduction of 293T cells with TMPRSS2-encoding (generated using Addgene plasmid #170390, a gift from Nir Hacohen and ACE2-encoding (generated using Addgene plasmid #154981, a gift from Sonja Best) lentiviruses and selection with hygromycin (250 μg/mL) and blasticidin (10 μg/mL) for 10 days, respectively. ACE2 and TMPRSS2 expression was verified by western blot.

Neutralization Assays: Each heat inactivated serum sample was serially diluted at 1:20 to 1:20480 dilution ratios in complete DMEM media prior to incubation (1 hr at 37° C.) with 40 μL VLP with total volume of 50 μL. The mixtures were then plated onto receiver cells (50000 293T ACE2-TMPRSS2 cells) and 24 hr later luciferase readouts were taken. Neutralization (NT50) was estimated by interpolating the dilution of serum at which 50% infectivity was reduced.

Serum samples: Serum samples from individuals not exposed to SARS-CoV-2 (pre-COVID, control), exposed to SARS-CoV-2 (post-COVID), and those vaccinated with either two doses of elasomeran (Moderna), two doses of tozinameran (Pfizer/BioNTech) vaccine or one dose of Johnson & Johnson vaccine were collected through a clinical trial led by Curative. Table 1 lists some of the properties of serum samples from different trail participants.

TABLE 1
Serum samples from clinical trial participants used in VLP assays

| Subject | ELISA | Total IgG | |
|---|---|---|---|
| ID | Status | (ug/ml) | Sample Type |
| CUR01 | Negative | / | pre-COVID serum |
| CUR02 | Negative | / | pre-COVID serum |
| CUR03 | Negative | / | pre-COVID serum |
| CUR04 | Negative | / | pre-COVID serum |
| CUR05 | Negative | / | pre-COVID serum |
| PC0002 | Positive | 4.45 | post-COVID serum |
| PC0003 | Positive | 0.44 | post-COVID serum |
| PC0006 | Positive | 2.29 | post-COVID serum |
| PC0007 | Positive | 1.19 | post-COVID serum |
| PC0008 | Positive | 2.16 | post-COVID serum |
| PC0009 | Positive | 1.19 | post-COVID serum |
| PC0011 | Positive | 39.8 | post-COVID serum |
| PC0013 | Positive | 1.03 | post-COVID serum |
| PF0002 | Positive | 9.67 | Pfizer vaccinee serum - 2 doses |
| PF0004 | Positive | 9.32 | Pfizer vaccinee serum - 2 doses |
| PF0005 | Positive | 9.36 | Pfizer vaccinee serum - 2 doses |
| PF0006 | Positive | 5.05 | Pfizer vaccinee serum - 2 doses |
| PF0007 | Positive | 8.85 | Pfizer vaccinee serum - 2 doses |
| PF0009 | Positive | 8.21 | Pfizer vaccinee serum - 2 doses |
| PF0011 | Positive | 9.66 | Pfizer vaccinee serum - 2 doses |
| PF0012 | Positive | 7.01 | Pfizer vaccinee serum - 2 doses |
| PF0013 | Positive | 6.41 | Pfizer vaccinee serum - 2 doses |
| PF0016 | Positive | 1.79 | Pfizer vaccinee serum - 2 doses |
| PF0017 | Positive | 7.72 | Pfizer vaccinee serum - 2 doses |
| M0002 | Positive | 91.77 | Moderna vaccinee serum - 2 doses |
| M0003 | Positive | 14.5 | Moderna vaccinee serum - 2 doses |
| M0004 | Positive | 71.94 | Moderna vaccinee serum - 2 doses |
| M0005 | Positive | 9.88 | Moderna vaccinee serum - 2 doses |
| M0006 | Positive | 8.5 | Moderna vaccinee serum - 2 doses |
| M0007 | Positive | 10.5 | Moderna vaccinee serum - 2 doses |
| M0008 | Positive | 21.38 | Moderna vaccinee serum - 2 doses |
| M0009 | Positive | 10.2 | Moderna vaccinee serum - 2 doses |
| M0010 | Positive | 15.65 | Moderna vaccinee serum - 2 doses |
| M0011 | Positive | 15.08 | Moderna vaccinee serum - 2 doses |
| JJ0002 | Positive | 1.09 | J + J vaccinee serum - 1 dose |
| JJ0003 | Positive | 1.63 | J + J vaccinee serum - 1 dose |
| JJ0005 | Positive | 1.29 | J + J vaccinee serum - 1 dose |
| JJ0006 | Positive | 2.09 | J + J vaccinee serum - 1 dose |
| JJ0007 | Positive | 1.19 | J + J vaccinee serum - 1 dose |
| JJ0008 | Positive | 1.84 | J + J vaccinee serum - 1 dose |
| JJ0009 | Positive | 0.57 | J + J vaccinee serum - 1 dose |
| JJ0010 | Positive | 0.55 | J + J vaccinee serum - 1 dose |
| JJ0011 | Positive | 1.68 | J + J vaccinee serum - 1 dose |

Post-COVID samples reflect non vaccinated participant samples that were collected within 4-6 weeks of the original positive test and were negative by PCR at the time of serum collection. Serum from vaccinated participants was collected 4-6 weeks post vaccination following final dose. The clinical trial protocol was approved by Advarra under Pro00054108 for a study designed to investigate immune escape by SARS-CoV-2 variants. The trial has been submitted to clinicaltrials.gov registry (NCT ID pending, Unique Protocol ID: PTL-2021-0007). Sample specimens were collected from adult individuals aged 18 to 50 years who either had been vaccinated for COVID-19 and/or had a history of COVID-19. Vulnerable populations were excluded from enrollment. Patients signed consent forms held by Curative. Participants were enrolled from individuals that tested with Curative in Los Angeles County and were sent an IRB-approved email enrollment script. Those who were interested were contacted by the Curative Clinical Trials research team (CITI trained) and those who consented to the study were scheduled for sample collection by a clinician who went to their residence. Participants underwent a standard venipuncture procedure Briefly, licensed phlebotomists collected a maximum of 15 ml whole blood. Once collected, the sample was left at ambient temperature for 30-60 min to coagulate, then was centrifuged at 2200-2500 rpm for 15 min at room temperature. Samples were then placed on ice until delivered to the laboratory site where the serum was aliquoted to appropriate volumes for storage at −80° C. until use. A quantitative SARS-CoV-2 IgG ELISA was performed on serum specimens (EuroImmun, Anti-SARS-CoV-2 ELISA (IgG), 2606-9621G, New Jersey). To quantify SARS-CoV-2 IgG antibodies, an S1-specific monoclonal IgG antibody with no known cross-reactivity to the S2 domain of the spike protein was used as a reference antibody. A standard curve was developed using a monoclonal IgG antibody targeting the S1 antigen of SARS-CoV-2 at different concentrations with a polynomial regression curve-fitting model. The standard curve was used to calculate the sample IgG antibody concentration. Serum samples were heat inactivated at 56° C. for 30 mins prior to use in VLP assays. Pre-COVID sera was pooled into one sample.

### Example 2: Identification of the SARS-CoV-2 Packaging Signal

The inventors hypothesized that the SARS-CoV-2 packaging signal might reside within genomic fragment “T20” (nucleotides 20080-22222) encoding non-structural protein 15 (nsp15) and nsp16 (FIG. 1A).

A sequence for the SARS-CoV-2 nsp15 protein is available as accession number YP_009725310 at the NCBI website and is provided below as SEQ ID NO:32.

| | |
|---|---|
| 1 | SLENVAFNVV NKGHEDGQQG EVPVSIINNT VYTKVDGVDV |
| 41 | ELFENKTTLP VNVAFELWAK RNIKPVPEVK ILNNLGVDIA |
| 81 | ANTVIWDYKR DAPAHISTIG VCSMTDIAKK PTETICAPLT |
| 121 | VEFDGRVDGQ VDLERNARNG VLITEGSVKG LQPSVGPKQA |
| 161 | SLNGVTLIGE AVKTQFNYYK KVDGVVQQLP ETYFTQSRNL |
| 201 | QEFKPRSQME IDFLELAMDE FIERYKLEGY AFEHIVYGDE |
| 241 | SHSQLGGLHL LIGLAKRFKE SPFELEDFIP MDSTVKNYFI |
| 281 | TDAQTGSSKC VCSVIDLLLD DEVEIIKSQD LSVVSKVVKV |
| 321 | TIDYTEISFM LWCKDGHVET FYPKLQ |

A sequence for the SARS-CoV-2 nsp16 protein is available as NCBI accession number 6YZ1_A and is provided below as SEQ ID NO:33.

| | |
|---|---|
| 1 | MSSQAWQPGV AMPNLYKMQR MLLEKCDLQN YGDSATLPKG |
| 41 | IMMNVAKYTQ LCQYLNTLTL AVPYNMRVIH FGAGSDKGVA |
| 81 | PGTAVLRQWL PTGTLLVDSD LNDFVSDADS TLIGDCATVH |
| 121 | TANKWDLIIS DMYDPKTKNV TKENDSKEGF FTYICGFIQQ |
| 161 | KLALGGSVAI KITEHSWNAD LYKLMGHFAW WTAFVINVNA |
| 201 | SSSEAFLIGC NYLGKPREQI DGYVMHANYI FWRNINPIQL |
| 241 | SSYSLFDMSK FPLKLRGTAV MSLKEGQIND MILSLLSKGR |
| 281 | LIIRENNRVV ISSDVLVNN |

SARS-CoV-2 sequences can vary without significantly reducing their function. Hence, the foregoing sequences can have one or more substitutions, deletions, or insertions.

A transfer plasmid was designed encoding a luciferase transcript containing the T20 region within its 3′ untranslated region (UTR) (FIG. 1L). The transfer plasmid was then tested for SARS-CoV-2 virus-like-particle production by co-transfecting the transfer plasmid into packaging cells (HEK293T) along with plasmids encoding the virus structural proteins (FIG. 1A-1B). Supernatant secreted from these packaging cells was filtered and incubated with receiver 293T cells co-expressing SARS-CoV-2 entry factors ACE2 and TMPRSS2 (FIG. 1B).

Luciferase expression was observed in receiver cells only in the presence of all four SARS-CoV-2 structural proteins (S, M, N, E) as well as the T20-containing reporter transcript (FIG. 1C). Substituting any one of the structural proteins or the luciferase-T20 transcript with a luciferase-only transcript decreased luminescence in receiver cells by >200-fold and 63-fold respectively (FIG. 1C).

This experiment was also conducted using Vero E6-TMPRSS2 cells that endogenously express ACE2. Once again robust luciferase expression was observed when all five components were present but significantly lower luciferase expression was observed when any one of the SARS-CoV-2 structural proteins (S, M, N, E) or the T20-containing reporter transcript was missing (FIG. 1J).

The approach required two key modifications compared to previous work on SARS-CoV-2 VLPs. First, although affinity sequence tags on N were tolerated, untagged native M protein was required for SC2-VLP-mediated reporter gene expression because tags on the M protein dramatically reduced VLP formation (FIG. 1D). Tags on S and E proteins were not evaluated. Second, luciferase expression in receiver cells was most efficient within a narrow range of Spike expression and at surprisingly low ratios of Spike expression plasmid relative to the other plasmids (FIG. 1E). However, the N and S proteins were detected within pelleted VLP material but VLP formation was dependent on the amount or ratio of Spike protein (FIGS. 1F-1G). These results indicate that particles produced under less stringent conditions are not competent for delivering RNA to receiver/receptor cells. This may explain why exogenous RNA delivery has not been observed previously for SARS-CoV-2 VLPs.

Further analysis showed that SARS-CoV-2 VLPs (SC2-VLPs) are stable against ribonuclease A, resistant to freeze-thaw (FT) treatment (FIG. 1M) and can be concentrated by precipitation, ultrafiltration and ultracentrifugation through a 20% sucrose cushion (FIG. 1H-1I, 1N). Analysis of SC2-VLPs fractionated using 10-40% sucrose gradient ultracentrifugation showed that large dense particles are responsible for inducing luciferase expression (FIG. 1H-1I, 1N). These data support the conclusion that SC2-VLPs are formed under the experimental conditions and deliver selectively packaged transcripts by receptor-mediated cell entry into receptor/receiver cells.

The SC2-VLPs were then used to locate more accurately the SARS-CoV-2 RNA packaging signal. A library of 28 two kilobase overlapping tiled segments (T1-T28) were generated from the SARS-CoV-2 genome and these nucleic acid segments were individually inserted into a luciferase-encoding plasmid (FIG. 2A). SC2-VLPs were generated using a luciferase-encoding plasmid and plasmids that included all regions of ORF1ab from SARS-CoV-2. These SC2-VLPs produced luminescence detectable in this assay, indicating that packaging does not rely entirely on one contiguous RNA sequence (FIG. 2B-2C). However, luciferase-encoding plasmids that included fragments T24-28 resulted in lower luciferase expression (FIG. 2B-2C), consistent with natural exclusion of subgenomic viral transcripts containing these sequences to avoid generation of replication-defective virus particles. Overall, packaging was most efficient using T20 (nucleotides 20080-22222) located near the 3′ end of ORF1ab (FIG. 2B-2E). A sequence for the T20 (nucleotides 20080-22222) region is shown below as SEQ ID NO:2.

| | |
|---|---|
| 20080 | T |
| 20081 | CTGTAGGTCC CAAACAAGCT AGTCTTAATG GAGTCACATT |
| 20121 | AATTGGAGAA GCCGTAAAAA CACAGTTCAA TTATTATAAG |
| 20161 | AAAGTTGATG GTGTIGTCCA ACAATTACCT GAAACTTACT |
| 20201 | TTACTCAGAG TAGAAATTTA CAAGAATTTA AACCCAGGAG |
| 20241 | TCAAATGGAA ATTGATTTCT TAGAATTAGC TATGGATGAA |
| 20281 | TTCATTGAAC GGTATAAATT AGAAGGCTAT GCCTTCGAAC |
| 20321 | ATATCGTTTA TGGAGATTTT AGTCATAGTC AGTTAGGTGG |
| 20361 | TITACATCTA CTGATTGGAC TAGCTAAACG TTTTAAGGAA |
| 20401 | TCACCITTTG AATTAGAAGA TTTTATTCCT ATGGACAGTA |
| 20441 | CAGTTAAAAA CTATTTCATA ACAGATGCGC AAACAGGTTC |
| 20481 | ATCTAAGTGT GTGTGTTCTG TTATTGATTT ATTACTTGAT |
| 20521 | GATTTTGTTG AAATAATAAA ATCCCAAGAT TTATCTGTAG |
| 20561 | TTTCTAAGGT TGTCAAAGTG ACTATTGACT ATACAGAAAT |
| 20601 | TTCATTTATG CTTTGGTGTA AAGATGGCCA TGTAGAAACA |
| 20641 | TTTTACCCAA AATTACAATC TAGTCAAGCG TGGCAACCGG |
| 20681 | GTGTTGCTAT GCCTAATCTT TACAAAATGC AAAGAATGCT |
| 20721 | ATTAGAAAAG TGTGACCTTC AAAATTATGG TGATAGTGCA |
| 20761 | ACATTACCTA AAGGCATAAT GATGAATGTC GCAAAATATA |
| 20801 | CTCAACTGTG TCAATATTTA AACACATTAA CATTAGCTGT |
| 20841 | ACCCTATAAT ATGAGAGTTA TACATTTTGG TGCTGGTTCT |
| 20881 | GATAAAGGAG TTGCACCAGG TACAGCTGTT TTAAGACAGT |
| 20921 | GGTTGCCTAC GGGTACGCTG CTTGTCGATT CAGATCTTAA |
| 20961 | TGACTITGTC TCTGATGCAG ATTCAACTTT GATTGGTGAT |
| 21001 | TGTGCAACTG TACATACAGC TAATAAATGG GATCTCATTA |
| 21041 | TTAGTGATAT GTACGACCCT AAGACTAAAA ATGTTACAAA |
| 21081 | AGAAAATGAC TCTAAAGAGG GTTTTTTCAC TTACATTTGT |
| 21121 | GGGTTTATAC AACAAAAGCT AGCTCTTGGA GGTTCCGTGG |
| 21161 | CTATAAAGAT AACAGAACAT TCTTGGAATG CTGATCTTTA |
| 21201 | TAAGCTCATG GGACACTICG CATGGIGGAC AGCCTTTGTT |
| 21241 | ACTAATGTGA ATGCGTCATC ATCTGAAGCA TTTTTAATTG |
| 21281 | GATGTAATTA TCTTGGCAAA CCACGCGAAC AAATAGATGG |
| 21321 | TTATGTCATG CATGCAAATT ACATATTTTG GAGGAATACA |
| 21361 | AATCCAATTC AGTIGTCTTC CTATTCTTTA TTTGACATGA |
| 21401 | GTAAATTTCC CCTTAAATTA AGGGGTACTG CTGTTATGTC |
| 21441 | TTTAAAAGAA GGTCAAATCA ATGATATGAT TTTATCTCTT |
| 21481 | CTTAGTAAAG GTAGACTTAT AATTAGAGAA AACAACAGAG |
| 21521 | TTGTTATTTC TAGTGATGTT CTTGTTAACA ACTAAACGAA |
| 21561 | CAATGTTIGT TTTTCTTGTT TTATTGCCAC TAGTCTCTAG |
| 21601 | TCAGTGTGTT AATCTTACAA CCAGAACTCA ATTACCCCCT |
| 21641 | GCATACACTA ATTCTTTCAC ACGTGGTGTT TATTACCCTG |
| 21681 | ACAAAGTTTT CAGATCCTCA GITTTACATT CAACTCAGGA |
| 21721 | CTTGTTCTTA CCTTTCTTIT CCAATGTTAC TTGGTTCCAT |
| 21761 | GCTATACATG TCTCTGGGAC CAATGGTACT AAGAGGTTTG |
| 21801 | ATAACCCTGT CCTACCATTT AATGATGGTG TTTATTTTGC |
| 21841 | TTCCACTGAG AAGTCTAACA TAATAAGAGG CTGGATTTTT |
| 21881 | GGTACTACTT TAGATTCGAA GACCCAGTCC CTACTTATTG |
| 21921 | TTAATAACGC TACTAATGTT GTTATTAAAG TCTGTGAATT |
| 21961 | TCAATTTIGT AATGATCCAT TTTTGGGTGT TTATTACCAC |
| 22001 | AAAAACAACA AAAGTTGGAT GGAAAGTGAG TTCAGAGTTT |
| 22041 | ATTCTAGTGC GAATAATTGC ACTTTTGAAT ATGTCTCTCA |
| 22081 | GCCTTTTCTT ATGGACCTTG AAGGAAAACA GGGTAATTTC |
| 22121 | AAAAATCITA GGGAATTIGT GITTAAGAAT ATTGATGGTT |
| 22161 | ATTTTAAAAT ATATTCTAAG CACACGCCTA TTAATTTAGT |
| 22201 | GCGTGATCTC CCTCAGGGTT TT |

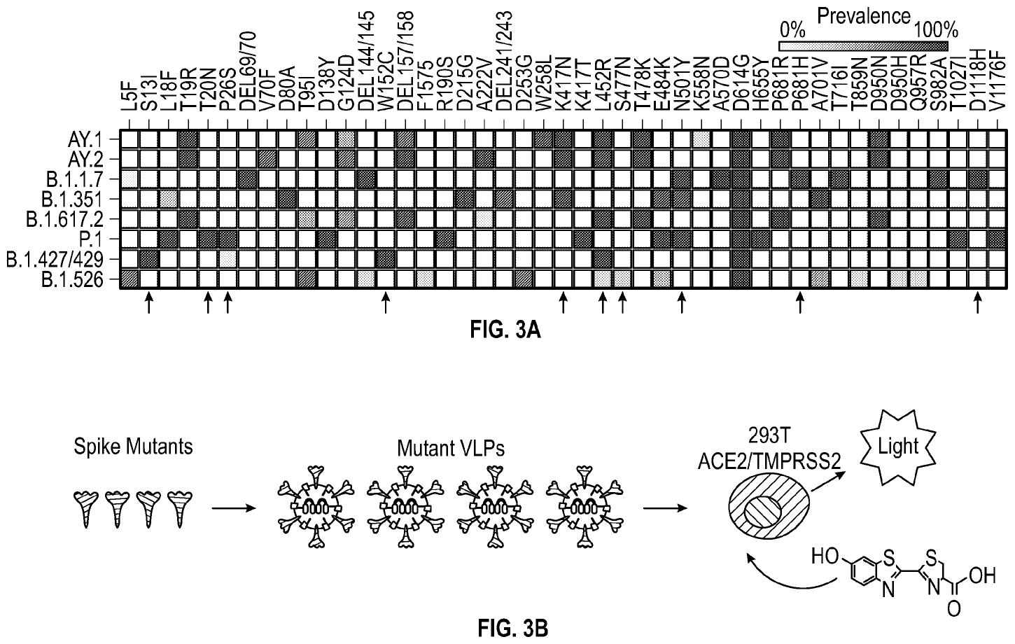

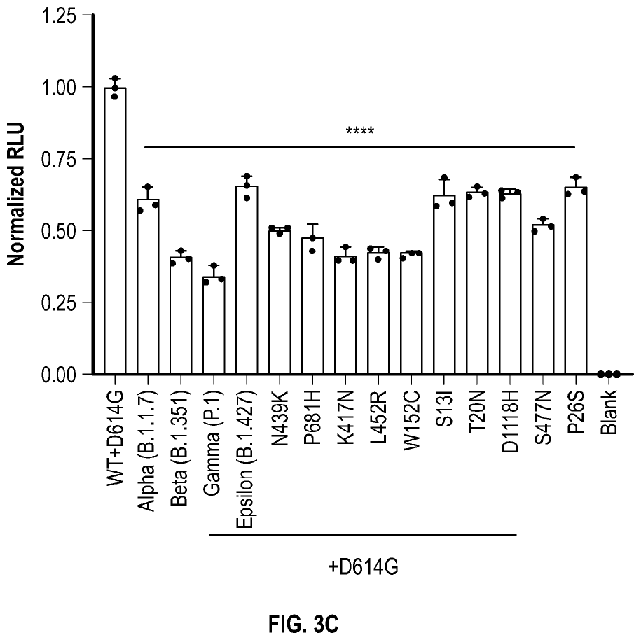

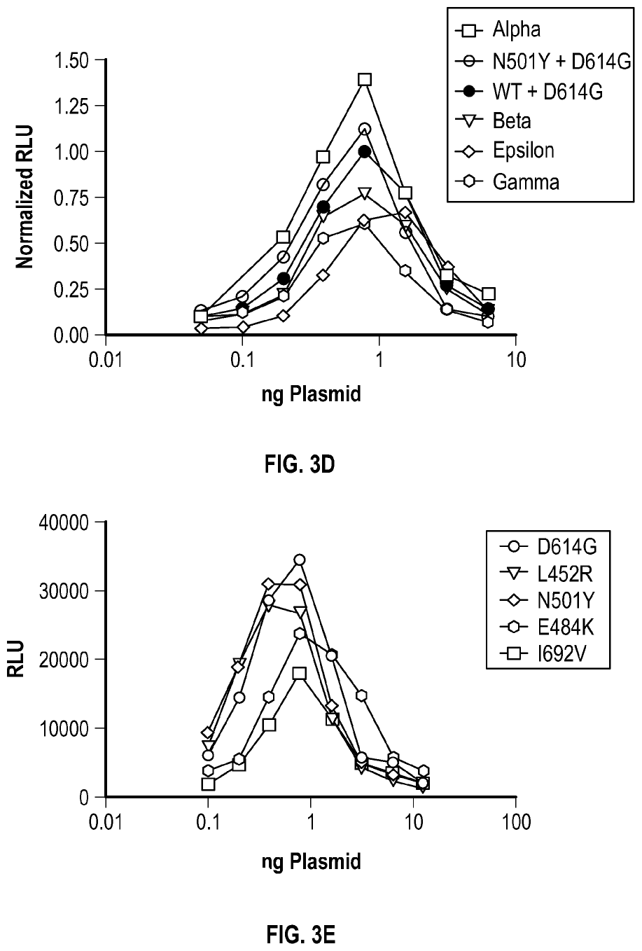

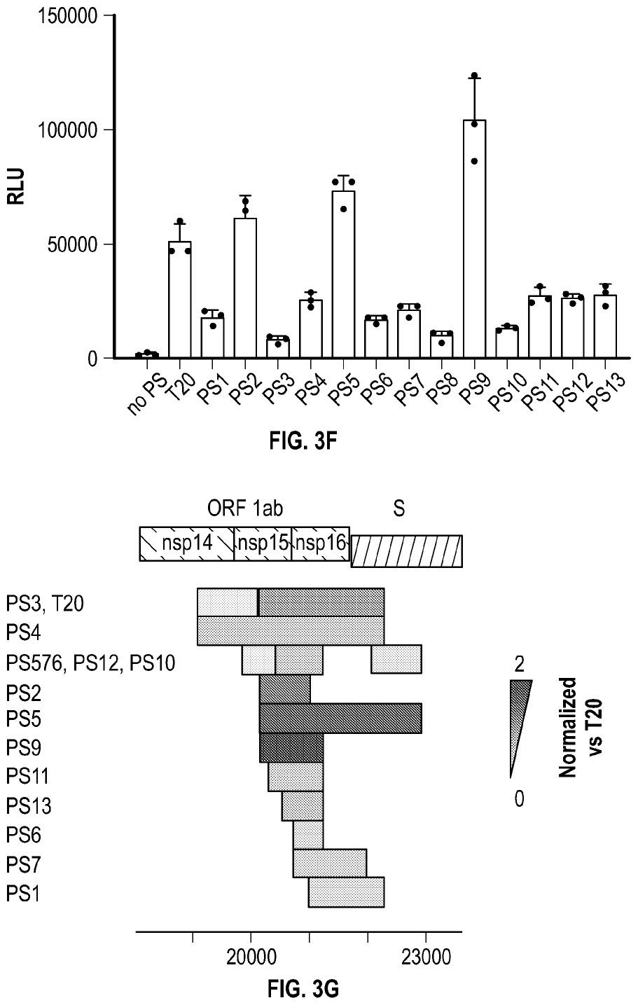

The T20 region partially but not completely overlapped with PS580 (19785-20348), which was predicted to be the packaging signal for SARS-CoV-1 based on structural similarity to known coronavirus packaging signals (Hsieh et al. J. Virol. 79, 13848-13855 (2005)). To further define the packaging sequence, truncations and additions to T20 were evaluated, including PS580 from SARS-CoV-1. As shown in FIG. 2D, use of PS576 and many other segments resulted in lower luciferase expression compared to T20 (FIG. 2D-2E; FIG. 3F-3G).

Unexpectedly, the highest luciferase expression level resulted from SC2-VLPs encoding the nucleotide sequence 20080-21171 (termed PS9), and further truncations of this sequence reduced expression (FIG. 2D-2E; FIG. 3F-3G). A sequence for nucleotides 20080-21171 (PS9) is shown below at SEQ ID NO:3.

| | |
|---|---|
| 20080 | T |
| 20081 | CTGTAGGTCC CAAACAAGCT AGTCITAATG GAGTCACATT |
| 20121 | AATTGGAGAA GCCGTAAAAA CACAGTTCAA TTATTATAAG |
| 20161 | AAAGTIGATG GTGTTGTCCA ACAATTACCT GAAACTTACT |
| 20201 | TTACTCAGAG TAGAAATTTA CAAGAATITA AACCCAGGAG |
| 20241 | TCAAATGGAA ATTGATTTCT TAGAATTAGC TATGGATGAA |
| 20281 | TTCATTGAAC GGTATAAATT AGAAGGCTAT GCCTTCGAAC |
| 20321 | ATATCGTTTA TGGAGATTTT AGTCATAGTC AGTTAGGTGG |
| 20361 | TTTACATCTA CTGATTGGAC TAGCTAAACG TTTTAAGGAA |
| 20401 | TCACCTTTTG AATTAGAAGA TITTATTCCT ATGGACAGTA |
| 20441 | CAGITAAAAA CTATTTCATA ACAGATGCGC AAACAGGTTC |
| 20481 | ATCTAAGTGT GTGTGTTCTG TTATTGATTT ATTACTTGAT |
| 20521 | GATTTTGTTG AAATAATAAA ATCCCAAGAT TTATCTGTAG |
| 20561 | TTTCTAAGGT TGTCAAAGTG ACTATTGACT ATACAGAAAT |
| 20601 | TTCATTTATG CTTTGGTGTA AAGATGGCCA TGTAGAAACA |
| 20641 | TTTTACCCAA AATTACAATC TAGTCAAGCG TGGCAACCGG |
| 20681 | GTGTTGCTAT GCCTAATCTT TACAAAATGC AAAGAATGCT |
| 20721 | ATTAGAAAAG TGTGACCTTC AAAATTATGG TGATAGTGCA |
| 20761 | ACATTACCIA AAGGCATAAT GATGAATGTC GCAAAATATA |
| 20801 | CTCAACTGTG TCAATATTTA AACACATTAA CATTAGCTGT |
| 20841 | ACCCTATAAT ATGAGAGTTA TACATTTTGG TGCTGGTTCT |
| 20881 | GATAAAGGAG TTGCACCAGG TACAGCTGTT TTAAGACAGT |
| 20921 | GGTTGCCTAC GGGTACGCTG CTTGTCGATT CAGATCTTAA |
| 20961 | TGACTITGTC TCTGATGCAG ATTCAACTTT GATTGGTGAT |
| 21001 | TGTGCAACTG TACATACAGC TAATAAATGG GATCTCATTA |
| 21041 | TTAGTGATAT GTACGACCCT AAGACTAAAA ATGTTACAAA |
| 21081 | AGAAAATGAC TCTAAAGAGG GTTTTTTCAC TTACATTTGT |
| 21121 | GGGITTATAC AACAAAAGCT AGCTCTTGGA GGTTCCGTGG |
| 21161 | CTATAAAGAT A |

VLPs were also generated that encoded GFP. Such VLPs induced GFP expression in receiver cells in the presence of PS9 (FIG. 2F).

These data indicate that PS9 (nucleotides 20080-21171) is a cis-acting element that enhances RNA packaging in the presence of SARS-CoV-2 structural proteins.

### Example 3: Spike Protein Variant Analysis

SARS-CoV-2 VLPs provide a new and more physiological model compared to pseudotyped viruses for testing mutations in all four viral structural proteins (S, E, M, N) for effects on assembly, packaging and cell entry.

SARS-CoV-2 VLPs were generated with fifteen different Spike protein mutations, including four with combined Spike mutations found in the Alpha, Beta, Gamma and Epsilon variants. Because nearly all circulating variants contain the D614G mutation in the spike protein, all mutants were compared to the ancestral spike protein modified to include G614 (termed WT+D614G).

Surprisingly, as shown in FIG. 3A-3C, improved luciferase expression was not observed from any of the SARS-CoV-2 VLPs with these spike mutations. Minor changes in Spike expression between mutants could have been a confounding factor in the absence of differences in the luciferase expression because SARS-CoV-2 VLPs mediate luciferase expression optimally in a narrow range of Spike expression. Over a range of 6.25 ng to 50 pg per well of Spike-encoding plasmid (total 1 μg of DNA used in each condition), none of the tested S mutations produced more than a 2-fold improvement in luciferase expression (FIG. 3D-3E). Only slightly increased luciferase expression occurred with the Spike sequence derived from the Alpha variant (B.1.1.7) and in the Spike protein containing the mutation N501Y within the receptor binding domain (FIG. 3D).

These results contrast with prior results obtained using S-pseudotyped lentiviruses, where enhanced entry was reported for some Spike mutations including S:N501Y (Deng et al. Cell. 184, 3426-3437.e8 (2021); Kuzmina et al. Cell Host & Microbe. 29 pp. 522-528.e2 (2021)). However, Spike mutations tested in the context of SARS-CoV-2 infectious clones have shown mixed effects, indicating that complex or indirect connections may play a role between SARS-CoV-2 spike protein and infectivity (Liu et al. bioRxiv (2021), Motozono et al. Cell Host Microbe. 29, 1124-1136.e11 (2021)).

### Example 4: N Protein Variant Analysis

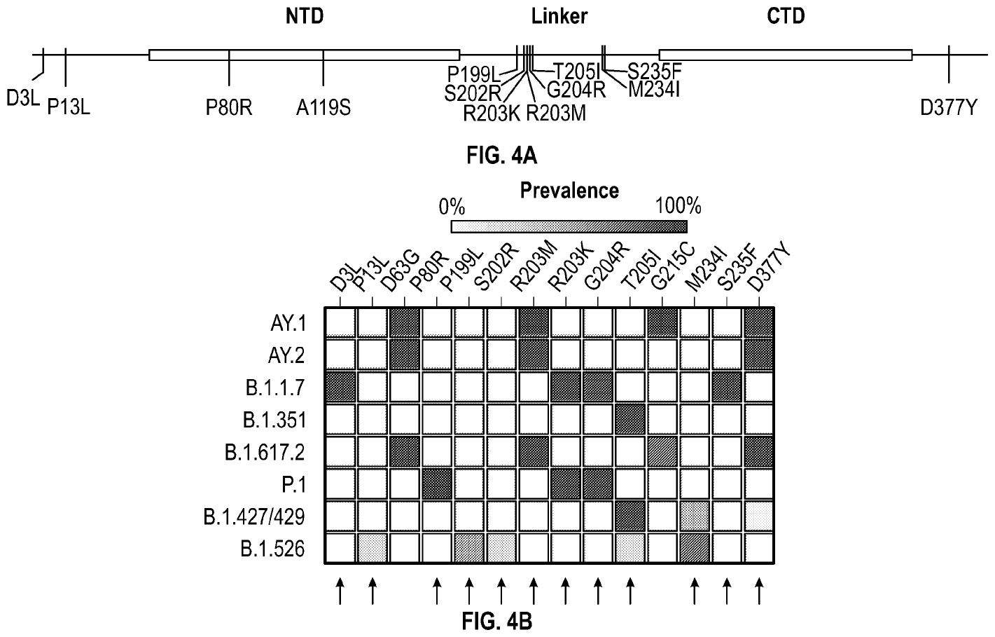

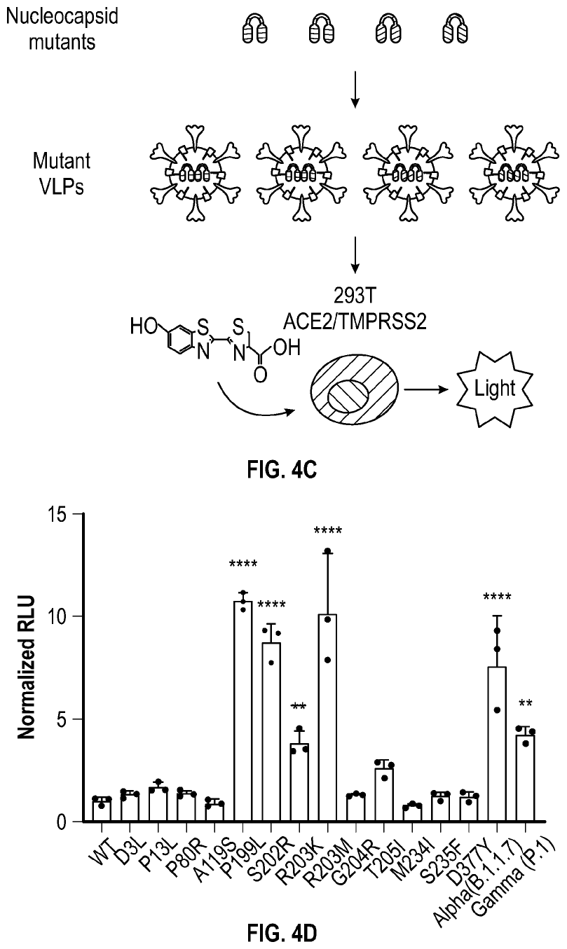

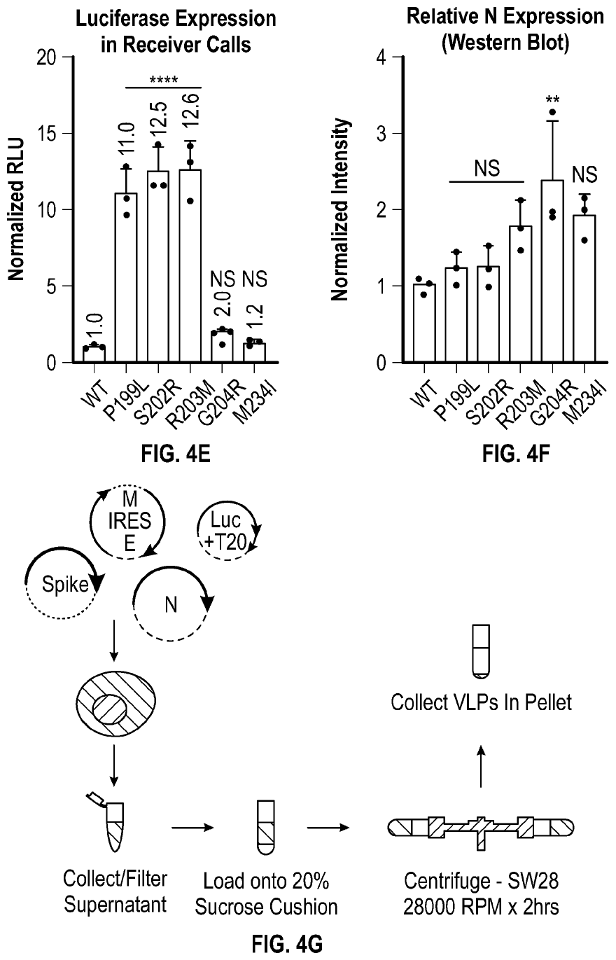

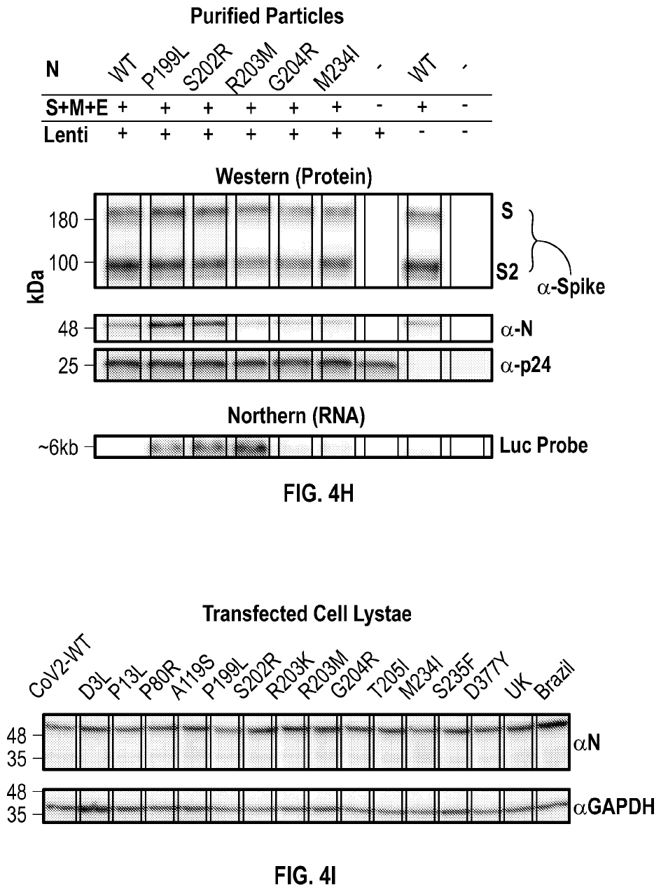

Due to the lack of observed lack of differences between different SARS-CoV-2 Spike protein mutants, the inventors decided to examine mutations in the N protein. Interestingly, half of the amino acid changes found in circulating SARS-CoV-2 variants occur within a seven amino acid region (aa199-205) of the central disordered region (termed the “linker” region, FIG. 4A-4B). Fifteen N protein mutations were tested including two combinations of mutations corresponding to the Alpha and Gamma variants that contain the co-occurring R203K/G204R mutations (FIG. 4B-4C). The N protein mutants were tested to evaluate whether such mutations result in improved viral particle assembly, RNA delivery, and/or reporter gene expression using SARS-CoV-2 VLPs.

The Alpha and Gamma variant N protein increased luciferase expression in receiver cells by 7.5-fold and 4.2-fold respectively relative to the ancestral Wuhan Hu-1 N-protein (FIG. 4D). In addition, four single amino acid changes in the N protein improved luciferase expression: P199L, S202R, R203K and R203M. Two of these amino acid changes do not change the overall charge (P199L, R203K) in that region of the N protein. However, one of the four N protein mutations resulted in a more positive charge (S202R,) and another one of the mutations resulted in a more negative charge (R203M). These results indicate that the improvement in luciferase expression is not likely due simply to electrostatics. Western blotting revealed no correlation between N protein expression levels and luciferase induction, indicating that these N mutations enhance luciferase induction through a different mechanism (FIG. 4E-4F, 4H-4I).

Further analysis of six of these N variants was conducted to determine whether these mutations affect SC2-VLP assembly efficiency, RNA packaging, or RNA uncoating prior to expression. Three of the N protein mutants exhibited increased luciferase expression (P199L, S202R, R203M) of about 10-fold Two N protein mutants did not increase luciferase expression significantly (G204R, M2341) compared to wild type (FIG. 4E) in this preliminary screen. Variation of N protein expression levels in packaging cells also did not significantly affect luciferase expression in receiver cells transduced with SC2-VLPs bearing the N protein mutations. For example, the G204R mutation exhibited increased N expression in packaging cells but this did not result in a statistically significant increase in luciferase production in receiver cells (FIG. 4E-4F).

Purified SC2-VLPs containing each N mutation were then prepared (FIG. 4G). As shown in FIG. 4H, particles with N mutations containing P199L and S202R mutation exhibited increased levels of Spike and N protein (both RNA and protein). Particles with the R203M mutation exhibited increased RNA only when compared to the mutants that did not demonstrate enhanced luciferase induction (FIG. 4G-4H).

These results indicate that mutations within the N linker domain improve the assembly of SC2-VLPs, leading either to greater overall VLP production (a larger fraction of VLPs that contain RNA) or to higher RNA content per particle. In either case, these results provide a previously unanticipated explanation for the increased fitness and spread of SARS-CoV-2 variants of concern.

In summary, new methods are described herein for rapidly generating and measuring SARS-CoV-2 VLPs that package and deliver exogenous RNA. This approach allows examination of viral assembly, budding, stability, maturation, entry and genome uncoating involving all of the viral structural proteins (S, E, M, N) without generating replication-competent virus. Such a strategy is useful not only for dissecting the molecular virology of SARS-CoV-2 but also for future development and screening of therapeutics targeting assembly, budding, maturation and entry. This strategy is ideally suited for the development of new antivirals targeting SARS-CoV-2 as it is highly sensitive, quantitative and scalable to high-throughput workflows.

The data shown herein also identify an RNA sequence within the SARS-CoV-2 genome capable of triggering packaging of exogenous transcripts. Such a packaging signal may enable the engineering of SARS-CoV-2 vaccines or therapeutics. Silent mutations can also be introduced within the packaging signal sequence to generate weakened strains of SARS-CoV-2 for use as an infectious vaccine or to generate defective genomes that package more efficiently than the original virus for use as a therapeutic strategy.

In addition, the unexpected finding of improved RNA packaging and luciferase induction by mutations within the N protein point to a previously unknown strategy for coronaviruses to evolve improved viral fitness. Although the mechanism for this improvement remains unclear, this finding is consistent with recent reports that the Delta variant (containing N:R203M) generates 1000-fold higher levels of RNA within patients. The results described herein point to a new and unanticipated mechanism that could explain why the SARS-CoV-2 Delta variant demonstrates improved viral fitness.

### Example 5: SARS-CoV-2 B.1, Delta and Omicron Variant Spike Protein

Using the SC2-VLP system described herein, a set of plasmid constructs was first generated that encoded the S, N, M and E structural proteins derived from the B.1, B.1.1, Delta and Omicron SARS-CoV-2 viral variants. The mutations in different Spike protein domains of these variants are listed in Table 2, where NTD refers to the N-terminal domain, RBD refers to the receptor binding domain, and CTD refers to the C-terminal domain.

TABLE 2
List of Spike protein mutations of SARS-CoV-2 variants

| | NTD | RBD | CTD |
|---|---|---|---|
| B.1 | | | D614G |
| B.1.1 | | | D614G |
| Delta | A67V, G142D, | L452R, T478K | D614G, P681R, |
| | E156G, Δ157-158 | | D950N |
| Omicron | A67V, Δ69-70, | G339D, S371L, | T547K, D614G, |
| | T951, G142D, | S373P, S375F, | H655Y, N679K, |
| | Δ143-145, Δ211, | K417N, N440K, | P681H, N764K, |
| | L212I, | G446S, S477N, | D796Y, N856K, |
| | ins214-EPE | T478K, E484A, | Q954H, N969K, |
| | | Q493K, G496S, | L981F |
| | | Q498R, N501Y, | |
| | | Y505H | |
| OmC1 | A67V, Δ69-70, | K417N, G496S, | T547K, D614G, |
| | T95I, G142D, | Q498R, N501Y | H655Y, N679K, |
| | Δ143-145, Δ211, | | P681H, N764K, |
| | L212I, | | D796Y, N856K, |
| | ins214-EPE | | Q954H, N969K, |
| | | | L981F |
| OmC3 | A67V, Δ69-70, | N440K, G446S, | T547k, D614G, |
| | T95I, G142D, | G496S, Q498R | H655Y, N679K, |
| | Δ143-145, Δ211, | | P681H, N764K, |
| | L212I, | | D796Y, N856K, |
| | ins214-EPE | | Q954H, N969K, |
| | | | L981F |

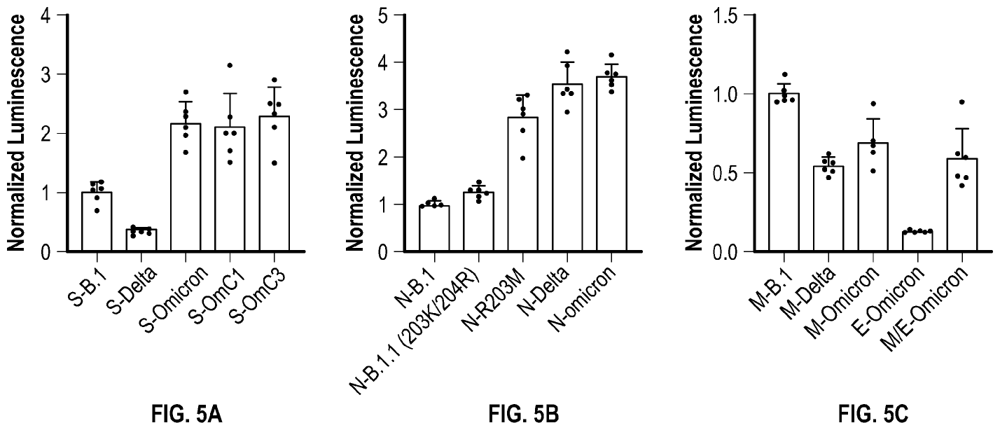

SC2-VLPs were generated by co-transfecting packaging cells (HEK293T cells) with three plasmids encoding these structural proteins and a fourth plasmid encoding luciferase mRNA linked to a SARS-CoV-2 packaging signal using methods described in Example 1. Hence, Particles secreted from these packaging cells were filtered and incubated with receiver 293T cells stably co-expressing ACE2 and TMPRSS2 (FIG. 1A-1B). To compare the effects of the different structural gene variants on infectivity, the structural genes from SARS-CoV-2 B.1 were used as the point of reference for the individual variant structural genes because the SARS-CoV-2 B.1 strain is ancestral to all currently circulating variants. For each combination of structural proteins, luciferase expression was evaluated in receiver cells, the expression level of the S and N proteins was evaluated in packaging cells, and the abundance of the S and N proteins and luciferase RNA was evaluated in the secreted VLPs (FIG. 5A-5C).

The effects on the infectivity of VLPs displaying variant S proteins was first evaluated in cells that otherwise expressed the SARS-CoV-2 B.1 structural proteins. As illustrated in FIG. 5A, the Delta variant spike protein produced VLPs that were only 20% as infectious as VLPs displaying the SARS-CoV-2 B.1 spike protein.

In contrast, the Omicron S protein in the context of the B. 1 background generated VLPs that were at least as infectious as VLPs displaying the ancestral B.1 Spike protein (FIG. 5A).

Only mutations within the spike protein receptor binding domain (RBD) have previously been shown to inhibit binding by Class 1 (417N, 496S, 498R, 501Y) or Class 3 (440K, 446S, 496S, 498R) antibodies (Greaney et al., Cell Host Microbe. 29, 44-57.e9 (2021). VLPs were generated from variants containing Omicron spike protein mutations outside the receptor binding domain (RBD) (see Table 2 for variant sequences).

As shown in FIG. 5A, these Omicron spike protein variants displayed moderately enhanced infectivity at levels of 1.8- and 1.5-fold (S-OmC1, S-OmC3). Such results indicate that genetic variations in the SARS-CoV-2 Spike protein can affect the ability of viral particles to transduce cells, and also that some S gene mutations, such as those in Omicron variants, may dominate cell infectivity outcomes.

### Example 6: Effects of N, M or E SARS-CoV-2 Variants on VLP Infectivity

This Example describes the comparative effects of N, M or E viral variants on infectivity of VLPs generated in a background of SARS-CoV-2 B.1 genes. The inventors have shown that N gene variants can influence SARS-CoV-2 infectivity and RNA packaging efficiency (Syed et al. Science, eab16184 (2021)). The N protein is required for replication, RNA binding, packaging, stabilization and release. The N protein includes a seven amino acid mutational hotspot (N:199-205) in a region linking the N-terminal and C-terminal domains. Notably, B.1.1, Delta and Omicron variants, but not the ancestral B.1 strain, include mutations at R203 that were found to enhance VLP infectivity and RNA packaging. Table 3 lists N protein mutations that are found in various SARS-CoV-2 variants, where NTD refers to the N protein N-terminal region, SR refers to the N protein seven-amino acid hotspot, linker refers to the region linking the N protein N-terminal and C-terminal regions, and CTD refers to the N protein C-terminal region.

TABLE 3
N protein Mutations in Various SARS-CoV-2 variants

| | NTD | SR | linker | CTD |
|---|---|---|---|---|
| B.1 | | | | |
| B.1.1 | | R203K, | | |
| | | G204R | | |
| Delta | D63G | R203M | G215C | D377Y |
| Omicron | P13L, Δ31-33, | R203K, | | |
| | D63G | G204R | | |

VLPs were generated from N protein variants and SARS-CoV-2 B.1 structural proteins that included luciferase-T20 transcript. The infectivity of these N protein-containing VLPs was then evaluated as described above by detecting light generated by luciferase, which was only expressed in the VLP-infected cells.

As illustrated in FIG. 5B, the N protein-Delta and N protein-Omicron variants generated VLPs with robust infectivity that was enhanced relative to VLPs displaying the B.1 and B.1.1 strain N proteins.

These results are consistent with a conclusion that the N protein plays a central role in viral packaging and cell transduction efficiency.

Omicron contains three mutations in the M protein and one mutation in the E protein relative to B.1 and Delta SARS-CoV-2 variants. Tables 4 and 5 show the mutations in the M and E proteins of Delta and Omicron variants.

TABLE 4
M Protein Mutations in SARS-CoV-2 Variants

| | |
|---|---|
| B.1 | |
| B.1.1 | |
| Delta | I82T |
| Omicron | D3G, Q19E, A63T |

TABLE 5
E Protein Mutations in SARS-CoV-2 Variants

| | |
|---|---|
| B.1 | |
| B.1.1 | |
| Delta | |
| Omicron | T9I |

As shown in FIG. 5C, VLPs generated using the Omicron M or E proteins, but with B.1 versions of the other structural components, showed levels of infectivity that were reduced relative to those measured for VLPs having the B.1 SARS-CoV-2 M and E proteins.

These results indicate that some Omicron mutations reduce viral fitness, at least on their own. To test if these effects are mitigated by mutations in other structural proteins, VLPs were generated using combinations of different structural protein mutations for each variant. The results indicate that Omicron VLPs were twice as infectious as VLPs generated using Delta or B.1.1 structural proteins and 12-fold more infectious than VLPs generated using B.1 VLPs.

### Example 7: VLPs are Useful for Detecting and Evaluating Anti-Sera from SARS-CoV-2 Vaccinated and/or Infected Individuals

This Example illustrates that the VLPs described herein are useful for detecting SARS-CoV-2 infections and for evaluating the neutralization capability of anti-sera from individuals that have been vaccinated with SARS-CoV-2 vaccines.

Antisera was collected from 38 individuals 4-6 weeks post-vaccination with Pfizer/BioNTech, Moderna or Johnson & Johnson vaccines. Convalescent sera was obtained from unvaccinated COVID-19 survivors. The antisera were collected from participants aged 18-50 years enrolled in a clinical trial led by Curative, and SARS-CoV-2 IgG antibodies were quantified with an ELISA (Table 1).

VLPs were generated with B.1 structural genes except for the N protein R203M variant, which the inventors had found to enhance assembly and increase the dynamic range of the neutralization assay. The serum described in the previous paragraph was heat-inactivated at 56° C. for 30 mins and then incubated with VLPs at dilutions of 1/20, 1/80, 1/320, 1/1280, 1/5120 and 1/20480 for a total of six dilutions.

In initial experiments using B.1 spike, the inventors found that sera from both Pfizer/BioNTech and Moderna vaccinated individuals yielded high neutralization titers with medians of 549 and 490 respectively (Table 6). Sera from Johnson and Johnson vaccinated and convalescent patients had lower titers with median of 25 and 35 respectively (Table 6) matching the low levels of SARS-CoV-2 IgG antibodies detected in this cohort (Table 1). Note that the numbers in Table 6 indicate dilution factors that yields 50% neutralization. Higher numbers indicate better neutralization. Red shading indicates undetectable neutralization at the lowest (1/20) dilution.

TABLE 6
Neutralization titers against S-variants of serum
from vaccinated or convalescent individuals

| | B.1 | Delta | Omicron | OmC1 | OmC3 |
|---|---|---|---|---|---|
| PF0002 | 5900 | 880 | 768 | 4006 | 2435 |
| PF0004 | 4396 | 1248 | 204 | 1206 | 1244 |
| PF0005 | 549 | 185 | 20 | 172 | 130 |
| PF0006 | 194 | 52 | 17 | 34 | 68 |
| PF0007 | 752 | 319 | 30 | 190 | 357 |
| PF0009 | 1159 | 204 | 178 | 483 | 475 |
| PF0011 | 824 | 241 | 43 | 166 | 289 |
| PF0012 | 282 | 108 | 19 | 57 | 140 |
| PF0013 | 152 | 110 | 18 | 45 | 85 |
| PF0016 | 37 | 1 | 17 | 9 | 31 |
| PF0017 | 295 | 118 | 37 | 110 | 151 |
| M0002 | 3830 | 727 | 692 | 3185 | 1771 |
| M0003 | 375 | 75 | 26 | 102 | 173 |
| M0004 | 25608 | 6105 | 3524 | 15008 | 10995 |
| M0005 | 376 | 130 | 54 | 133 | 174 |
| M0006 | 450 | 80 | 24 | 229 | 178 |
| M0007 | 531 | 131 | 41 | 205 | 215 |
| M0008 | 186 | 76 | 17 | 94 | 111 |
| M0009 | 608 | 168 | 41 | 205 | 245 |
| M0010 | 171 | 35 | 2 | 47 | 60 |
| M0011 | 823 | 158 | 53 | 238 | 232 |
| JJ0002 | 60 | 2 | 16 | 16 | 11 |
| JJ0003 | 58 | 10 | 15 | 6 | 13 |
| JJ0005 | 26 | 7 | 19 | 35 | 15 |
| JJ0006 | 26 | 9 | 16 | 10 | 13 |
| JJ0007 | 11 | 12 | 14 | 7 | 18 |
| JJ0008 | 25 | 16 | 55 | 14 | 19 |
| JJ0009 | 10 | 8 | 14 | 0 | 14 |
| JJ0010 | 15 | 7 | 20 | 6 | 7 |
| JJ0011 | 20 | 5 | 12 | 3 | 12 |
| PC0002 | 51 | 44 | 43 | 19 | 12 |
| PC0003 | 7 | 22 | 20 | 9 | 25 |
| PC0006 | 5 | 0 | 15 | 5 | 5 |
| PC0007 | 31 | 0 | 24 | 12 | 24 |
| PC0008 | 39 | 323 | 27 | 14 | 26 |
| PC0009 | 268 | 113 | 24 | 104 | 14 |
| PC0011 | 432 | 19044 | 77 | 44 | 291 |
| PC0013 | 0 | 112 | 8 | 0 | 30 |
| Naïve | 5 | 11 | 19 | 9 | 2 |

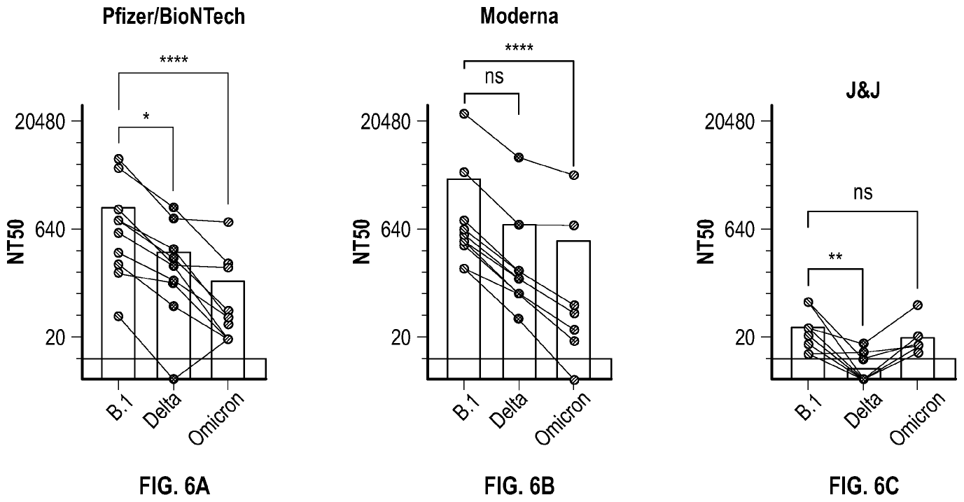

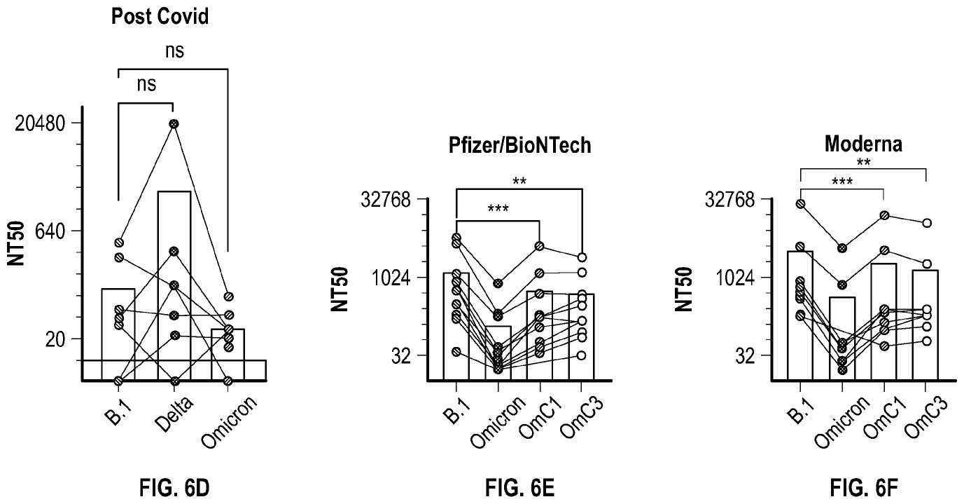

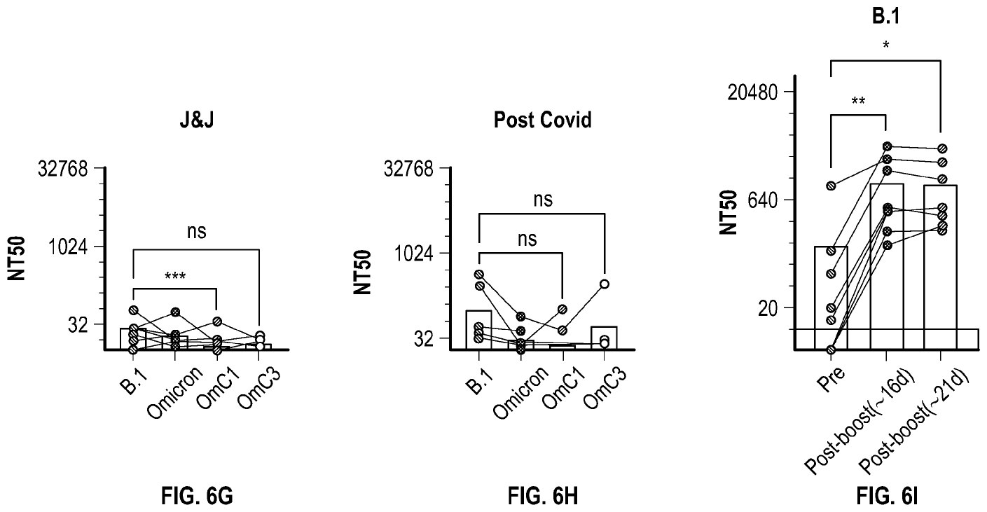

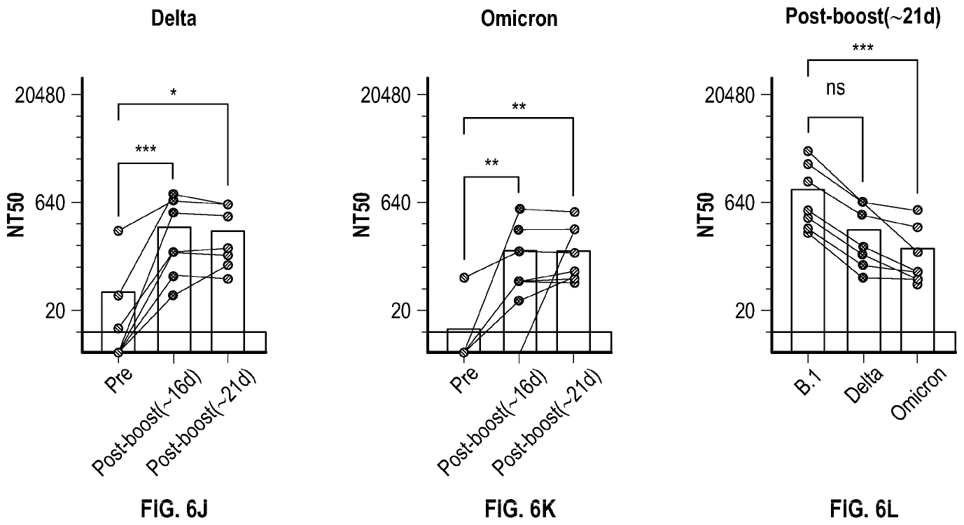

VLPs with Spike-protein variants were then tested as they have varying mutations in the receptor binding domain (RBD) that can affect neutralization. The neutralization capacity of each patient's serum was tested against VLPs displaying Spike proteins from B.1, Delta or Omicron viral variants. As shown in FIG. 6A-6D, there was a pronounced decrease of 15-fold to 18-fold in potency of subjects' anti-sera when tested against VLPs having the Omicron Spike proteins, with intermediate potency of the anti-sera against VLPs having Delta Spike proteins. The anti-sera from mRNA (Pfizer/Moderna) vaccine recipients were most effective against VLPs displaying the B.1 Spike protein (FIG. 6A-6D, Table 6). Limited efficacy was detected for sera from those vaccinated with the adenovirus based Johnson and Johnson vaccine and variable neutralization was observed for COVID-19 survivors (FIG. 6A-6D, Table 6).

The Spike protein Class 1 mutations (417N, 496S, 498R, 501Y) and Class 3 mutations (440K, 446S, 496S, 498R) associated with Omicron variants were next examined to ascertain whether they were responsible for reduced neutralization in patient anti-sera. Intermediate neutralization by antisera was observed for both Spike protein Omicron Class 1 (OmC1) and Omicron Class 3 (OmC3) cases, indicating that neutralization escape from patient sera is a function of several mutations acting in concert (FIG. 6E-6H).

Third-dose vaccinations with the Pfizer vaccine increased titers against all variants including Omicron (FIG. 6I-6L; Table 7) as measured at 16 and 21 days after the third dose. All 8 sera from this third-dose cohort had low (median 64) neutralization titers against Omicron at 21 days after their third dose while only 1 out 8 had detectable neutralization prior to boosting (FIG. 6K). However, even after such third dose boosting, an 8-fold reduction in neutralizing titers was observed against Omicron compared to B.1, indicating that Omicron is able to partially escape neutralizing antibodies induced by vaccination with the ancestral B.1 spike protein (FIG. 6L; Table 7).

TABLE 7
Neutralization titers against S-variants of individuals vaccinated with two or three doses of the Pfizer vaccine

| | | | | | | | | | Time Lapsed | | |
|---|---|---|---|---|---|---|---|---|---|---|---|
| | | | | | | | | | Between Samples | | |
| | | | | | | | | | T0 | | |
| NT50 against | | | NT50 against | | | NT50 against | | | (Third dose- | T1 | T2 |
| B.1 Spike | | | Delta Spike | | | Omicron Spike | | | Second Dose) | (Days post | (Days post |
| Pre-boost | T1 | T2 | Pre-boost | T1 | T2 | Pre-boost | T1 | T2 | days | booster shot) | booster shot) |
| 9 | 222 | 238 | 2 | 60 | 55 | 0 | 55 | 54 | 239 | 14 | 20 |
| 977 | 2251 | 2070 | 254 | 664 | 593 | 58 | 135 | 126 | 194 | 17 | 21 |
| 120 | 3311 | 3213 | 31 | 816 | 631 | 5 | 512 | 474 | 215 | 17 | 20 |
| 0 | 139 | 274 | 0 | 32 | 84 | 1 | 27 | 58 | 197 | 19 | 22 |
| 13 | 473 | 378 | 0 | 138 | 127 | 2 | 52 | 53 | 190 | 16 | 21 |
| 3 | 448 | 432 | 0 | 124 | 116 | 0 | 47 | 46 | 212 | 17 | 20 |
| 57 | 1537 | 1197 | 6 | 444 | 404 | 3 | 260 | 274 | 200 | 17 | 20 |
| 19 | 404 | 477 | 11 | 130 | 147 | 0 | 46 | 69 | 239 | 17 | 22 |

Note that for Table 7, each row represents one subject. Numbers indicate dilution factors that yield 50% neutralization, hence higher numbers indicate better neutralization. Red shading indicates undetectable neutralization at the lowest (1/20) dilution. Last three columns indicate the time elapsed between doses for each individual.

### Example 8: VLPs Show Commercially Available Antibody Treatments are not Effective Against Omicron

This Example describes evaluation of the effectiveness of monoclonal antibodies generated against the ancestral SARS-CoV-2 S protein against at Omicron neutralization.

VLPs were generated using the Omicron, OmC1 or OmC3 S genes, and transduction assays were conducted in the presence or absence of Class 1 (Casirivimab) or Class 3 (Imdevimab) monoclonal antibodies.

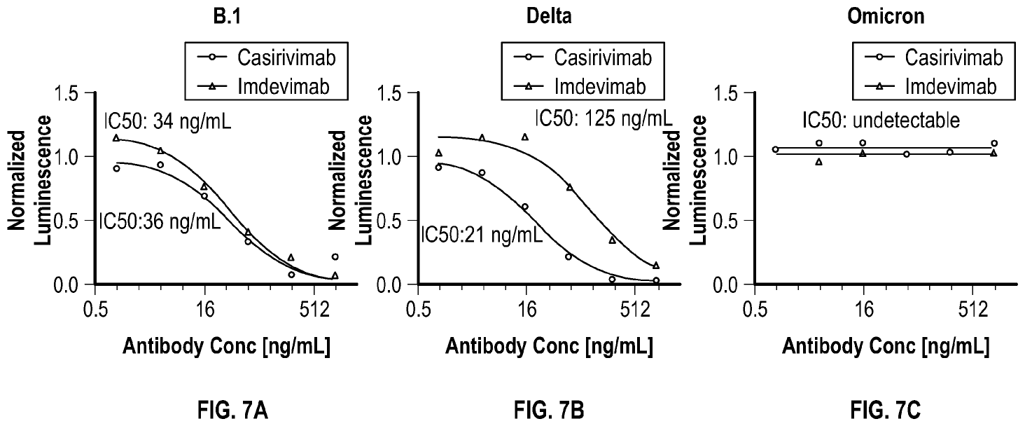

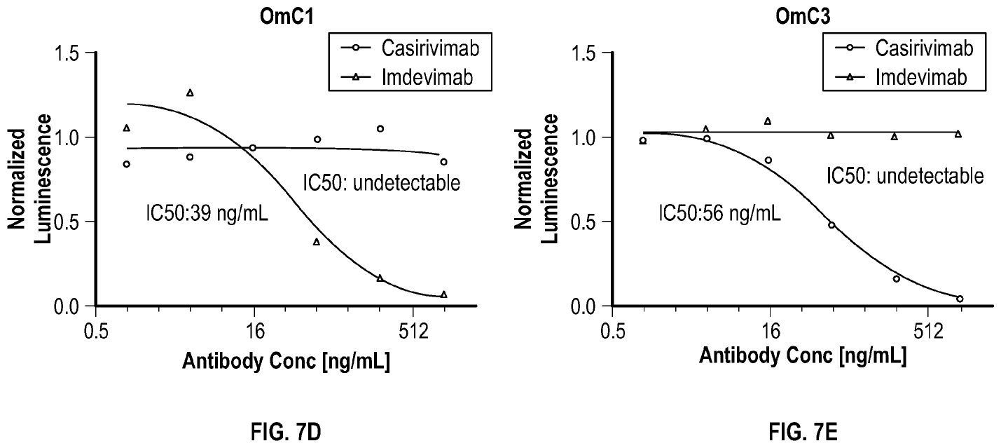

As shown in FIG. 7A-7E and Table 8, although both types of antibodies exhibited robust neutralization activity against B.1.1 or Delta VLPs, no activity was detected for either antibody preparation against Omicron VLPs. When the Omicron Class1 (OmC1) or Omicron Class 3 (OmC3) versions of the S gene were tested in the VLP assay, Casirivimab was able to neutralize OmC3 but not OmC1, while Imdevimab was able to neutralize OmC1 but not OmC3. These results indicate that the six mutations within the Omicron RBD (K417N, N440K, G446S, G496S, Q498R, N501Y) are largely responsible for the failure of these monoclonal antibodies to neutralize Omicron, which has these mutation in its Spike protein.

TABLE 8
IC50 of Casirivimab and Imdevimab against S variants (ng/mL)

| | Casirivimab | Imdevimab |
|---|---|---|
| B.1 | 36 | 34 |
| Delta | 21 | 125 |
| Omicron | >1000 | >1000 |
| OmC1 | >1000 | 39 |
| OmC3 | 56 | >1000 |

Smaller numbers in Table 8 indicate better neutralization. The shading indicates undetectable neutralization in the assay for dilutions of more than 1000 ng/mL.

In summary, SARS-CoV-2 virus-like particles that transduce reporter mRNA into ACE2- and TMPRSS2-expressing receptor cells enable a rapid and comprehensive comparison of structural protein (S, E, M, N) variant effects on both particle infectivity and antibody neutralization. As shown herein this system showed that the Omicron versions of both S and N enhance VLP infectivity relative to ancestral viral variants including the Delta variant. Omicron maintains mutations in the N mutational hotspot that were shown to confer markedly enhanced VLP infectivity. Surprisingly, the Omicron M and E gene variants appear to compromise infectivity, at least in the context of ancestral versions of the other structural genes, indicating that genes including S and N override less-fit versions of M, E and perhaps other genes in the intact virus.

Notably, all antisera from vaccinated individuals or convalescent sera from COVID-19 survivors showed reduced neutralization of Omicron VLPs relative to ancestral variants including Delta, with mRNA vaccines far surpassing a viral vector vaccine or natural infection in initial potency. These data do not account for T cell-based immunity induced by vaccination or prior infection. As also described herein, Omicron Spike mutations interfere with Class 1 and Class 3 monoclonal antibody binding, rendering some commercially available therapeutic antibodies completely ineffective. These results indicate that prior to vaccine boosting, antibodies produced by mRNA vaccines have 15- to 18-fold reduced efficacy against Omicron, and that the Johnson and Johnson vaccine produces limited neutralizing antibodies against any SARS-CoV-2 variant. Booster shots increase neutralization titers against Omicron but the titers remain much lower than for previous variants. These results support the use of mRNA vaccine boosters to enhance antibody-based protection against Omicron infection, in lieu of vaccines tailored to Omicron itself.

### Example 9: Neutralizing Antibody Levels in Vaccinated Individuals Wane Over Time and are Reduced Against Delta and Omicron Variants

SARS-CoV-2 VLP and live virus neutralization assays were performed in parallel on 143 plasma samples collected from 68 subjects enrolled in a prospectively enrolled longitudinal cohort (the UMPIRE, “UCSF employee and community immune response study”), fifteen (22.1%) of whom had received a booster and none of whom were previously infected.

Serum samples from the earliest and most recent time points were collected from each subject at 14 or more days after the last vaccine dose for neutralization testing. Sample collection dates for fully vaccinated, unboosted individuals (n=48) ranged from 14 to 305 days (median=91 days) following completion of the primary series of 2 doses for an mRNA vaccine (BNT162b2 from Pfizer or mRNA-1273 from Moderna) or 1 dose of the adenovirus vector vaccine (Ad26.CoV2.S from Johnson and Johnson). For boosted individuals (n=15), collection dates ranged from 2 to 74 days (median=23 days) following the booster dose.

Neutralizing antibody titers were expressed as the titers that neutralized 50% of VLP activity and referred to as “neutralization titers 50” (NT50).

Overall, median neutralizing antibody titers were 2.5-fold lower in assays using live viruses compared to assays using VLPs. However, the downward trends of neutralizing antibody levels for wild type compared to those for variant SARS-CoV-2 were similar.

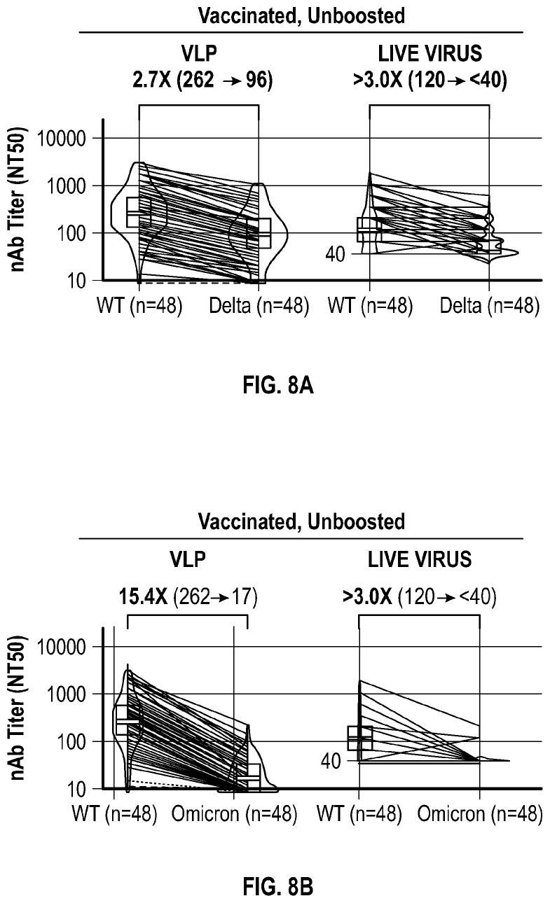

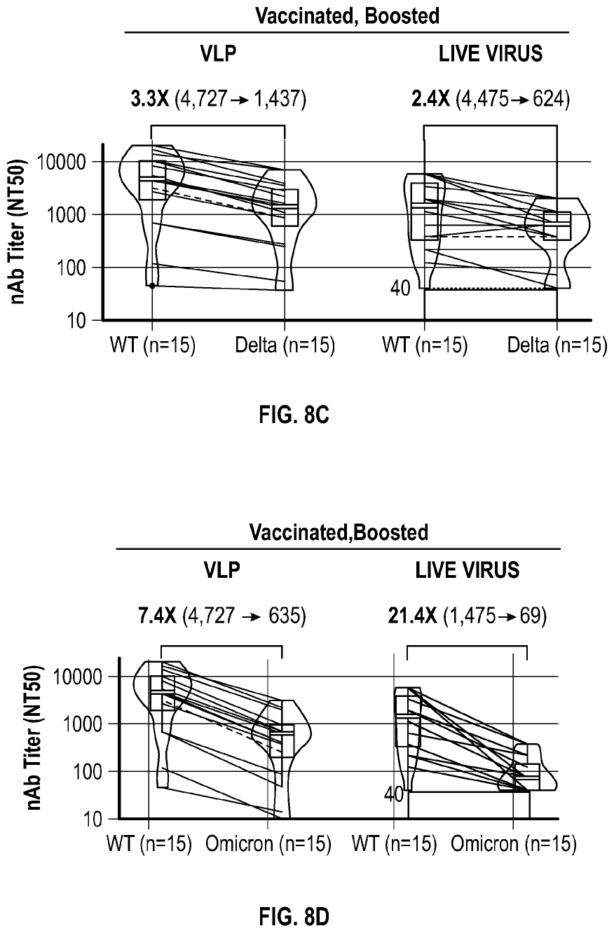

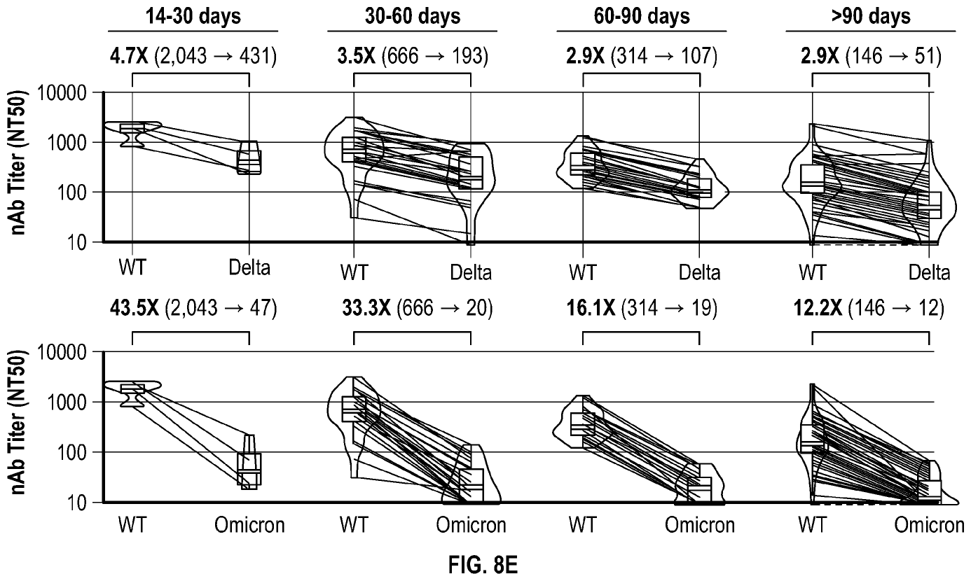

In unboosted vaccinated individuals, median VLP-neutralizing antibody titers to Delta and Omicron SARS-CoV-2 variants relative to wild type were reduced 2.7-fold (262/96) and 15.4-fold (262/17), respectively (FIG. 8A-8B, left). In comparison, live virus neutralization titers against Delta and Omicron were reduced at least 3.0-fold (120/<40) (FIG. 8A-8B, right).

VLP neutralization assays exhibited a lower limit of detection (NT50=10) than live virus neutralization assays (NT50=40). Using VLPs, the proportion of unboosted vaccinated individuals with Omicron neutralizing antibody levels above an NT50 cutoff of 40 was about 20%, as compared with about 80% and about 95% for Delta and wild type, respectively (FIG. 8B, left). When using live virus neutralization assays the proportion of individuals with Omicron neutralizing antibodies above an NT50 cutoff of 40 was about 5%, as compared with about 45% and about 75% for Delta and wild type, respectively (FIG. 8B, right).

As shown in FIG. 8C-8D (left), in boosted individuals VLP titers against wild type SARS-CoV-2 were 18-fold higher (4,727) than in unboosted individuals (262) (FIG. 8A-8B, left). Decreases in titers against Delta and Omicron relative to wild type SARS-CoV-2 were more modest at 3.3-fold and 7.4-fold, respectively, when individuals were boosted (FIG. 8C-8D, left). The VLP neutralization titers for boosted individuals indicated that more than 93% of the boosted individuals had neutralizing antibodies against all three SARS-CoV-2 lineages above an NT50 cutoff of 40.

In contrast, live virus neutralization titers in boosted individuals showed 21.4-fold lower titers against Omicron (69) relative to wild type (1,475) (FIG. 8D, right), indicating that only 62% of boosted individuals had neutralizing antibodies against Omicron.

At 90 or more days following vaccination, median VLP neutralization titers against wild type SARS-CoV-2 decreased by 93% (14-fold, from 2,043 to 146), with relative decreases in titers against Delta and Omicron ranging from 2.9- to 4.7-fold and 12.2- to 43.5-fold, respectively, compared with wild type SARS-CoV-2 (FIG. 8E).

Further studies showed that following Delta breakthrough infection, titers against wild type SARS-CoV-2 rose 57-fold and 3.1-fold compared with uninfected boosted and unboosted individuals, respectively, versus only a 5.8-fold increase and 3.1-fold decrease for Omicron breakthrough infection. Among immunocompetent, unboosted patients, Delta breakthrough infections induced 10.8-fold higher titers against wild type SARS-CoV-2 compared with Omicron (p=0.037). Decreased antibody responses in Omicron breakthrough infections relative to Delta were potentially related to a higher proportion of asymptomatic or mild breakthrough infections (55.0% versus 28.6%, respectively), which exhibited 12.3-fold lower titers against wild type SARS-CoV-2 compared with moderate to severe infections (p=0.020). Following either Delta or Omicron breakthrough infection, limited variant-specific cross-neutralizing immunity was observed. These results indicate that Omicron breakthrough infections are less immunogenic than Delta, thus providing reduced protection against reinfection or infection from future variants.

### REFERENCES

1. X. Xie, A. Muruato, K. G. Lokugamage, K. Narayanan, X. Zhang, J. Zou, J. Liu, C. Schindewolf, N. E. Bopp, P. V. Aguilar, K. S. Plante, S. C. Weaver, S. Makino, J. W. LeDuc, V. D. Menachery, P.-Y. Shi, An Infectious cDNA Clone of SARS-CoV-2. Cell Host Microbe. 27, 841-848.e3 (2020).
2. S. Torii, C. Ono, R. Suzuki, Y. Morioka, I. Anzai, Y. Fauzyah, Y. Maeda, W. Kamitani, T. Fukuhara, Y. Matsuura, Establishment of a reverse genetics system for SARS-CoV-2 using circular polymerase extension reaction. Cell Rep. 35, 109014 (2021).
3. C. Ye, K. Chiem, J.-G. Park, F. Oladunni, R. N. Platt 2nd, T. Anderson, F. Almazan, J. C. de la Torre, L. Martinez-Sobrido, Rescue of SARS-CoV-2 from a Single Bacterial Artificial Chromosome. MBio. 11 (2020), doi: 10.1128/mBio.02168-20.
4. X Xie, K. G. Lokugamage, X. Zhang, M. N. Vu, A. E. Muruato, V. D. Menachery, P.-Y. Shi, Engineering SARS-CoV-2 using a reverse genetic system. Nat. Protoc. 16, 1761-1784 (2021).
5. S. J. Rihn, A. Merits, S. Bakshi, M. L. Turnbull, A. Wickenhagen, A. J. T. Alexander, C. Baillie, B. Brennan, F. Brown, K. Brunker, S. R. Bryden, K. A. Burness, S. Carmichael, S. J. Cole, V. M. Cowton, P. Davies, C. Davis, G. De Lorenzo, C. L. Donald, M. Dorward, J. I. Dunlop, M. Elliott, M. Fares, A. da Silva Filipe, J. R. Freitas, W. Furnon, R. J. Gestuveo, A. Geyer, D. Giesel, D. M. Goldfarb, N. Goodman, R. Gunson, C. J. Hastie, V. Herder, J. Hughes, C. Johnson, N. Johnson, A. Kohl, K. Kerr, H. Leech, L. S. Lello, K. Li, G. Lieber, X. Liu, R. Lingala, C. Loney, D. Mair, M. J. McElwee, S. McFarlane, J. Nichols, K. Nomikou, A. Orr, R. J. Orton, M. Palmarini, Y. A. Parr, R. M. Pinto, S. Raggett, E. Reid, D. L. Robertson, J. Royle, N. Cameron-Ruiz, J. G. Shepherd, K. Smollett, D. G. Stewart, M. Stewart, E. Sugrue, A. M. Szemiel, A. Taggart, E. C. Thomson, L. Tong, L. S. Torrie, R. Toth, M. Varjak, S. Wang, S. G. Wilkinson, P. G. Wyatt, E. Zusinaite, D. R. Alessi, A. H. Patel, A. Zaid, S. J. Wilson, S. Mahalingam, A plasmid DNA-launched SARS-CoV-2 reverse genetics system and coronavirus toolkit for COVID-19 research. PLOS Biol. 19, e3001091 (2021).
6. J. A. Plante, Y. Liu, J. Liu, H. Xia, B. A. Johnson, K. G. Lokugamage, X. Zhang, A. E. Muruato, J. Zou, C. R. Fontes-Garfias, D. Mirchandani, D. Scharton, J. P. Bilello, Z. Ku, Z. An, B. Kalveram, A. N. Freiberg, V. D. Menachery, X. Xie, K. S. Plante, S. C. Weaver, P.-Y. Shi, Spike mutation D614G alters SARS-CoV-2 fitness. Nature. 592, 116-121 (2021).
7. K. H. D. Crawford, R. Eguia, A. S. Dingens, A. N. Loes, K. D. Malone, C. R. Wolf, H. Y. Chu, M. A. Tortorici, D. Veesler, M. Murphy, D. Pettie, N. P. King, A. B. Balazs, J. D. Bloom, Protocol and Reagents for Pseudotyping Lentiviral Particles with SARS-CoV-2 Spike Protein for Neutralization Assays. Viruses. 12 (2020), doi: 10.3390/v12050513.
8. Alaa Abdel Latif, Julia L. Mullen, Manar Alkuzweny, Ginger Tsueng, Marco Cano, Emily Haag, Jerry Zhou, Mark Zeller, Emory Hufbauer, Nate Matteson, Chunlei Wu, Kristian G. Andersen, Andrew I. Su, Karthik Gangavarapu, Laura D. Hughes, and the Center for Viral Systems Biology, Lineage Comparison.
9. W. Zeng, G. Liu, H. Ma, D. Zhao, Y. Yang, M. Liu, A. Mohammed, C. Zhao, Y. Yang, J. Xie, C. Ding, X. Ma, J. Weng, Y. Gao, H. He, T. Jin, Biochemical characterization of SARS-CoV-2 nucleocapsid protein. Biochem. Biophys. Res. Commm. 527, 618-623 (2020).
10. J. Cubuk, J. J. Alston, J. J. Incicco, S. Singh, M. D. Stuchell-Brereton, M. D. Ward, M. I. Zimmerman, N. Vithani, D. Griffith, J. A. Wagoner, G. R. Bowman, K. B. Hall, A. Soranno, A. S. Holehouse, The SARS-CoV-2 nucleocapsid protein is dynamic, disordered, and phase separates with RNA. Nat. Commun. 12, 1936 (2021).
11. T. M. Perdikari, A. C. Murthy, V. H. Ryan, S. Watters, M. T. Naik, N. L. Fawzi, SARS-CoV-2 nucleocapsid protein phase-separates with RNA and with human hnRNPs. EMBO J. 39, e106478 (2020).
12. C. B. Plescia, E. A. David, D. Patra, R. Sengupta, S. Amiar, Y. Su, R. V. Stahelin, SARS-CoV-2 viral budding and entry can be modeled using BSL-2 level virus-like particles. J. Biol. Chem. 296, 100103 (2021)
13. H. Swann, A. Sharma, B. Preece, A. Peterson, C. Eldredge, D. M. Belnap, M. Vershinin, S. Saffarian, Minimal system for assembly of SARS-CoV-2 virus like particles. Sci. Rep. 10, 21877 (2020).
14. J. Lu, G. Lu, S. Tan, J. Xia, H. Xiong, X. Yu, Q. Qi, X. Yu, L. Li, H. Yu, N. Xia, T. Zhang, Y. Xu, J. Lin, A COVID-19 mRNA vaccine encoding SARS-CoV-2 virus-like particles induces a strong antiviral-like immune response in mice. Cell Research. 30 (2020), pp. 936-939.
15. Y. L. Siu, K. T. Teoh, J. Lo, C. M. Chan, F. Kien, N. Escriou, S. W. Tsao, J. M. Nicholls, R. Altmeyer, J. S. M. Peiris, R. Bruzzone, B. Nal, The M, E, and N Structural Proteins of the Severe Acute Respiratory Syndrome Coronavirus Are Required for Efficient Assembly, Trafficking, and Release of Virus-Like Particles. Journal of Virology. 82 (2008), pp. 11318-11330.
16. P.-K. Hsieh, S. C. Chang, C.-C. Huang, T.-T. Lee, C.-W. Hsiao, Y.-H. Kou, I.-Y. Chen, C.-K. Chang, T-H. Huang, M.-F. Chang, Assembly of severe acute respiratory syndrome coronavirus RNA packaging signal into virus-like particles is nucleocapsid dependent. J. Virol. 79, 13848-13855 (2005).
17. S. Dent, B. W. Neuman, Purification of Coronavirus Virions for Cryo-EM and Proteomic Analysis. Coronaviruses (2015), pp. 99-108
18. X Lu, Y. Chen, B. Bai, H. Hu, L. Tao, J. Yang, J Chen, Z. Chen, Z. Hu, H. Wang, Immune responses against severe acute respiratory syndrome coronavirus induced by virus-like particles in mice. Immunology. 122, 496-502 (2007)
19. L. Kuo, P. S. Masters, Functional analysis of the murine coronavirus genomic RNA packaging signal. J. Virol. 87, 5182-5192 (2013).
20. K. Woo, M. Joo, K. Narayanan, K. H. Kim, S. Makino, Murine coronavirus packaging signal confers packaging to nonviral RNA. J. Virol. 71, 824-827 (1997).
21. J. A. Fosmire, K. Hwang, S. Makino, Identification and characterization of a coronavirus packaging signal. J. Virol. 66, 3522-3530 (1992).
22. X. Deng, M. A. Garcia-Knight, M. M. Khalid, V. Servellita, C. Wang, M. K. Morris, A. Sotomayor-González, D. R. Glasner, K. R. Reyes, A. S. Gliwa, N. P. Reddy, C. Sanchez San Martin, S. Federman, J. Cheng, J. Balcerek, J. Taylor, J. A. Streithorst, S. Miller, B. Sreekumar, P.-Y. Chen, U. Schulze-Gahmen, T. Y. Taha, J. M. Hayashi, C. R. Simoneau, G. R. Kumar, S. McMahon, P. V. Lidsky, Y. Xiao, P. Hemarajata, N. M. Green, A. Espinosa, C. Kath, M. Haw, J. Bell, J K. Hacker, C. Hanson, D. A. Wadford, C. Anaya, D. Ferguson, P. A. Frankino, H. Shivram, L. F. Lareau, S. K. Wyman, M. Ott, R. Andino, C. Y. Chiu, Transmission, infectivity, and neutralization of a spike L452R SARS-CoV-2 variant. Cell. 184, 3426-3437.e8 (2021).
23. A. Kuzmina, Y. Khalaila, O. Voloshin, A. Keren-Naus, L. Boehm-Cohen, Y. Raviv, Y. Shemer-Avni, E. Rosenberg, R. Taube, SARS-CoV-2 spike variants exhibit differential infectivity and neutralization resistance to convalescent or post-vaccination sera. Cell Host & Microbe. 29 (2021), pp. 522-528.e2.
24. Y. Liu, J. Liu, K. S. Plante, J. A. Plante, X. Xie, X. Zhang, Z. Ku, Z. An, D. Scharton, C. Schindewolf, V. D. Menachery, P.-Y. Shi, S. C. Weaver, The N501Y spike substitution enhances SARS-CoV-2 transmission. bioRxiv (2021), doi: 10.1101/2021.03.08.434499.
25. C. Motozono, M. Toyoda, J. Zahradnik, A. Saito, H. Nasser, T. S. Tan, I. Ngare, I. Kimura, K. Uriu, Y. Kosugi, Y. Yue, R. Shimizu, J. Ito, S. Torii, A. Yonekawa, N. Shimono, Y. Nagasaki, R. Minami, T. Toya, N. Sekiya, T. Fukuhara, Y. Matsuura, G. Schreiber, Genotype to Phenotype Japan (G2P-Japan) Consortium, T. Ikeda, S. Nakagawa, T. Ueno, K. Sato, SARS-CoV-2 spike L452R variant evades cellular immunity and increases infectivity. Cell Host Microbe. 29, 1124-1136.e11 (2021).
26. B. Li, A. Deng, K. Li, Y. Hu, Z. Li, Q Xiong, Z. Liu, Q. Guo, L Zou, H. Zhang, M. Zhang, F. Ouyang, J. Su, W. Su, J. Xu, H. Lin, J. Sun, J. Peng, H. Jiang, P. Zhou, T. Hu, M. Luo, Y. Zhang, H. Zheng, J. Xiao, T. Liu, R. Che, H. Zeng, Z. Zheng, Y. Huang, J. Yu, L. Yi, J Wu, J. Chen, H. Zhong, X. Deng, M. Kang, O. G. Pybus, M. Hall, K. A. Lythgoe, Y. Li, J. Yuan, J. He, J. Lu, Viral infection and Transmission in a large well-traced outbreak caused by the Delta SARS-CoV-2 variant, doi: 10.1101/2021.07.07.21260122.

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The following statements are intended to describe and summarize various embodiments of the invention according to the foregoing description in the specification.

### Statements:

1. A nucleic acid comprising a SARS-CoV-2 packaging signal sequence segment linked to a heterologous nucleic acid.
2. The nucleic acid of statement 1, further comprising a promoter or internal ribosome entry site (IRES) operably linked to the SARS-CoV-2 packaging signal sequence segment and to the heterologous nucleic acid.
3. The nucleic acid of statement 1 or 2, wherein the SARS-CoV-2 packaging signal sequence is a nucleic acid segment comprising positions 20080-21171 of the SARS-CoV-2 genome (termed herein the PS9 region).
4. The nucleic acid of any of statements 1-3, wherein the heterologous nucleic acid encodes a heterologous protein.
5. The nucleic acid of any of statements 1-4, wherein the heterologous nucleic acid encodes a detectable signal protein.
6. The nucleic acid of any of statements 1-4, wherein the heterologous nucleic acid encodes a therapeutic agent, an antigen, an antibody or antibody fragment.
7. The nucleic acid of statement 6, wherein the antibody or antibody fragment is an anti-Spike antibody or antibody fragment.
8. The nucleic acid of any of statements 1-4, wherein the heterologous nucleic acid encodes one or more viral replication proteins.
9. The nucleic acid of any of statements 1-3, wherein the heterologous nucleic acid encodes an inhibitory nucleic acid that binds to a segment of a SARS-CoV-2 RNA.
10. A cell comprising the nucleic acid of any of statements 1-9.
11. The cell of statement 10, that further expresses a SARS-CoV-2 SARS-CoV-2 spike (S) protein, SARS-CoV-2 membrane (M) protein, SARS-CoV-2 envelope (E) protein, and SARS-CoV-2 nucleocapsid (N) protein.
12. The cell of statement 10, wherein one or more of the SARS-CoV-2 SARS-CoV-2 spike (S) protein, the SARS-CoV-2 membrane (M) protein, the SARS-CoV-2 envelope (E) protein, or the SARS-CoV-2 nucleocapsid (N) protein has a mutation compared to a reference ancestral SARS-CoV-2 SARS-CoV-2 spike (S) protein, SARS-CoV-2 membrane (M) protein, SARS-CoV-2 envelope (E) protein, or SARS-CoV-2 nucleocapsid (N) protein sequence.
13. The cell of statement 10, 11 or 12, wherein one or more of the SARS-CoV-2 SARS-CoV-2 spike (S) coding region, the SARS-CoV-2 membrane (M) coding region, the SARS-CoV-2 envelope (E) coding region, or the SARS-CoV-2 nucleocapsid (N) coding region has a mutation compared to a SARS-CoV-2 SARS-CoV-2 spike (S) coding region, the SARS-CoV-2 membrane (M) coding region, the SARS-CoV-2 envelope (E) coding region, or the SARS-CoV-2 nucleocapsid (N) coding region in SEQ ID NO:1.
14. The cell of statement 10, 11 or 12, wherein the SARS-CoV-2 SARS-CoV-2 spike (S) protein has a mutation compared to a SARS-CoV-2 SARS-CoV-2 spike (S) protein with a D614G mutation.
15. The cell of any one of statements 10-14, which produces virus-like particles (VLPs).
16. The cell of statement 15, wherein the virus-like particles (VLPs) can undergo at least one round of replication.
17. An expression system comprising one or more expression cassettes, each expression cassette comprising a promoter or an internal ribosome entry site (IRES) operably linked to one or more of the following nucleic acids that encode:
   a. an RNA comprising a SARS-CoV-2 packaging signal sequence segment linked to a heterologous nucleic acid;
   b. a SARS-CoV-2 spike (S) protein;
   c. a SARS-CoV-2 membrane (M) protein;
   d. a SARS-CoV-2 envelope (E) protein; and
   e. a SARS-CoV-2 nucleocapsid (N) protein.
18. The expression system of statement 17, wherein the heterologous nucleic acid is a segment encoding a detectable signal protein.
19. The expression system of statement 17 or 18, wherein the heterologous nucleic acid also encodes one or more viral replication proteins.
20. The expression system of any of statements 17-19, wherein the SARS-CoV-2 packaging signal sequence is a nucleic acid segment comprising positions 20080-21171 of the SARS-CoV-2 genome (termed herein PS9).
21. The expression system of any one of statements 17-20, wherein at least one or at least two of the SARS-CoV-2 spike (S) protein, the SARS-CoV-2 membrane (M) protein, the SARS-CoV-2 envelope (E) protein, and the SARS-CoV-2 nucleocapsid (N) protein are expressed from separate expression cassettes or expression vectors.
22. A kit comprising one or more containers containing one or more components of the expression system of any one of statements 17-21.
23. A method comprising transfecting a host cell with at least one expression cassette or expression vector, wherein the at least one expression cassette or expression vector comprises a promoter or internal ribosome entry site (IRES) operably linked to at least one of the following heterologous nucleic acids:
   a. a nucleic acid comprising a SARS-CoV-2 packaging signal sequence segment linked to a heterologous nucleic acid;
   b. a nucleic acid encoding SARS-CoV-2 SARS-CoV-2 spike (S) protein;
   c. a nucleic acid encoding SARS-CoV-2 membrane (M) protein;
   d. a nucleic acid encoding SARS-CoV-2 envelope (E) protein;
   e. a nucleic acid encoding SARS-CoV-2 nucleocapsid (N) protein;
   f. or a combination thereof.
24. The method of statement 23, wherein the SARS-CoV-2 packaging signal sequence is a nucleic acid segment comprising positions 20080-21171 of the SARS-CoV-2 genome (termed herein the PS9 region).
25. The method of any of statements 23 or 24, wherein the heterologous nucleic acid encodes a heterologous protein.
26. The method of any of statements 23-25, wherein the heterologous nucleic acid encodes a detectable signal protein.
27. The method of any of statements 23-26, wherein the heterologous nucleic acid encodes a therapeutic agent, an antigen, an antibody or antibody fragment.
28. The method of statement 27, wherein the antibody or antibody fragment is an anti-Spike antibody or antibody fragment.
29. The method of any of statements 23-28, wherein the heterologous nucleic acid also encodes one or more viral replication proteins.
30. The method of any of statements 23-29, which produces virus-like particles (VLPs).
31. The method of statement 30, wherein the virus-like particles (VLPs) can undergo at least one round of replication.
32. The method of any of statements 23 or 24, wherein the heterologous nucleic acid encodes an inhibitory nucleic acid that binds to a segment of a SARS-CoV-2 RNA.
33. The method of any one of statements 23-32, wherein the host cell expresses at least one, at least two, at least three, or at least four, or five of the following:
   a. an RNA comprising a SARS-CoV-2 packaging signal sequence segment linked to the heterologous nucleic acid;
   b. a SARS-CoV-2 spike (S) protein;
   c. a SARS-CoV-2 membrane (M) protein;
   d. a SARS-CoV-2 envelope (E) protein;
   e. a SARS-CoV-2 nucleocapsid (N) protein; or
   f. a combination thereof.
34. The method of any one of statements 23-33, wherein one or more of the SARS-CoV-2 spike (S) protein, the SARS-CoV-2 membrane (M) protein, the SARS-CoV-2 envelope (E) protein, or the SARS-CoV-2 nucleocapsid
(N) protein can have a mutation.
35. The method of any one of statements 23-34, which generates SARS-CoV-2 virus-like-particles
36. The method of any one of statements 23-35, wherein the signal protein provides a detectable signal.
37. The method of statement 36, wherein the signal level is a measure of the extent of virus-like-particle assembly, packaging, and/or cellular entry.
38. A composition comprising SARS-CoV-2 virus-like-particles, the particles comprising an RNA comprising a SARS-CoV-2 packaging signal sequence segment linked to the heterologous nucleic acid, SARS-CoV-2 spike (S) proteins, SARS-CoV-2 membrane (M) proteins, SARS-CoV-2 envelope (E) proteins, and SARS-CoV-2 nucleocapsid (N) proteins.
39. The composition of statement 38, wherein the heterologous nucleic acid encodes a heterologous protein.
40. The composition of statement 38 or 39, wherein the heterologous nucleic acid encodes a detectable signal protein.
41. The composition of any of statements 38-40, wherein the heterologous nucleic acid encodes a therapeutic agent, an antigen, an antibody or antibody fragment.
42. The composition of statement 41, wherein the antibody or antibody fragment is an anti-Spike antibody or antibody fragment.
43. The composition of any of statements 38-42, wherein the heterologous nucleic acid encodes viral replication proteins.
44. The composition of statement 38, wherein the heterologous nucleic acid encodes an inhibitory nucleic acid that binds to a segment of a SARS-CoV-2 RNA.
45. The composition of any of statements 38-44, wherein one or more of the SARS-CoV-2 spike (S) protein, the SARS-CoV-2 membrane (M) protein, the SARS-CoV-2 envelope (E) protein, or the SARS-CoV-2 nucleocapsid (N) protein has a mutation.
46. The composition of statement 45, wherein the one or more mutation is compared to a SARS-CoV-2 spike (S) coding region, the SARS-CoV-2 membrane (M) coding region, the SARS-CoV-2 envelope (E) coding region, or the SARS-CoV-2 nucleocapsid (N) coding region in SEQ ID NO:1.
47. The composition of statement 45, wherein the spike protein does not have a SEQ ID NO:5, 34, or 35 sequence, the N protein does not have a SEQ ID NO:26 sequence, the M protein does not have a SEQ ID NO:7 or 21 sequence, and the E does not have a SEQ ID NO:20 sequence.

The specific methods and compositions described herein are representative of preferred embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention.

The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

As used herein and in the appended claims, the singular forms “a,” “an,” and “the” include plural reference unless the context clearly dictates otherwise. Thus, for example, a reference to “a nucleic acid” or “a protein” or “a cell” includes a plurality of such nucleic acids, proteins, or cells (for example, a solution or dried preparation of nucleic acids or expression cassettes, a solution of proteins, or a population of cells), and so forth. In this document, the term “or” is used to refer to a nonexclusive or, such that “A or B” includes “A but not B,” “B but not A,” and “A and B.” unless otherwise indicated.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

What is claimed:

1. A composition comprising SARS-CoV-2 virus-like-particles, the particles comprising at least one RNA comprising a SARS-CoV-2 packaging signal sequence segment linked to a heterologous nucleic acid, SARS-CoV-2 spike (S) proteins, SARS-CoV-2 membrane (M) proteins, SARS-CoV-2 envelope (E) proteins, and SARS-CoV-2 nucleocapsid (N) proteins.

2. The composition of claim 1, wherein the SARS-CoV-2 packaging signal sequence has at least 95% sequence identity to SEQ ID NO:2 or SEQ ID NO:3.

3. The composition of claim 1, wherein the heterologous nucleic acid encodes a heterologous protein.

4. The composition of claim 1, wherein the heterologous nucleic acid encodes a detectable signal protein.

5. The composition of claim 1, wherein the heterologous nucleic acid encodes a therapeutic agent, an antigen, an antibody or an antibody fragment.

6. The composition of claim 5, wherein the antibody or antibody fragment is an anti-Spike antibody or antibody fragment.

7. The composition of claim 1, wherein the heterologous nucleic acid encodes an inhibitory nucleic acid that binds to a segment of a SARS-CoV-2 RNA.

8. The composition of claim 1, wherein one or more of the SARS-CoV-2 spike (S) proteins, the SARS-CoV-2 membrane (M) proteins, the SARS-CoV-2 envelope (E) proteins, or the SARS-CoV-2 nucleocapsid (N) proteins has a mutation.

9. The composition of claim 8, wherein the one or more mutation is compared to a SARS-CoV-2 spike (S) coding region, the SARS-CoV-2 membrane (M) coding region, the SARS-CoV-2 envelope (E) coding region, or the SARS-CoV-2 nucleocapsid (N) coding region in SEQ ID NO:1.

10. An expression system comprising one or more expression cassettes, each expression cassette comprising a promoter or an internal ribosome entry site (IRES) operably linked to one or more of the following viral nucleic acids that encode:
a. an RNA comprising a SARS-CoV-2 packaging signal sequence segment linked to a heterologous nucleic acid;
b. a SARS-CoV-2 spike (S) protein;
c. a SARS-CoV-2 membrane (M) protein;
d. a SARS-CoV-2 envelope (E) protein; and
e. a SARS-CoV-2 nucleocapsid (N) protein.

11. The expression system of claim 10, wherein the SARS-CoV-2 packaging signal sequence has at least 95% sequence identity to SEQ ID NO:2 or SEQ ID NO:3.

12. The expression system of claim 10, wherein the heterologous nucleic acid encodes a detectable signal protein.

13. The expression system of claim 10, wherein the heterologous nucleic acid encodes a therapeutic agent, an antigen, an antibody or an antibody fragment.

14. The expression system of claim 10, wherein at least one or at least two of the SARS-CoV-2 spike (S) protein, the SARS-CoV-2 membrane (M) protein, the SARS-CoV-2 envelope (E) protein, and the SARS-CoV-2 nucleocapsid (N) protein are expressed from separate expression cassettes or expression vectors.

15. The expression system of claim 10, wherein one or more of the SARS-CoV-2 spike (S) protein, the SARS-CoV-2 membrane (M) protein, the SARS-CoV-2 envelope (E) protein, and the SARS-CoV-2 nucleocapsid (N) protein has a mutation.

16. A method comprising transfecting one or more host cells with at least one expression cassette or expression vector, wherein the at least one expression cassette or expression vector comprises a promoter or internal ribosome entry site (IRES) operably linked to at least one of the following nucleic acids:
a. a nucleic acid comprising a SARS-CoV-2 packaging signal sequence segment linked to a heterologous nucleic acid;
b. a viral nucleic acid encoding SARS-CoV-2 spike (S) protein;
c. a viral nucleic acid encoding SARS-CoV-2 membrane (M) protein;
d. a viral nucleic acid encoding SARS-CoV-2 envelope (E) protein;
e. a viral nucleic acid encoding SARS-CoV-2 nucleocapsid (N) protein;
f. or a combination thereof;
to thereby generate one or more transfected cells.

17. The method of claim 16, wherein the SARS-CoV-2 packaging signal sequence has at least 95% sequence identity to SEQ ID NO:2 or SEQ ID NO:3.

18. The method of claim 16, wherein the heterologous nucleic acid encodes a detectable signal protein.

19. The nucleic of claim 16, wherein the heterologous nucleic acid encodes a therapeutic agent, an antigenic protein, an antibody, or an antibody fragment.

20. The method of claim 19, wherein the antibody or antibody fragment is an anti-Spike antibody or antibody fragment.

21. The method of claim 16, wherein one or more of the transfected cells expresses at least one of the following:
a. an RNA comprising a SARS-CoV-2 packaging signal sequence segment linked to the heterologous nucleic acid;
b. a SARS-CoV-2 spike (S) protein;
c. a SARS-CoV-2 membrane (M) protein;
d. a SARS-CoV-2 envelope (E) protein;
e. a SARS-CoV-2 nucleocapsid (N) protein; or
f. a combination thereof.

22. The method of claim 16, wherein one or more of the SARS-CoV-2 spike (S) protein, the SARS-CoV-2 membrane (M) protein, the SARS-CoV-2 envelope (E) protein, or the SARS-CoV-2 nucleocapsid (N) protein has a mutation.

23. The method of claim 16, which generates SARS-CoV-2 virus-like-particles from the transfected cells.

24. The method of claim 23, further comprising collecting SARS-CoV-2 virus-like-particles from the transfected cells.

25. The method of claim 24, further comprising contacting the SARS-CoV-2 virus-like-particles, the transfected cells, or a combination thereof with one or more receptor cells that comprise a receptor for SARS-CoV-2.

26. The method of claim 25, wherein the one or more receptor cells comprises a population of receptor cells.

27. The method of claim 26, wherein one or more of the receptor cells in the population emit a detectable signal produced by a detectable signal protein encoded by the heterologous nucleic acid.

28. The method of claim 27, wherein the detectable signal or number of receptor cells emitting the detectable signal is a measure of the extent of virus-like-particle cellular entry in the population of receptor cells.

29. The method of claim 28, further comprising measuring a detectable signal levels from at least one of the populations of receptor cells that emit the detectable signal.

30. The method of claim 28, further comprising contacting at least one population of receptor cells with at least one test agent to form at least one assay mixture and measuring a detectable signal in the assay mixture.

31. The method of claim 30, wherein the at least one test agent is one or more small molecules, antibodies, nucleic acids, carbohydrates, proteins, peptides, or a combination thereof.

32. The method of claim 30, wherein the test agent comprises antibodies from one or more subjects.

33. The method of claim 32, further comprising administering a composition to one or more subjects whose antibodies emit a lower detectable signal level than a control or cut-off signal level.

34. The method of claim 33, wherein the control or cut-off signal level is a mean or medium signal level of antibodies from a population of subjects vaccinated against SARS-CoV-2.

35. The method of claim 33, wherein the composition is a vaccine against SARS-CoV-2.

36. The method of claim 33, wherein the vaccine comprises an mRNA that does not have a SEQ ID NO:34 sequence and does not encode a spike protein with a SEQ ID NO:5 or 35 sequence.

37. A method comprising (a) contacting SARS-CoV-2 virus-like-particles with a serum sample from a subject, and a population of receptor cells to form an assay mixture; and (b) measuring detectable signal levels produced by detectable signal protein;
the SARS-CoV-2 virus-like-particles comprising at least one RNA comprising a SARS-CoV-2 packaging signal sequence segment linked to a heterologous nucleic acid encoding the detectable signal protein, SARS-CoV-2 spike (S) proteins, SARS-CoV-2 membrane (M) proteins, SARS-CoV-2 envelope (E) proteins, and SARS-CoV-2 nucleocapsid (N) proteins.

38. The method of claim 37, further comprising administering a SARS-CoV-2 vaccine to one or more subjects whose assay mixtures emit lower detectable signal levels than a control or cut-off signal level.

39. The method of claim 38, wherein the control or cut-off signal level is a mean or medium signal level of assay mixtures from a population of subjects vaccinated against SARS-CoV-2.

40. The method of claim 38, wherein the vaccine comprises an mRNA that does not have a SEQ ID NO:34 sequence and does not encode a spike protein with a SEQ ID NO:5 or 35 sequence.
